(19) United States
(12) Patent Application Publication
(10) Pub. No.: US 2024/0315989 A1
(43) Pub. Date: Sep. 26, 2024

(54) METHODS OF TREATING COVID-19

(71) Applicant: Washington University, St. Louis, MO (US)

(72) Inventors: Angela M. Reiersen, St. Louis, MO (US); Eric Lenze, St. Louis, MO (US)

(21) Appl. No.: 17/698,361

(22) Filed: Mar. 18, 2022

Related U.S. Application Data
(60) Provisional application No. 63/163,224, filed on Mar. 19, 2021.

(51) Int. Cl. A61K 31/137; A61P 31/00

(52) U.S. Cl. CPC A61K 31/137; A61P 31/00

(57) ABSTRACT

Among the various aspects of the present disclosure is the provision of methods of treating or preventing COVID-19 or effects thereof in a subject comprising: administering a therapeutically effective amount of a pharmaceutical agent comprising one or more of a sigma-1 receptor (S1R) agonist or antagonist, a cationic amphiphilic drug, a selective serotonin reuptake inhibitor (SSRI), or functional inhibitor of acid sphingomyelinase (FIASMA), such as fluvoxamine.

### CROSS-REFERENCE TO RELATED APPLICATIONS

This application claims priority from U.S. Provisional Application Ser. No. 63/163,224 filed on 19 Mar. 2021, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE INVENTION

The present disclosure generally relates to COVID-19 therapy and prevention of long COVID or serious side effects or complications due to SARS-CoV-2 infection.

### SUMMARY OF THE INVENTION

Among the various aspects of the present disclosure is the provision of methods of treating or preventing COVID-19 or complications thereof, such as clinical deterioration, hospitalization, death, in a subject comprising: administering a therapeutically effective amount of a pharmaceutical agent comprising one or more of a sigma-1 receptor (S1R) agonist or antagonist, a cationic amphiphilic drug, a selective serotonin reuptake inhibitor (SSRI), or functional inhibitor of acid sphingomyelinase (FIASMA), such as fluvoxamine. An aspect of the present disclosure provides for a method of preventing progression to severe COVID-19 or hospitalization, or treating or preventing SARS-CoV-2 infection, COVID-19, or COVID-19 symptoms in a subject. In some embodiments, the method comprises administering a therapeutically effective amount of a pharmaceutical agent comprising an antidepressant medication, an anxiolytic medication, a psychotropic, a sigma-1 receptor (S1R) antagonist or agonist, a cationic amphiphilic drug, a selective serotonin reuptake inhibitor (SSRI), or functional inhibitor of acid sphingomyelinase (FIASMA), or a combination thereof. In some embodiments, the pharmaceutical agent has S1R binding activity. In some embodiments, the pharmaceutical agent has a Ki value of at least 3000 nM to S1R. In some embodiments, the pharmaceutical agent is one or more antidepressants selected from the group consisting of: Fluoxetine; Fluvoxamine; Citalopram; Chlorpromazine; Escitalopram; Paroxetine; Sertraline; Vilazodone; Vortioxetine; Amitriptyline; Clomipramine; Desipramine; Doxepin; Imipramine; Nortriptyline; Nefazodone; Trazodone; Desvenlafaxine; Duloxetine; Levomilnacipran; Venlafaxine; Bupropion; or Mirtazapine. In some embodiments, the pharmaceutical agent is a selective serotonin reuptake inhibitor (SSRI). In some embodiments, the pharmaceutical agent is a SSRI selected from Citalopram, Escitalopram, Fluoxetine, Fluvoxamine, Paroxetine, or Sertraline. In some embodiments, the SSRI is a selective serotonin (5-HT) reuptake inhibitor (SSRI) of the 2-aminoethyl oxime ethers of aralkylketones chemical series. In some embodiments, the pharmaceutical agent is a sigma-1 receptor (S1R) agonist which activates the sigma-1 receptor (S1R). In some embodiments, the pharmaceutical agent is: an antidepressant medication; and/or a sigma-1 receptor (S1R) agonist, a cationic amphiphilic drug, a selective serotonin reuptake inhibitor (SSRI), and/or a functional inhibitor of acid sphingomyelinase (FIASMA). In some embodiments, the pharmaceutical agent is fluvoxamine. In some embodiments, the pharmaceutical agent is a norepinephrine-dopamine reuptake inhibitor (NDRI). In some embodiments, the NDRI is bupropion. In some embodiments, the antidepressant is a tricyclic antidepressant. In some embodiments, the tricyclic antidepressant is Amitriptyline, Clomipramine, Desipramine, Doxepin, Imipramine, or Nortriptyline. In some embodiments, the antidepressant is a phenylpiperazine. In some embodiments, the phenylpiperazine is nefazodone or Trazodone. In some embodiments, the antidepressant is a Serotonin Norepinephrine Reuptake Inhibitors (SNRI). In some embodiments, the SNRI is Desvenlafaxine, Duloxetine, Levomilnacipran, or Venlafaxine. In some embodiments, the antidepressant is a tetracyclic piperazine-azepine. In some embodiments, the tetracyclic piperazine-azepine is Mirtazapine. In some embodiments, the anxiolytic medication is a Benzodiazepine. In some embodiments, the Benzodiazepine is Alprazolam, Chlordiazepoxide, Clorazepate, Diazepam, Flurazepam, Lorazepam, Oxazepam, Temazepam, or Triazolam. In some embodiments, the pharmaceutical agent is a psychotropic. In some embodiments, the psychotropic is rimonabant, an inverse agonist of CB1 cannabinoid receptor. In some embodiments, the pharmaceutical agent is an S1R ligand selected from high-affinity agonists: fluoxetine or fluvoxamine; intermediate-affinity agonists: escitalopram or citalopram; or low-affinity agonist: paroxetine; or antagonist: sertraline. In some embodiments, the pharmaceutical agent is antidepressants with FIASMA activity. In some embodiments, the pharmaceutical agent is functional inhibitor of acid sphingomyelinase (FIASMA) having FIASMA activity, defined as showing an in vitro functional inhibition effect on acid sphingomyelinase (ASM). In some embodiments, the FIASMA is selected from one or more selected from the group consisting of: amitriptyline, citalopram, clomipramine, desipramine, doxepin, escitalopram, fluoxetine, fluvoxamine, imipramine, nortriptyline, paroxetine, sertraline, and/or venlafaxine. In some embodiments, the pharmaceutical agent is selected from bupropion, desvenlafaxine, duloxetine, levomilnacipran, mirtazapine, nefazodone, trazodone, vilazodone, or vortioxetine. In some embodiments, the pharmaceutical agent is selected from fluoxetine, paroxetine, escitalopram, venlafaxine, and/or mirtazapine. In some embodiments, the subject: has a positive test result for SARS-CoV-2 viral testing; has, is suspected of having, or is at risk for contracting COVID-19 or developing a SARS-CoV-2 infection; has early symptomatic COVID-19 disease; is in an acute phase of COVID-19 illness; has Post-Acute Sequelae of SARS-CoV-2 infection (PASC); is exposed to COVID-19 or at risk of COVID-19 exposure; is ambulatory having COVID-19; has had COVID-19 symptoms for less than or equal to 7 days; has had COVID-19 symptoms for less than or equal to 10 days; has had COVID-19 symptoms for between 7 to 10 days; or has had COVID-19 symptoms for greater than 5 days. In some embodiments, the subject is at high risk for progressing to severe disease and/or hospitalization. In some embodiments, the subject is high risk, wherein a high risk subject has one or more selected from: diabetes; systemic arterial hypertension requiring at least one oral medication for treatment; known cardiovascular disease (optionally, heart failure, congenital heart disease, valve disease, coronary artery disease, cardiomyopathies being treated, clinically manifested heart disease and/or with clinical repercussion); symptomatic lung disease or treatment for such (optionally, emphysema, fibrosing diseases); symptomatic asthma requiring chronic use of agents to control symptoms; smoking; obesity, defined as body-mass index greater than 30 kg/m²; having had a transplant; stage IV chronic kidney disease or on dialysis; immunosuppression or use of corticosteroid therapy (optionally, equivalent to at least 10 mg of prednisone per day) or immunosuppressive therapy; history of cancer in the last 0.5 years or undergoing current cancer treatment or aged 50 years or older; or unvaccinated status. In some embodiments, the subject has risk factors for disease progression selected from one or more of: age ≥50 years, diabetes, hypertension, obesity, smoking, conditions associated with immunosuppression, unvaccinated status, or comorbidities, optionally selected from cancer, cardiovascular, pulmonary, or kidney disorders. In some embodiments, the subject is not being prescribed an SSRI or being administered an SSRI at the time of COVID-19 infection or symptom onset. In some embodiments, the subject is administered the SSRI prophylactically. In some embodiments, the subject has not been diagnosed with a mental illness, mood disorder, anxiety disorder (optionally, OCD, depression), other psychiatric disorders or prescribed medicine for a mental illness, mood disorder, anxiety disorder (optionally, OCD, depression), or other psychiatric disorders prior to being treated for COVID-19 or prevention of COVID-19. In some embodiments, the subject has hypoxia or dyspnea. In some embodiments, the subject has severe COVID-19 necessitating interventional care (optionally, dexamethasone, supplemental oxygen). In some embodiments, the subject has fever, cough, shortness of breath, fatigue or weakness, chills, nausea, body aches, diarrhea, loss of appetite, difficulty with sense of smell, or difficulty with sense of taste. In some embodiments, the subject has tinnitus or vaccine-associated tinnitus or post-vaccine long-COVID-like symptoms. In some embodiments, the therapeutically effective amount of the pharmaceutical agent results in reducing or preventing: clinical deterioration; intubation or death; excessive immune response associated with a COVID-19 infection; an inflammatory response in the subject; short or long term complications of COVID-19; severe lung damage; damage from an inflammatory response; or shortness of breath. In some embodiments, the therapeutically effective amount of the pharmaceutical agent results in reducing the risk of: developing severe long-term post-COVID symptoms; developing COVID acute respiratory distress syndrome (ARDS); developing post-acute sequelae of SARS-CoV-2 infection (PASC); death; declining or deteriorating health; hospitalization; progression to severe disease with hypoxia ≤92%; being admitted in an emergency setting or retention for greater than 6 h; developing severe disease or illness; or developing respiratory deterioration. In some embodiments, the therapeutically effective amount of the pharmaceutical agent results in reducing: recovery time; levels of cytokines in the subject; levels of inflammatory molecules in the subject; or severity of Post-Acute Sequelae of SARS-CoV-2 infection (PASC). In some embodiments, administrating the pharmaceutical agent to a subject prior to SARS-CoV-2 infection results in: reduced relative risk of mortality, wherein the pharmaceutical agent is an SSRI, optionally, fluoxetine orfluvoxamine; or reduced risk of an emergency department (ED) visit or hospital admission in ambulatory patients infected with SARS-CoV-2 re-illness use of wherein the pharmaceutical agent is an antidepressant or an SSRI. In some embodiments, administrating the pharmaceutical agent results in the subject having improved recovery between about three months after infection to about 1 year after infection compared to a subject not receiving the pharmaceutical agent. In some embodiments, hospitalization is defined as either retention in a COVID-19 emergency setting or transfer to tertiary hospital from COVID-19 up to 28 days. In some embodiments, clinical deterioration is defined as emergency department (ED) visitation or hospital admission. In some embodiments, progression to severe COVID-19 is defined by hypoxia <93% with dyspnea and/or requiring hospitalization. In some embodiments, the therapeutically effective amount is an amount effective to: prevent or reduce the risk of clinical deterioration, defined as shortness of breath, hospitalization for shortness of breath or pneumonia, oxygen saturation less than 92% on room air, or need for supplemental oxygen to achieve oxygen saturation of 92% or greater. In some embodiments, the therapeutically effective amount is an amount effective to reduce or prevent symptoms or negative outcomes associated with COVID-19 compared to a placebo. In some embodiments, the subject is administered the pharmaceutical agent within the first 7 to 10 days of illness with COVID-19 or between day one and day 7 to day 10 of COVID-19 symptom onset. In some embodiments, the subject is administered the pharmaceutical agent until COVID-19 symptoms resolve. In some embodiments, the subject is administered the pharmaceutical agent comprising an SSRI prior to contracting SARS-CoV-2 at a dose of at least 20 mg fluoxetine-equivalents or greater than or equal to 40 mg fluoxetine-equivalents. In some embodiments, the subject is administered the pharmaceutical agent or a duration of 10-15 days through at least 15 days of illness or symptoms. In some embodiments, the subject is administered the pharmaceutical agent comprising fluvoxamine at 50 mg twice a day; 200 mg/day (100 mg twice daily); 300 mg/day (100 mg three times daily); or a daily total of 300 mg. In some embodiments, the subject is administered the pharmaceutical agent comprising fluvoxamine at 50 mg on day one, then 100 mg twice a day; 50 mg on day 1, then for 2 days at a dose of 100 mg twice daily, and for the duration of treatment, a dose of 100 mg 3 times daily; 100 mg three times daily; 50 mg to 100 mg two times daily; 100 mg twice daily; a single dose of 150-200 mg daily; or 100 mg two times daily for 3 days, then 50 mg two times daily for 3 days. In some embodiments, the pharmaceutical agent is administered for an amount of time between 10 and 15 days, for a duration of 15 days, or for the duration of illness or until symptom resolution, as tolerated; and if intolerance is a problem, the dose can be reduced to 50 mg twice daily. In some embodiments, the pharmaceutical agent is administered to the subject in an amount of about 100 mg at a frequency of 3× per day. In some embodiments, the pharmaceutical agent is administered to the subject for about 15 days or before or at about 14 days post symptom onset. In some embodiments, the pharmaceutical agent comprises an antidepressant at a dose of: at least 20 mg fluoxetine-equivalents, greater than or equal to 20 mg fluoxetine-equivalents, greater than or equal to 40 mg equivalent, or between 20.0 mg and 44.4 mg fluoxetine-equivalents. In some embodiments, the antidepressant is selected from the following and the fluoxetine-equivalent is calculated using a conversion factor associated with the antidepressant. In some embodiments, the pharmaceutical agent is fluvoxamine and is administered in tablet form in 25 mg, 50 mg, or 100 mg strengths. In some embodiments, the pharmaceutical agent comprises Bupropion at any dose. In some embodiments, the pharmaceutical agent is administered orally or intravenously. In some embodiments, the method further comprises administering a second pharmaceutical agent comprising a: S1R agonist (optionally, Chlorpromazine, Fluoxetine), S1R antagonist, antihistamine, bromhexine, serotonin antagonist (optionally, cyproheptadine, bromohexine), an antibiotic, an anti-inflammatory, a steroid, a glucocorticoid, a serotonin antagonist, a virus entry inhibitor, an anti-viral (optionally, remdesivir, Nirmatrelvir (Paxlovid), molnupiravir (Lagevrio), lopinavir, ritonavir, favipiravir), anti-SARS-CoV-2 mAb product (optionally, Bamlanivimab (LY-CoV555), etesevimab, casirivimab, imdevimab (REGEN-COV), sotrovimab, or combinations thereof), itraconazole, hydroxychloroquine, metformin, niclosamide, ivermectin, fluvoxamine, doxasozin, pegylated interferon lambda, anti-inflammatory drugs, such as colchicine, corticosteroids, or budesonide. If the combination is with another pharmaceutical composition that is in the same class as the first, it will be a “second” pharmaceutical composition, such as a second SSRI or a second S1R agonist.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A-FIG. 1B. Graphical abstract (A). Enrollment and Patient Flow. (B) COVID-19, coronavirus disease 2019; SARS-CoV-2, severe acute respiratory syndrome coronavirus 2. ^{a}Did not speak English, lived outside delivery area of the study, or unable to provide data via phone or internet. ^{b}lnterstitial lung disease, immunocompromised, actively suicidal or psychotic, cognitive impairment (dementia or Alzheimer's disease), metastatic cancer, or end-stage congestive heart failure. ^{c}Prednisone dose greater than 20 mg/d (most common exclusionary medication), azithromycin (not allowed at start of the study, but later allowed), hydroxychloroquine (not allowed at start of study, but later allowed), or some immunosuppressant biologic medications (such as belimumab). ^{d}COVID-19 suspected and patient either had a negative test result or unable to obtain test. ^{e}Staff unable to contact potential participants. ^{f}Received medication and study supplies, but then research staff were unable to contact participants further. ^{g}Included in analysis but censored early.

FIG. 2. Time to Clinical Deterioration in the Fluvoxamine and Placebo Groups The median observation time was 15 days (interquartile range, 15-15 days) for the fluvoxamine group and 15 days (interquartile range, 15-15 days) for the placebo group. Study day 0 indicates the day of randomization.

FIG. 3. Improvement in most severe baseline COVID-19 symptom. This box and whiskers plot shows the highest daily score for each participant of their most severe baseline symptom. We did not pursue this examination further because the curves showed no substantial differences and because the baseline most severe symptom was heterogeneous across participants (see TABLE 1) and therefore did not adequately capture overall symptom burden.

FIG. 4. Anxiety ratings in the fluvoxamine vs. placebo groups during the 15-day RCT. To determine whether fluvoxamine (an SSRI that is FDA-approved for treating anxiety disorders) produced any effect on anxiety, we tracked anxiety on a 0-10 scale twice daily during the 15 day RCT. Below is shown a box and whiskers plot of mean anxiety symptoms of time (highest score for participant on each day). Note: anxiety was not assessed at baseline (day 0). Note: The boxplot at the bottom of the figure was not shown because most assessed values were “0”; hence, then non-zero symptom scores appeared to be far-outliers in the graph.

FIG. 5. Distributions of baseline oxygen saturation in the fluvoxamine and placebo groups and clinical deterioration as a function of baseline oxygen saturation. Shown are bell curve distributions of the baseline (day 0) oxygen saturation score in the two groups. In the placebo group, the six cases of clinical deterioration are marked in orange: 6/23 (26%) of those with baseline oxygen saturation ≤96% deteriorated, vs. 0/49 (0%) of those with baseline oxygen saturation >96%. In the fluvoxamine group, the corresponding numbers were 0/22 and 0/58. TABLE 2 shows the median baseline oxygen saturation in the two groups which was not significantly different (97% fluvoxamine, 97% placebo).

FIG. 6. STOP COVID 1 & 2: Self-reported recovery at long-term follow up. Self-rated recovered back to usual health.

FIG. 7. Graphical abstract.

FIG. 8. Up to a 17-day course (median, 16) of symptoms for 10 randomly selected participants on a per-symptom basis. Each row includes data for 1 participant. Time points are ˜12 hours apart on up to 17 days. Note the “saw tooth” pattern for many participants, indicating symptoms that wax and wane.

FIG. 9. Representative courses of coronavirus disease 2019 symptoms across up to 17 days (median, 16 days). Each time point represents the average of 3 days, except that Time 4 included up to 5 days, but more typically 3 or 4. The Average line represents the average trajectory across the entire data set. The Rapid improvement and Slower improvement lines are the average for the 10 participants, with the strongest linear slope in the improving and worsening directions. For diarrhea, the quadratic slope was used as the linear slope had no variance. A worsening linear slope did not always translate to symptoms being aggravated overall because trajectories were curvilinear. Slower improvement lines are not depicted for appetite or chills because too few participants experienced worsening in this symptom as indexed by the linear slope. The Deteriorated lines provide the slope for the single participant who deteriorated midway through the trial yet provided a full set of data.

FIG. 10. On the left, the average course of 5 common symptoms across up to 17 days (median number of days, 16). On the right, 9 randomly selected participants (of those who had all 5 symptoms) and their individual trajectories. Note that because the model fits a curve to data points, the curve can transiently go above the response scale.

FIG. 11. Panel A depicts the number of person-level means at each level for self-report symptoms, with decimal values rounded to a whole number to better depict the pattern. Each participant has a mean for each symptom, and the circle shows the number of participants with a mean at that level. The most common mean for most symptoms was zero. Panel B shows the mode (most common response) for people who ever had that symptom. The most common mode was zero for each symptom even when restricted to participants who ever had that symptom.

FIG. 12. Raw symptom trajectories of patients who provided at least 2 days of EMA data (n=150).

FIG. 13. Individual-level slopes for participants with at least 2 data points and at least 1 slope estimated (n=147). Top left (red outline) is group-level average slope across all participants.

FIG. 14. TOGETHER Trial profile.

FIG. 15A-FIG. 15B. Probability of efficacy and Bayesian relative risk of hospitalization defined as either retention in a COVID-19 emergency setting or transfer to tertiary hospital due to COVID-19 for fluvoxamine versus placebo. BCI=Bayesian credible interval.

FIG. 16. Subgroup analyses of fluvoxamine versus placebo in the TOGETHER Trial.

FIG. 17. Time to hospitalization or extended emergency room visit due to COVID-19.

FIG. 18. Time to symptom resolution (WHO symptom index scale).

FIG. 19. Probability of viral detection (Days 0, 3, 7).

FIG. 20. Together Trial Outcomes by Intent to Treat (ITT) and Per Protocol (PP) Analysis.

FIG. 21. Potential anti-COVID-19 mechanisms of action of fluvoxamine. Figure created using Biorender.

FIG. 22. Recommendations for research activities.

FIG. 23. Biological mechanisms proposed by Carpinteiro et al. underlying the potential inhibition by Functional Inhibitors of Acid.

FIG. 24. Study cohort. *A participant may receive two or more FIASMA medications at baseline. COVID-19, coronavirus disease 2019; FIASMA, Functional Inhibitors of Acid Sphingomyelinase; ICU, intensive care unit; RT-PCR, reverse-transcriptase-polymerase-chain-reaction.

FIG. 25. Propensity score (PS) distribution in patients taking versus not taking a FIASMA medication at baseline. The mid-tone of grey refers to the area where both groups share PS values.

FIG. 26. Kaplan-Meier curves for the composite endpoint of intubation or death in the full sample crude analysis (N=2846) (a), in the full sample analysis with IPW (N=2846) (b), and in the matched analytic sample using a 1:1 ratio (N=554) (c) among patients hospitalized for severe COVID-19, according to FIASMA medication use at baseline. The shaded areas represent pointwise 95% confidence intervals. COVID-19, coronavirus disease 2019; IPW, inverse probability weighting; FIASMA, Functional Inhibitors of Acid Sphingomyelinase Activity.

FIG. 27. Comparison of Simulated plasma free FLV and FXT concentrations to S1R Ki and HEK293T-ACE2-TMPRSS2 cell IC₅₀. Ratio of fluvoxamine (FLV; black) and fluoxetine (FXT: yellow) free concentration in plasma to (A) S1R Ki (FLV=15-64 ng/mL and FXT=82-99 ng/mL) [semi-log scale] and (B) HEK293T-ACE2-TMPRSS2 cell IC₅₀ (FLV=4579 ng/mL and FXT=2072 ng/mL) [linear scale]. Shaded intervals represent simulated concentrations following administration of FLV (grey) or FXT (yellow) at label-recommended dosages (FLV 100 mg q24h, 100 mg q12h and 100 mg q8h; FXT 20 mg q24h, 40 mg q24h and 80 mg q24h). FLV clinical dose from the TOGETHER trial (100 mg q12h) and a commonly prescribed dose for FXT (40 mg q24h) are depicted as solid lines. Legend: FLV=fluvoxamine, FXT=fluoxetine, q8h=every eight hours, q12h=every twelve hours, q24h every twenty-four hours, S1R=Sigma-1 receptor; Ki=inhibitory constant, IC₅₀=concentration of half-maximal inhibition.

FIG. 28. Frequentist Random Effects Meta-Analysis.

FIG. 29. PRISMA diagram.

FIG. 30. Probability densities.

FIG. 31. SSRIs deplete platelets of serotonin and inhibit platelet activation. Platelets from COVID-19 patients are hyper-reactive to thrombin (credit: Dr. Farid Jalali). As a patient develops COVID Acute Respiratory Distress Syndrome (ARDS), serotonin levels increase.

### DETAILED DESCRIPTION OF THE INVENTION

The present disclosure is based, at least in part, on the discovery that Outpatients treated with fluvoxamine early in the course of symptomatic COVID-19 had a lower likelihood of clinical deterioration over 15 days.

As described herein, Stop COVID was a double-blind, randomized, placebo-controlled, fully-remote (contactless) clinical trial of fluvoxamine. Participants included 152 adult outpatients with confirmed SARS-CoV-2 infection, with symptom onset within 7 days. They were randomized to receive fluvoxamine 100 mg (n=80), or placebo (n=72), three times daily for 15 days. The primary outcome was clinical deterioration over 15 days, defined by meeting both of the following: (1) shortness of breath and/or hospitalization for shortness of breath or pneumonia, (2) oxygen saturation <92% on room air or need for supplemental oxygen to achieve oxygen saturation ≥92%.

As described in the Examples, 152 participants were randomized in the modified intention to treat group. No patients (0/80, 0%) in the fluvoxamine group clinically deteriorated, compared to 8.3% (6/72) in the placebo group (log-rank chi-square 6.8, p=0.009).

It is presently believed that COVID-19 may lead to an excessive inflammatory response resulting in cardiopulmonary complications and lung injury. Described herein is evidence that Fluvoxamine can prevent this clinical deterioration through its action on the sigma1 receptor, which can down-regulate cytokine production.

Also of note, fluvoxamine is listed in NIH guidelines (they recommend neither for nor against use for COVID-19). Fluvoxamine is also included in guidelines or protocols at Johns Hopkins, Washington University, Harvard, and the province of Ontario in Canada. Fluvoxamine is being used to treat COVID in other countries and some are planning to report some observational cohort studies. Others are conducting or planning clinical trials of fluvoxamine—in some cases in combination with other drugs. The state of Florida has listed fluvoxamine as a treatment option on some public service announcements as well. It has also been reported that some physicians and patients report benefits in long COVID.

Since the inventors' discovery, the inventors and others have been extensively studying and spearheading the advancement of the clinical use of fluvoxamine and other drugs to treat COVID-19.

For example, an emergency use authorization (EUA) request application that was sent to the FDA in December of 2021 for new use of fluvoxamine to treat COVID-19, as described in Example 8.

As another example, a retrospective study discovered that various antidepressants can reduce on risk for adverse COVID outcomes and have a protective effect at specific doses (see e.g., Example 6).

Other relevant studies include the TOGETHER Trial, which published in The Lancet Global Health (described in Example 7). Here, a randomized clinical trial replication, performed in Brazil, supports use of fluvoxamine as a treatment for COVID.

Fluvoxamine mechanisms paper was published in Frontiers in Pharmacology which reviews mechanism of action and its role in COVID-19 (see e.g., Example 9) (see also, Hoshimoto et al. 2022 Mechanisms of action of fluvoxamine for COVID-19: a historical review, Expert Review Nature Mol. Psych.). A review regarding fluvoxamine for treatment of COVID published in Drugs, see also Example 10.

An observational study focusing on FIASMA drugs is described in Example 11.

Recent data published in Lancet Global Health includes pharmacokinetic modeling (see e.g., Example 12) relevant to dosing.

A fluvoxamine cost-consequence models showed that Fluvoxamine is cost-saving for COVID-19 outpatient therapy (Mills et al, preprint, December 2021, DOI:10.1101/2021.12.23.21268352):

Furthermore, several additional meta-analyses, whether preprint stage or final publications, draw similar conclusions that fluvoxamine is effective in treating COVID) (see e.g., Wen et al. Ann Med. 2022 December; 54(1):516-523; Lee et al. (see also Example 13); Guo et al., Am J Trop Med Hyg. 2022 Mar. 9). Opportunities for Drug Repurposing of Serotonin Reuptake Inhibitors, such as Potential Uses in Inflammation, Infection, Cancer, Neuroprotection, and Alzheimer's Disease Prevention are discussed in Nykamp et al. Pharmacopsychiatry 2022 January; 55(1):24-29.

### Pharmaceutical Compositions

As described herein a pharmaceutical composition for use to treat, prevent, or reduce the effects of a SARS-CoV-2 infection can be a repurposed antidepressant medication, a psychotropic, a sigma-1 receptor (S1R) ligand antagonist or agonist, a cationic amphiphilic drug, a selective serotonin reuptake inhibitor (SSRI), or a functional inhibitor of acid sphingomyelinase (FIASMA), or a combination thereof.

Among the SSRIs, the order of affinity for sigma-1 receptors is as follows: fluvoxamine>sertraline>fluoxetine>escitalopram>citalopram>>paroxetine. Reports of Ki values for the following can fluctuate on the order of tens of nanomolar, but the following are reported in a 2015 publication in J. Pharm. Sci. by Hashimoto et al.: fluvoxamine (Ki=17.0 nM)>sertraline (Ki=31.6 nM)>fluoxetine (Ki=191.2 nM)>escitalopram (Ki=288.3 nM)>citalopram (Ki=403.8 nM)>>paroxetine (Ki=2041 nM).

Other studies have shown IC₅₀ values for fluoxetine (IC₅₀=5.992 μM), citalopram (IC₅₀=27.51 μM), paroxetine (IC₅₀=12.55 μM), fluvoxamine (IC₅₀=10.54 μM), venlafaxine (IC₅₀=36.35 μM), reboxetine (IC₅₀=17.69 μM), clomipramine (IC₅₀=0.75 μM), imipramine (IC₅₀=3 μM), and desipramine (IC₅₀=8.097 μM).

Fluvoxamine, showing the utmost potent effectiveness in the treatment of psychotic depression, has the highest affinity for σ-1 receptors among SSRIs (Narita et al., 1996). Fluvoxamine, which is an antidepressant medication, a psychotropic, a sigma-1 receptor (S1R) agonist, a cationic amphiphilic drug, a selective serotonin reuptake inhibitor (SSRI), and functional inhibitor of acid sphingomyelinase (FIASMA) showed promise for use as a treatment for COVID-19. Thus, the inventors identified other drugs for use as well.

For example, a pharmaceutical composition can comprise a psychotropic medication having protective effects against COVID-19. The five main types of psychotropic medications can include antidepressants, anti-anxiety medications (anxiolytic medications), stimulants, antipsychotics, and mood stabilizers. Antidepressants are used to treat depression. Antidepressants are used to treat depression. There are many different types of antidepressants. Some types are less frequently used than others but may work for you in consultation with your doctor. The most common antidepressants are selective serotonin reuptake inhibitors (SSRIs), which steadily increase the amount of serotonin in your brain. Serotonin is a powerful neurotransmitter that regulates your mood, bowel movements, sleep, blood clotting, and more; selective norepinephrine reuptake inhibitors (SNRIs), which gradually increase the amount of norepinephrine in your brain. Norepinephrine makes you feel awake and alert; or Bupropion, which promotes important brain activity and can be used to treat seasonal affective disorder (SAD) or to help people quit smoking.

Pharmaceutical agents as described herein can be psychotropic medications, such as:

Abilify (aripiprazole)—atypical antipsychotic used to treat schizophrenia, bipolar disorder, and irritability associated with autism.

Adderall (mixed amphetamine salts)—a stimulant used to treat ADHD.

Ambien (zolpidem)—used as a sleep aid.

Amitriptyline

Anafranil (clomipramine)—a tricyclic antidepressant; mostly used to treat OCD.

Aricept (donepezil)—used to slow the progression of Alzheimer's disease.

Ativan (lorazepam)—a benzodiazepine, used to treat anxiety.

Azstarys (Serdexmethylphenidate/Dexmethylphenidate)—a long-acting stimulant used to treat ADHD (works for 13 hours).

Benperidol—an antipsychotic primarily used to control antisocial hypersexual behavior.

Buspar (buspirone)—an anxiolytic used to treat generalized anxiety disorder.

Belsomra (Suvorexant)—used to treat insomnia.

Celexa (citalopram)—an antidepressant of the selective serotonin reuptake inhibitor class.

clobazam (Frisium, Onfi, Tapclob, Urbanol)—a benzodiazepine that has been marketed as an anxiolytic since 1975 and as an anticonvulsant since 1984.

clorazepate (Novo-Clopate, Tranxene)—a benzodiazepine with anxiolytic, anticonvulsant, sedative, hypnotic, and skeletal muscle relaxant properties.

chlordiazepoxide (Librium)—a sedative and hypnotic benzodiazepine used to treat anxiety, insomnia, and withdrawal symptoms.

Clozaril (clozapine)—atypical antipsychotic used to treat resistant schizophrenia.

Concerta (methylphenidate)—an extended release form of methylphenidate.

Contrave (naltrexone/bupropion)—a combination drug used for weight loss in those that are either obese or overweight with some weight-related illnesses.

Cymbalta (duloxetine)—an antidepressant of the serotonin-norepinephrine reuptake inhibitors class.

Depakote (valproic acid/sodium valproate)—an antiepileptic and mood stabilizer used to treat bipolar disorder, neuropathic pain, and others, sometimes called an antimanic medication. Depakene is the trade name for the same drug prepared without sodium.

Desyrel (trazodone)—an atypical antidepressant used to treat depression and insomnia.

Desoxyn (methamphetamine hydrochloride)—used to treat attention deficit hyperactivity disorder and exogenous obesity.

Dexedrine (dextroamphetamine sulfate)—used to treat attention deficit hyperactivity disorder and narcolepsy.

dextromethorphan (Delsym, DM, DXM, Robitussin)—an antitussive drug that is used as a recreational drug similar to other dissociative anesthetics such as ketamine and phencyclidine.

disulfiram (Antabuse)—inhibits enzyme acetaldehyde dehydrogenase, causing acetaldehyde poisoning when ethanol is consumed; used to cause severe hangover when drinking; increases liver, kidney, and brain damage from drinking.

doxepin (Aponal, Quitaxon, Sinequan)—a tricyclic antidepressant used to treat nerve pain, insomnia; similar to imipramine.

Effexor and Effexor XR (venlafaxine)—an antidepressant of the SNRI class.

Elavil (amitriptyline)—a tricyclic antidepressant used as a first-line treatment for neuropathic pain.

estazolam (Prosom, Eurodin)—a benzodiazepine derivative with anxiolytic, anticonvulsant, hypnotic, sedative and skeletal muscle relaxant properties, commonly prescribed for short-term treatment of insomnia.

Fetzima (levomilnacipran)—an antidepressant of the SNRI class.

Fodiss (fluoxetine)—an antidepressant of the SSRI class.

Geodon (ziprasidone)—atypical antipsychotic used to treat schizophrenia and bipolar mania.

Gabitril (tiagabine)—used off-label in the treatment of anxiety disorders and panic disorder.

Haldol (haloperidol)—typical antipsychotic.

Imovane (zopiclone)—a non-benzodiazepine hypnotic.

Inderal (propranolol)—a beta blocker; it is used for acute anxiety, panic attacks, hypertension.

Invega (paliperidone)—atypical antipsychotic used to treat schizophrenia and schizoaffective disorder.

Keppra (levetiracetam)—an anticonvulsant drug which is sometimes used as a mood stabilizer and has potential benefits for other psychiatric and neurologic conditions such as Tourette syndrome, anxiety disorder, and Alzheimer's disease.

Klonopin (clonazepam)—anti-anxiety and anti-epileptic medication of the benzodiazepine class.

Lamictal (lamotrigine)—an anticonvulsant used as a mood stabilizer.

Latuda (lurasidone)—an atypical antipsychotic.

Lexapro (escitalopram)—an antidepressant of the SSRI class.

Librium (chlordiazepoxide)—a benzodiazepine used to treat acute alcohol withdrawal.

Lithium (Lithobid, Eskalith)—a mood stabilizer.

Lunesta (eszopiclone)—a non-benzodiazepine hypnotic.

Luvox (fluvoxamine)—an antidepressant of the SSRI class.

Melatonin—a hypnotic used to treat insomnia.

Minipress (Prazosin)—atypical psychotropic used to treat PTSD.

Naltrexone (ReVia)—an opioid antagonist primarily used in the management of alcohol dependence, opioid dependence, or other impulse control/addictive behaviors such as habitual self-mutilation, also used in formulation with bupropion (naltrexone/bupropion) to treat obesity.

Neurontin (gabapentin)—an anticonvulsant which is sometimes used as a mood stabilizer, anti-anxiety agent, or to treat chronic pain, particularly diabetic neuropathy.

Pamelor—a tricyclic antidepressant.

Paxil (paroxetine)—an antidepressant of the SSRI class.

Phenelzine (Nardil)—an antidepressant of the MAOI class used to treat depression.

Pimozide (Orap)—a typical antipsychotic used to treat tic disorders.

Pristiq (desvenlafaxine)—an antidepressant of the SNRI class.

Prolixin (fluphenazine)—typical antipsychotic.

Provigil (modafinil)—used to treat excessive sleepiness and narcolepsy Prozac (fluoxetine)—an antidepressant of the SSRI class.

Phenobarbital (Luminal)—a barbiturate, sedative, and hypnotic properties.

Remeron (mirtazapine)—an atypical antidepressant which is often used as a sleep aid.

Restoril (temazepam)—a benzodiazepine used to treat insomnia.

Risperdal (risperidone)—atypical antipsychotic used to treat schizophrenia, bipolar disorder, and irritability associated with autism.

Ritalin (methylphenidate)—a stimulant used to treat ADHD.

Reminyl (galantamine)—used to slow the progression of Alzheimer's dementia.

ReVia (naltrexone)—used for opioid addiction and dependence.

Rexulti (brexpiprazole)—atypical antipsychotic used to treat depression.

Saphris (asenapine)—atypical antipsychotic used to treat schizophrenia and bipolar disorder.

Serax (oxazepam)—anti-anxiety medication of the benzodiazepine class, often used to help during detoxification from alcohol or other addictive substances.

Seroquel and Seroquel XR (quetiapine)—atypical antipsychotic used to treat schizophrenia and bipolar disorder and used off-label (in low doses) to treat insomnia.

Sonata (zaleplon)—a non-benzodiazepine hypnotic.

Spravato (esketamine)—a rapid-acting antidepressant of the NMDA receptor antagonist class; enantiomer of ketamine.

Stelazine (trifluoperazine)—a typical antipsychotic, only rarely used nowadays.

Strattera (atomoxetine)—a non-stimulant medication used to treat ADHD.

Thorazine—(chlorpromazine) the first antipsychotic although highly effective, it is rarely used nowadays because of the high rate of serious side effects.

Tofranil (imipramine)—a tricyclic antidepressant used to treat depression, anxiety, agitation, panic disorder, and bedwetting.

Topamax (topiramate)—an anticonvulsant used to treat epilepsy and migraine headaches.

Trileptal (oxcarbazepine)—an anticonvulsant used as a mood stabilizer.

Trintellix (vortioxetine)—an antidepressant of the serotonin modulator and stimulator class.

Tegretol (carbamazepine)—an anticonvulsant used as a mood stabilizer.

Trilafon (Perphenazine)—an antipsychotic used to treat schizophrenia.

Valium (diazepam)—a benzodiazepine used to treat anxiety.

Vistaril (hydroxyzine)—an antihistamine for the treatment of itches and irritations, an antiemetic, as a weak analgesic, an opioid potentiator, and as an anxiolytic.

Vyvanse (lisdexamfetamine)—a stimulant used to treat attention deficit hyperactivity disorder and binge eating disorder; Vyvanse is converted into Dexedrine in vivo.

Viibryd (vilazodone)—an antidepressant of the serotonin modulator and stimulators class.

Vraylar (cariprazine)—atypical antipsychotic used to treat schizophrenia and bipolar mania.

Wellbutrin SR or XL (bupropion)—an antidepressant of the norepinephrine-dopamine reuptake inhibitor class, used to treat depression and seasonal affective disorder.

Zyban—the same medication but marketed as a smoking cessation aid.

Xanax (alprazolam)—a benzodiazepine used to treat anxiety.

Zoloft (sertraline)—an antidepressant of the SSRI class.

Zulresso (brexanolone)—GABA ModulatorAntidepressants

Zyprexa (olanzapine)—atypical antipsychotic used to treat schizophrenia and bipolar disorder.

The efficacy of antipsychotics and antidepressants for the
treatment of COVID-19 (see e.g., Mueller et al., 2022).

| Title | Design | Intervention | Location | Status |
|---|---|---|---|---|
| Repurposing of Chlorpromazine | open | Chlorpromazine (CPZ) | Centre Hospitalier | Not yet |
| in COVID-19 Treatment | | Combination Product: | St Anne | recruiting |
| (reCoVery) | | Standard of Care (SOC) | Paris, France | |
| Administration of Chlorpromazine | open | Chlorpromazine | Cairo University | Not yet |
| as a Treatment for COVID-19 | | | Cairo, Egypt | recruiting |
| Fluoxetine to Reduce Intubation | open | Fluoxetine | University of | Recruiting |
| and Death After COVID19 Infection | | | Toledo Health | |
| | | | Science Campus | |
| | | | Toledo (USA) | |
| TDCS in Pediatric and Teenage | double- | Transcranial Direct | Mexico | Recruiting |
| Patients With Major Depressive | blind | Current Stimulation | | |
| Disorder During COVID-19 Pandemic | | Fluoxetine | | |
| Fluvoxamine for Early Treatment | double- | Fluvoxamine Placebo | Washington | Active, not |
| of COVID-19 (Stop COVID 2) | blind | | University School | recruiting |
| | | | of Medicine. | |
| | | | Chicago, USA | |
| Fluvoxamine Administration in | double- | Placebo | Budapest, | Recruiting |
| Moderate SARS-CoV-2 (COVID-19) | blind | Fluvoxamine | Hungary, | |
| Infected Patients | | | | |
| ACTIV-6: COVID-19 Study of | double- | Ivermectin | USA | Recruiting |
| Repurposed Medications | blind | Fluvoxamine | | |
| | | Fluticasone | | |
| | | Placebo | | |
| Outpatient Treatment of SARS- | double- | Metformin | University of | Recruiting |
| CoV-2 With Ivermectin, | blind | Placebo | Minnesota | |
| Fluvoxamine, and Metformin | | Fluvoxamine | Minneapolis, USA | |
| (COVID-19) | | Ivermectin | | |
| Repurposed Approved and | double- | Fluvoxamine | Brazil | Recruiting |
| Under Development Therapies | blind | Doxazosin | | |
| for Patients With Early-Onset | | Ivermectin | | |
| COVID-19 and Mild Symptoms | | Placebo | | |
| | | Peginterferon | | |
| | | Lambda-1a | | |
| | | Peginterferon Beta-1A | | |
| Valproate Alone or in | double- | Valproate | University of Miami | Not yet |
| Combination With Quetiapine for | blind | Quetiapine | Miami, USA | recruiting |
| Severe COVID-19 Pneumonia | | Standard of Care | | |
| With Agitated Delirium | | | | |
| Cannabidiol for COVID-19 | double- | Cannabidiol | University of | Active, not |
| patients With Mild to Moderate | blind | Placebo | Sao Paulo | recruiting |
| Symptoms (CANDIDATE) | | | Sao Paulo, Brazil | |
| Cannabidiol Treatment for | open | Cannabidiol | Rabin Medical Center | Recruiting |
| Severe and Critical Coronavirus | | 150 mg twice daily | | |
| (COVID-19) Pulmonary Infection | | during 14 days | | |
| Cannabidiol in Patients With | double- | Cannabidiol | USA | Recruiting |
| COVID-19 and Cardiovascular | blind | Placebo | Sponsor: Cardiol | |
| Disease or Risk Factors | | | Therapeutics Inc. | |
| Synthetic CBD as a Therapy for | double- | Cannabidiol | Sheba Medical | Not yet |
| COVID-19 | blind | Placebo | Center | recruiting |
| | | | Tel-Hashomer, Israel | |

A summary of the various psychotropic drugs for the management of COVID-19.

| | | | |
|---|---|---|---|
| | | Benefit vs. SARS- | |
| Drug name | Mode of action | CoV-2 infection | Cited references |

Hypnotics

| | | | |
|---|---|---|---|
| Melatonin | Inducing RORα-dependent brain | Lowering the | Loh, 2020 |
| | and BMAL1 expression | inflammatory | |
| | Generating BMAL1-dependent | response during | |
| | NAMPT | | |
| | Provoking the synthesis of | SARS-CoV-2 | |
| | NAD + employed by SIRT-1 for | infection | |
| | deacetylating p65 | | |
| | Blocking the 2 main pathways of the | Recovering the | Loh, 2020; Artigas |
| | innate immunity of NF-κB and NLRP3 | mitochondrial | et al., 2020 |
| | inflammasome | homeostasis within | |
| | | SARS-CoV-2 sepsis | |
| | Affecting epidermal growth factor | Antiviral impacts | Feitosa et al., 2020 |
| | signaling | against SARS-CoV-2 | |
| | Inhibiting SARS-CoV-2 main protease | | |
| | Repression of both HIF-1α and mTOR | Reversing aerobic | Reiter et al., 2020 |
| | Disinhibiting PDC activity | glycolysis | |
| | Permitting acetyl coenzyme A | | |
| | synthesis | | |
| | Guaranteeing locally-produced | | |
| | melatonin generation | | |
| | Raising peripheral blood CD4 + T | Improving the | Juybari et al., 2020 |
| | cells and IgG-expressing B cells | immune response | Grifoni et al., 2020 |
| | | to vaccines | |
| | Antioxidant properties | Avoiding the | Maestroni, 2020 |
| | Pleiotropic impacts on the immune | adverse effects | |
| | system | of the vaccine | |
| Mood stabilizers | | | |
| Lithium | Lowering CRP | Lowering the | Murru et al., 2020 |
| carbonate | | cytokine storm | |
| | Increasing the number of | Enhancing the | |
| | lymphocytes, NLR, and PLR levels | clinical status | |
| | (as the inflammation markers) | of a patient | |
| | | Decreasing the | |
| | | ventilation need | |
| | | Providing early | |
| | | discharge | |
| | Prevention of the GSK-3β | Antiviral impacts | Rajkumar, 2020 |
| | Preventing the phosphorylation of | against SARS-CoV-2 | Spuch et al., 2020 |
| | the cofactors required for viral | | Murru et al., 2020 |
| | RNA polymerase activity | | |
| Valproate | Preventing the generation of the | Anti-inflammatory | Unal et al., 2020; |
| | TNF-α, NF-κB, and IL-6 | effects | Ichiyama et al., 2000; |
| | Obstructing the migration of | | Pitt et al., 2021 |
| | macrophages | | |
| | Provoking T cells' differentiation | | |
| | toward Th2/M2 instead of Th1/M1 | | |
| | Stimulating the production of the | | |
| | regulatory T cells, which reduces | | |
| | the CD8 + T lymphocytes percentage | | |
| | via caspase-3 activation and | | |
| | triggering the apoptosis | | |
| | Inhibiting RNA dependent RNA | Antiviral impacts | Unal et al., 2020 |
| | polymerase (nsp12) of COVID-19 | against SARS-CoV-2 | Bhargava et al., 2020 |

Antipsychotics

| | | | |
|---|---|---|---|
| Haloperidol | Sigma receptor regulation | Antiviral impacts | Gordon et al., 2020 |
| | | against SARS-CoV-2 | |
| Olanzapine and | H1 antagonist activity | Anti-inflammatory | Richelson & |
| quetiapine | Reducing IL-6 levels | effects | Souder, 2000 |
| | | | Altschuler & Kast, |
| | | | 2005 Altschuler & |
| | | | Kast, 2020 |

Antidepressants

| | | | |
|---|---|---|---|
| Fluoxetine, | Decreasing the excessive generation | Anti-inflammatory | Hamed & Hagag, 2020 |
| escitalopram, | of TNF-α, CRP, IL-6, and IL-1β | effects | Hoertel et al., 2020 |
| and sertraline | Affecting TMPRSS2 activity and/or | | Schloer et al., 2020 |
| | virus-membrane fusion capacity | | Hoffmann et al., 2020 |
| | | | Zimniak et al., 2020 |
| | Inhibiting FIASMA | Preventing SARS- | Hoffmann et al., 2020 |
| | | CoV-2 cell entry | |
| | Inhibiting SARS-CoV-2 replication | Antiviral impacts | Zimniak et al., 2020 |
| | at a concentration of 0.8 μg/ml | against SARS-CoV-2 | |
| Fluvoxamine | S1R agonist activity | Anti-inflammatory | Lenze et al., 2014 |
| | | effects | |
| | Lysosomotropic properties | Antiviral impacts | Homolak & |
| | Modulation of the IRE1 effects | against SARS-CoV-2 | Kodvanj, 2020 |
| | on autophagy | | Fung & Liu, 2019 |
| | Inhibition of platelet activation | | Schlienger & |
| | | | Meier, 2003 |

Other psychotropic drugs

| | | | |
|---|---|---|---|
| Cannabidiol | Down-regulating ACE2 and | Preventing SARS- | Anil et al., 2021 |
| | TMPRSS2 receptors | CoV-2 cell entry | Wang et al., 2020 |
| | Acting at adenosine A2 receptor site | Anti-inflammatory | Anil et al., 2021 |
| | Reduction of leukocyte migration | effects | Wang et al., 2020 |
| | into the lung | | Nagoor et al., 2021 |
| | Marked inhibition of both | | |
| | proinflammatory cytokines (TNF-α | | |
| | and IL-6) and chemokines (MCP-1 | | |
| | and MIP-2) release | | |
| | Interaction with the PPARγ | | |
| | PPARγ agonist activity | Inhibit SARS-CoV-2 | O'Sullivan & Kendall, 2010 |
| | | replication | Huang et al., 2019 |
| | Regulating fibroblast/myofibroblast | Limiting the onset | Milam et al., 2008 |
| | activation and collagen secretion | of late-onset | Esposito et al., 2020 |
| | in murine models | pulmonary fibrosis | |

Pharmacology of some Usual σ₁ Receptor Ligands (Cobos et al., 2008)

| | | | | |
|---|---|---|---|---|
| | Subtype | Affinity for | Function on | |
| Compound | Selectivity | σ₁ Site* | σ₁ Site | Other Activities |

Benzomorphans

| | | | | |
|---|---|---|---|---|
| (+)-Pentazocine | σ₁ | +++ | Agonist | — |
| (−)-Pentazocine | σ₁/σ₂ | ++ | Agonist | κ₁ agonist, μ₁, μ₂, ligand, low |
| | | | | affinity δ, and κ₃ opioid ligand |
| (+)-SKF-10,047 | σ₁ | +++ | Agonist | NMDA receptor ligand |

Antipsychotics

| | | | | |
|---|---|---|---|---|
| Chlorpromazine | σ₁/σ₂ | ++ | ^{?} | Dopamine D₂ antagonist |
| Haloperidol | σ₁/σ₂ | +++ | Antagonist | Dopamine D₂ and D₃ |
| | | | | antagonist; σ₂ agonist |
| Nemonapride | σ₁/σ₂^{?} | +++ | ^{?} | Dopamine D₂ antagonist |

Antidepressants

| | | | | |
|---|---|---|---|---|
| Clorgyline | σ₁ | +++ | Agonist^{?} | Irreversible monoamine oxidase |
| | | | | A inhibitor |
| Fluoxetine | σ₁ | + | Agonist | Selective 5-HT reuptake |
| | | | | inhibitor |
| Fluvoxamine | σ₁ | +++ | Agonist | Selective 5-HT reuptake |
| | | | | inhibitor |
| Imipramine | σ₁ | ++ | Agonist | Monoamine reuptake inhibitor |
| Sertraline | σ₁ | ++ | Agonist | Selective 5-HT reuptake |
| | | | | inhibitor |

Antitussives

| | | | | |
|---|---|---|---|---|
| Carbetapentane | σ₁/σ₂ | +++ | Agonist | Muscarinic antagonist |
| Dextromethorphan | σ₁ | ++ | Agonist | NMDA receptor allosteric |
| | | | | antagonist |
| Dimemorfan | σ₁/σ₂ | ++ | Agonist | ^{?} |

Parkinson's and/or Alzheimer's disease

| | | | | |
|---|---|---|---|---|
| Amantadine | ^{?} | + | Agonist^{?} | NMDA antagonist, antiviral |
| | | | | properties |
| Donepezil | σ₁/σ₂^{?} | +++^{?} | Agonist | Cholinesterase inhibitor |
| Memantine | ^{?} | + | Agonist^{?} | NMDA antagonist, antiviral |
| | | | | properties |

Drugs of abuse

| | | | | |
|---|---|---|---|---|
| Cocaine | σ₁/σ₂ | + | Agonist | Monoamine transporters |
| | | | | inhibitor, amongst other actions |
| MDMA | σ₁/σ₂ | + | ^{?} | Preferential SERT inhibitor, |
| | | | | among other actions |
| Metamphetamine | σ₁/σ₂ | + | ^{?} | Preferential DAT inhibitor, |
| | | | | amongst other actions |

Putative endogenous ligands (neurosteroids)

| | | | | |
|---|---|---|---|---|
| DHEAS | σ₁ | + | Agonist | GABA_{A} negative modulator |
| Pregnenolone | σ₁ | + | Agonist | NMDA positive/GABA_{A} negative |
| sulfate | | | | modulator |
| Progesterone | σ₁ | + | Antagonist | NMDA negative/GABA_{A} positive |
| | | | | modulator |

Anticonvulsants

| | | | | |
|---|---|---|---|---|
| Phenytoin (DPH) | σ₁ | Not | Allosteric | Delayed rectifier K⁺ channel |
| | | applicable | Modulator | blocker; T-type Ca²⁺ current |
| | | | | inhibitor; Na⁺ current inhibitor |
| Ropizine | σ₁ | Not | Allosteric | ^{?} |
| | | applicable | modulator | |

Other σ drugs

| | | | | |
|---|---|---|---|---|
| BD 737 | σ₁/σ₂ | +++ | Agonist | — |
| BD 1008 | σ₁/σ₂ | +++ | Antagonist | σ₂ agonist^{?} |
| BD 1047 | σ₁ | +++ | Antagonist | α adrenoceptor ligand |
| BD 1063 | σ₁ | +++ | Antagonist | — |
| BMY 14802 | σ₁/σ₂ | ++ | Antagonist | 5-HT_{1A} agonist |
| DTG | σ₁/σ₂ | +++ | ^{?} | σ₂ agonist |
| Dup 734 | σ₁ | +++ | | 5-HT₂ antagonist |
| Eliprodil (SL- | σ₁/σ₂ | ++ | ^{?} | NMDA antagonist, α₁ |
| 82.0715) | | | | adrenoceptor ligand |
| E-5842 | σ₁ | +++ | Antagonist | Low to moderate affinity for |
| | | | | dopamine, 5-HT and glutamate |
| | | | | receptors |
| Haloperidol | σ₁ | ++ | Antagonist | — |
| Metabolite I | | | | |
| Haloperidol | σ₁/σ₂ | +++ | Irreversible | Dopamine D₂ and D₃ ligand |
| Metabolite II | | | antagonist | |
| 4-IBP | σ₁/σ₂ | +++ | Agonist | Dopamine D₂ ligand |
| JO-1784 (Igmesine) | σ₁ | +++ | Agonist | — |
| Metaphit | σ₁/σ₂ | ++ | Irreversible | Acylator of PCP and σ₂ binding |
| | | | antagonist | sites |
| (+)-MR 200 | σ₁/σ₂ | +++ | Antagonist | — |
| MS-377 | σ₁ | +++ | Antagonist | — |
| NE-100 | σ₁ | +++ | Antagonist | — |
| OPC-14523 | σ₁/σ₂ | +++ | Agonist | Agonist of pre- and post- |
| | | | | synaptic 5-HT_{1A} receptors; |
| | | | | SERT inhibitor |
| Panamesine (EMD | σ₁/σ₂^{?} | +++^{?} | Antagonist | One of its metabolites is a |
| 57445) | | | | dopaminergic antagonist |
| (+)-3-PPP | σ₁/σ₂ | ++ | Agonist | σ₂ agonist; NMDA receptor |
| | | | | ligand; dopaminergic agonist |
| PRE 084 | σ₁ | +++ | Agonist | — |
| Rimcazole (BW-234U) | σ₁/σ₂ | + | Antagonist | DAT inhibitor |
| SA4503 | σ₁ | +++ | Agonist | — |
| SR 31742A | ^{?} | +++ | ^{?} | High affinity for C8-C7 sterol |
| | | | | isomerase |

*Kᵢ or K_{D} values:
+++ <50 nM;
++ <500 nM;
+ <10 μM.
^{?}not studied or unclear at the moment.
—: no other pharmacological target has been described.

Evidence that other antidepressants/SIR agonists/FIASMAs/drugs affecting serotonin systems may also be effective in COVID-19 is included in the following:

Hoertel et al. Repurposing antidepressants inhibiting the sphingomyelinase acid/ceramide system against COVID-19: current evidence and potential mechanisms Mol Psychiatry. 2021 December; 26(12):7098-7099. doi: 10.1038/s41380-021-01254-3. Epub 2021 Aug. 12 (inventor-authors).

Hoertel et al. Association Between FIASMAs and Reduced Risk of Intubation or Death in Individuals Hospitalized for Severe COVID-19: An Observational Multicenter Study Clin Pharmacol Ther. 2021 December; 110(6):1498-1511. doi: 10.1002/cpt.2317. Epub 2021 Jul. 2 (inventor-authors).

Hoertel et al. Association between antidepressant use and reduced risk of intubation or death in hospitalized patients with COVID-19: results from an observational study, Mol Psychiatry. 2021 September; 26(9):5199-5212. doi: 10.1038/s41380-021-01021-4.

Hoertel et al. Risk of death in individuals hospitalized for COVID-19 with and without psychiatric disorders: an observational multicenter study in France Biol Psychiatry Glob Open Sci. 2022 Jan. 4. doi: 10.1016/j.bpsgos.2021.12.007.

Mueller et al. Neuropsychiatric Drugs Against COVID-19: What is the Clinical Evidence? Pharmacopsychiatry. 2022 January; 55(1):7-15. doi: 10.1055/a-1717-2381. Epub 2022 Jan. 25.

Oskotsky et al. Mortality Risk Among Patients With COVID-19 Prescribed Selective Serotonin Reuptake Inhibitor Antidepressants. JAMA Netw Open. 2021; 4(11):e2133090. doi:10.1001/jamanetworkopen.2021.33090. This study showed that for patients who were prescribed SSRIs, a reduced relative risk of mortality was found to be associated with the use of SSRIs—specifically fluoxetine—compared with patients who were not prescribed SSRIs.

Hoertel, Do the Selective Serotonin Reuptake Inhibitor Antidepressants Fluoxetine and Fluvoxamine Reduce Mortality Among Patients With COVID-19?JAMA Netw Open. 2021; 4(11):e2136510.

Kornhuber et al. Mol Psychiatry. The acid sphingomyelinase/ceramide system in COVID-19, 2021 Oct. 4; 1-8. doi: 10.1038/s41380-021-01309-5. The results showed that psychotropic drugs such as melatonin, lithium carbonate, valproate, olanzapine, quetiapine, clozapine, fluoxetine, escitalopram, fluvoxamine, and cannabidiol could help lower the mortality due to SARS-CoV-2 infection.

Khosravi, Candidate Psychotropics against SARS-CoV-2: A Narrative Review. Pharmacopsychiatry. 2022 January; 55(1):16-23. doi: 10.1055/a-1551-3756. Epub 2021 Aug. 16.

Kovacs, Antidepressants as antiviral drugs? Neuropsychopharmacol Hung. 2021 Jun. 1; 23(2):240-248.

Brimson et al. Drugs that offer the potential to reduce hospitalization and mortality from SARS-CoV-2 infection: The possible role of the sigma-1 receptor and autophagy. Expert Opin Ther Targets. 2021 June; 25(6):435-449. doi: 10.1080/14728222.2021.1952987. Epub 2021 Jul. 15. Drugs with sigma affinity potentially offer protection against the most severe symptoms of SARS-CoV-2 via interactions with the sigma-1 receptor. Agonists of the sigma-1 receptor may provide protection of the mitochondria, activate mitophagy to remove damaged and leaking mitochondria, prevent ER stress, manage calcium ion transport, and induce autophagy to prevent cell death in response to infection (e.g., chlorpromazine; donepezil; fluoxetine; fluvoxamine).

Carpinteiro et al. Pharmacological Inhibition of Acid Sphingomyelinase Prevents Uptake of SARS-CoV-2 by Epithelial Cells. Cell Rep Med. 2020 Nov. 17; 1(8):100142. doi: 10.1016/j.xcrm.2020.100142. Epub 2020 Oct. 29. The data justify clinical studies investigating whether amitriptyline, a safe drug used clinically for almost 60 years, or other antidepressants that functionally block acid sphingomyelinase prevent SARS-CoV-2 infection.

Becker et al. Ex vivo assay to evaluate the efficacy of drugs targeting sphingolipids in preventing SARS-CoV-2 infection of nasal epithelial cells. STAR Protoc. 2021 Mar. 19; 2(1):100356. doi: 10.1016/j.xpro.2021.100356. Epub 2021 Feb. 3. After treating human volunteers with amitriptyline, an approved antidepressant and inhibitor of the acid sphingomyelinase, freshly isolated nasal epithelial cells were infected ex vivo and infection levels were quantified. This protocol offers the possibility to rapidly test the efficacy of potential drugs in the fight against COVID-19. For complete details on the use and execution of this protocol, please refer to Carpinteiro et al. (2020).

Ostrov et al. Highly Specific Sigma Receptor Ligands Exhibit Anti-Viral Properties in SARS-CoV-2 Infected Cells Pathogens. 2021 Nov. 20; 10(11):1514. doi: 10.3390/pathogens10111514. This study found antiviral activity associated with agonism of the sigma-1 receptor (e.g., SA4503), ligation of the sigma-2 receptor (e.g., CM398), and a combination of the two pathways (e.g., AZ66).

Pandey et al. Insights into the biased activity of dextromethorphan and haloperidol towards SARS-CoV-2 NSP6: in silico binding mechanistic analysis. J Mol Med (Berl). 2020 December; 98(12):1659-1673. doi: 10.1007/s00109-020-01980-1. Epub 2020 Sep. 23.

Bejan et al. entitled “DrugWAS: Drug-wide Association Studies for COVID-19 Drug Repurposing” published in 2021 has some interesting secondary analyses regarding antidepressants, SSRIs, S1R agonists. Specifically, nonsteroidal anti-inflammatory drugs, antidepressants, selective serotonin reuptake inhibitors, and omega-3 supplements show a protective effect. It was shown that sigma-1 receptor agonists are significantly associated with a reduced risk for all COVID-19 outcomes. Furthermore, nonsteroidal anti-inflammatory drugs, antidepressants, selective serotonin reuptake inhibitors, and omega-3 supplements show a protective effect for some of the outcomes.

Exploratory analysis of drug class associations (Bejan et al. 2021 ASCPT).

| | | | | | | |
|---|---|---|---|---|---|---|
| Drug class | TCe | TCu | SRe | SRu | OR | 95% CI |

Primary outcome: all-cause of death

| | | | | | | |
|---|---|---|---|---|---|---|
| Antidepressants | 2,227 | 6,979 | 1.5 | 2.6 | 0.61 | (0.31-1.18) |
| Antihistamines | 2,941 | 6,265 | 1.5 | 2.1 | 0.88 | (0.50-1.58) |
| NSAIDs | 4,495 | 4,711 | 1.1 | 1.6 | 0.53 | (0.28-1.00) |
| Omega-3 supplements | 687 | 8,519 | 2.9 | 5.1 | 0.59 | (0.30-1.18) |
| Sigma-1 receptor agonists | 1,448 | 7,758 | 1 | 2.4 | 0.44 | (0.20-0.97) |
| SNRIs | 512 | 8,694 | 2.4 | 3.8 | 0.75 | (0.31-1.83) |
| SSRIs | 1,381 | 7,825 | 1.9 | 2.4 | 0.73 | (0.36-1.48) |
| Tricyclic antidepressants | 316 | 8,890 | 1.5 | 3.4 | 0.41 | (0.11-1.50) |

Secondary outcome: on ventilator, cumulative severity

| | | | | | | |
|---|---|---|---|---|---|---|
| Antidepressants | 2,232 | 6,997 | 1.7 | 3.2 | 0.54 | (0.29-0.98) |
| Antihistamines | 2,945 | 6,284 | 1.6 | 2.6 | 0.72 | (0.42-1.22) |
| NSAIDs | 4,507 | 4,722 | 1.3 | 2 | 0.54 | (0.31-0.96) |
| Omega-3 supplements | 691 | 8,538 | 3.4 | 5.6 | 0.62 | (0.33-1.19) |
| Sigma-1 receptor agonists | 1,448 | 7,781 | 1 | 2.8 | 0.36 | (0.17-0.76) |
| SNRIs | 514 | 8,715 | 2.8 | 4.4 | 0.71 | (0.31-1.64) |
| SSRIs | 1,384 | 7,845 | 2.1 | 3 | 0.66 | (0.34-1.27) |
| Tricyclic antidepressants | 318 | 8,911 | 2.2 | 3.9 | 0.51 | (0.16-1.57) |

Secondary outcome: in ICU, cumulative severity

| | | | | | | |
|---|---|---|---|---|---|---|
| Antidepressants | 2,242 | 7,025 | 2.1 | 3.9 | 0.53 | (0.31-0.92) |
| Antihistamines | 2,960 | 6,307 | 2 | 3.1 | 0.74 | (0.46-1.19) |
| NSAIDs | 4,528 | 4,739 | 1.6 | 2.5 | 0.53 | (0.31-0.88) |
| Omega-3 supplements | 694 | 8,573 | 3.8 | 6.5 | 0.58 | (0.31-1.06) |
| Sigma-1 receptor agonists | 1,451 | 7,816 | 1.2 | 3.4 | 0.36 | (0.18-0.72) |
| SNRIs | 516 | 8,751 | 3.2 | 5.1 | 0.66 | (0.30-1.42) |
| SSRIs | 1,389 | 7,878 | 2.5 | 3.6 | 0.65 | (0.35-1.18) |
| Tricyclic antidepressants | 320 | 8,947 | 2.8 | 4.7 | 0.53 | (0.19-1.47) |

Secondary outcome: hospitalized-mild, cumulative severity

| | | | | | | |
|---|---|---|---|---|---|---|
| Antidepressants | 2,414 | 7,334 | 7.9 | 9.9 | 0.81 | (0.60-1.10) |
| Antihistamines | 3,147 | 6,601 | 7.1 | 8.7 | 0.86 | (0.66-1.13) |
| NSAIDs | 4,820 | 4,928 | 6 | 7.5 | 0.74 | (0.56-0.98) |
| Omega-3 supplements | 757 | 8,991 | 11.1 | 15.4 | 0.67 | (0.46-0.97) |
| Sigma-1 receptor agonists | 1,520 | 8,228 | 5.5 | 9.1 | 0.56 | (0.39-0.80) |
| SNRIs | 572 | 9,176 | 12.1 | 12.9 | 0.93 | (0.60-1.43) |
| SSRIs | 1,472 | 8,276 | 7.8 | 10.3 | 0.7 | (0.49-0.99) |
| Tricyclic antidepressants | 355 | 9,393 | 12.1 | 12 | 1.19 | (0.70-2.03) |

Secondary outcome: on ventilator, exclusive severity

| | | | | | | |
|---|---|---|---|---|---|---|
| Antidepressants | 2,203 | 6,949 | 0.9 | 1.8 | 0.46 | (0.21-1.02) |
| Antihistamines | 2,915 | 6,237 | 0.8 | 1.4 | 0.63 | (0.31-1.28) |
| NSAIDs | 4,457 | 4,695 | 0.6 | 1.1 | 0.55 | (0.26-1.17) |
| Omega-3 supplements | 682 | 8,470 | 2.3 | 2.3 | 1.07 | (0.46-2.48) |
| Sigma-1 receptor agonists | 1,437 | 7,715 | 0.3 | 1.6 | 0.21 | (0.07-0.68) |
| SNRIs | 507 | 8,645 | 1.7 | 2 | 0.87 | (0.29-2.60) |
| SSRIs | 1,365 | 7,787 | 0.9 | 1.7 | 0.48 | (0.20-1.17) |

Secondary outcome: in ICU, exclusive severity

| | | | | | | |
|---|---|---|---|---|---|---|
| Antidepressants | 2,208 | 6,965 | 1 | 2.3 | 0.41 | (0.20-0.85) |
| Antihistamines | 2,927 | 6,246 | 1.1 | 1.7 | 0.76 | (0.41-1.40) |
| NSAIDs | 4,470 | 4,703 | 0.8 | 1.4 | 0.47 | (0.24-0.93) |
| Omega-3 supplements | 679 | 8,494 | 1.9 | 3.2 | 0.6 | (0.26-1.37) |
| Sigma-1 receptor agonists | 1,442 | 7,731 | 0.6 | 1.9 | 0.35 | (0.14-0.86) |
| SNRIs | 508 | 8,665 | 1.8 | 2.6 | 0.68 | (0.25-1.85) |
| SSRIs | 1,367 | 7,806 | 1 | 2 | 0.43 | (0.18-1.01) |
| Tricyclic antidepressants | 316 | 8,857 | 1.5 | 2.5 | 0.52 | (0.14-1.95) |

Secondary outcome: hospitalized-mild, exclusive severity

| | | | | | | |
|---|---|---|---|---|---|---|
| Antidepressants | 2,350 | 7,199 | 6 | 6.5 | 0.96 | (0.68-1.36) |
| Antihistamines | 3,073 | 6,476 | 5.2 | 6 | 0.92 | (0.68-1.24) |
| NSAIDs | 4,698 | 4,851 | 4.5 | 5.3 | 0.8 | (0.59-1.09) |
| Omega-3 supplements | 727 | 8,822 | 7.8 | 10.5 | 0.71 | (0.46-1.08) |
| Sigma-1 receptor agonists | 1,501 | 8,048 | 4.4 | 6.4 | 0.66 | (0.44-0.98) |
| SNRIs | 554 | 8,995 | 9.4 | 8.7 | 1.09 | (0.67-1.76) |
| SSRIs | 1,435 | 8,114 | 5.6 | 7.3 | 0.72 | (0.48-1.07) |
| Tricyclic antidepressants | 346 | 9,203 | 9.8 | 8.3 | 1.41 | (0.78-2.55) |

Acronyms.
NSAID, nonsteroidal anti-inflammatory drug; SNRI, serotonin, and norepinephrine reuptake inhibitor; SSRI, selective serotonin reuptake inhibitor.

It was reported that a viral protein called NSP6 associates with the S1R. It is believed that this could have something to do with action of S1R ligand antiviral effects if they are present, but it is believed that the main mechanism is anti-inflammatory. Also, in vitro antiviral effects of some S1R ligands may be due to phospholipidosis rather than the S1R binding (see e.g., Tummino et al. Drug-induced phospholipidosis confounds drug repurposing for SARS-CoV-2. Science. 2021 Jul. 30; 373(6554):541-547. doi: 10.1126/science.abi4708. Epub 2021 Jun. 22). Many S1R ligands (both agonists and antagonists) are also cationic amphiphilic drugs (CADs). These drugs usually tend to accumulate in lysosomes, are often functional inhibitors of acid sphingomyelinase, and also tend to cause phospholipidosis at high doses. Phospholipidosis is toxic to cells so could reduce viral replication due to the host cells being unhealthy. This may lead to apparent antiviral effects in vitro but not in animals or humans. Phospholipidosis is a confound that appears to only affect drugs repurposed for direct antiviral activity—it is irrelevant for drugs like dexamethasone and fluvoxamine that have been repurposed for immunomodulation in COVID-19, and it is also irrelevant for CADs whose antiviral activity is well below the concentration range where phospholipidosis occurs.

Animal and cell studies with fluvoxamine suggest that anti-inflammatory effects are likely more important than antiviral effects. The results appear to support S1R-related anti-inflammatory effects as the mechanism. Some researchers favor a FIASMA antiviral hypothesis and some favor the S1R anti-inflammatory hypothesis and others favor the SSRI anti-platelet effects of fluvoxamine as the main mechanism (SSRIs may help to prevent excessive platelet serotonin release in COVID). Furthermore, recent studies have shown that NSP6 viral protein binds to S1R (Gordon et al., Science 370, 1181 (2020)).

### Combination Treatments

The pharmaceutical compositions described herein can be used in combination treatments or in combination with other medications, such as those being used and tested for treatment of COVID-19, such as other drugs which are thought to inhibit virus entry into the cell which may not be recited here, but are proposed or being tested in trials. Also, combination with certain serotonin antagonists are being tested to evaluate treatments, Fluvoxamine, Bromhexine, Cyproheptadine, and Niclosamide suitable for use in the community for treating COVID-like-illness that might help people recover sooner and prevent hospitalization (ClinicalTrials.gov Identifier: NCT05087381). Part of the effect of SSRI action is to inhibit loading of serotonin onto platelets and inhibit platelet activation. One of the things this does is prevent platelets from releasing excessive serotonin. If too much serotonin has already been released (such as if a person is already well into the inflammatory phase of treatment with fluvoxamine or similar drugs), then it may be best also add one or more drugs that could counteract the adverse effects of platelet serotonin. This could include various serotonin antagonist drugs, such as cyproheptadine (which is also a 5-HT 2 A, B, and C receptor antagonist, a sigma1 agonist, and an H1 antagonist), odansetron (5HT3 antagonist, which could also counteract nausea side effect from fluvoxamine), bupropion (an antidepressant which is also a 5HT3 allosteric modulator), and mirtazapine (one of its actions is as a serotonin antagonist action at 5HT2A, and it also inhibits platelets by binding to both 5HT2A and alpha2-adrenergic receptors on platelets). Furthermore, combinations with aspirin or other NSAIDs, or with famotidine (H2 agonist), or any type of antihistamine (H1 or H2 blockers) can be used since there are reports that these drugs are useful in reducing symptoms thought to be related to mast cell hyperactivation in acute and long COVID. Other combinations with a number of treatments that are currently being used to treat long COVID will likely be tested in clinical trials soon. For example, long COVID clinics are using include ivermectin, fluvoxamine, cyproheptadine, maraviroc (an HIV drug), H1 and H2 blockers (especially the H2 blocker famotidine), low-dose naltrexone, Cromolyn sodium (intranasal), modafinil, or armodafinil (for fatigue/sleepiness), and statins (particularly pravastatin due to less interactions with some other drugs being used to treat long COVID). Combination with cyproheptadine or antihistamines in general (H1 & H2 blockers) are thought to be useful.

For example, there are several clinical trials directed to studying cyproheptadine and one in Thailand is the one that combines fluvoxamine with cyproheptadine (ClinicalTrials.gov Identifier: NCT05087381).

This trial also includes fluvoxamine+bromhexine (Bromhexine is thought to inactivate the TMPRSS2 in order to prevent the entry of a virus into cell, which is through a mechanism different from the FIASMA mechanism).

### S1R Binding Agent

One aspect of the present disclosure provides for targeting sigma 1 receptor (S1R). The present disclosure provides methods of treating or preventing COVID-19 and associated symptoms, conditions, and syndromes thereof based on the discovery that fluvoxamine is a S1R binding agent, agonist, or antagonist.

As described herein, inhibitors, agonists, or antagonists of S1R (e.g., antidepressants) can reduce or prevent COVID-19 and clinical deterioration. An S1R binding agent can be any agent that can inhibit, block, antagonize, agonize, etc. S1R, activity, or signaling thereof, downregulate S1R expression, or knockdown S1R expression.

The value Ki is the dissociation constant describing the binding affinity between the inhibitor and the enzyme, while IC₅₀ is the concentration of inhibitor required to reduce the activity to half of the uninhibited value. Ki can be used to measure the strength of the S1R binding or dissociation. Inhibition of agents as described herein can be determined by standard pharmaceutical procedures in assays or cell cultures for determining the IC₅₀. The half maximal inhibitory concentration (IC₅₀) is a measure of the potency of a substance in inhibiting a specific biological or biochemical function. The IC₅₀ is a quantitative measure that indicates how much of a particular inhibitory substance (e.g., pharmaceutical agent or drug) is needed to inhibit, in vitro, a given biological process or biological component by 50%. The biological component could be an enzyme, cell, cell receptor, or microorganism, for example. IC₅₀ values are typically expressed as molar concentration. IC₅₀ is generally used as a measure of antagonist drug potency in pharmacological research. IC₅₀ is comparable to other measures of potency, such as EC₅₀ for excitatory drugs. EC₅₀ represents the dose or plasma concentration required for obtaining 50% of a maximum effect in vivo. IC₅₀ can be determined with functional assays or with competition binding assays.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing COVID-19 or symptoms associated with COVID-19 or COVID-19 vaccine (e.g., a SARS-CoV-2 infection, asymptomatic COVID-19, symptomatic COVID-19, pre-symptomatic COVID-19, post-symptomatic COVID-19, acute COVID-19, post-acute sequelae of SARS-CoV-2 infection (PASC), respiratory deterioration), or reducing the risk of serious effects (e.g., hospitalization, deterioration, etc.) in a subject in need of administration of a therapeutically effective amount of a pharmaceutical agent (e.g., sigma-1 receptor (S1R) agonist, SSRIs, such as fluvoxamine), and other repurposed medications, so as to substantially inhibit a SARS-CoV-2 infection, slow the progress of a SARS-CoV-2 infection, or limit the development of a SARS-CoV-2 infection.

Symptoms, disorders, or conditions that can be treated or prevented using the pharmaceutical compositions described herein can be hypoxia, dyspnea, post-vaccine long-COVID-like symptoms or other vaccine adverse effects, treating, preventing, or reversing COVID-19 or symptoms associated with COVID-19 or COVID-19 vaccine (e.g., a SARS-CoV-2 infection, asymptomatic COVID-19, symptomatic COVID-19, pre-symptomatic COVID-19, post-symptomatic COVID-19, acute COVID-19, post-acute sequelae of SARS-CoV-2 infection (PASC), respiratory deterioration), reducing the risk of serious effects (e.g., hospitalization, deterioration, etc.), preventing progression to severe COVID-19 or hospitalization, or treating or preventing SARS-CoV-2 infection, COVID-19, or COVID-19 symptoms, reduce the risk of necessitating interventional care (optionally, dexamethasone, supplemental oxygen), reduce the risk of clinical deterioration; intubation or death; excessive immune response associated with a COVID-19 infection; an inflammatory response in the subject; short and long term complications of COVID-19; severe lung damage; damage from an inflammatory response; or shortness of breath; or reducing the risk of developing severe long-term post-COVID symptoms; developing COVID acute respiratory distress syndrome (ARDS); developing post-acute sequelae of SARS-CoV-2 infection (PASC); death; declining or deteriorating health; hospitalization; progression to severe disease with hypoxia 592%; being admitted in an emergency setting or retention for greater than 6 h; developing severe disease or illness; or developing respiratory deterioration.

As described herein, the mechanism in which SSRIs such as fluvoxamine treats and prevents COVID-19 and prevents serious effects of COVID-19, it is also believed that fluvoxamine can be used to prevent or treat post-vaccine long-COVID-like symptoms or other vaccine adverse effects because it is thought that post-vaccine long-COVID-like symptoms are caused by similar inflammatory and perhaps autoimmune mechanisms as acute/long COVID. Although not well-studied, there is wider acknowledgement of some vaccine adverse events. SSRIs, other antidepressants, and other repurposed medications as described herein, such as fluvoxamine, can be used to prevent or treat these effects.

Methods described herein are generally performed on a subject in need thereof. A subject in need of the therapeutic methods described herein can be a subject having, diagnosed with, suspected of having, or at risk for developing COVID-19. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a pharmaceutical agent such as an S1R agonist is, for example, an amount that would cause the desired therapeutic effect in a subject while minimizing undesired side effects. In various embodiments, an effective amount of a pharmaceutical agent described herein can substantially reduce likelihood of severe illness, reduce likelihood of severe lung damage, reduce COVID-19 symptoms, reduce symptoms, reduce the amount of recovery time, inhibit a SARS-CoV-2 infection, slow the progress of a SARS-CoV-2 infection, or limit the development of a SARS-CoV-2 infection.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a pharmaceutical agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to substantially inhibit a SARS-CoV-2 infection, slow the progress of a SARS-CoV-2 infection, or limit the development of a SARS-CoV-2 infection.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of a pharmaceutical agent can occur as a single event or over a time course of treatment. For example, a pharmaceutical agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for a SARS-CoV-2 infection or COVID-19, or post-COVID conditions such as long COVID, long-haul COVID, post-acute COVID-19, long-term effects of COVID, or chronic COVID. Long COVID—or post-COVID conditions—is a wide range of new, returning or ongoing health problems people may experience more than four weeks.

A pharmaceutical agent can be administered simultaneously or sequentially with another agent, such as an antibiotic, an anti-inflammatory, or another agent. For example, a pharmaceutical agent can be administered simultaneously with another agent, such as an antibiotic or an anti-inflammatory. Simultaneous administration can occur through administration of separate compositions, each containing one or more of a pharmaceutical agent, an antibiotic, an anti-inflammatory, a steroid, an anti-viral, or another agent. Simultaneous administration can occur through administration of one composition containing two or more of a pharmaceutical agent, an antibiotic, an anti-inflammatory, a steroid, an anti-viral, or another agent. A pharmaceutical agent can be administered sequentially with an antibiotic, an anti-inflammatory, or another agent. For example, a pharmaceutical agent can be administered before or after administration of an antibiotic, an anti-inflammatory, a steroid, an anti-viral, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Dose and Duration

As described herein, there are many different options for effectively dosing a subject with the pharmaceutical compositions of interest (e.g., an antidepressant, such as fluvoxamine or another drug having a fluoxetine-equivalent).

It is believed that subjects being administered between at least 150 mg and 200 mg total daily dose will benefit. It is also believed that some may need to go up to a maximum total daily dose of about 300 mg (e.g., 150 BID, or some may prefer majority of dose at bedtime or morning, depending on effects on sleep). Another example, the dose can be can be up to 100 mg AM and up to 200 mg HS. As another example, a subject can take 50 mg AM and 100 mg for long COVID or up to 300 mg for acute COVID, and gradually decrease dose or decrease as symptoms decrease. As another example, if tolerated up to 300 mg/day can be administered. Doses can be adjusted based on any criteria or biomarker, such as C-reactive protein (CRP), symptoms, etc. For example, as CRP reduces the dose may be reduced. If CRP is stable or increases, the dose can be increased. As another example, starting at a dose of at least 50 mg/day can be administered prior to symptoms, at positive PCR test, or notification of exposure or outbreak (see e.g., Seftel & Boulware prospective cohort study that started medication early, at positive PCR test during an occupational outbreak, even if no symptoms yet). As another example, observational studies suggest appropriate dose for reduction in risk for hospitalization or death is at least 20 mg fluoxetine equivalents. Additional evidence that up to 300 mg total daily dose may sometimes be more appropriate is demonstrated by the observational cohort study in Croatia that compared ICU patients treated with 100 mg TID (three times per day) fluvoxamine with matched controls.

Additional evidence, as described herein, supports the use of the pharmaceutical agents at various doses that are effective to treat or prevent COVID-19 or effects thereof, such as hospitalization or progression of illness, for example. For example, Calusic et al. 2021 BJCP entitled “Safety and efficacy of fluvoxamine in COVID-19 ICU patients: An open label, prospective cohort trial with matched controls” showed overall mortality was lower in the fluvoxamine group given 100 mg three times daily for 15 days.

As described herein, many dosing regimens have been found to be effective. For example, the dose (or fluoxetine-equivalent) can be between about 1 mg and about 600 mg; about 1 mg; about 10 mg; about 20 mg; about 30 mg; about 40 mg; about 50 mg; about 60 mg; about 70 mg; about 80 mg; about 90 mg; about 100 mg; about 110 mg; about 120 mg; about 130 mg; about 140 mg; about 150 mg; about 160 mg; about 170 mg; about 180 mg; about 190 mg; about 200 mg; about 210 mg; about 220 mg; about 230 mg; about 240 mg; about 250 mg; about 260 mg; about 270 mg; about 280 mg; about 290 mg; about 300 mg; about 310 mg; about 320 mg; about 330 mg; about 340 mg; about 350 mg; about 360 mg; about 370 mg; about 380 mg; about 390 mg; about 400 mg; about 410 mg; about 420 mg; about 430 mg; about 440 mg; about 450 mg; about 460 mg; about 470 mg; about 480 mg; about 490 mg; about 500 mg; about 510 mg; about 520 mg; about 530 mg; about 540 mg; about 550 mg; about 560 mg; about 570 mg; about 580 mg; about 590 mg; about 600 mg; about 610 mg; about 620 mg; about 630 mg; about 640 mg; about 650 mg; about 660 mg; about 670 mg; about 680 mg; about 690 mg; about 700 mg; about 710 mg; about 720 mg; about 730 mg; about 740 mg; about 750 mg; about 760 mg; about 770 mg; about 780 mg; about 790 mg; about 800 mg; about 810 mg; about 820 mg; about 830 mg; about 840 mg; about 850 mg; about 860 mg; about 870 mg; about 880 mg; about 890 mg; about 900 mg; about 910 mg; about 920 mg; about 930 mg; about 940 mg; about 950 mg; about 960 mg; about 970 mg; about 980 mg; about 990 mg; or about 1000 mg. The dose can be given all at once or in increments of once per day, twice per day, three times per day, four times per day, or more as tolerated. The dose can be administered over a time course of at least 1 day, 2 days, about 3 days; about 4 days; about 5 days; about 6 days; about 7 days; about 8 days; about 9 days; about 10 days; about 11 days; about 12 days; about 13 days; about 14 days; about 15 days; about 16 days; about 17 days; about 18 days; about 19 days; about 20 days; about 21 days; about 22 days; about 23 days; about 24 days; about 25 days; about 26 days; about 27 days; about 28 days; about 29 days; or about 30 days as tolerated. A range of K values for S1R binding optimal for SSRI to treat COVID are possible. For example, the S1R binding can be less than 3000 nM, between about 1 nM and about 3000 nM, or not greater than 10 μM. As another example, the Ki value can be less than, greater than, equal to, or between about the following values: about 1 nM; about 10 nM; about 20 nM; about 30 nM; about 40 nM; about 50 nM; about 60 nM; about 70 nM; about 80 nM; about 90 nM; about 100 nM; about 110 nM; about 120 nM; about 130 nM; about 140 nM; about 150 nM; about 160 nM; about 170 nM; about 180 nM; about 190 nM; about 200 nM; about 210 nM; about 220 nM; about 230 nM; about 240 nM; about 250 nM; about 260 nM; about 270 nM; about 280 nM; about 290 nM; about 300 nM; about 310 nM; about 320 nM; about 330 nM; about 340 nM; about 350 nM; about 360 nM; about 370 nM; about 380 nM; about 390 nM; about 400 nM; about 410 nM; about 420 nM; about 430 nM; about 440 nM; about 450 nM; about 460 nM; about 470 nM; about 480 nM; about 490 nM; about 500 nM; about 510 nM; about 520 nM; about 530 nM; about 540 nM; about 550 nM; about 560 nM; about 570 nM; about 580 nM; about 590 nM; about 600 nM; about 610 nM; about 620 nM; about 630 nM; about 640 nM; about 650 nM; about 660 nM; about 670 nM; about 680 nM; about 690 nM; about 700 nM; about 710 nM; about 720 nM; about 730 nM; about 740 nM; about 750 nM; about 760 nM; about 770 nM; about 780 nM; about 790 nM; about 800 nM; about 810 nM; about 820 nM; about 830 nM; about 840 nM; about 850 nM; about 860 nM; about 870 nM; about 880 nM; about 890 nM; about 900 nM; about 910 nM; about 920 nM; about 930 nM; about 940 nM; about 950 nM; about 960 nM; about 970 nM; about 980 nM; about 990 nM; or about 1000 nM; about 1 μM; about 10 μM; about 20 μM; about 30 μM; about 40 μM; about 50 μM; about 60 μM; about 70 μM; about 80 μM; about 90 μM; about 100 μM; about 110 μM; about 120 μM; about 130 μM; about 140 μM; about 150 μM; about 160 μM; about 170 μM; about 180 μM; about 190 μM; about 200 μM; about 210 μM; about 220 μM; about 230 μM; about 240 μM; about 250 μM; about 260 μM; about 270 μM; about 280 μM; about 290 μM; about 300 μM; about 310 μM; about 320 μM; about 330 μM; about 340 μM; about 350 μM; about 360 μM; about 370 μM; about 380 μM; about 390 μM; about 400 μM; about 410 μM; about 420 μM; about 430 μM; about 440 μM; about 450 μM; about 460 μM; about 470 μM; about 480 μM; about 490 μM; about 500 μM; about 510 μM; about 520 μM; about 530 μM; about 540 μM; about 550 μM; about 560 μM; about 570 μM; about 580 μM; about 590 μM; about 600 μM; about 610 μM; about 620 μM; about 630 μM; about 640 μM; about 650 μM; about 660 μM; about 670 μM; about 680 μM; about 690 μM; about 700 μM; about 710 μM; about 720 μM; about 730 μM; about 740 μM; about 750 μM; about 760 μM; about 770 μM; about 780 μM; about 790 μM; about 800 μM; about 810 μM; about 820 μM; about 830 μM; about 840 μM; about 850 μM; about 860 μM; about 870 μM; about 880 μM; about 890 μM; about 900 μM; about 910 μM; about 920 μM; about 930 μM; about 940 μM; about 950 μM; about 960 μM; about 970 μM; about 980 μM; or about 990 μM. As another example, the IC₅₀ value can be less than, greater than, equal to, or between about the following values: about 1 μM; about 10 μM; about 20 μM; about 30 μM; about 40 μM; about 50 μM; about 60 μM; about 70 μM; about 80 μM; about 90 μM; about 100 μM; about 110 μM; about 120 μM; about 130 μM; about 140 μM; about 150 μM; about 160 μM; about 170 μM; about 180 μM; about 190 μM; about 200 μM; about 210 μM; about 220 μM; about 230 μM; about 240 μM; about 250 μM; about 260 μM; about 270 μM; about 280 μM; about 290 μM; about 300 μM; about 310 μM; about 320 μM; about 330 μM; about 340 μM; about 350 μM; about 360 μM; about 370 μM; about 380 μM; about 390 μM; about 400 μM; about 410 μM; about 420 μM; about 430 μM; about 440 μM; about 450 μM; about 460 μM; about 470 μM; about 480 μM; about 490 μM; about 500 μM; about 510 μM; about 520 μM; about 530 μM; about 540 μM; about 550 μM; about 560 μM; about 570 μM; about 580 μM; about 590 μM; about 600 μM; about 610 μM; about 620 μM; about 630 μM; about 640 μM; about 650 μM; about 660 μM; about 670 μM; about 680 μM; about 690 μM; about 700 μM; about 710 μM; about 720 μM; about 730 μM; about 740 μM; about 750 μM; about 760 μM; about 770 μM; about 780 μM; about 790 μM; about 800 μM; about 810 μM; about 820 μM; about 830 μM; about 840 μM; about 850 μM; about 860 μM; about 870 μM; about 880 μM; about 890 μM; about 900 μM; about 910 μM; about 920 μM; about 930 μM; about 940 μM; about 950 μM; about 960 μM; about 970 μM; about 980 μM; about 990 μM; about 1 nM; about 10 nM; about 20 nM; about 30 nM; about 40 nM; about 50 nM; about 60 nM; about 70 nM; about 80 nM; about 90 nM; about 100 nM; about 110 nM; about 120 nM; about 130 nM; about 140 nM; about 150 nM; about 160 nM; about 170 nM; about 180 nM; about 190 nM; about 200 nM; about 210 nM; about 220 nM; about 230 nM; about 240 nM; about 250 nM; about 260 nM; about 270 nM; about 280 nM; about 290 nM; about 300 nM; about 310 nM; about 320 nM; about 330 nM; about 340 nM; about 350 nM; about 360 nM; about 370 nM; about 380 nM; about 390 nM; about 400 nM; about 410 nM; about 420 nM; about 430 nM; about 440 nM; about 450 nM; about 460 nM; about 470 nM; about 480 nM; about 490 nM; about 500 nM; about 510 nM; about 520 nM; about 530 nM; about 540 nM; about 550 nM; about 560 nM; about 570 nM; about 580 nM; about 590 nM; about 600 nM; about 610 nM; about 620 nM; about 630 nM; about 640 nM; about 650 nM; about 660 nM; about 670 nM; about 680 nM; about 690 nM; about 700 nM; about 710 nM; about 720 nM; about 730 nM; about 740 nM; about 750 nM; about 760 nM; about 770 nM; about 780 nM; about 790 nM; about 800 nM; about 810 nM; about 820 nM; about 830 nM; about 840 nM; about 850 nM; about 860 nM; about 870 nM; about 880 nM; about 890 nM; about 900 nM; about 910 nM; about 920 nM; about 930 nM; about 940 nM; about 950 nM; about 960 nM; about 970 nM; about 980 nM; about 990 nM; or about 1000 nM; about 1 μM; about 10 μM; about 20 μM; about 30 μM; about 40 μM; about 50 μM; about 60 μM; about 70 μM; about 80 μM; about 90 μM; about 100 μM; about 110 μM; about 120 μM; about 130 μM; about 140 μM; about 150 μM; about 160 μM; about 170 μM; about 180 μM; about 190 μM; about 200 μM; about 210 μM; about 220 μM; about 230 μM; about 240 μM; about 250 μM; about 260 μM; about 270 μM; about 280 μM; about 290 μM; about 300 μM; about 310 μM; about 320 μM; about 330 μM; about 340 μM; about 350 μM; about 360 μM; about 370 μM; about 380 μM; about 390 μM; about 400 μM; about 410 μM; about 420 μM; about 430 μM; about 440 μM; about 450 μM; about 460 μM; about 470 μM; about 480 μM; about 490 μM; about 500 μM; about 510 μM; about 520 μM; about 530 μM; about 540 μM; about 550 μM; about 560 μM; about 570 μM; about 580 μM; about 590 μM; about 600 μM; about 610 μM; about 620 μM; about 630 μM; about 640 μM; about 650 μM; about 660 μM; about 670 μM; about 680 μM; about 690 μM; about 700 μM; about 710 μM; about 720 μM; about 730 μM; about 740 μM; about 750 μM; about 760 μM; about 770 μM; about 780 μM; about 790 μM; about 800 μM; about 810 μM; about 820 μM; about 830 μM; about 840 μM; about 850 μM; about 860 μM; about 870 μM; about 880 μM; about 890 μM; about 900 μM; about 910 μM; about 920 μM; about 930 μM; about 940 μM; about 950 μM; about 960 μM; about 970 μM; about 980 μM; about 990 μM; about 1 mM; about 10 mM; about 20 mM; about 30 mM; about 40 mM; about 50 mM; about 60 mM; about 70 mM; about 80 mM; about 90 mM; about 100 mM; about 110 mM; about 120 mM; about 130 mM; about 140 mM; about 150 mM; about 160 mM; about 170 mM; about 180 mM; about 190 mM; about 200 mM; about 210 mM; about 220 mM; about 230 mM; about 240 mM; about 250 mM; about 260 mM; about 270 mM; about 280 mM; about 290 mM; about 300 mM; about 310 mM; about 320 mM; about 330 mM; about 340 mM; about 350 mM; about 360 mM; about 370 mM; about 380 mM; about 390 mM; about 400 mM; about 410 mM; about 420 mM; about 430 mM; about 440 mM; about 450 mM; about 460 mM; about 470 mM; about 480 mM; about 490 mM; about 500 mM; about 510 mM; about 520 mM; about 530 mM; about 540 mM; about 550 mM; about 560 mM; about 570 mM; about 580 mM; about 590 mM; about 600 mM; about 610 mM; about 620 mM; about 630 mM; about 640 mM; about 650 mM; about 660 mM; about 670 mM; about 680 mM; about 690 mM; about 700 mM; about 710 mM; about 720 mM; about 730 mM; about 740 mM; about 750 mM; about 760 mM; about 770 mM; about 780 mM; about 790 mM; about 800 mM; about 810 mM; about 820 mM; about 830 mM; about 840 mM; about 850 mM; about 860 mM; about 870 mM; about 880 mM; about 890 mM; about 900 mM; about 910 mM; about 920 mM; about 930 mM; about 940 mM; about 950 mM; about 960 mM; about 970 mM; about 980 mM; about 990 mM; or about 1 M. Recitation of each of these discrete values is understood to include ranges between each value. Recitation of each range is understood to include discrete values within the range.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to an S1R agonist, such as fluvoxamine. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Fluvoxamine for Treatment of COVID-19

This example describes a novel method of treating COVID-19. Disclosed herein is evidence that fluvoxamine can prevent respiratory deterioration in patients with acute COVID-19 illness and also reduce Post-Acute Sequelae of SARS-CoV-2 infection (PASC).

Fluvoxamine maleate (also known as Luvox and other brand names) is a selective serotonin reuptake inhibitor (SSRI) and a sigma-1 receptor (S1R) agonist, which has an FDA indication for treatment of obsessions and compulsions in children (age 8-17) and adults with Obsessive-Compulsive Disorder (OCD). An extended release form (Luvox CR) has an additional indication for Social Anxiety Disorder, but according to information on the drugs@FDA website, this formulation has been discontinued.

Here is disclosed the discovery of an entirely novel use of fluvoxamine maleate: treatment of Coronavirus disease 2019 (COVID-19). There is evidence that some patients with COVID-19 experience deterioration around the second week of illness due to an excessive inflammatory response (Mehta et al, 2020; Prasad & Prasad, 2020), which can sometimes be characterized by elevated levels of cytokines and other inflammatory molecules. Excessive inflammation during acute illness can result in severe disease, sometimes including respiratory deterioration, need for hospitalization, and death. Inflammation may also contribute to long-term symptoms after the acute phase of COVID-19 illness (Ostergaard, 2021). Fluvoxamine maleate may be able to prevent or ameliorate the body's excessive inflammatory response, preventing respiratory deterioration and other short and long term complications of COViD-19. Preclinical research supporting this hypothesis includes a past study demonstrating that fluvoxamine was beneficial in animal models of inflammation and sepsis (Rosen et al., 2019). That study also found evidence that the benefit came from fluvoxamine's ability to activate the sigma-1 receptor (S1R), which leads to inhibition of the cytokine production that can occur in response to inflammatory triggers. Fluvoxamine also has additional mechanisms that may be helpful for COVID-19. Since coronaviruses make use of the endoplasmic reticulum (ER) stress response during infection of cells (Fung & Liu, 2019), and the S1R regulates the ER stress response (Hayashi, 2019), the S1R agonist activity of fluvoxamine may lead to some anti-viral activity. SARS-CoV-2 proteins have been shown to physically interact with the S1R (Gordon et al., 2020), though the significance of this is not clear. Fluvoxamine is also a cationic amphiphilic drug with lysosomotropic effects (Homolak & Kodvanj, 2020). Because of this, it accumulates in lysosomes, which may interfere with viral assembly within the lysosomes or with the escape of the virus from the cell through the fusion of these lysosomes with the cell membrane (Homolak & Kodvanj, 2020; Gosh et al., 2020). Fluvoxamine is also a functional inhibitor of acid sphingomyelinase (FIASMA), which can result in reduced ceramide in the cell membrane (Hoertel, 2021). This reduction of ceramide in the cell membrane could interfere with coronavirus entry into human cells (Carpinteiro, 2020). Some symptoms of COVID-19 may be due to hyperactivation of platelets and mast cells (Theoharides et al, 2020; Theoharides & Conti, 2020). SSRIs in general inhibit the activation of platelets and mast cells (Schlienger R G & Meier, 2003; Chen et al., 2008), so fluvoxamine may be beneficial in treating this feature of COVID-19. Fluvoxamine also inhibits the metabolism of melatonin in the liver (Harpsoe et al., 2015), which increases the level of melatonin in the body. This may be helpful because melatonin also has its own anti-inflammatory effects (Shneider et al., 2020).

To test the hypothesis that fluvoxamine could be beneficial in the treatment of COVID-19, a double-blind, randomized controlled trial of fluvoxamine versus placebo in outpatients with COVID-19 was conducted.

Patients were given fluvoxamine or placebo for 15 days. The primary outcome measure was respiratory deterioration defined by BOTH of 1) shortness of breath or hospitalization for pneumonia, or 2) blood oxygen saturation drop below 92% or requirement for supplemental oxygen to prevent this. At the end of the trial none of 80 patients receiving fluvoxamine experienced respiratory deterioration, but 6 of 72 patients taking placebo deteriorated. This was a statistically significant difference (absolute difference, 8.7% [95% CI, 1.8%-16.4%] from survival analysis; log-rank P=0.009). The main results of this study and some secondary analysis regarding symptom data have been published (Lenze et al., 2020, Rodebaugh et al., 2020). A larger trial is currently being conducted to confirm the findings of the first trial. Analysis of some long-term follow-up data is in progress (see e.g., Example 4), and there is evidence that fluvoxamine given during the acute phase may reduce the chance of severe long-term post-COVID symptoms. At 113-229 days after the start of their participation in the clinical trial, 102 participants were asked what percent recovered they felt they were. Fifteen percent of the placebo group reported being less than 60% recovered, but only 6% of the fluvoxamine group felt they were less than 60% recovered. This suggests fluvoxamine may prevent or reduce the severity of Post-Acute Sequelae of SARS-CoV-2 infection (PASC). A trial is being planned to confirm that fluvoxamine started during the post-acute phase helps treat PASC.

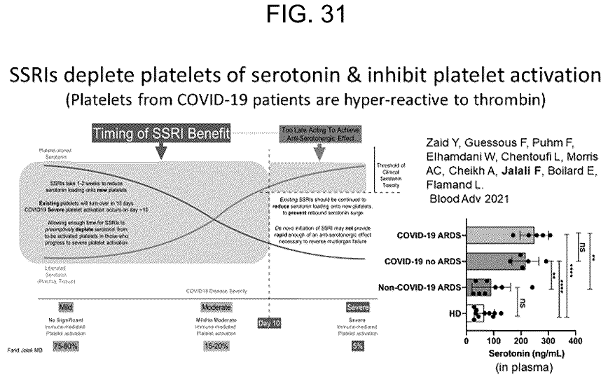

An illustration of the proposed mechanism of fluvoxamine and its platelet inhibition effects (FIG. 31 courtesy of Dr. Farid Jalali). Dr. Jalil believes serotonin release from platelets has a large role in COVID-19. The right graph (FIG. 31) is from a paper Dr. Farid Jalali co-authored with some others. Certain drugs that appear to be beneficial in these medical-records-based observational studies are serotonin antagonists or are negative allosteric modulators of a serotonin receptor. It is possible that this benefit is related to their ability to counteract effects of excessive serotonin release from platelets, and/or displace auto-antibodies that bind to and activate serotonin receptors (e.g., 5HT2A antibodies which activate the receptor are common in severe COVID).

### Review of Fluvoxamine for COVID-19

Here is discussed a review of the current evidence and discussion of practical issues to help physicians who are considering whether to prescribe fluvoxamine for COVID-19.

Fluvoxamine is a selective serotonin reuptake inhibitor (an SSRI) commonly used to treat psychiatric conditions, but it can also reduce the inflammation triggered by infections through its action on the sigma1 receptor 9S1R) inside our cells. Fluvoxamine is FDA-approved for Obsessive-Compulsive Disorder, but it is not currently FDA-approved for treatment of COVID-19. At this time, any use of fluvoxamine for treatment of COVID-19 would be considered off-label use. Pharmaceuticals must go through a testing process to demonstrate their safety and efficacy for specific indications—that way, physicians and regulatory agencies like the FDA become comfortable recommending drugs for specific purposes. We will start today by describing some of the relevant trials related to fluvoxamine in COVID-19.

### Stop COVID Trials

The STOP COVID 1 trial was a randomized, placebo-controlled trial for outpatients with COVID-19 and less than 1 week of symptoms.

This study showed a benefit of fluvoxamine with NONE of the 80 patients in the fluvoxamine group experiencing respiratory deterioration and 8.3 percent of the 72 patients in the placebo group deteriorating. These results were published in JAMA in November 2020.

The STOP COVID 2 trial was similar but enriched for participants with higher risk factors for severe COVID19 illness. This trial stopped recruitment early due to lower than expected rates of the primary clinical deterioration outcome, which meant this study would have low power to detect a difference between fluvoxamine and placebo, even if the sample size was substantially increased. Also, there were difficulties with recruitment after the vaccine rollout. Stop COVID 2 data analysis is ongoing and some of the main results have already been shared with NIH and WHO.

### Together Trial

Recently, the Together Trial, in Brazil, completed its fluvoxamine arm. This study had over 700 people in the fluvoxamine group and over 700 in the placebo group. There was an estimated 32% reduction in risk for extended COVID-19 emergency center visits and tertiary care hospitalizations, with even better results in the subset who took at least 80% of their pills.

These highly adherent patients had a 66% reduction in risk for hospitalization and a 91% reduction in risk of death. These results were published in The LANCET Global Health in October 2021.

Prospective Cohort Studies that Added to the Evidence for Fluvoxamine

Seftel & Boulware study: Around the time our SC1 paper was published in JAMA (see Example 2), there was an occupational outbreak of COVID-19 at the Golden Gate Fields horse racetrack in Berkeley, California. Having read our STOP COVID trial results, Dr. David Seftel, decided to test all the employees at the racetrack for COVID19, and, if positive, offer them treatment with fluvoxamine. None of the 65 patients that took fluvoxamine required hospitalization, but 12 and a half percent (0.5%) of the 48 patients who chose NOT to take fluvoxamine needed to be hospitalized, and one died. Although this was not a randomized trial, the results provided additional support for fluvoxamine as an early treatment for COVID-19. The findings were published in Open Forum Infectious Disease in February 2021 (see e.g., Example 5). Also, there was a study of ICU patients in Croatia, which showed patients treated with fluvoxamine at a high dose, 100 mg three times daily. In the fluvoxamine group 30 of 51 (58.8%) patients died. In the matched control group, 39 of 51 (76.5%) died. So, this was a 42% reduced risk of death in the fluvoxamine group (HR 0.58, 95% CI (0.36-0.94, P=0.027). This was published in the British Journal of Clinical Pharmacology in November 2021.

### Evidence for Treatment of COVID-19

At this point in time, there is substantial evidence supporting the use of fluvoxamine for treatment of high-risk outpatients with early COVID-19, to prevent respiratory deterioration, need for hospitalization, and death. In the US, fluvoxamine can be prescribed off-label for early symptomatic COVID-19 NOW, under principles of shared decision-making, in which the level of evidence, risks, and potential benefits are discussed, and patients provide informed consent to treatment.

Fluvoxamine has recently appeared as a treatment option in Florida public service announcements and is on some university and hospital guidelines for outpatient COVID treatment, including at Johns Hopkins University.

Fluvoxamine researchers, led by Dr. David Boulware at University of Minnesota, submitted to the FDA on December 21, an application for Emergency Use Authorization (or EUA) for the use of fluvoxamine for treatment of outpatient adults with COVID-19. There is also a letter to the FDA that healthcare workers can sign to show support for the EUA (see e.g., Example 8).

An urgent clinical need exists for an oral medicine that can treat early symptomatic COVID-19 disease to stop clinical deterioration and prevent hospitalization. The omicron variant, which is not neutralized by most currently available monoclonal antibodies, is rapidly spreading in the USA thus defeating the only currently authorized outpatient interventions shown to reduce hospitalization and mortality. Fluvoxamine, whose putative mechanism is not contingent upon variant type and is widely available, appears to meet an urgent medical need right now.

### Proposed Dosing Strategy for EUA Application:

For COVID-19 Adults: Recommended starting dose is 50 mg once increasing to 100 mg twice daily for 10 to 15 days in adults aged >24 years of age. A duration of dosing through at least 15 days of symptomatic illness is recommended.

If tolerability of fluvoxamine is a problem, a dose reduction to 50 mg twice daily is recommended.

The completed RCTs have been done with outpatients with symptoms less than 7 days. It is recommended to start soon as possible after symptom onset. Idea is to start early enough to prevent the inflammatory phase.

Fluvoxamine may also be beneficial in dampening down inflammation once the inflammatory phase has started. In hospitalized patients, there is one observational cohort study of ICU patients in Croatia showing benefit for patients given fluvoxamine compared to matched controls.

In long COVID patients, there is anecdotal reports of benefit in long haulers. RCTs are needed. For now, with the EUA application, it is only suggested to use it for early acute COVID-19 for adults more than 24 years of age, and I think it could be particularly important for those who are at high risk for clinical deterioration.

Here, it was shown that hospitalized patients who took 20 mg fluoxetine per day did better than those who did not take fluvoxamine (Nemeth et al. Ideggyogy Sz. 2021 Nov. 30; 74(11-12):389-396).

### Common Side Effects of Fluvoxamine

The most common side effects are annoying but not dangerous. Some of them can include: mild nausea, dizziness, or changes in sleep (insomnia or sleepiness). Usually these side effects go away after a few days as a person gets used to the medication.

### Drug Interactions

Fluvoxamine inhibits some enzymes in the liver that break down other drugs, so levels of these drugs may increase when a person is taking fluvoxamine. Here is a list of some of the more important ones (this is not a comprehensive list).

Melatonin: This interaction may be a good thing since increased melatonin may be helpful in treating COVID (melatonin has anti-inflammatory effects through a mechanism that is different from fluvoxamine's anti-inflammatory mechanism. Thus it is possible that the co-administration of fluvoxamine and timed melatonin might be especially beneficial (Anderson 2021 Fluvoxamine, melatonin and COVID-19 Psychopharmacology volume 238, page 611 (2021)).

Caffeine: Caffeine should be avoided when taking fluvoxamine. Fluvoxamine can make caffeine stay in the body 5 times longer than expected, which could lead to jitteriness and insomnia.

Prescription drugs with risk for serious drug interactions: This includes theophylline, clozapine, olanzapine tizanadine. People who take one of these should avoid taking fluvoxamine or talk to their doctor about how to avoid a potentially dangerous drug interaction.

Other psychiatric medications: Antidepressants and other psychiatric drugs could interact with fluvoxamine, and fluvoxamine may affect psychiatric symptoms in people who already have a psychiatric disorder. For example, people with bipolar disorder can sometimes develop manic symptoms when taking an antidepressant. People with history of any psychiatric diagnosis or taking any psychiatric medications should talk to their doctor about this before taking fluvoxamine.

Other drugs: If a patient is taking other medications, it is advisable to check for any drug interactions using a drug-interaction checker (such as those found on various websites with drug information, or those built into some electronic medical records systems).

Some should not take it due to taking medications that are contraindicated with fluvoxamine. There is also a relative contraindication for people with bipolar disorder. Individuals with bipolar disorder can sometimes develop manic symptoms when taking an antidepressant. People with a history of any psychiatric diagnosis or taking any psychiatric medications should talk to their doctor about this before taking fluvoxamine.

### Hypothesis

Clinical deterioration in COVID-19 seemed to be due to an excessive inflammatory response to the SARS-CoV-2 virus. I knew that the selective serotonin reuptake inhibitor, fluvoxamine, which is FDA-approved for treatment of Obsessive-Compulsive Disorder, also has anti-inflammatory and immunomodulatory properties through action on the signal receptor, a protein INSIDE our cells that regulates cellular responses to stress and inflammatory triggers such as infection. It was thought fluvoxamine might be helpful for preventing clinical deterioration in COVID-19 and it was decided to run a randomized clinical trial of fluvoxamine versus placebo for treatment of outpatients with early COVID-19. That first trial was started on Apr. 10, 2020.

Dosing from Trials/Other Studies

Together Trial: 100 mg BID x10 days. We expect this to be standard dosing in most protocols that include FLV since this was the dosing used in the published Phase 3 trial.

STOP COVID 1 (SC-1): tried to get up to 100 mg TID x15 days (50% got up to full dose, most others 100 BID).

Seftel & Boulware: 50 mg BID×14 days.

STOP COVID 2 (SC-2): 100 mg BID x15 days

COVID-OUT: 50 mg BID

ACTIV-6: 50 mg BID

ICU study in Croatia: 100 mg TID

Suggested Dosing for Outpatients with Early COVID-19

Start in first week of symptoms.

100 mg BID x10-15 days

OK to reduce to 50 mg BID or 100 mg qHS if poorly tolerated.

Some may need increase to 100 mg TID if more severe disease (ICU study used this dose).

Total daily dose of 300 mg is FDA maximum recommended dose for OCD.

### Timing of Treatment

The completed RCTs have been done with outpatients with symptoms less than 7 days.

Best to start soon as possible after symptom onset

Idea is to start early enough to prevent the inflammatory phase

May also be beneficial in dampening down inflammation once inflammatory phase has started

Hospitalized patients: There is one observational cohort study of ICU patients in Croatia showing benefit for patients given fluvoxamine compared to matched controls.

Long COVID: There are anecdotal reports of benefit in long haulers. RCTs are needed.

### Common Side Effects

Mild nausea, dizziness, changes in sleep (insomnia or sleepiness). Usually these side effects are mild and go away after a few days as a person gets used to the medication. Here is a drug label with full prescribing information for fluvoxamine (brand name Luvox) at the drugs@FDA website: https://www.accessdata.fda.qov/drugsatfda_docs/label/2008/022235lbl.pdf

### REFERENCES

Mehta P, McAuley D F, Brown M, et al. COVID-19: consider cytokine storm syndromes and immunosuppression. Lancet. 2020; 395(10229):1033-1034.
Nicol G E, Karp J F, Reiersen A M, et al. “What were you before the war?” J Clin Psychiatry. Published online Apr. 7, 2020. doi:10.4088/JCP.20com13373
Østergaard L. SARS CoV-2 related microvascular damage and symptoms during and after COVID-19: Consequences of capillary transit-time changes, tissue hypoxia and inflammation. Physiol Rep. 2021 February; 9(3):e14726. doi: 10.14814/phy2.14726.
Prasad A, Prasad M. Single virus targeting multiple organs. Front Med (Lausanne). 2020; 7:370.
Rosen D A, Seki S M, Fernéndez-Castañeda A, et al. Modulation of the sigma-1 receptor-IRE1 pathway is beneficial in preclinical models of inflammation and sepsis. Sci Transl Med. 2019; 11 (478):eaau5266.
Fung S, Liu D X. The ER stress sensor IRE1 and MAP kinase ERK modulate autophagy induction in cells infected with coronavirus infectious bronchitis virus. Virology 2019 July; 533:34-44. doi: 10.1016/j.virol.2019.05.002. Epub 2019 May 2.
Gordon, et al. Comparative host-coronavirus protein interaction networks reveal pan-viral disease mechanisms. Science. 2020 Dec. 4; 370(6521):eabe9403. doi: 10.1126/science.abe9403. Epub 2020 Oct. 15.
Hayashi T. The Sigma-1 Receptor in Cellular Stress Signaling. Front Neurosci. 2019 Jul. 16; 13:733. doi: 10.3389/fnins.2019.00733. eCollection 2019.
Homolak J, Kodvanj I. Widely available lysosome targeting agents should be considered as potential therapy for COVID-19. Int J Antimicrob Agents. 2020; 56(2):106044.
Ghosh S, Dellibovi-Ragheb T A, Kerviel A, Pak E, Qiu Q, Fisher M, Takvorian P M, Bleck C, Hsu V W, Fehr A R, Perlman S, Achar S R, Straus M R, Whittaker G R, de Haan C A M, Kehrl J, Altan-Bonnet G, Altan-Bonnet N. Beta-Coronaviruses Use Lysosomes for Egress Instead of the Biosynthetic Secretory Pathway. Cell. 2020 Dec. 10; 183(6):1520-1535.e14. doi: 10.1016/j.cell.2020.10.039. Epub 2020 Oct. 27.
Hoertel N, Sánchez-Rico M, Gulbins E, Kornhuber J, Carpinteiro A, Lenze E J, Reiersen A M, Abellen M, de la Muela P, Vernet R, Blanco C, Beeker N, Neuraz A, Gorwood P, Alvarado J M, Meneton P, Limosin F. Association between Functional Inhibitors of Acid Sphingomyelinase and Reduced Risk of Intubation or Death in Individuals Hospitalized for Severe COVID-19: results from an observational multicenter study. medRxiv 2021.02.22.21252209; doi: https://doi.org/10.1101/2021.02.22.21252209 (pre-print).
Carpinteiro A, Edwards M J, Hoffmann M, Kochs G, Gripp B, Weigang S, Adams C, Carpinteiro E, Gulbins A, Keitsch S, Sehl C, Soddemann M, Wilker B, Kamler M, Bertsch T, Lang K S, Patel S, Wilson G C, Walter S, Hengel H, Pohlmann S, Lang P A, Kornhuber J, Becker K A, Ahmad S A, Fassbender K, Gulbins E. Pharmacological Inhibition of Acid Sphingomyelinase Prevents Uptake of SARS-CoV-2 by Epithelial Cells. Cell Rep Med. 2020 Nov. 17; 1(8):100142. doi: 10.1016/j.xcrm.2020.100142. Epub 2020 Oct. 29.
Theoharides T C, Antonopoulou S, Demopoulos C A. Coronavirus 2019, Microthromboses, and Platelet Activating Factor Clin Ther. 2020 October; 42(10):1850-1852. doi: 10.1016/j.clinthera.2020.08.006. Epub 2020 Aug. 17.
Theoharides T C, Conti P. COVID-19 and Multisystem Inflammatory Syndrome, or is it Mast Cell Activation Syndrome? J Biol Regul Homeost Agents. 2020 September-October; 34(5):1633-1636. doi: 10.23812/20-EDIT3.
Chen Z, Xiao L, Chen J, Luo H, Wang G, Huang Y, Wang X. Effects of fluoxetine on mast cell morphology and protease-1 expression in gastric antrum in a rat model of depression. World J Gastroenterol. 2008 Dec. 7; 14(45):6993-8. doi: 10.3748/wjg.14.6993.
Schlienger R G, Meier C R. Effect of selective serotonin reuptake inhibitors on platelet activation: can they prevent acute myocardial infarction? Am J Cardiovasc Drugs. 2003; 3(3):149-62. doi: 10.2165/00129784-200303030-00001.
Harpsoe N G, Andersen L P H, Gogenur I, Rosenberg J. Clinical pharmacokinetics of melatonin: a systematic review. Eur J Clin Pharmacol. 2015 August; 71(8):901-9. doi: 10.1007/s00228-015-1873-4. Epub 2015 May 27.
Shneider A, Kudriavtsev A, Vakhrusheva A. Can melatonin reduce the severity of COVID-19 pandemic? Int Rev Immunol. 2020; 39(4):153-162. doi: 10.1080/08830185.2020.1756284. Epub 2020 Apr. 29.
Lenze E J, Mattar C, Zorumski C F, Stevens A, Schweiger J, Nicol G E, Miller J P, Yang L, Yingling M, Avidan M S, Reiersen A M. Fluvoxamine vs Placebo and Clinical Deterioration in Outpatients With Symptomatic COVID-19: A Randomized Clinical Trial. JAMA. 2020 Dec. 8; 324(22):2292-2300. doi: 10.1001/jama.2020.22760.
Rodebaugh T L, Frumkin M R, Reiersen A M, Lenze E J, Avidan M S, Miller J P, Piccirillo J F, Zorumski C F, Mattar C. Acute symptoms of mild to moderate COVID-19 are highly heterogeneous across individuals and over time. Open Forum Infectious Diseases, ofab090, https://doi.org/10.1093/ofid/ofab090 Published: 1 Mar. 2021

### Example 2: Fluvoxamine Vs Placebo and Clinical Deterioration in Outpatients with Symptomatic COVID-19: A Randomized Clinical Trial

Coronavirus disease 2019 (COVID-19) may lead to serious illness as a result of an excessive immune response. Fluvoxamine was shown to prevent clinical deterioration by stimulating the σ-1 receptor, which regulates cytokine production.

### Key Points

Question: Does fluvoxamine, a selective serotonin reuptake inhibitor (SSRI) and σ-1 receptor (S1R) agonist, prevent clinical deterioration in outpatients with acute coronavirus disease 2019 (COVID-19)?

Findings: In this randomized trial that included 152 adult outpatients with confirmed COVID-19 and symptom onset within 7 days, clinical deterioration occurred in 0 patients treated with fluvoxamine vs 6 (8.3%) patients treated with placebo over 15 days, a difference that was statistically significant.

Meaning: In this preliminary study, adult outpatients with symptomatic COVID-19 treated with fluvoxamine, compared with placebo, had a lower likelihood of clinical deterioration over 15 days; however, determination of clinical efficacy would require larger randomized trials with more definitive outcome measures.

### Abstract

Importance: Coronavirus disease 2019 (COVID-19) may lead to serious illness as a result of an excessive immune response. Fluvoxamine may prevent clinical deterioration by stimulating the 6-1 receptor, which regulates cytokine production.

Objective: To determine whether fluvoxamine, given during mild COVID-19 illness, prevents clinical deterioration and decreases the severity of disease.

Design, Setting, and Participants: Double-blind, randomized, fully remote (contactless) clinical trial of fluvoxamine vs placebo. Participants were community-living, nonhospitalized adults with confirmed severe acute respiratory syndrome coronavirus 2 infection, with COVID-19 symptom onset within 7 days and oxygen saturation of 92% or greater. One hundred fifty-two participants were enrolled from the St Louis metropolitan area (Missouri and Illinois) from Apr. 10, 2020, to Aug. 5, 2020. The final date of follow-up was Sep. 19, 2020.

Interventions: Participants were randomly assigned to receive 100 mg of fluvoxamine (n=80) or placebo (n=72) 3 times daily for 15 days.

Main Outcomes and Measures: The primary outcome was clinical deterioration within 15 days of randomization defined by meeting both criteria of (1) shortness of breath or hospitalization for shortness of breath or pneumonia and (2) oxygen saturation less than 92% on room air or need for supplemental oxygen to achieve oxygen saturation of 92% or greater.

Results: Of 152 patients who were randomized (mean [SD] age, 46 [13] years; 109 [72%] women), 115 (76%) completed the trial. Clinical deterioration occurred in 0 of 80 patients in the fluvoxamine group and in 6 of 72 patients in the placebo group (absolute difference, 8.7% [95% CI, 1.8%-16.4%] from survival analysis; log-rank P=0.009). The fluvoxamine group had 1 serious adverse event and 11 other adverse events, whereas the placebo group had 6 serious adverse events and 12 other adverse events.

Conclusions and Relevance: In this preliminary study of adult outpatients with symptomatic COVID-19, patients treated with fluvoxamine, compared with placebo, had a lower likelihood of clinical deterioration over 15 days. However, the study is limited by a small sample size and short follow-up duration, and determination of clinical efficacy would require larger randomized trials with more definitive outcome measures.

Trial Registration: ClinicalTrials.gov Identifier: NCT04342663

### Introduction

Coronavirus disease 2019 (COVID-19), caused by infection with the novel severe acute respiratory syndrome coronavirus 2 (SARS-CoV-2), can result in serious illness leading to hospitalization, intensive care unit admission, and death.¹ Clinical deterioration typically occurs during the second week of illness.

Early studies of COVID-19 found that hospitalization most often occurs within 8 to 10 days of initially mild to moderate symptoms.²⁴ Further evidence suggested that lung damage from COVID-19 was related to an excessive inflammatory response, prompting numerous trials of immunomodulatory drugs.^{5,6}

A potential mechanism for immune modulation is 6-1 receptor (S1R) agonism.⁷ The S1R is an endoplasmic reticulum chaperone protein with various cellular functions, including regulation of cytokine production through its interaction with the endoplasmic reticulum stress sensor inositol-requiring enzyme 1a (IRE1). Previous studies have shown that fluvoxamine, a selective serotonin reuptake inhibitor (SSRI) with high affinity for the S1R,⁶ reduced damaging aspects of the inflammatory response during sepsis through the S1R-IRE1 pathway, and decreased shock in murine sepsis models.⁹

Fluvoxamine is a strong S1R agonist,^{10,11} is highly lipophilic, and has rapid intracellular uptake.¹² This study tested whether fluvoxamine, given as early treatment in individuals with mild COVID-19 illness, may prevent clinical deterioration.

### Methods

This was a double-blind, placebo-controlled, randomized clinical trial that compared fluvoxamine with placebo in adult outpatients with confirmed SARS-CoV-2 infection. The trial protocol and statistical analysis plan appear in Example 3. The study was approved by the institutional review board at Washington University in St Louis and was conducted in compliance with the Declaration of Helsinki,¹³ the Good Clinical Practice guidelines, and local regulatory requirements. All participants provided informed consent via e-consent or written consent.

### Study Design

This trial was conducted in the greater St Louis metropolitan area (eastern Missouri and southern Illinois). Patients were recruited from Apr. 10, 2020, to Aug. 5, 2020. The 30-day postrandomization follow-up assessment was completed on Sep. 19, 2020. This was a fully remote (contactless) clinical trial.¹⁴ Participants were recruited via electronic health records, physician and other health professional referrals, study advertisements near COVID-19 testing centers and in emergency departments, referrals by colleagues, a study website, and communication in local television and newspapers. Participants were enrolled without regard to sex, race, ethnicity, or religion. Potential participants underwent screenings by email and phone, and provided informed consent, typically electronically.

Study supplies were delivered to self-quarantined study patients as a package left at their door and the study materials consisted of the study medication, an oxygen saturation monitor, an automated blood pressure monitor, and a thermometer. Participants then self-assessed using the equipment provided and confirmed vital signs within range (systolic blood pressure between 80 mm Hg and 200 mm Hg, diastolic blood pressure between 40 mm Hg and 120 mm Hg, and pulse rate between 50 beats/min and 120 beats/min), pregnancy status when indicated, and oxygen saturation of 92% or greater. Study staff called participants, informed them of eligibility, and instructed them to take the study medication. The study medication was targeted to start on the same day that participants were first contacted and screened by the research team.

All data collection was done by twice-daily REDCap surveys sent to patients via email, with phone-based data collection as backup to ensure that individuals without internet access were able to participate. The surveys recorded oxygen saturation, vital signs, medication adherence, and COVID-19 symptoms. Fixed race and ethnicity categories were used by interviewers as part of the demographic information collected to characterize the sample.

Dyspnea (shortness of breath) was measured using a continuous scale (0=symptom is not present and 10=symptom is very severe) with the Ecological Momentary Assessment¹⁵ (i.e., “how bad is your symptom right now?”). Phone contact was attempted daily during the first 3 days of the trial to address participants' questions, address any medication-related issues, and encourage assessment completion. Additional phone calls were conducted on a case-by-case basis when participants' survey data indicated values outside the ranges. For participants who had worsening COVID-19 illness, study staff recommended they seek medical attention.

### Participants

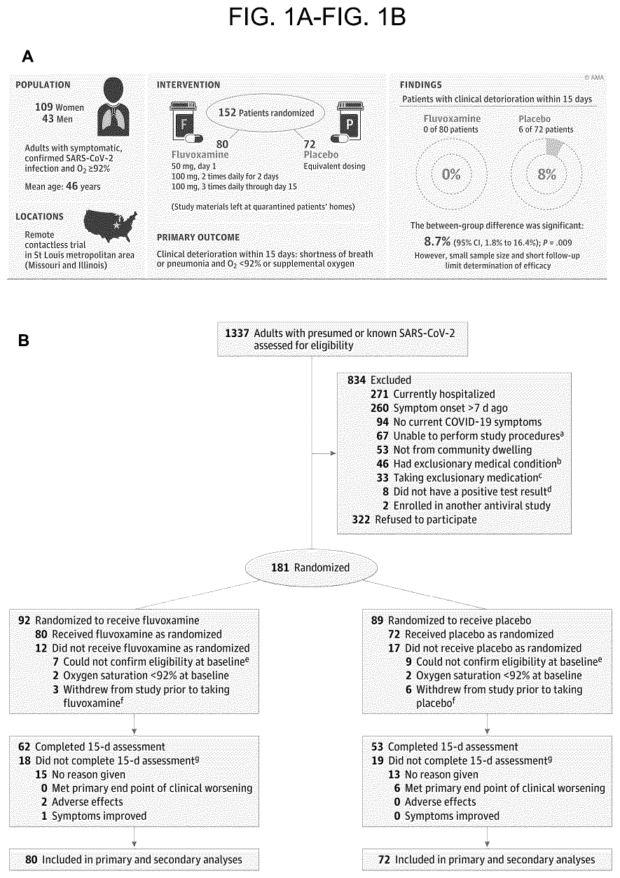

The study included adults living in the community with SARS-CoV-2 infection confirmed by polymerase chain reaction assay and who were symptomatic within 7 days of the first dose of study medication (FIG. 1B). Exclusion criteria included having COVID-19 that required hospitalization or evidence of the primary end point with oxygen saturation less than 92% on room air at the time of randomization. Other exclusion criteria were severe underlying lung disease (e.g., chronic obstructive pulmonary disease or required home oxygen, interstitial lung disease, pulmonary hypertension), decompensated cirrhosis, congestive heart failure (New York Heart Association class III or IV), or immunocompromised (e.g., solid organ transplant recipient or donor, bone marrow transplant recipient, AIDS, or taking immunosuppressant biologic drugs or high-dose corticosteroids [>20 mg/d of prednisone]; additional details appear in Example 3).

### Randomization

Patients were randomized 1:1 to fluvoxamine or matching placebo capsules. Randomization schedules were generated that stratified by age (18-44, 45-54, 55-64, and >65 years)¹⁶ and sex. Treatments were randomly allocated using alternating block sizes of 2 and 4. Randomization allocation was conducted via REDCap, which displayed randomization assignment to the laboratory manager (J.S.), who prepared the study materials, including the study drug or placebo. All outcome assessors, investigators, and research staff who were in contact with participants were blinded to participant treatment assignment.

### Intervention

Participants received a dose of 50 mg of fluvoxamine (or matching placebo) in the evening immediately after the baseline assessment and confirmation of eligibility, then for 2 days at a dose of 100 mg twice daily as tolerated, and then increasing to a dose of 100 mg 3 times daily as tolerated through day 15 then stopped (additional details appear in Example 3). This dose range was determined based on the binding affinity of fluvoxamine for the S1R.¹⁷ After the completion of 15 days of fluvoxamine or placebo, participants were given the option to receive a 6-day open-label course of fluvoxamine. This optional open-label phase was a change from the original study protocol.

### Primary and Secondary End Points

The primary end point was clinical deterioration defined by both the (1) presence of dyspnea (i.e., shortness of breath) or hospitalization for shortness of breath or pneumonia and (2) decrease in oxygen saturation (<92%) on room air or supplemental oxygen requirement to maintain oxygen saturation of 92% or greater. The primary end point was corroborated by phone discussion with participants and review of the medical records.

For the secondary end points, episodes of clinical deterioration were rated on a novel 7-point scale with 0 indicating none; 1, shortness of breath and oxygen saturation less than 92% but no supplemental oxygen needed; 2, shortness of breath and oxygen saturation less than 92% plus supplemental oxygen needed; 3, oxygen saturation less than 92% plus supplemental oxygen needed and hospitalization related to dyspnea or hypoxia; 4, oxygen saturation less than 92% plus supplemental oxygen needed and hospitalization related to dyspnea or hypoxia plus ventilator support needed for less than 3 days; 5, oxygen saturation less than 92% plus supplemental oxygen needed and hospitalization related to dyspnea or hypoxia plus ventilator support needed for at least 3 days; and 6, death. The number of days requiring supplemental oxygen, hospitalization, and ventilator support also were assessed.

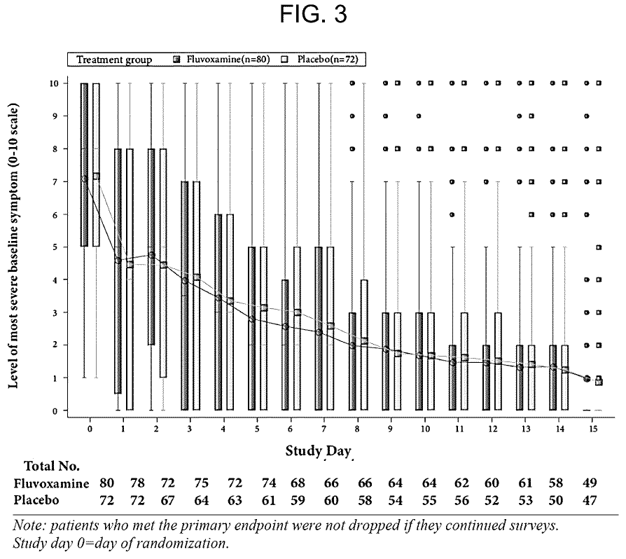

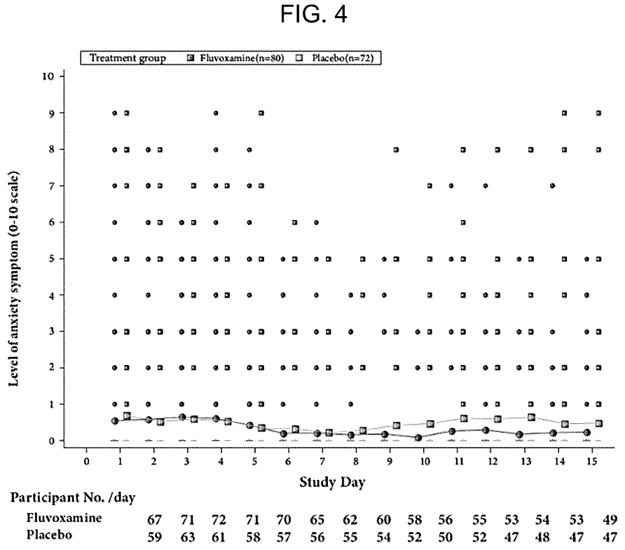

A prespecified secondary end point in the study protocol was symptomatic severity during the 15 days of the trial using a continuous scale of each patient's most severe baseline symptom on an 11-point scale (0=symptom is not present and 10=symptom is very severe). This analytic strategy was flawed (FIG. 3) and we did not pursue further analyses. As a post hoc analysis, self-reported anxiety levels were examined and were measured on the same 11-point scale because anxiety may relate to shortness of breath (FIG. 4). Clinical deterioration was ranked using the World Health Organization ordinal scale for COVID-19 trials (TABLE 1).¹⁸

| | Fluvoxamine | | | |
|---|---|---|---|---|
| | group | | Placebo group | |
| Outcomes | (n = 80) | | (n = 72) | |
| Clinical deterioration, No (%) | | | | |
| 0 = None | 80 | (100%) | 66 | (91.7%) |
| 1 = O₂ sat <92% but no supplemental O₂ | 0 | (0%) | 2 | (2.8%) |
| 2 = Above + supplemental O₂ needed | 0 | (0%) | 0 | (0%) |
| 3 = Above + hospitalization needed | 0 | (0%) | 3 | (4.2%) |
| 4 = Above + ventilator needed (<3 days) | 0 | (0%) | 0 | (0%) |
| 5 = Above + ventilator needed (≥3 days) | 0 | (0%) | 1 | (1.4%) |
| 6 = Death | 0 | (0%) | 0 | (0%) |
| Total participants with values >0 | 0 | (0%) | 6 | (8.3%) |

| | Fluvoxamine | | | |
|---|---|---|---|---|
| | group | | Placebo group | |
| Outcomes | (n = 80) | | (n = 72) | |
| WHO scale, No (%)^{a} | | | | |
| 0-1 = uninfected or ambulatory | 80 | (100%) | 66 | (91.7%) |
| 2 = limitation of activities | 0 | (0%) | 2 | (2.8%) |
| 3 = hospitalized, no O₂ needed | 0 | (0%) | 0 | (0%) |
| 4 = hospitalized, O₂ needed | 0 | (0%) | 3 | (4.2%) |
| 5 = non-invasive vent or high-flow O₂ | 0 | (0%) | 0 | (0%) |
| 6 = ventilator needed | 0 | (0%) | 1 | (1.4%) |
| 7 = ventilator + additional organ | 0 | (0%) | 0 | (0%) |
| support | | | | |
| 8 = Death | 0 | (0%) | 0 | (0%) |
| Total participants with values >1 | 0 | (0%) | 6 | (8.3%) |

The primary and secondary end points were measured using participants' self-reported responses on twice-daily surveys during the 15 days after randomization that were corroborated by research staff with phone contact. For participants who had stopped responding to the surveys prior to day 15 or who had met the primary end point, medical records and subsequent calls to these participants were used to determine whether they met the primary end point. For participants who met the primary end point, hospital records were used to confirm specific health care use (e.g., supplemental oxygen use, hospital length of stay, ventilator support). Adverse events and serious adverse events were recorded each day via participant self-report for 15 days after randomization.

At 30 days after the conclusion of the 15-day trial, a follow-up survey was performed asking, “Have you visited a hospital or emergency department since your last study survey 30 days ago?” This nonprespecified end point was confirmed by phone, email, or electronic medical record review.

### Statistical Analysis

Patients were analyzed according to randomization group. Based on 80% power, an a level of 0.05, a rate of 20% for clinical deterioration in the placebo group, and a reduction of 75% in the risk of clinical deterioration in the fluvoxamine group, a total sample size of 152 participants was required. This magnitude of risk reduction was chosen because discovery of a large effect would be of major clinical importance and warrant further study.

As prespecified in the study protocol, the full analysis set included only participants who were confirmed eligible and started taking the study medication, which is consistent with the principles of infectious disease clinical trials.¹⁹ A study statistician (L.Y.) conducted the blinded analysis under the supervision of a senior biostatistician (J.P.M.) prior to unblinding. No interim analysis was conducted.

The primary analysis was the survival analysis for the primary outcome (clinical deterioration) using a log-rank test. This analysis treated participants a priori as censored on the day that they met the primary outcome, or on the last day that they filled out an outcome assessment. The rate of missingness for survey completion was measured. To determine if missingness was nonrandom, the available scores immediately before and after each missing score and their mean were compared with the total mean score for both shortness of breath and oxygen saturation.

Because of the potential for type I error due to multiple comparisons, the analysis of the secondary end points was exploratory. SAS version 9.4 (SAS Institute Inc) was used for all the analyses. Significance was set as a 2-tailed a level of 0.05.

### Results

### Patient Characteristics

Of 1337 patients screened, 834 (62%) were excluded, 322 (24%) were contacted and declined participation, and 181 (14%) were randomized and provided with study materials (FIG. 1B). Of the 181 patients randomized, 20 were excluded (9 in the fluvoxamine group and 11 in the placebo group), 9 never began taking the study medication (3 in the fluvoxamine group and 6 in the placebo group), and 152 started the study and constituted the primary analysis set. Among the 152 patients, 140 (92%) took the first dose of study medication on the same day they were first contacted by study staff (the rest started it the day after contact). A total of 35 participants opted to take open-label fluvoxamine after the double-blind phase, but no data collection was conducted for this phase.

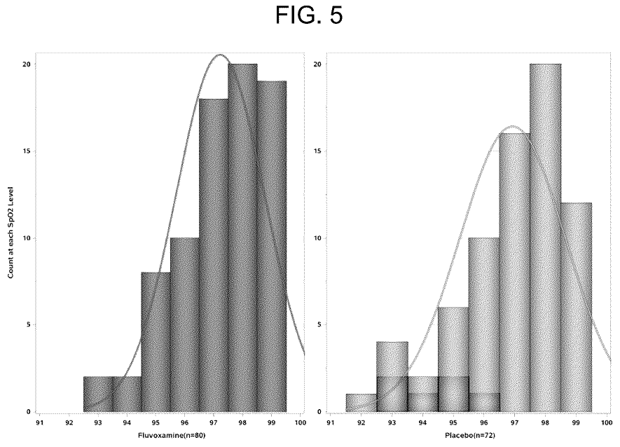

Participants were well matched in demographic and clinical characteristics (TABLE 2). Of the 152 participants, 38 (25%) were Black adults and the mean age was 46 years (SD, 13 years). The most severe presenting COVID-19 symptom varied, with fatigue (23%) and loss of sense of smell (29%) being the most common. The baseline oxygen saturation level did not differ between the groups (median of 97% [interquartile range, 96%-98%] for fluvoxamine vs 97% [interquartile range, 96%-98%] for placebo (distributions shown in FIG. 5).

TABLE 2
Baseline Characteristics.

| | Fluvoxamine (n = 80) | | Placebo (n = 72) | |
|---|---|---|---|---|
| Age, median (IQR) [range], y | 46 (35-58) [20-75] | | 45 (36-54) [21-69] | |
| Sex at birth, No. (%) | | | | |
| Female | 56 | (70) | 53 | (74) |
| Male | 24 | (30) | 19 | (26) |
| Race, No. (%)^{a} | | | | |
| White | 56 | (70) | 50 | (69) |
| Black | 18 | (23) | 20 | (28) |
| Asian | 3 | (4) | 1 | (1) |
| Other | 2 | (3) | 1 | (1) |
| Unknown | 1 | (1) | 0 | |
| American Indian/Alaska Native | 0 | | 1 | (1) |
| Ethnicity, No. (%)^{a} | | | | |
| Non-Hispanic/Non-Latino | 75 | (94) | 66 | (92) |
| Hispanic/Latino | 3 | (4) | 2 | (3) |
| Unknown/not reported | 2 | (3) | 4 | (5) |
| Coexisting conditions, No. (%)^{a} | | | | |
| Asthma | 17 | (21) | 9 | (13) |
| Hypertension | 15 | (19) | 15 | (21) |
| Diabetes | 9 | (11) | 8 | (11) |
| High cholesterol | 7 | (9) | 7 | (10) |
| Hyperthyroidism | 6 | (8) | 6 | (8) |
| Anxiety | 5 | (6) | 1 | (1) |
| Arthritis^{b} | 4 | (5) | 3 | (4) |
| Depression | 1 | (1) | 4 | (6) |
| Body mass index category, No. (%)^{c} | | | | |
| Underweight (<18.5) | 1 | (1) | 1 | (1) |
| Normal (18.5-24.9) | 14 | (18) | 7 | (10) |
| Overweight (25-29.9) | 22 | (28) | 22 | (31) |
| Obese (≥30) | 43 | (54) | 42 | (58) |
| Duration of COVID-19 symptoms, | 4 (3-5) [1-7] | | 4 (3-5) [1-7] | |
| median (IQR) [range], d^{a} | | | | |
| Oxygen saturation, | 97 (96-98) [93-99] | | 97 (96-98) [92-99] | |
| median (IQR) [range], % | | | | |
| Most severe COVID-19 symptom | | | | |
| at baseline, No. (%)^{a} | | | | |
| Loss of sense of smell | 26 | (33) | 18 | (25) |
| Fatigue | 17 | (21) | 18 | (25) |
| Body aches | 9 | (11) | 13 | (18) |
| Cough | 9 | (11) | 1 | (1) |
| Subjective fever | 8 | (10) | 4 | (6) |
| Loss of appetite | 3 | (4) | 8 | (11) |
| Chills | 3 | (4) | 6 | (8) |
| Shortness of breath | 2 | (3) | 1 | (1) |
| Loss of taste | 2 | (3) | 2 | (3) |
| Nausea | 1 | (1) | 1 | (1) |

Abbreviations:
COVID-19, coronavirus disease 2019; IQR, interquartile range.
Abbreviations:
COVID-19, coronavirus disease 2019; IQR, interquartile range.
^{a}Per participant self-report.
^{b}Osteoarthritis or rheumatoid arthritis;
^{c}Calculated as weight in kilograms divided by height in meters squared.

TABLE 3
Most severe COVID-19 symptom at baseline,
by group (data are from TABLE 2).

| | | |
|---|---|---|
| Most severe symptom | Fluvoxamine | Placebo |
| at baseline^{a}, No (%) | (n = 80) | (n = 72) |

| | | | | |
|---|---|---|---|---|
| Loss of smell | 26 | (32.5%) | 18 | (25%) |
| Fatigue | 17 | (21.3%) | 18 | (25%) |
| Body aches | 9 | (11.3%) | 13 | (18.1%) |
| Cough | 9 | (11.3%) | 1 | (1.4%) |
| Subjective fever | 8 | (10%) | 4 | (5.6%) |
| Loss of appetite | 3 | (3.8%) | 8 | (11.1%) |
| Chills | 3 | (3.8%) | 6 | (8.3%) |
| Shortness of breath | 2 | (2.5%) | 1 | (1.4%) |
| Loss of taste | 2 | (2.5%) | 2 | (2.8%) |
| Nausea | 1 | (1.3%) | 1 | (1.4%) |

^{a}per patient self-report

### Efficacy of Fluvoxamine vs Placebo

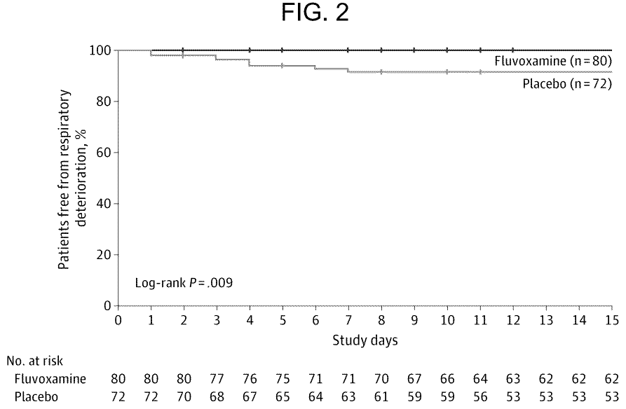

Clinical deterioration occurred in 0 of 80 patients in the fluvoxamine group and in 6 of 72 (8.3%) patients in the placebo group (absolute difference, 8.7% [95% CI, 1.8%-16.4%] by survival analysis, log-rank χ²=6.8 and P=0.009; TABLE 3 and FIG. 2). In the placebo group, cases of clinical deterioration ranged from 1 to 7 days after randomization and from 3 to 12 days after the onset of COVID-19 symptoms. Four of 6 patients were hospitalized for COVID-19 illness, with the length of stay ranging from 4 to 21 days. One patient required mechanical ventilation for 10 days (TABLE 3) and no patients died. Detailed vignettes of clinical deterioration appear below.

Summaries of the Six Participants Who Showed Clinical Deterioration (all Placebo, all Who were Hospitalized were Also Considered SAEs)

Note: an age range, rather than exact age, is given to protect participant identity

1. Low oxygen saturation plus shortness of breath: A 35-39 year-old, white, Hispanic male participant entered the study with a reported baseline oxygen saturation of 95% and a shortness of breath rating of 0 out of 10. He reported low oxygen saturation on the Day 7 morning survey of 90% as well as a shortness of breath rating of 5/10. Study staff called the participant and a re-check on the pulse oximeter showed an oxygen saturation of 91%. The participant went to the ER, where his oxygen saturation and shortness of breath reportedly improved and he was discharged home. Clinical deterioration occurred 12 days after symptom onset.

2. Low oxygen saturation plus shortness of breath: A 30-34 year-old white, non-Hispanic male entered the study with a baseline oxygen saturation of 93% and a shortness of breath rating of 7 out of 10. The participant reported oxygen saturation of 90% and shortness of breath at 6/10 on the Day 6 Evening Survey. On the Day 7 Morning Survey he reported oxygen saturation of 91% with shortness of breath at 6/10. Study staff called him and he said that when he re-checked, his oxygen was 89% so he called his PCP. His PCP had him check again while she was on the phone with him and his oxygen saturation was 91%. His PCP told him to go to the ER if his oxygen saturation dipped below 90% again. Clinical deterioration occurred 10 days after symptom onset.

3. Acute on chronic respiratory failure with hypoxia and hypercapnia: A 55-59 year-old black, non-Hispanic female entered the study with an oxygen saturation of 94% and a shortness of breath rating of 0/10. She began study medication that evening. The following morning she recorded an oxygen saturation of 75% on her Morning Survey, though shortness of breath score remained at 0/10. Study staff called and asked her to re-check. Her oxygen saturations remained low at 75% and she complained of weakness and dizziness. She was advised to go to the ER. Records state that in the ambulance she had an oxygen saturation of 74% and received supplemental oxygen. A chest x-ray showed bilateral infiltrates. She was admitted and hospitalized a total of 21 days. During that hospital stay, she was intubated and on a ventilator for 10 days. No other organ support (e.g., pressors, renal replacement therapy, ECMO). Clinical deterioration occurred 3 days after symptom onset.

4. Pneumonia-bilateral, multifocal: A 30-34 year-old black, non-Hispanic female entered the study with a baseline oxygen saturation of 95% and a shortness of breath rating of 0/10. On her Day 4 evening survey, she reported an oxygen saturation of 90% and a shortness of breath rating of 4/10.

Her Day 5 morning survey showed an oxygen saturation of 91% and shortness of breath at 5/10. Study staff tried calling multiple times without success. The participant recorded a Day 5 evening survey with an oxygen saturation of 89% and 6/10 shortness of breath. Staff was finally able to reach her the morning of Day 6 and recommended she call her PCP or go to the ER. After some hesitancy the participant went to the ER that same day and was subsequently hospitalized. A chest x-ray showed bilateral, multi-focal pneumonia. Records indicate that her room air oxygen saturation when admitted ranged from 89-92%, and she was (2 L/minute). She was hospitalized for 4 days. Clinical deterioration occurred 10 days after symptom onset.

5. Exacerbation of COVID symptoms (nausea/vomiting/fever/low oxygen saturation): A 55-59 year-old white, non-Hispanic female entered the study with a baseline oxygen saturation of 93% and a shortness of breath score of 0/10. The participant reported on her Day 4 Morning survey an oxygen saturation of 94% with 0/10 shortness of breath, but moderate to severe symptoms of nausea, vomiting, diarrhea and a fever. She went to the ER and was admitted later that day. Imaging revealed pneumonia. Records indicate that when on room air, her oxygen saturations during admission were in the 80 s, so she received supplemental oxygen (3 L/minute) up until the last day of her hospitalization. She was admitted a total of 6 days. Clinical deterioration occurred 8 days after symptom onset.

6. Exacerbation of COVID symptoms (nausea/fever/pneumonia): A 65-69 year-old black, non-Hispanic male entered the study with an oxygen saturation of 96% and a shortness of breath rating of 0/10. The participant reported an oxygen saturation of 95% on his morning and evening Day 2 surveys, but a shortness of breath score of 5/10 in the morning and 8/10 in the evening. The following day he was admitted to the hospital for fever and nausea. He underwent a chest x-ray which showed opacities. He was hospitalized a total of 8 days, and received supplemental oxygen for 3 of those days to keep oxygen (5 L/minute) at or above 92%. Clinical deterioration occurred 6 days after symptom onset.

Additional Serious Adverse Events During 15-Day RCT (that Did not Meet Endpoint Criteria).

### Placebo Group:

1. Exacerbation of COVID symptoms (nausea, vomiting, chest pain): A 50-54 year-old black, non-Hispanic female participant did not send survey data for Day 2, but went that day to the ER for nausea, vomiting and chest pain. She was then admitted to the hospital for 2 days. Oxygen saturation at admission was 97% and does not appear to have gone under 92%. She was not given supplemental oxygen during her admission. Chest x-ray showed pneumonia of both lungs.

2. Flank Pain: Same participant (same person as immediately above) went to ER with flank pain and was hospitalized for one day. Her oxygen saturation stayed above 92% and she did not receive any supplemental oxygen.

### Additional Details of Adverse Events in Both Groups

One fluvoxamine participant was hospitalized for dehydration (serious adverse event), but they never had an oxygen saturation measurement below 92%. One fluvoxamine participant had both shortness of breath and gastroenteritis (counted as one non-serious adverse event). Ten other fluvoxamine participants had only one simple non-serious adverse event (only one symptom/problem reported per event). One of these ten participants reported shortness of breath as their non-serious adverse event, but never had an oxygen saturation measurement below 92%. Two placebo group participants had only one non-serious adverse event (chest pain or vomiting). One placebo group participant had three separate non-serious adverse events (pneumonia, vomiting, migraine headache). Two placebo group participants had an event with low oxygen saturation and shortness of breath without need for hospitalization (non-serious adverse events, but met criteria for clinical deterioration). One of these had worsening shortness of breath without low oxygen saturation as an earlier non-serious adverse event. One placebo group participant had four separate non-serious adverse events (nausea/vomiting, shortness of breath, pneumonia, chest tightness), then had an exacerbation of COVID19 symptoms with hospitalization for nausea, vomiting, and chest pain (serious adverse event) plus a second hospitalization for flank pain (second serious adverse event), but never had oxygen saturation below 92%. One placebo participant had exacerbation of COVID symptoms with hospitalization for nausea, vomiting, fever, low oxygen saturation, and pneumonia (serious adverse event). One placebo participant had no shortness of breath but was hospitalized for acute on chronic respiratory failure with hypoxia and hypercapnia, and had bilateral infiltrates on chest x-ray (serious adverse event). One placebo participant had hospitalization for bilateral multi-focal pneumonia with shortness of breath and low oxygen saturation (serious adverse event). One placebo participant was hospitalized for exacerbation of COVID symptoms with nausea, fever, with requirement of supplemental oxygen to maintain oxygen saturation above 92% (serious adverse event).

### Fluvoxamine Group:

1. Dehydration: A 70-74 year-old white, non-Hispanic male participant called an ambulance after feeling weak and faint on Day 3. His oxygen saturations on both surveys that day were 94%. He was admitted to the hospital for dehydration for 2 days. His oxygen saturation did not drop <92% and he was not given supplemental oxygen. There was also no disruption of study medication.

Additional Hospitalizations/ER Visits During 30-Day Post-RCT Observation period

### Placebo Group:

1. Chest pain: A 45-49 year-old white, non-Hispanic female participant went to the Emergency Room complaining of chest pain and light-headedness. This occurred 27 days after the end of the double-blind phase. She was discharged home after treatment for costochondritis COVID-19 sequela. A chest x-ray was clear and no respiratory problems or hypoxia were noted.

### Fluvoxamine Group:

1. Headache: A 40-44 year old white, non-Hispanic female was hospitalized for a headache 2 days after completing the RCT phase of the study, and approximately 11 days after discontinuing study medication (took fluvoxamine for 6 days in the RCT). She was differential diagnosis was migraine vs. post-COVID headache. No respiratory problems or hypoxia were noted.

TABLE 3
Primary, Secondary, and Nonprespecified Outcomes
Table 2. Primary, Secondary, and Nonprespecified Outcomes

| | Fluvoxamine | | Placebo | | Absolute difference | | |
|---|---|---|---|---|---|---|---|
| | (n = 80) | | (n = 72) | | (95% CI)^{a} | | P value^{b} |
| Primary end point | | | | | | | |
| Clinical deterioration (met both criteria), No. (%)^{c} | 0 | | 6 | (8.3) | 8.7 | (1.8 to 16.4) | .009 |
| Secondary end points | | | | | | | |
| Clinical status on 7-point scale, No. (%)^{d} | | | | | | | |
| 0 (none) | 80 | (100) | 66 | (91.7) | 8.3 | (0.6 to 18.4) | .009 |
| Any nonzero value | 0 | | 6 | (8.3) | −8.3 | (−18.4 to −0.5) | .009 |
| 1 (shortness of breath and oxygen saturation | 0 | | 2 | (2.8) | −2.8 | (−10.8 to 3.5) | .15 |
| <92% but no supplemental oxygen needed) | | | | | | | |
| 3 (oxygen saturation <92% plus supplemental oxygen | 0 | | 3 | (4.2) | −4.2 | (−13.2 to 2.0) | .07 |
| needed and hospitalization related to dyspnea or hypoxia) | | | | | | | |
| 5 (oxygen saturation <92% plus supplemental oxygen | 0 | | 1 | (1.4) | −1.4 | (−8.4 to 4.4) | .36 |
| needed and hospitalization related to dyspnea or | | | | | | | |
| hypoxia plus ventilator support needed for ≥3 days) | | | | | | | |
| Clinical status on 7-point scale, mean (SD) | 0 | | 0.22 | (0.84) | −0.22 | (−0.41 to −0.04) | .02 |
| Clinical deterioration, No. of days^{e} | NA | | NA | | NA | | NA |
| Most severe baseline symptom change score | −5.6 | | −5.8 | | 0.3 | (−0.8 to 1.4) | .63 |
| (difference between baseline and final rating)^{f} | | | | | | | |
| Nonprespecified end points | | | | | | | |
| 30-d post trial observation events (emergency | 1 | (1.3) | 1 | (1.4) | −0.1 | (−6.7 to 5.1) | >.99 |
| department visit, hospitalization, or both)^{g} | | | | | | | |

Abbreviation:
NA, not applicable (see footnote “e” for explanation).
^{a}For outcomes reported as No. (%), the absolute difference is a difference in proportions. For other variables, the difference between group means is reported. Most analyses were conducted using BinomCI from the R package ExactCIdiff.
^{b}Most were calculated using the exact.test from the R package Exact. The log-rank χ² was used (χ² = 6.8) for the primary end point. The t test was used for clinical status on 7-point scale (t = −2.4) and the most severe baseline symptom change (t = 0.5).
^{c}Shortness of breath or hospitalization for shortness of breath or pneumonia and oxygen saturation dropped below 92% or supplemental oxygen was required to keep oxygen saturation at or above 92%. The prespecified primary outcome analysis was determined instead by survival analysis (time to clinical worsening). The absolute difference and 95% CI are for the Kaplan-Meier estimate of the placebo group at day 15. The test of difference is the log-rank statistic (χ² = 6.8).
^{d}No study participants were rated 2 (shortness of breath and oxygen saturation <92% plus supplemental oxygen needed), 4 (oxygen saturation <92% plus supplemental oxygen needed and hospitalization related to dyspnea or hypoxia plus ventilator support needed for <3 days), or 6 (death).
^{e}The protocol included a plan to examine number of days (1) requiring oxygen, (2) requiring hospitalization, and (3) requiring ventilator support. This type of outcome measure turned out to be invalid for this study because few patients required these interventions; therefore, a statistical analysis comparing the number of days was not appropriate.
^{f}Change from day 0 to day 15. The mean of the highest daily symptom score for each participant that was reported most severe at baseline (62 for fluvoxamine group and 54 for placebo group). This analysis was not pursued further because the curves showed no substantial differences and because the baseline most severe symptom was heterogeneous across participants (Table 1) and likely did not adequately capture overall symptom burden. eFIG. 1 in Supplement 2 is a box and whisker plot of the symptom data over the 15 days.
^{g}During the 30-day observation period after the 15-day randomized clinical trial, 1 participant from the fluvoxamine group was hospitalized for post-COVID headache and 1 participant from the placebo group had an emergency department visit for chest pain (costochondritis COVID-19 sequela). Details appear in eResults 2 in Supplement 2.

Among fluvoxamine-treated participants, 18 of 80 stopped responding to the surveys prior to day 15 compared with 19 of 72 who were randomized to placebo. For the nonprespecified outcome of hospital or emergency department care received during the 30 days after day 15 of the trial, among fluvoxamine-treated participants, 1 of 80 received care (hospitalized for headache) compared with 1 of 72 placebo-treated participants (emergency department visit for costochondritis) (see Results).

### Adverse Events

The fluvoxamine group had 1 serious adverse event and 11 other adverse events, whereas the placebo group had 6 serious adverse events and 12 other adverse events (TABLE 4). Pneumonia and gastrointestinal symptoms (such as nausea and vomiting) occurred more often in the placebo group compared with those who received fluvoxamine.

TABLE 4
Adverse Events^{a} In some cases, there was more than 1 symptom
or problem that occurred as part of 1 adverse event.

| | No. of adverse events (%)^{a} | |
|---|---|---|
| | Fluvoxamine | Placebo |
| | (n = 80) | (n = 72) |
| Pneumonia | 3 (3.8) | 6 (8.3) |
| Shortness of breath | 2 (2.5) | 4 (5.6) |
| Headache or head pain | 2 (2.5) | 1 (1.4) |
| Gastroenteritis, nausea, or vomiting | 1 (1.3) | 5 (6.9) |
| Muscle aches | 1 (1.3) | 0 |
| Bacterial infection | 1 (1.3) | 0 |
| Vasovagal syncope | 1 (1.3) | 0 |
| Teeth chattering | 1 (1.3) | 0 |
| Dehydration | 1 (1.3) | 0 |
| Low oxygen saturation or hypoxia | 0 | 6 (8.3) |
| Chest pain or tightness | 0 | 2 (2.8) |
| Fever | 0 | 2 (2.8) |
| Acute respiratory failure | 0 | 1 (1.4) |
| Hypercapnia | 0 | 1 (1.4) |
| Flank pain | 0 | 1 (1.4) |
| By No. of patients | | |
| Serious adverse events^{b} | 1 (1.3) | 5 (6.9) |
| Other adverse events^{c} | 11 (13.8) | 6 (8.3) |

^{a}In some cases, there was more than 1 symptom or problem that occurred as part of 1 adverse event. Additional details of adverse events appear in eResults 1 in Supplement 2.
^{b}One patient in the placebo group had more than 1 serious adverse event. The total No. of serious adverse events was 1 in the fluvoxamine group and 6 in the placebo group.
^{c}There were patients in the placebo group who had more than 1 other adverse event. The total No. of other adverse events was 11 in the fluvoxamine group and 12 in the placebo group.

### Missing Data

In terms of missing data, 517 of 3943 follow-up surveys (13%) were not filled out by participants. The mean score for those with missing data (0.80) was not different from the overall mean score for shortness of breath (0.83) and the median was 0 for both missing data and overall. The mean score for oxygen saturation was 97.3% for both those with missing data and overall and the median was 98% for both. Therefore, the data appeared to be missing at random and no data imputation was conducted. For the participants who stopped responding to the surveys prior to day 15 because they met the primary end point or for other reasons (FIG. 1B), the data were censored. In 31 individuals who stopped responding to the surveys prior to day 15 for other reasons, we confirmed that none received medical care at a hospital or emergency department for worsening COVID-19. However, for 6 of these individuals, we could not exclude the possibility that they received care at an urgent care center that was outside the major regional hospital systems.

### Discussion

In this preliminary randomized clinical trial, fluvoxamine (an S1R agonist) was associated with a reduction in clinical deterioration in adult outpatients with COVID-19. No fluvoxamine-treated patients met criteria for clinical deterioration as defined in the study, whereas 8.3% of patients taking placebo met this end point. However, because of study limitations, these findings need to be interpreted as hypothesis generating rather than as a demonstration of efficacy.

This double-blind, placebo-controlled, randomized clinical trial demonstrated the feasibility of a fully remote (contactless) study during the COVID-19 pandemic. Adult outpatients with COVID-19 are in self-quarantine, but few studies have focused on the care of this vulnerable population. This design included a short time from symptom onset to first dose of medication (median, 4 days), efficient study treatment initiation (92% took the first dose on the same day as they were contacted), and representative sample of race and sex.²⁰ The study required approximately 4500 hours of staff time and 30 hours of time per participant.

If fluvoxamine is determined to be effective in treating COVID-19, the underlying mechanism needs further clarification. The study was prompted by a hypothesis involving the influence of fluvoxamine on the S1R-IRE1 pathway. Anti-inflammatory (cytokine reduction) actions resulting from S1R activation would fit with recent findings of benefits of other anti-inflammatory drugs, such as colchicine and corticosteroids, for COVID-19.^{21,22} However, a recent study found lower levels of cytokines in patients with severe COVID-19 vs patients with bacterial sepsis.²³ Alternative mechanisms of a potential fluvoxamine benefit include direct antiviral effects via its lysosomotropic properties,²⁴ modulation of the effect of IRE1 effects on autophagy,²⁵ and SSRI inhibition of platelet activation.²⁶

The potential advantages of fluvoxamine for outpatient treatment of COVID-19 include its safety,²⁷ widespread availability, low cost, and oral administration. Fluvoxamine does not promote QT prolongation unlike other SSRIs.²⁸ However, fluvoxamine has adverse effects and can cause drug-drug interactions, particularly via inhibition of cytochromes P450 1A2 and 2C19.²⁹

### Limitations

This study has several limitations. First, it was a small study and it was conducted within a single geographic area, so these findings should be regarded as preliminary. The study needs to be replicated in larger trials with a more heterogeneous study population.

Second, there was a small number of end point events, which makes the findings extremely fragile. Third, it is possible that the differences in clinical deterioration may have been a reflection of the comparative baseline distributions of oxygen saturation rather than an effect of treatment.

Fourth, the method of measuring the most severe baseline symptom over time did not appear to provide valid data, so potential effects of fluvoxamine on symptomatic improvement are unknown. Fifth, 20% of study participants stopped responding to surveys during the 15-day trial. Although it was confirmed that none of these participants required medical care, such as hospitalization or an emergency department visit, it is possible that some received care at an urgent care center outside the major regional hospital systems.

Sixth, the follow-up duration was short and did not measure the effect of fluvoxamine on persistent symptoms or late deterioration. For example, individuals with COVID-19 may develop cardiac injury,³⁰ which may be common and persistent, even in otherwise mild or recovered cases.³¹ Because S1R agonists have cardioprotective effects in rodents³² and protective effects in other tissues,³³ future COVID-19 treatment trials should examine long-term outcomes and measures of cardiopulmonary function. Seventh, the 7-point ordinal scale created for this study to classify clinical deterioration has not been validated.

### Conclusions

In this preliminary study of adult outpatients with symptomatic COVID-19, patients treated with fluvoxamine, compared with placebo, had a lower likelihood of clinical deterioration over 15 days. However, the study is limited by a small sample size and short follow-up duration, and determination of clinical efficacy would require larger randomized trials with more definitive outcome measures. Trial registration: ClinicalTrials.gov Identifier: NCT04342663, see also Example 3.

### REFERENCES

1. Wiersinga W J, Rhodes A, Cheng A C, et al. Pathophysiology, transmission, diagnosis, and treatment of coronavirus disease 2019 (COVID-19). JAMA. 2020; 324(8):782-793.
2. Guan W J, Ni Z Y, Hu Y, et al. Clinical characteristics of coronavirus disease 2019 in China. N Engl J Med. 2020; 382(18):1708-1720.
3. Huang C, Wang Y, Li X, et al. Clinical features of patients infected with 2019 novel coronavirus in Wuhan, China. Lancet. 2020; 395(10223):497-506.
4. Lescure F X, Bouadma L, Nguyen D, et al. Clinical and virological data of the first cases of COVID-19 in Europe. Lancet Infect Dis. 2020; 20(6):697-706.
5. Mehta P, McAuley D F, Brown M, et al. COVID-19: consider cytokine storm syndromes and immunosuppression. Lancet. 2020; 395(10229):1033-1034.
6. Prasad A, Prasad M. Single virus targeting multiple organs. Front Med (Lausanne). 2020; 7:370.
7. Nicol G E, Karp J F, Reiersen A M, et al. “What were you before the war?” J Clin Psychiatry. Published online Apr. 7, 2020.
8. Ishima T, Fujita Y, Hashimoto K. Interaction of new antidepressants with sigma-1 receptor chaperones and their potentiation of neurite outgrowth in PC12 cells. Eur J Pharmacol. 2014; 727:167-173.
9. Rosen D A, Seki S M, Fernéndez-Castañeda A, et al. Modulation of the sigma-1 receptor-IRE1 pathway is beneficial in preclinical models of inflammation and sepsis. Sci Transl Med. 2019; 11(478):eaau5266.
10. Hashimoto K. Activation of sigma-1 receptor chaperone in the treatment of neuropsychiatric diseases and its clinical implication. J Pharmacol Sci. 2015; 127(1):6-9.
11. Cobos E J, Entrena J M, Nieto F R, et al. Pharmacology and therapeutic potential of sigma(1) receptor ligands. Curr Neuropharmacol. 2008; 6(4):344-366.
12. Hallifax D, Houston J B. Saturable uptake of lipophilic amine drugs into isolated hepatocytes. Drug Metab Dispos. 2007; 35(8):1325-1332.
13. World Medical Association. World Medical Association Declaration of Helsinki. JAMA. 2013; 310(20):2191-2194.
14. Nicol G E, Piccirillo J F, Mulsant B H, Lenze E J. Action at a distance. J Am Geriatr Soc. 2020; 68(5):922-925.
15. Mofsen A M, Rodebaugh T L, Nicol G E, et al. When all else fails, listen to the patient. JMIR Ment Health. 2019; 6(5):e11845.
16. CDC COVID-19 Response Team. Severe outcomes among patients with coronavirus disease 2019 (COVID-19). Morbid Mortal Weekly Rep. 2020; 69(12):343-346.
17. Ishikawa M, Ishiwata K, Ishii K, et al. High occupancy of sigma-1 receptors in the human brain after single oral administration of fluvoxamine. Biol Psychiatry. 2007; 62(8):878-883.
18. World Health Organization. Novel coronavirus. Accessed Nov. 2, 2020. https://www.who.int/blueprint/priority-diseases/key-action/COVID-19_Treatment_Trial_Design_Master_Protocol_synopsis_Final_18022020.pdf
19. Gillings D, Koch G. The application of the principle of intention-to-treat to the analysis of clinical trials. Drug Information J. 1991; 25:411-424.
20. Chastain D B, Osae S P, Henao-Martinez A F, et al. Racial disproportionality in Covid clinical trials. N Engl J Med. 2020; 383(9):e59.
21. Deftereos S G, Giannopoulos G, Vrachatis D A, et al. Effect of colchicine vs standard care on cardiac and inflammatory biomarkers and clinical outcomes in patients hospitalized with coronavirus disease 2019. JAMA Netw Open. 2020; 3(6):e2013136-e2013136.
22. Sterne J A C, Murthy S, Diaz J V, et al. Association between administration of systemic corticosteroids and mortality among critically ill patients with COVID-19. JAMA. 2020; 324(13):1330-1341.
23. Kox M, Waalders N J B, Kooistra E J, et al. Cytokine levels in critically ill patients with COVID-19 and other conditions. JAMA. 2020; 324(15):1565-1567.
24. Homolak J, Kodvanj I. Widely available lysosome targeting agents should be considered as potential therapy for COVID-19. Int J Antimicrob Agents. 2020; 56(2):106044.
25. Fung T S, Liu D X. The ER stress sensor IRE1 and MAP kinase ERK modulate autophagy induction in cells infected with coronavirus infectious bronchitis virus. Virology. 2019; 533:34-44.
26. Schlienger R G, Meier C R. Effect of selective serotonin reuptake inhibitors on platelet activation. Am J Cardiovasc Drugs. 2003; 3(3):149-162.
27. Omori I M, Watanabe N, Nakagawa A, et al. Efficacy, tolerability and side-effect profile of fluvoxamine for major depression. J Psychopharmacol. 2009; 23(5):539-550.
28. Assimon M M, Brookhart M A, Flythe J E. Comparative cardiac safety of selective serotonin reuptake inhibitors among individuals receiving maintenance hemodialysis. J Am Soc Nephrol. 2019; 30(4):611-623.
29. Christensen M, Tybring G, Mihara K, et al. Low daily 10-mg and 20-mg doses of fluvoxamine inhibit the metabolism of both caffeine (cytochrome P4501A2) and omeprazole (cytochrome P4502C19). Clin Pharmacol Ther. 2002; 71(3):141-152.
30. Lippi G, Lavie C J, Sanchis-Gomar F. Cardiac troponin I in patients with coronavirus disease 2019 (COVID-19). Prog Cardiovasc Dis. 2020; 63(3):390-391.
31. Puntmann V O, Carerj M L, Wieters I, et al. Outcomes of cardiovascular magnetic resonance imaging in patients recently recovered from coronavirus disease 2019 (COVID-19). JAMA Cardiol. Published online Jul. 27, 2020.
32. Hashimoto K. Sigma-1 receptor chaperone and brain-derived neurotrophic factor. Prog Neurobiol. 2013; 100:15-29.
33. Hosszu A, Antal Z, Lenart L, et al. σ1-receptor agonism protects against renal ischemia-reperfusion injury. J Am Soc Nephrol. 2017; 28(1):152-165.

### Example 3: A Double-Blind, Placebo-Controlled Clinical Trial of Fluvoxamine for Symptomatic Individuals with COVID-19 Infection (Stop COVID)

Brief Summary: The purpose of this research study is to determine if a drug called fluvoxamine can be used early in the course of the COVID-19 infection to prevent more serious complications like shortness of breath. Fluvoxamine is an anti-depressant drug approved by the FDA for the treatment of obsessive-compulsive disorder. The use of fluvoxamine for the treatment of COVID-19 is considered investigational, which means the US Food and Drug Administration has not approved it for this use.

This study is fully-remote, which means that there is no face-to-face contact; study materials including study drug will be shipped to participants' houses. Only residents of Missouri and Illinois may participate.

Detailed Description: We will consent approximately 152 participants, age 18 and older, who have tested positive for COVID-19 and are currently experiencing mild symptoms. All interactions for this study will be conducted remotely by videoconferencing, email, or phone.

Screening: All participants will first complete a pre-screen to see if they may be eligible for the study. Once a participant is confirmed eligible and consented, the study team will send the study materials. These materials will consist of study medication and self-monitoring equipment, including a pregnancy test (for females of childbearing age not using contraception), an oxygen saturation monitor, blood pressure monitor, and thermometer. Once the study team has finalized the screening process, the participant will begin taking the study medication.

RCT: Participants will be randomly assigned (1:1) to take either fluvoxamine or a placebo. This phase of the study will last approximately 15 days and is double-blinded. Participants will take 100 mg of fluvoxamine or placebo by mouth three times a day for a daily total of 300 mg. They will continue this dose for approximately 15 days. Depending on tolerability, the dose may be adjusted. Participants will also complete short 10-15 minute assessments daily to assess symptoms, results of self-monitoring (including oxygen level, blood pressure, and temperature) and any adverse events.

Open-label Phase: After completing the randomization phase, participants will then participate in an open-label phase (participant will definitely receive fluvoxamine) that will last up to 15 days. Those randomized to placebo will have the opportunity to try fluvoxamine during this time. Those randomized to fluvoxamine will continue this medication while slowly decreasing the drug. The participant may opt out of this phase. The dosage during this time will be 50-100 mg two times daily until discontinuing the drug. Participants will also continue completing short 10-15 minute daily assessments to assess physical symptoms, vitals, and any adverse events.

Follow-up Phase: We will follow participants for approximately 30 days after the end of the randomized phase. During this time, they may continue to complete daily study assessments to monitor physical symptoms, vitals, and adverse events. If needed, the study team will review medical records to determine the clinical course of participants.

| Arms | Assigned Interventions |
|---|---|
| Experimental: Fluvoxamine | Drug: Fluvoxamine |
| Start fluvoxamine 100 mg capsules, three | Randomized to either fluvoxamine or placebo |
| times daily. May reduce dose (or start at | for approximately 15 days. Will take up to 300 |
| reduced dose) for tolerability reasons. Will be | mg per day (3 capsules per day) as tolerated. |
| followed in the RCT for approximately 15 days. | Other Names: Luvox |
| Placebo Comparator: Placebo | Drug: Placebo |
| Start placebo one capsule, three times daily. | Randomized to either fluvoxamine or placebo |
| May reduce dose (or start at reduced dose) for | for approximately 15 days. Will take up to 3 |
| tolerability reasons. Will be followed in RCT for | capsules per day as tolerated. |
| approximately 15 days. | |

### Primary Outcome Measures:

1. Time to clinical worsening [Time Frame: RCT (approximately 15 days)]

Clinical worsening is defined meeting both of the following: (1) presence of dyspnea and/or hospitalization for shortness of breath or pneumonia, plus (2) decrease in O2 saturation (<92%) on room air and/or supplemental oxygen requirement in order to keep O2 saturation >92%.

### Secondary Outcome Measures:

1. clinical deterioration on a Likert-type scale (1-6) [Time Frame: RCT (approximately 15 days)]

(1) moderate severity of illness as defined by O2 saturation <92% but no supplemental oxygen requirement; (2) O2 saturation plus supplemental oxygen requirement; (3) O2 saturation <92% plus hospitalization (related to dyspnea/hypoxia); (4) the above, plus ventilator support requirement; (5) the above, plus ventilator support for at least 3 days; (6) death.
2. clinical deterioration measured by number of days [Time Frame: RCT (approximately 15 days)]

(1) requiring supplemental oxygen; (2) requiring hospitalization; (3) requiring ventilator support.
3. Symptomatic severity on a likert scale (0-10 where 0=none and 10=very severe) [Time Frame: RCT (approximately 15 days)]

Outcomes will be collected daily, with symptomatic data collected approximately twice daily. The most severe symptom at baseline will be the focus.

### Eligibility:

Minimum Age: 18 Years; Sex: All; Accepts Healthy Volunteers: No

### Trial Protocol

A Double-Blind, Placebo-Controlled Clinical Trial of Fluvoxamine for Symptomatic Individuals with COVID-19 Infection

### Background:

The biphasic evolution of COVID-19 infection: The clinical course of COVID-19 infection varies, with approximately 20% of individuals developing serious illness including lung damage, hypoxia, and cardiac damage. This leads to hospitalization, ICU admission, and in some cases fatality.

Many infected individuals show a biphasic evolution of the illness, in which clinical deterioration often develops during the second week of illness

https://www.cdc.gov/coronavirus/2019-ncov/hcp/clinical-guidance-management-patients.html. In China, one report of 41 hospitalized patients found that 55% developed dyspnea 8 days (median; range 5-13) after illness onset https://www.ncbi.nlm.nih.gov/pubmed/31986264. Also, a data extraction of 1099 patients found the mean time from illness onset to hospital admission with pneumonia was 9 days. https://www.ncbi.nlm.nih.gov/pubmed/32109013. The first cases in Europe had the same course, with a deterioration leading to requirement of supplemental oxygen 10-11 days after the onset of mild symptoms and in spite of a decreasing viral load, and it was postulated that “the lung damage is more related to immunopathological lesions, resulting from an excessive pro-inflammatory host response, rather than to uncontrolled viral replication” https://www.thelancet.com/action/showPdf?pii=S1473-3099%2820%2930200-0.

Thus, many patients with COVID-19 develop lung damage resulting from excessive inflammatory responses, or “cytokine storm”. These individuals often develop cardiac injury, further highlighting the short-term and long-term risks and complications from infection https://www.ncbi.nlm.nih.gov/pubmed/32169400. This cytokine storm led to recommendations to trial hydroxychloroquine which could suppress T cell activation https://www.ncbi.nlm.nih.gov/pubmed/32196083 as well as other immunosuppressive strategies https://www.ncbi.nlm.nih.gov/pubmed/32192578. Many of these drugs have significant toxicities, and to date most clinical trials have focused on individuals who already have serious to critical illness.

Sigma-1 receptor agonism as a pathway to preventing the cytokine storm: A 2019 study showed that SSRI fluvoxamine reduces damaging aspects of the inflammatory response during sepsis, and protects mice from lethal septic shock https://www.ncbi.nlm.nih.gov/pubmed/30728287. Fluvoxamine binds to the sigma-1 receptor (S1R), which regulates inflammation by inhibiting cytokine production. Fluvoxamine may also induce the S1R https://www.ncbi.nlm.nih.gov/pmc/articles/PMC4123092/. The S1R restricts the endonuclease activity of an Endoplasmic Reticulum (ER) stress sensor called Inositol-Requiring Enzyme1 (IRE1) and reduces cytokine expression, without inhibiting classical inflammatory pathways.

IRE1 is also necessary for induction of autophagy during infection of cells by a coronavirus that causes infectious bronchitis in animals https://www.ncbi.nlm.nih.gov/pubmed/31082732. S1R agonists (including fluvoxamine) also have cardioprotective effects in rodents https://www.ncbi.nlm.nih.gov/pubmed/23044468 and protective effects in other tissues https://www-ncbi-nlm-nih-gov.beckerproxy.wustl.edu/pubmed/27056295.

Repurposing antidepressants for COVID-19: A recent preprint entitled, “A SARS-CoV-2-Human Protein-Protein Interaction Map Reveals Drug Targets and Potential Drug Repurposing” argued that a key drug repurposing strategy was “modulation of the ER stress and the protein unfolding response pathway by targeting the Sigma1 and Sigma2 receptor” https://www.biorxiv.org/content/10.1101/2020.03.22.002386v1.full.pdf.

Many antidepressants (except sertraline) are S1R agonists, and a recent article on drug repurposing has suggested SSRIs may have value in treating COVID-19 https://www.nature.com/articles/s41421-020-0153-3. SSRIs in particular are excellent candidates for drug repurposing because of their ease of use, including high safety margin, good tolerability, widespread availability, and low cost such that primary care physicians and other providers could prescribe them.

Fluvoxamine has biochemical properties favorable for repurposing as an S1R agonist against COVID-19 infection. It has the strongest S1R agonist effect of any SSRI https://www.ncbi.nlm.nih.gov/pubmed/24508523. A PET study found that a single dose of 150-200 mg produced 60-63% S1R binding in brain regions (https://www.ncbi.nlm.nih.gov/pubmed/17662961). This suggests that fluvoxamine would have clinically meaningful S1R agonist effects within its clinical dosing range (up to 300 mg/day is the maximum per FDA). It is also highly lipophilic and has demonstrated to have rapid, substantial intracellular uptake (http://dmd.aspetjournals.org.beckerproxy.wustl.edu/content/35/8/1325 #sec-4).

Fluvoxamine also is an ideal drug for repurposing in outpatients with COVID-19. It is safe, inexpensive, and already widely available and FDA-approved. Our team has prescribed it across the age span including in older adults at maximum therapeutic doses, finding it safe and well-tolerated. Fluvoxamine does not promote QT prolongation unlike some SSRIs (https://www.ncbi.nlm.nih.gov/pubmed/30885935), and it can be rapidly initiated at high therapeutic doses. It is devoid of off-target effects, having only SSRI and S1R binding at clinically approved doses. In our review of all of the available, FDA-approved drugs that are agonists of S1R, fluvoxamine appears have the desirable combination of potent S1R agonism at clinically approved dosing, and ease of use including safety and tolerability.

These features of fluvoxamine also make it an ideal drug for a pragmatic trial in outpatients. Given risks from face-to-face contact, pragmatic fully-remote trials are a mandate. Fluvoxamine requires no therapeutic drug monitoring or baseline or follow-up laboratory testing, even in older adults. Our research group has expertise with such trials, and with secondary prevention studies (e.g., https://www.ncbi.nlm.nih.gov/pubmed/31147244, https://www.ncbi.nlm.nih.gov/pubmed/23680817, https://www.ncbi.nlm.nih.gov/pubmed/16540613) with SSRIs. In fact, our ongoing antidepressant trial, OPTIMUM, has been minimally affected by the ongoing pandemic because of the ability to continue the trial fully remotely with work-from-home staff and investigators (https://www.ncbi.nlm.nih.gov/pubmed/32207542).

Summary: In the fight against COVID-19, we need a simple, benign drug that could be provided widely to outpatients early in the course of mild illness, to prevent the clinical deterioration to serious and life-threatening cardiopulmonary problems. Existing clinical trials have been appropriately focused on treating more serious cases, but with few if any focusing on this clinical space. S1R agonists—of which fluvoxamine is a particularly attractive candidate—could be given early in the course of symptomatic illness, to outpatients with mild symptoms, to decrease the excessive inflammatory response with COVID-19, and potentially prevent the evolution to more serious disease. A search (on March 28; repeated April 9) of clinicaltrials.gov and the WHO International Registry of Clinical Trials (https://www.who.int/ictrp/en/) finds no trials—yet—of fluvoxamine, or any S1R agonist.

### Human Participants Involvement, Characteristics, and Design

### Subject Involvement:

The proposed study is a pragmatic trial that will rapidly deliver drug/placebo and other supplies to (quarantined) patients, including thermometers, blood pressure cuffs, pregnancy tests (to confirm pregnancy exclusion in childbearing-capable females), and O2 saturation monitors. The risk pertaining to face-to-face contact is with research staff, not participants (who are already COVID-19 positive), so we will conduct the trial via a “no contact” method that will not require PPE. Because of the large, well-trained research staff in the Healthy Mind Lab, we will be able to recruit, screen, e-consent, and start intervention in each participant rapidly. All staff will be trained in COVID-19 precautions using the WHO training for health care providers (https://www.who.int/emergencies/diseases/novel-coronavirus-2019/training/online-training) and supervised by physician faculty. The Healthy Mind Lab has extensive experience conducting clinical trials in adults including older adults, including pragmatic trials with antidepressants.

Recruitment & retention: For the present study, participants will be recruited through EPIC, physician referrers, doctor's hotlines, COVID-19 Testing Centers, and Emergency Rooms (microbiology lab). They may also be recruited, as needed, via word of mouth, referrals by colleagues, the Healthy Mind Lab website (www.healthymind.wustl.edu), flyers, and email notification. Participants will be considered eligible per the inclusion/exclusion criteria and at the PI's determination. Both genders will be enrolled without regard to race, ethnicity, or religion. Potential participants undergo screens by email and phone/videoconference.

The proposed study will require voluntary participation of individual persons and will abide by all federal regulations related to human subject protection, inclusion of women and minorities, and privacy of individually identifiable health information. As with any study that collects sensitive information, breach of confidentiality is a potential risk. To mitigate this risk, all study personnel will undergo training specific to the use of human participants in research (CITI), will be Health Insurance Portability and Accountability Act (HIPAA) and Good Clinical Practices (GCP) trained, and will be approved by the Washington University in St. Louis Institutional Review Board (IRB). Throughout the study, we will confirm plans to assure: 1) accurate, complete, and verifiable data collection and 2) the rights and well-being of human subjects will be protected, in accordance with 45 CFR 46 (Protection of Human Subjects) and, as applicable, 21 CFR part 50 (Protection of Human Subjects). Additionally, all data will be maintained strictly according to the HIPAA “two lock” policy. Only study personnel will have access to this data.

Participation in the proposed study-supported activities will be entirely voluntary and permitted only following completion of all consent-related procedures. We view informed consent as an ongoing process, and will continue the informed consent conversation with subjects throughout their participation.

### Inclusion and Exclusion Criteria

Inclusion criteria:
1) men and woman age 18 and older,
2) Outpatients (living in the community)
3) Proven SARS-CoV-2 positive (per lab or physician report).
4) Currently symptomatic with one or more of one or more of the following symptoms: fever, cough, myalgia, mild dyspnea, diarrhea, vomiting, anosmia (inability to smell), ageusia (inability to taste), sore throat.
5) Able to provide informed consent.

Exclusion criteria:
1) Illness severe enough to require hospitalization or already meeting study's primary endpoint for clinical worsening.
2) Unstable medical comorbidities including, but not limited to: Severe underlying lung disease (COPD on home oxygen, interstitial lung disease, pulmonary hypertension), decompensated cirrhosis, Congestive heart failure (stage 3 or 4 per patient report and/or medical records).
3) Immunocompromised (solid organ transplant, BMT, AIDS, on biologics and/or high dose steroids (>20 mg prednisone per day)
4) Already enrolled in another COVID 19 trial, or currently taking chloroquine, hydroxychloroquine, azithromycin or colchicine
5) Unable to provide informed consent (e.g. moderate-severe dementia diagnosis)
6) Unable to perform the study procedures

Rationale for inclusion & Exclusion Criteria: Adults 18 and older who are not already medically compromised (eg severe lung disease) are at risk for developing clinical deterioration late in their clinical course. The Inclusion criteria will ensure that we collect as generalizable sample as possible of at-risk adults, while the exclusion criteria will ensure that we do not recruit individuals who are likely to need immediate hospitalization or are otherwise too medically unstable to participate in an outpatient study with remote monitoring. Older adults will be included, exclusion criteria will exclude individuals who are highly frail, medically compromised, or highly cognitively impaired.

### Study Procedures, Materials, and Potential Risks

### Sources of Materials

Research materials will be obtained by remote interviews, self-reports, medical records (when available), and remote self-monitoring. All materials will be obtained solely for the purposes of the research study. All data collected from the participants enrolled in the study will be stored and maintained confidentially and no identifying information, such as participant names, will be disclosed in any published documents.

### Study Procedures

After passing an initial prescreening, potential participants will provide informed consent and be administered emailed, phone or videoconference-based screening assessments by trained research staff to assess the above inclusion and exclusion criteria. Participants who qualify after completing all screening assessments will then be sent or provided the study materials. This consists of study medication and self-monitoring equipment, including, as needed, a pregnancy test [for females of childbearing age not using contraception], oxygen saturation monitor, automated blood pressure monitor, and thermometer. We will finalize screening (ie confirming medical stability via oxygen saturation monitor, and vital sign measurement) prior to instructing participants to begin taking study medication, after which point (after taking first dose) they are considered “randomized”, consistent with Intention to Treat principles.

### Study Design

This study will randomize approximately n=152 participants. This study has an initial double-blind phase (to test the drug's effectiveness) followed by an open-label phase (to provide all participants with exposure to the drug, and allow a taper off of it for those initially taking active drug).

Assignment will be 1:1; (1) Active treatment: start and maintain fluvoxamine 100 mg, three times daily. May reduce dose (or start at reduced dose) for tolerability reasons; (2) Control: placebo, three times daily. This drug vs placebo phase will last approximately 15 days and will be followed by an open-label fluvoxamine phase that will typically last 6-7 days but could last up to 15 days: 100 mg two times daily for 3 days, then 50 mg two times daily for 3 days, then discontinue.

Randomization will be stratified by age (we anticipate: 18-44, 45-54, 55-64 and >65 years, respectively, to reflect the age distribution of ascending risk for severe symptoms requiring hospitalization (https://www.cdc.gov/mmwr/volumes/69/wr/mm6912e2.htm#F2_down) and sex. Final determination of stratification will be made by the biostatistician (Phil Miller). Stratified randomization schedules will be generated by statistician Michael Yingling.

After the randomized phase, all participants will be followed for approximately 30 days to collect clinical data (i.e., on hospitalization and other clinical outcomes). Thus, total study participation is approximately 45 days (15 days randomized, 30 days follow-up).

Management: To protect research staff, there will be no face-to-face contact with participants, all

interactions will be fully-remote via Zoom videoconference and/or phone/text/email, as well as

REDCap surveys pushed out to patients via their smartphones or other devices.

For consented participants who start protocolized treatment we will inform their relevant medical providers, such as a PCP, towards the goal of an informed and collaborative management strategy.

As this is a “prevention of clinical deterioration” study, it is anticipated that a minority (˜10-15%) of participants will become hospitalized during the study, and that this will most likely occur during the randomized phase. Research staff under the physician investigators' supervision will provide support including engagement with participants' caregivers and physicians to ensure timely medical contact and emergency care (including calling 911).

If a study participant develops a decrease in oxygen saturation to less than 90% on room air on >2 readings, persistent increase in respiratory rate to >30 breaths per minute, persistent increase in Heart Rate to >120 beats per minute, alteration in mentation, or severe worsening in shortness of breath, the research staff will direct them to seek emergency medical care at the nearest emergency department. If none of the above conditions are met, but the research staff still feel that the participant is unwell, one of the physician investigators will evaluate the participant via phone/telehealth and direct them for further care if needed. When a participant is directed to seek emergency medical care, they will be instructed to use a mask if available, and to identify themselves to EMS or to the Emergency Department staff as having been diagnosed with COVID-19.

### Safety Assessments

We will assess for adverse events (including serious adverse events) daily via participant self-report during the first 15 days, and then again at the end of the study.

### Outcome Assessments

As this is a prevention (of clinical deterioration) trial, the primary endpoint will be a “time to” clinical worsening analysis. Following the intention to treat principle, all participants who are confirmed to have taken at least one dose of study medication are included in the analyses.

Definition of clinical worsening: Clinical worsening is defined meeting both of the following: (1) presence of dyspnea and/or hospitalization for shortness of breath or pneumonia, plus (2) decrease in O2 saturation (<92%) on room air and/or supplemental oxygen requirement in order to keep O2 saturation >92%. A laboratory-confirmed clinical endpoint is consistent with World Health

Organization clinical trial recommendations (https://www.who.int/blueprint/15-01-2020-nfr-bp-wg-clinical-trials-ncov.pdf?ua=1).

Power analysis: The below table regards the primary endpoint. It assumes 80% power, alpha=0.05, and a 20% rate of progression to serious symptoms in the placebo group. The table shows sample size required based on reduction in that rate in the active (fluvoxamine) group. With total n=152 (76 per group), we would have 80% power to find a statistically significant difference in survival curves (i.e., time to clinical worsening) if the rate of progression with fluvoxamine is only 5% (i.e., a three-quarters reduction in the risk of clinical deterioration). This power calculation is conservative because it does not take into account the potentially improved power gained by stratification, or the enhanced power for Kaplan Meier vs. simple rates, therefore, we are likely to be sufficiently powered for a more modest treatment effect.

| Treatment Response | N (each group) |
|---|---|
| .05 | 76 |
| .06 | 90 |
| .07 | 108 |
| .08 | 131 |
| .09 | 160 |
| .10 | 199 |

We will also examine the following secondary endpoints:

1. clinical deterioration on a Likert-type scale: (1) moderate severity of illness as defined by O2 saturation <92% but no supplemental oxygen requirement; (2) O2 saturation plus supplemental oxygen requirement; (302 saturation <92% plus hospitalization (related to dyspnea/hypoxia); (4) the above, plus ventilator support requirement; (5) the above, plus ventilator support for at least 3 days; (6) death.

2. clinical deterioration measured by number of days: (1) requiring supplemental oxygen; (2) requiring hospitalization; (3) requiring ventilator support. These Likert-type assessments are based on our review of current COVID-19 treatment studies. They will be collected in retrospect via patient or family report and/or medical records.

3. Total symptomatic severity during the 15 days using a continuous scale of each patient's presenting symptoms on a 0-10 Likert scale (0=symptom not present, 10=symptom is very severe). Outcomes will be collected daily, with symptomatic data collected approximately twice daily via the technique of Ecological Momentary Assessment (ie “how bad is your symptom right now?”). The most severe symptom at baseline will be the focus. This data collection and analytic technique (with frequent measurement of the symptom[s] most relevant to the individual patient) should greatly increase precision and power (https://www.ncbi.nlm.nih.gov/pmc/articles/PMC6524455/).

Data management and analysis: Data will be collected in REDCap and outcomes will be collected by blinded research staff. The study statistician, Michael Yingling, will conduct blinded (ie “treatment 1 vs treatment 2”) analysis under the supervision of Phil Miller. We may conduct an interim “futility” analysis at approximately n=100 participants. The primary analysis will be survival analysis of the primary outcome (clinical worsening), while secondary endpoints (symptomatic severity) will be examined using appropriate (descriptive, Area Under the Curve (AUC), and trajectory) comparisons.

Sharing of de-identified data from this study will be critical because it will help characterize this patient population and, if positive results, would immediately lead to a potential new treatment for COVID-19. Therefore it is planned that a pre-print of the article along with de-identified data will be posted on a public repository such as arxiv (arxiv.org). This has been standard during the pandemic in order to share results as rapidly as possible.

### Additional Measurements:

Baseline: vital signs (pulse, blood pressure, temperature), O2 saturation, weight and height, symptoms, and symptomatic severity.

Follow-up: daily vital signs, O2 saturation, adverse events (participant self-report).

### Risks

Potential risks associated with study medication Fluvoxamine:

General comments: Fluvoxamine is an antidepressant drug that functions as a selective serotonin reuptake inhibitor (e.g., similar to escitalopram (Lexapro), fluoxetine (Prozac), sertraline (Zoloft), etc—among the most commonly prescribed drugs in the US). Its risk profile below is for chronic use in a psychiatrically ill population; the risks for short-term use in a non-psychiatric population are likely lower. The research team will carefully evaluate co-prescribed drugs as well as OTC medications and caffeine use, to mitigate drug-drug interactions. We anticipate approximately 20% of participants will be on current SSRI/SNRI use at recruitment—will include these individuals, so long as the participant can be safely switched over to fluvoxamine briefly.

Likely risks: none.

Less likely (1-10%): Nausea, Vomiting, Weight loss, Yawning, Loss of appetite, Agitation/Nervousness/Anxiety, Insomnia, Somnolence, Tremor, Headache, Dizziness, Palpitations, Tachycardia (high heart rate), Abdominal pain, Dyspepsia (indigestion), Diarrhea, Constipation, Hyperhidrosis (excess sweating), Asthenia (weakness), Malaise, Sexual dysfunction (including delayed ejaculation, erectile dysfunction, decreased libido, etc.), Xerostomia (dry mouth).

Rare (<1%): Arthralgia, Hallucination, Confusional state Extrapyramidal side effects (e.g. dystonia, parkinsonism, tremor, etc.), Orthostatic hypotension, Cutaneous hypersensitivity reactions (e.g. oedema [buildup of fluid in the tissues], rash, pruritus), Mania (elevated mood together with reduced need for sleep and increased energy), seizures, Abnormal hepatic (liver) function, Photosensitivity (being abnormally sensitive to light), Galactorrhoea (expulsion of breast milk unrelated to pregnancy or breastfeeding).

Minimizing risk:

Fluvoxamine is a commonly prescribed serotonin reuptake inhibitor which has been commonly prescribed in the US for more than 2 decades, and in this study it is provided only briefly. Therefore, moderate-severe and serious treatment-attributable AEs would be uncommon, but monitoring will include oversight by physicians with expertise in this drug class (Drs Lenze, and Nicol).

Changes from original study protocol, all made in April 2020:

1. Addition of Michael Avidan, MBBCh, as team member.

2. Making the open-label phase optional was a change from the original study protocol.

3. Removed exclusions for currently taking chloroquine, hydroxychloroquine, azithromycin, or colchicine.

4. Allowed for first dose of fluvoxamine/placebo to be 50 mg, to improve tolerability.

### Details on Drug and Placebo Encapsulation:

100 mg fluvoxamine maleate and 50 mg fluvoxamine maleate manufactured by Apotex was pulverized and encapsulated along with microcrystalline cellulose and silica gel, micronized. Matching placebo gelatin capsules micronized. All active drug and placebo preparation was performed by Medical Arts Pharmacy, St. Louis, Missouri.

### Example 4: Long Term Follow-Up Data from STOP COVID Trial

This example describes a patient report of recovery level months after the clinical trial detailed in Examples 2 and 3.

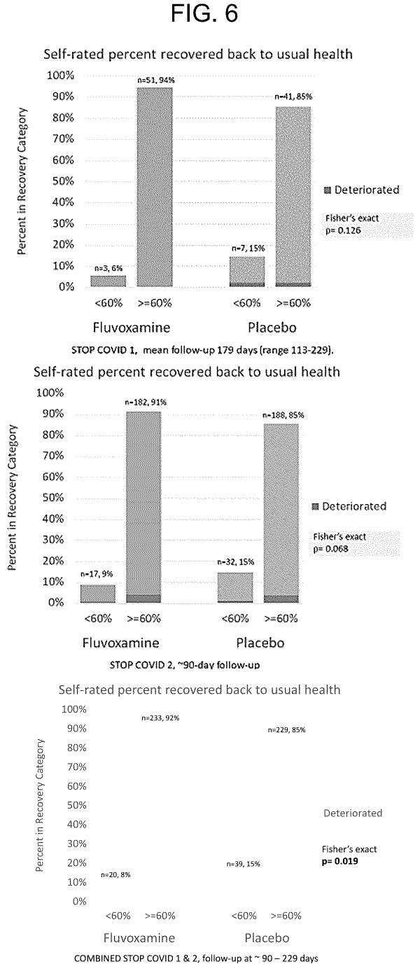

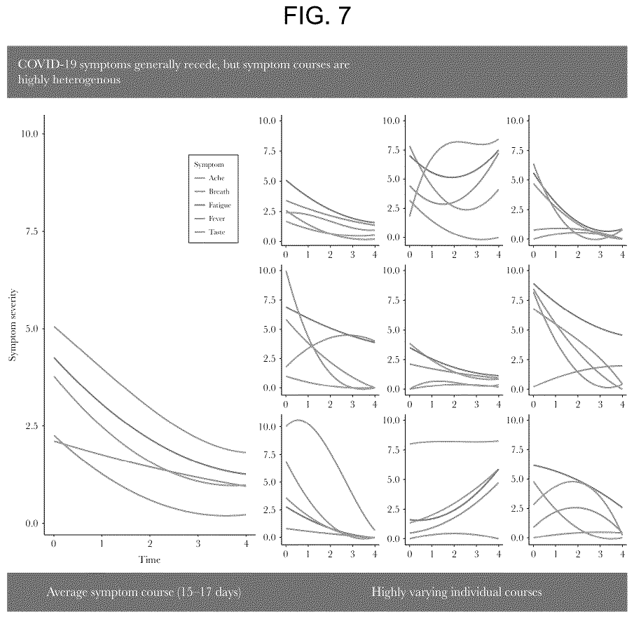

FIG. 6 (top): Long Term Follow-Up Data from STOP COVID 1 (Total N=102) and STOP COVID 2 (middle) (Total N=419). Participants in the fluvoxamine group were more likely to report themselves at least 60% recovered back to their usual baseline health at the time of follow-up. This is true even if participants who experienced respiratory deterioration during the 15-day clinical trial are excluded. Differences are not statistically significant for either trial by itself. Mean number of days from start of trial to time of follow up questionnaire was 179 days (range 113-229).

When STOP COVID 1 & 2 data are combined (FIG. 6 bottom) (Total N=521), the difference in the proportion at least 60% recovered is statistically significant.

A portion of the follow up questionnaire results is shown below. 70 symptom items were included. If controlling for multiple testing, there are no statistically significant differences in whether people ever had a symptom or whether they still had the symptom within the past 48 hours.

However, the symptom item that showed the most evidence of a statistical difference between groups was within this “Chest Discomfort” section. It is an item about chest wall ache (ribs/sternum). This symptom was meant to potentially detect costochondritis (an inflammation of the cartilage that connects a rib to the breastbone (sternum)). The reason this was included that specific item in the questionnaire was because it was observed that many long-haulers were reporting that this was a problem during acute COVID and for months afterward. This particular symptom is about twice as common in the placebo group compared to the fluvoxamine group and is thought to be an inflammatory symptom.

| Chest | Symptom present | | | Symptom present | | |
|---|---|---|---|---|---|---|
| Discomfort | during the course of | | | within past 48 hours at | | |
| (“Discomfort in | COVID-19, n (%) | | X² | time of follow-up, n (%) | | X² |
| your chest?”) | Fluvoxamine | Placebo | (p-value) | Fluvoxamine | Placebo | (p-value) |
| Chest pressure | 29 | 24 | 0.140 | 9 | 8 | 0.000 |
| (may feel like | (54%) | (50%) | (0.709) | (17%) | (17%) | (1.000) |
| something | | | | | | |
| heavy sitting on | | | | | | |
| chest) | | | | | | |
| Chest tightness | 29 | 28 | 0.221 | 9 | 8 | 0.0)00 |
| | (54%) | (58%) | (0.638) | (17%) | (17%) | (1.000 |
| Ache in chest | 18 | 30 | 8.677 | 6 | 11 | 2.550 |
| wall | (33%) | (63%) | (0.003)** | (11%) | (23%) | (0.110) |
| (ribs/sternum) | | | | | | |
| Sharp chest | 18 | 19 | 0.429 | 7 | 10 | 1.133 |
| pains | (33%) | (40%) | (0.512) | (13%) | (21%) | (0.287) |
| Pain deep | 17 | 19 | 0.730 | 5 | 8 | 1.254 |
| inside the chest | (31%) | (40%) | 0.393 | (9%) | (17%) | (0.263) |
| Chest pain that | 20 | 23 | 1.234 | 9 | 11 | 0.630 |
| worsens when | (37%) | (48%) | (0.267) | (17%) | (23%) | (0.427) |
| taking a deep | | | | | | |
| breath | | | | | | |

### Example 5: Acute Symptoms of Mild to Moderate COVID-19 are Highly Heterogeneous Across Individuals and Over Time

The aim of the current study is to characterize the temporal dynamics of COVID-19 symptoms in a sample of participants in a randomized controlled trial testing the medication fluvoxamine (vs. placebo) for early COVID-19 treatment (see Example 2). Participants reported on their experience of common COVID-19 symptoms, as well as blood oxygen level and other clinical signs, twice daily for a maximum of 31 times over the course of a maximum of 17 days.

### Abstract

Background: The symptoms of coronavirus disease 2019 (COVID-19) appear to be heterogenous, and the typical course of these symptoms is unknown. Our objectives were to characterize the common trajectories of COVID-19 symptoms and to assess how symptom course predicts other symptom changes as well as clinical deterioration.

Methods: One hundred sixty-two participants with acute COVID-19 responded to surveys up to 31 times for up to 17 days. Several statistical methods were used to characterize the temporal dynamics of these symptoms. Because 9 participants showed clinical deterioration, we explored whether these participants showed any differences in symptom profiles.

Results: Trajectories varied greatly between individuals, with many having persistently severe symptoms or developing new symptoms several days after being diagnosed. A typical trajectory was for a symptom to improve at a decremental rate, with most symptoms still persisting to some degree at the end of the reporting period. The pattern of symptoms over time suggested a fluctuating course for many patients. Participants who showed clinical deterioration were more likely to present with higher reports of severity of cough and diarrhea.

Conclusions: The course of symptoms during the initial weeks of COVID-19 is highly heterogeneous and is neither predictable nor easily characterized using typical survey methods. This has implications for clinical care and early-treatment clinical trials. Additional research is needed to determine whether the decelerating improvement pattern seen in our data is related to the phenomenon of patients reporting long-term symptoms and whether higher symptoms of diarrhea in early illness presages deterioration.

### Introduction

The World Health Organization (WHO) and the Centers for Disease Control and Prevention (CDC) have described common symptoms of the coronavirus disease 2019 (COVID-19), including fever, dry cough, and difficulty breathing [1, 2]. They also report that many people may have few or no symptoms despite infection. This characterization is based on studies that report cross-sectional or retrospective accounts of symptoms from chart review or patient interviews [3-10].

Thus, the temporal dynamics of COVID-19 symptoms are unclear. For example, in a patient currently experiencing fever, should the patient be concerned about more severe illness if the fever seems to resolve but returns a day later? Notably, a recent review of long-term COVID-19 symptoms revealed that some patients expected a gradual, linear recovery and were troubled by symptoms that instead waxed and waned [11]. Moreover, if COVID-19 symptoms have highly heterogeneous trajectories, this could be a barrier to measuring symptoms as a treatment outcome, in that reduction in symptoms on average could mask increases in specific symptoms, such that some patients could appear to improve (reduction in symptoms overall) while actually deteriorating (e.g., increase in shortness of breath alone).

The aim of the current study is to characterize the temporal dynamics of COVID-19 symptoms in a sample of participants in a randomized controlled trial testing the medication fluvoxamine (vs placebo) for early COVID-19 treatment [12]. Participants reported on their experience of common COVID-19 symptoms, as well as blood oxygen level and other clinical signs, twice daily for a maximum of 31 times over the course of a maximum of 17 days.

We used these frequently sampled data to model trajectories of the self-reported symptoms. That is, we characterized the course of symptoms using latent trajectory models (also called latent growth curve models). These models test how to best characterize the changes in self-reported symptoms across the course of the study in those participants who ever reported that symptom. These models are commonly used to study the development of symptoms over time [13, 14].

To model trajectories meaningfully, it is essential to obtain sequential data in real time, without reliance on retrospection. Asking people to recall events results in poor quality data even about events that seem highly memorable [15]. With frequently sampled data over time, a variety of statistical methods allows the researcher to characterize what is generally true in the sample over time, as well as to what extent participants vary from this average trajectory. Thus, we can determine the ways in which symptoms change over time, as well as whether changes in some symptoms tend to go together, as might be expected due to some symptoms being functionally related (e.g., problems with smell and taste).

### Methods

### Study Population

Participants were adults living in the community with severe acute respiratory syndrome coronavirus 2 (SARS-CoV-2) infection confirmed by polymerase chain reaction assay who were currently symptomatic with symptom onset <7 days before beginning survey responses. Exclusion criteria included COVID-19 severe enough to require hospitalization or meeting the study's primary end point for clinical deterioration at baseline (i.e., oxygen saturation of <92% on room air), medical comorbidities including severe underlying lung disease (chronic obstructive pulmonary disease or home oxygen, interstitial lung disease, pulmonary hypertension), decompensated cirrhosis, congestive heart failure (stage 3 or 4), and being immunocompromised (solid organ transplant, bone marrow transplant recipient, AIDS, on biological immunologic medications or high-dose steroids [>20 mg of prednisone per day]).

The clinical trial's primary outcome measure was clinical deterioration, defined by meeting both of the following: (1) presence of dyspnea (i.e., shortness of breath) and/or hospitalization for shortness of breath or pneumonia plus (2) decrease in oxygen saturation (<92%) on room air and/or supplemental oxygen requirement in order to keep oxygen saturation ≥92%. Participants typically stopped completing surveys on their symptoms once they met the primary outcome criteria. However, data are available from 1 participant who experienced moderate deterioration halfway through the trial and continued to provide data after deterioration. Notably, because we included all participants who provided any symptom survey data, we report on an additional 10 participants not included in the parent study [12]; these subjects were excluded from the parent study because they either could not be confirmed to have taken the study drug (n=7) or were deteriorated at baseline (n=3).

### Patient Consent Statement

The study was approved by Washington University's Institutional Review Board before initiating any recruitment, and all participants provided informed consent via an e-consent or written consent.

### Survey

Data collection occurred via REDCap surveys sent to participants via email, with telephone-based data collection as a backup to ensure that individuals without internet access were able to participate. The data collection used an ecological momentary assessment (EMA) framework. EMA (vs standard data collection methods) is thought to limit biased responding in participants due to retrospection because in EMA participants are asked about their current experience [16]. The surveys recorded COVID-19 symptoms, oxygen saturation, vital signs, and medication adherence. Participants completed a baseline survey before starting the study medication, followed by surveys approximately twice a day for 15 days after starting the study drug; occasional delayed responding or technical errors led to a maximum of 17 days. At each survey, participants were asked whether they were experiencing a symptom at all. When they indicated “no,” this was coded as a value of 0. When they reported “yes,” they were then asked to rate the perceived severity of the symptom on a scale from 1 to 10. The symptoms rated included perceived fever, cough, shortness of breath, fatigue or weakness, chills, nausea, body aches, diarrhea, loss of appetite, difficulty with sense of smell, and difficulty with sense of taste.

### Statistical Analysis

Analyses were conducted in Mplus [17], with visualizations produced in R. Because these analyses were not preregistered, we recommend considering them primarily descriptive and exploratory. Results are reported for the entire group of participants without reference to randomized controlled trial (RCT) group because there were no clear differences between groups on latent trajectory outcomes (the same 30 tests were conducted as reported below for demographic variables, yet the lowest P value was 0.015, whereas we considered 0.01 significant due to the large number of tests).

### Results

### Participant Characteristics

In total 162 participants completed at least some EMA surveys (n=162; median surveys answered, 23; median days covered, 16); demographic data are missing for 10. Participants who reported were mostly White (70%, n=106), although many participants reported that they were Black (25%, n=38), with an average age (SD) of 45.80 (13.04). The sample was primarily female, as judged by the number reporting being assigned female sex at birth (69%, n=109).

Symptom Frequency and Severity Across and within Patients Over Time

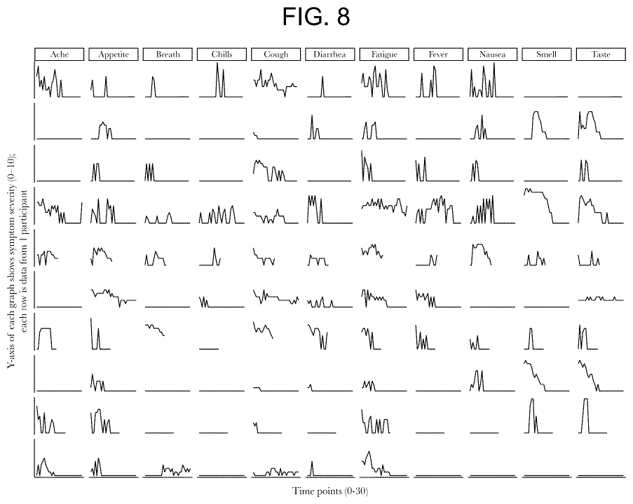

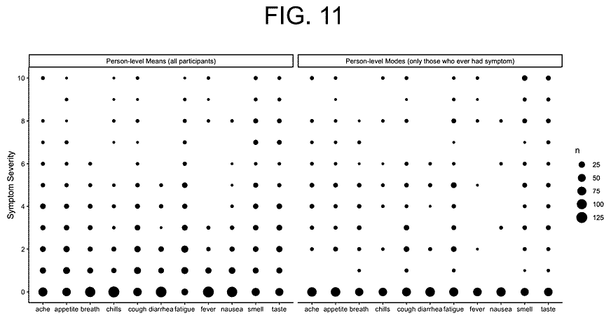

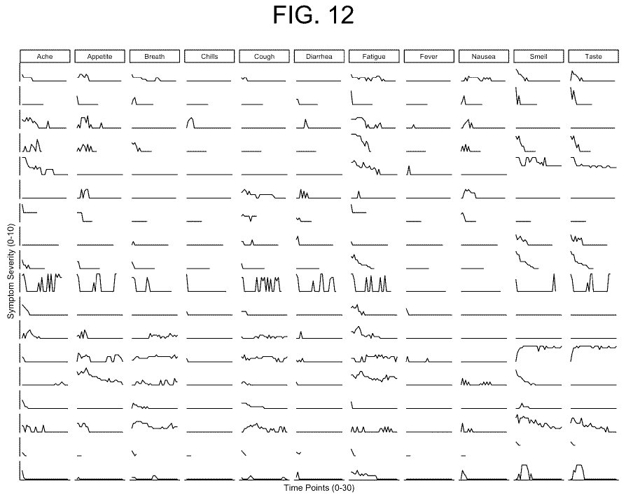

The most common rating for all symptoms was 0, or not present (TABLE 5; FIG. 11). FIG. 8, which depicts raw data for all symptoms for a random set of participants, demonstrates that the frequent ratings of 0 occurred in a number of patterns, including symptoms never occurring, occurring and then resolving, starting in the middle of the reporting period, and fluctuating during some portion of the reporting period. The frequent “saw tooth” patterns seen in FIG. 8 suggest symptoms waxing and waning. An expanded version of this figure is available in FIG. 12.

TABLE 5
Between- and with-person means and variation of symptoms.

| | | | Within Subjects | |
|---|---|---|---|---|
| | Between Subjects | | Range of Person- | Range of Person- |
| Symptom | Mean | SD | Level Means | Level Variance |
| Ache | 1.73 | 2.22 | 0-10 | 0-6.36 |
| Appetite | 1.73 | 2.09 | 0-10 | 0-6.36 |
| Breath | 0.93 | 1.48 | 0-6 | 0-3.28 |
| Chills | 0.7 | 1.7 | 0-10 | 0-4.17 |
| Cough | 1.7 | 2.09 | 0-10 | 0-3.81 |
| Diarrhea | 0.63 | 1.11 | 0-5 | 0-5.66 |
| Fatigue | 2.49 | 2.23 | 0-10 | 0-5.66 |
| Fever | 0.60 | 1.62 | 0-10 | 0-3.81 |
| Nausea | 0.66 | 1.26 | 0-8 | 0-6.36 |
| Smell | 2.75 | 2.93 | 0-10 | 0-4.87 |
| Taste | 2.53 | 2.74 | 0-10 | 0-4.95 |

Between and within-level statistics for the self-reported symptoms. All individual participant responses were included (that is, these are based on the raw data and not the data used for the latent trajectory analyses). The Between Subject Mean and SD shows the average across the entire group of participants and how wide the distribution was around the average. Within Subject statistics, on the other hand, provide us with information about each individual's mean level of symptoms and variance around that mean. Because all 162 individuals each have their own mean and variance, we provide the range of these means and variances in this table. The statistics for Ache thus tell us that although Ache was not highly rated across the entire sample, this is in part because some people never had the symptom (person-level mean of zero) whereas others had severe Ache through the entire reporting period. Further, the fact that the range of person-level variance ranges from 0 to 6.36 means that there was at least one person for whom Ache had no variance (e.g., as would be true for someone with a mean of 0 or only one observation), and at least one person with a very high variance of 6.36, which suggests that this person reported Ache values varying from very low to very high during the study.

### Heterogeneity of Symptom Trajectories

To handle the frequent instance of 0 s in the data, before proceeding with further analysis we (1) restricted further analysis to participants who ever had that symptom and (2) consolidated symptom reports to 5 time points (Times 0 through 4). This procedure had the effect of “smoothing out” the sawtooth pattern present for many participants, allowing analysis of overall tendencies over time.

We examined typical trajectories using latent trajectory modeling. These models focus on slopes and intercepts as a way to characterize the course of symptoms across the whole sample. The intercept refers to the participant's estimated level of the symptom at Time 0 (i.e., during the first 3 days of the study). For participants with only random fluctuations or a stable level of a symptom, the intercept would be enough to characterize their data, while the existence of a slope denotes that there are systematic changes in the symptom. A linear slope indicates a tendency up or down. A quadratic slope indicates some curve to the line's shape. A cubic slope indicates a second curvature of the line. The modeling allows us to determine whether the data are well characterized by the intercept and 1 or more slopes, as well as how many slopes are required to characterize the data of participants overall. The modeling also allows us to determine whether and how individual participants significantly vary from the average group trajectory. For example, the model might show that the mean slope is negative, indicating resolution of a symptom, but with significant variance, indicating that some participants are better characterized by an upward slope of symptoms.

Of the symptoms, only nausea was not reasonably well characterized by a latent trajectory model. As shown in TABLE 6, all other symptoms were best characterized by at least 1 curvilinear slope in addition to a linear slope, typically showing a decrease but with deceleration. Because we only analyzed those participants who reported the symptom at some point, the number for each model (vs 162 who supplied any EMA) shows how commonly the symptom was reported. The most common symptoms were fatigue, fever, reduced appetite, and problems with sense of taste. The linear slopes all had negative means. Thus, participants on average tended to show reductions in symptoms overtime, but the significant variance of most of the slopes indicates that many participants showed increases in symptoms. As a reminder, there was no indication that participants differed in these trajectories based on treatment group.

TABLE 6
Summary of Latent Trajectory Models

| | | | | | | Quadratic | | Cubic |
|---|---|---|---|---|---|---|---|---|
| | Participants | Intercept Mean | | Linear Slope Mean | | Slope Mean | | Slope Mean |
| Symptom | in Analysis | (Variance) | | (Variance) | | (Variance) | | (Variance) |
| Ache² | 120 | 3.80** | (6.45**) | −1.50** | (2.10*) | 0.20** | (0.07*) | |
| Appetite¹ | 121 | 3.85** | (4.45**) | −1.54** | (0.99*) | 0.18** | (0.04*) | |
| Breath² | 89 | 2.11** | (2.86**) | −0.37* | (1.33*) | 0.02 | (0.06*) | |
| Chill¹ | 74 | 2.25** | (3.63*) | −1.13** | (1.54*) | 0.15** | (0.04) | |
| Cough³ | 129 | 2.86** | (4.65**) | −0.51** | (0.94*) | 0.01 | (0.05*) | |
| Diarrhea² | 100 | 1.66** | (1.68*) | −0.66* | (0.00^{a}) | 0.08* | (0.01) | |
| Fatigue³ | 144 | 4.26** | (5.34**) | −1.35** | (1.61**) | 0.15** | (0.06**) | |
| Fever³ | 72 | 2.26** | (6.06**) | −1.15** | (2.84**) | 0.16** | (0.08*) | |
| Smell¹ | 112 | 5.80** | (5.79**) | −1.32** | (4.20*) | 0.10 | (0.16*) | |
| Taste³ | 119 | 5.06** | (9.20**) | −1.05* | (10.67**) | −0.07 | (1.89**) | 0.03 (0.04**) |

Latent trajectory models were conducted on participants (n = 162) who ever reported the symptom. The intercept, linear, and quadratic slopes are then given. Intercepts indicate where participants start, on average, whereas linear slope indicates the general tendency up or down. Additional slopes indicate the extent to which symptom courses frequently reversed, slowed, or accelerated. Unstandardized estimates are given because these are directly relevant to the response scale of 0 (not at all) to 10 (most severe). Statistically significant slopes indicate that participants showed a group tendency overall. Significant variances indicate that participants meaningfully differed in this tendency. Thus, for example, despite the linear slopes being negative, at least 1 participant had a positive slope where the variance was statistically significant.
*P < .05;
**P < .001.
^{1, 2, 3}Indicates number of fit indices showing good to excellent fit. When the number is higher, we can be more certain that the model describes the overall sample well.
^{a}Variance was fixed to 0 to permit estimation.

On the average, judging from the linear slope values, participants recovered the quickest from aches and low appetite and most slowly from cough and difficulty breathing. With the exception of problems with sense of taste, all other symptoms had a second quadratic term that was positive, which indicates that although their symptoms went down, the rate at which they dropped began to slow. Thus, the most typical course was an initially rapid recovery followed by some plateauing, or a pattern of decelerating improvement. Most symptoms had significant variance in both the linear and quadratic slope, which means that the curve could also move in a different direction for some participants. For example, some participants increased in symptoms and then decreased, whereas others had stable levels of symptoms or had increased symptoms that were maintained across the rest of the reporting period. The picture is even more complicated for taste, which had an additional cubic slope. This symptom thus showed at least 2 inflection points for many participants, as would be true if the symptom went up, down, and then up again.

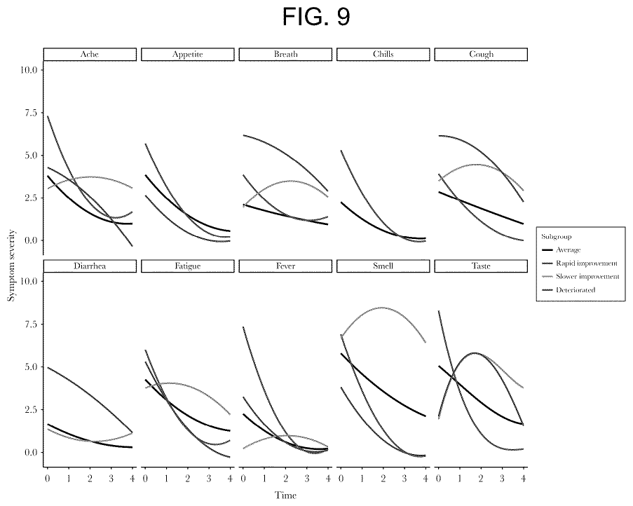

FIG. 9 displays illustrative curves for each symptom. These figures display the most typical (mean) course, alongside the typical course of (1) participants whose symptoms reduced rapidly, as well as (2) those whose symptoms either reduced more slowly or were exacerbated. In the latter 2 cases, the 10 participants with the most extreme linear slopes had their parameters averaged to depict a typical rapidly improving and slowly improving course (with the exception of diarrhea, for which the most extreme quadratic courses were averaged because the linear slope had no variance). Finally, the single participant who deteriorated according to study criteria yet also provided a full set of EMA data is also presented. This participant experienced moderate deterioration and visited the emergency room midway through the reporting period. Examining this participant's raw data revealed no obvious signs of the deterioration aside from 3 elevated ratings of shortness of breath (a 5 or 6 out of 10) that occurred around this period, with these ratings surrounded by ratings of 0.

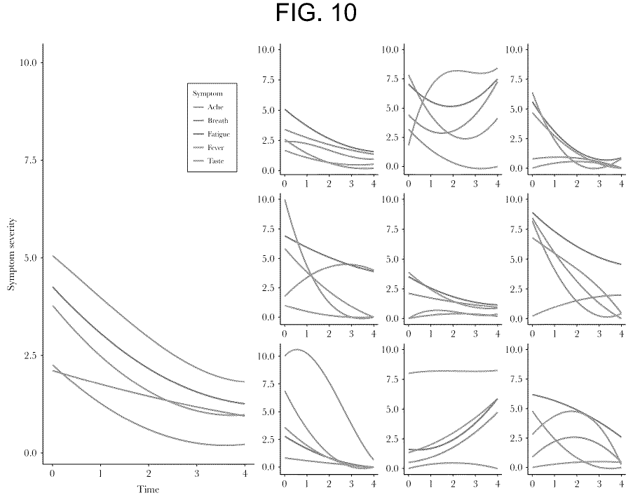

A further depiction of the wide variety of slopes is provided in FIG. 10. Here we depict trajectories for the 5 symptoms with the best-fitting models. On the left, we see the average course of all of these symptoms is an improvement in mild symptoms that slows down. On the right, 9 randomly selected participants show us that these average trajectories vary significantly across individuals, with symptoms rising and falling at different rates across people.

### How Symptoms Change Together Over Time

TABLE 7 shows the correlations among linear slopes. Each person's linear slope tells us to what extent their symptoms generally tend to go up or down over time. The correlation between these slopes tells us whether we should expect that participants have symptoms that go together as they improve or worsen. Some pairs stood out with particularly strong correlations. These include problems with taste and smell, cough and trouble breathing, chills and fever, and, to a lesser extent, ache and fatigue. Thus, for example, we would expect that for a participant whose symptoms included both cough and trouble breathing these symptoms would tend to go up or down together over time. In contrast, for a participant with fever and trouble breathing, we would have no reason to expect that a reduction in fever should necessarily go along with improved breathing, because the correlation is small and negative.

TABLE 7
Partial Correlations Between Pairs of Linear Slopes Controlling
for Intercepts With Number of Participants for Each Comparison

| | Ache | Appetite | Breath | Chill | Cough | Fatigue | Fever | Smell | Taste |
|---|---|---|---|---|---|---|---|---|---|
| Ache | | 97 | 75 | 68 | 100 | 114 | 63 | 90 | 93 |
| Appetite | 0.26** | | 73 | 65 | 100 | 115 | 61 | 89 | 97 |
| Breath | 0.39** | 0.09 | | 54 | 82 | 85 | 47 | 68 | 76 |
| Chill | 0.33** | −0.01 | 0.38** | | 62 | 72 | 50 | 50 | 61 |
| Cough | 0.36** | 0.13 | 0.68** | 0.23 | | 118 | 63 | 93 | 98 |
| Fatigue | 0.51** | 0.44** | 0.46** | 0.31** | 0.33** | | 69 | 104 | 111 |
| Fever | 0.06 | 0.10 | −0.04 | 0.65** | 0.09 | 0.15 | | 52 | 58 |
| Smell | 0.23* | 0.30** | 0.04 | 0.00 | 0.18 | 0.32** | −0.11 | | 104 |
| Taste | 0.23* | 0.35** | 0.07 | 0.19 | 0.23* | 0.31** | 0.09 | 0.74** | |

No. for each comparison is on the top diagonal. Positive correlations indicate that symptoms tend to go either or up or down together. Negative correlations indicate that the symptoms diverge in trajectories (as 1 goes up, the other goes down). Thus, problems with smell are very likely to resolve along with problems with taste. Diarrhea is not included in these analyses because its linear slope had a variance of 0, which means it cannot correlate with other slopes.
*P < .05;
**P < .01.

### Symptom Dynamics, Demographics, and Clinical Deterioration

Most participants who deteriorated did so in the first several days and stopped providing EMA data. Thus, most of the 9 participants who deteriorated provided data only for Time 0 for the trajectories presented. Accordingly, we examined differences between the 9 participants who deteriorated (i.e., developed dyspnea and hypoxia) and the rest of the sample only in the intercepts (the only meaningful trajectory information for participants who only reported during Time 0). Given the small sample of participants who deteriorated, Mann-Whitney U tests were conducted. Two effects retained statistical significance above a correction for the number of tests conducted in this analysis: Participants who deteriorated were far more likely to have an elevated intercept (i.e., higher initial levels) for both cough and diarrhea (all P=0.002).

We also examined demographic variables to see if symptom dynamics differed, adopting a P of 0.01 to balance multiple testing against discovery of potentially important findings. Men and women showed no differences in any intercepts or slopes. Black participants had a significantly higher intercept of chill and cough (all P<0.007), suggesting more severe symptoms at the start of the study. No other slopes or intercepts showed clearly significant differences. Body mass index (BMI) did not correlate significantly with any intercepts or slopes. Thus, there were few signs that demographic variables were related to the course of COVID-19 symptoms.

### Discussion

Our study characterizes the course of COVID-19 among community-dwelling patients who are recovering at home. This information is important because it informs clinicians, patients, and researchers about the high degree of heterogeneity, both between and within patients. We made the following 4 major observations: First, the COVID-19 symptom course is highly heterogenous. Second, early symptoms related to participant demographics and clinical deterioration. Third, symptoms often showed a pattern of decelerating improvement. Fourth, some symptoms are likely to improve in tandem, whereas others are not. We discuss each of these points further below.

The overall picture is of symptoms reducing for most participants, but often with a slowing of this reduction, often further complicated by waxing and waning of symptoms (e.g., across 12-hour periods). Further, problems with the sense of taste were characterized by additional curvilinearity, reflecting increased volatility. As a notable example, the participant who clinically deteriorated (developed dyspnea and hypoxia) did this in spite of symptoms showing average or rapidly improving course in some respects. Although this is a report of only a single participant who experienced moderate deterioration, it stands as a warning that deterioration in COVID-19 can be rapid and unpredictable.

We found no evidence that symptom dynamics varied meaningfully across the treatment groups from the parent study, nor any association with BMI. However, participants who deteriorated were more likely to have initially high levels of cough and diarrhea. This finding should be taken as exploratory and requiring confirmatory tests of whether higher levels of these symptoms in the initial days of illness might presage clinical deterioration, especially because 3 of our deteriorated participants were deteriorated at baseline. Importantly, our more global findings indicate that the time frame of assessment might be crucial. Our finding is that participants with higher self-report of cough and diarrhea near the beginning of their illness were more likely to experience deterioration at some point. Because symptom courses often wax and wane, asking participants if they ever experienced that symptom or experienced it later in the illness would not necessarily yield the same result. However, at least 1 report has found that diarrhea presaged clinical deterioration [18]. We expect that our finding regarding cough might simply reflect the fact that most of those who deteriorated did so early in the reporting period, but the possibility that severity of cough actually predicts deterioration seems worth investigating.

Our findings indicate that patients and health care providers can expect a variety of symptom courses, including the development of additional symptoms and a gradual and potentially stalling decline of some symptoms. The latter finding is consistent with another study showing that a minority of participants continue to experience symptoms, particularly cough, fatigue, and shortness of breath [6]. In our results, cough and shortness of breath showed the smallest slope downward on average, suggesting, in combination with previous results, that these symptoms in particular are likely to be prolonged for many patients.

Whether the decelerating improvement pattern is unique to COVID-19 is difficult to determine because few studies have examined other common infectious diseases in regard to common trajectories of symptoms. However, a report focusing on the development of a symptom measure for influenza reported the average course of several symptoms [19]. Although challenging to compare directly due to differences in methodology, our results seem to suggest (1) a longer course for significant symptoms of COVID-19 and (2) a greater deceleration in improvement across the second week. That is, for influenza symptoms, rapid recovery over the first 4 days (Time 0 to Time 1 in our analysis) was followed by a more gradual, approximately linear slope for the remainder of the days assessed. Our participants experienced more gradual initial improvement, followed, most commonly, by a greater deceleration of improvement.

Judging from correlated slopes, some pairs of symptoms are likely to reduce in tandem. The strongest candidate for such a pairing is problems with smell and taste, which should be expected given the functional connection between the 2 senses. Similarly, cough and shortness of breath, fever and chills, and, to a lesser extent, ache and fatigue are all pairs that show a tendency to decline overtime together.

### Limitations

The results of the study should be interpreted in light of its limitations. Patients were enrolled after diagnosis, which could have been up to 7 days after experiencing symptoms, and even longer after infection. This fact could explain some of the wide variety of trajectories, but not the observed waxing and waning or stalled improvement. Data were drawn from an RCT, with limited diversity in patient population, conducted in 1 Midwestern metropolitan area, during the initial 7 months of the COVID-19 outbreak. It is unclear to what extent we should expect prevalent symptoms of COVID-19 or their course to vary by virus variant, demographics, community, or even country. It is possible that although asking participants first whether they had a symptom or not may have reduced patient burden, it may also have inclined participants to answer “no” even if a symptom was present in a mild form. Participants provided self-report of symptoms, which especially must be kept in mind when attempting to apply results to objective conditions. For example, 1 report found that a significant proportion of participants with COVID-19 reporting olfactory dysfunction did not meet criteria upon evaluation [20]. Nevertheless, self-reported symptoms of problems with smell and taste have emerged as 2 of the best predictors of COVID-19 status [21, 22].

### Conclusions

In conclusion, COVID-19 is a heterogeneous illness in terms of subjective symptoms and course. When patients experience a given symptom, it may fluctuate but will typically follow a gradual improvement over the course of 2 weeks or more. Many patients, however, will develop new symptoms during the same course, and many symptoms may take considerably beyond a 2-week window to resolve completely. This tendency toward slowly improving symptoms may be related to the increasing reports of a more chronic form of the disease in certain populations [6, 8, 11]. It is important to warn patients, and clinicians caring for them, not to expect a linear decrease in all symptoms; some may be intermittent or get worse before they get better. Future studies following a cohort of patients with COVID-19 symptoms over a longer period of time could be useful in determining how, if at all, acute symptom presentation relates to chronic symptom experiences.

### Statistical Analyses Details

Intended analyses were pre-registered (https://osf.io/2jwnh/), but the self-reported symptoms proved too nonnormal for the intended analysis. Psychophysiological data could be analyzed under the pre-registered analyses and will thus be reported separately. The current analyses should be considered descriptive and exploratory and do not test specific a priori hypotheses.

To allow examination of symptom course, we created more normally-distributed symptom measures by computing the average rating for that symptom across three days (or typically four and up to five, in the case of the last set of days, because a minority of the sample provided ratings into a seventeenth day [median days covered=16]). This resulted in five reporting periods and produced variables for analysis that were less pronouncedly nonnormal. Latent trajectory models were then conducted for those participants who had that symptom during at least one time period. Including all participants (e.g., those who never had that symptom) would have resulted in extremely nonnormal data.

Models were fitted with linear slopes first, then quadratic, and finally cubic slopes. When small negative variances occurred for error variances, intercepts, and slopes, these were fixed to zero to permit proper estimation. The best-fitting model that converged appropriately was retained. In line with recommendations in the literature [18], we considered the comparative fit index (CFI) and Tucker-Lewis index (TLI) at or greater than 0.95, and a standardized root mean square residual SRMR of less than 0.08 to indicate good fit. We present all models but emphasize those in which at least one of these statistics indicated good fit. In some cases in which fit was not uniformly good, adding a cubic slope led to estimation errors, which may have occurred due to the somewhat modest sample size. It is thus possible that with a larger sample size more of the symptoms would have displayed a more systematically curvilinear course with improved fit indices.

### REFERENCES

1. Centers for Disease Control and Prevention. Symptoms of Coronavirus. https://www.cdc.gov/coronavirus/2019-ncov/symptoms-testing/symptoms.html. Accessed 16 Mar. 2021.
2. World Health Organization. Coronavirus disease (COVID-19). https://www.who.int/emergencies/diseases/novel-coronavirus-2019/question-and-answers-hub/q-a-detail/coronavirus-disease-covid-19. Accessed 16 Mar. 2021.
3. Lechien J R, Chiesa-Estomba C M, Place S, et al.; COVID-19 Task Force of YO-IFOS. Clinical and epidemiological characteristics of 1420 European patients with mild-to-moderate coronavirus disease 2019. J Intern Med 2020; 288:335-44.
4. Wiersinga W J, Rhodes A, Cheng A C, et al. Pathophysiology, transmission, diagnosis, and treatment of coronavirus disease 2019 (COVID-19): a review. JAMA 2020; 324:782-93.
5. Liu W, Liu Y, Xu Z, et al. Clinical characteristics and predictors of the duration of SARS-CoV-2 viral shedding in 140 healthcare workers. J Intern Med. In press.
6. Tenforde M W, Kim S S, Lindsell C J, et al.; IVY Network Investigators; CDC COVID-19 Response Team; IVY Network Investigators. Symptom duration and risk factors for delayed return to usual health among outpatients with COVID-19 in a multistate health care systems network—United States, March-June 2020. MMWR Morb Mortal Wkly Rep 2020; 69:993-8.
7. Pullen M F, Skipper C P, Hullsiek K H, et al. Symptoms of COVID-19 outpatients in the United States. Open Forum Infect Dis 2020; 7.
8. Cellai M, O'Keefe J B. Characterization of prolonged COVID-19 symptoms in an outpatient telemedicine clinic. Open Forum Infect Dis 2020; 7.
9. Xia L, Chen J, Friedemann T, et al. The course of mild and moderate COVID-19 infections—the unexpected long-lasting challenge. Open Forum Infect Dis 2020; 7.
10. Ng D H L, Choy C Y, Chan Y H, et al.; National Centre for Infectious Diseases COVID-19 Outbreak Research Team. Fever patterns, cytokine profiles, and outcomes in COVID-19. Open Forum Infect Dis 2020; 7.
11. Maxwell E. Living with COVID-19. 2020. Available from: https://evidence.nihr.ac.uk/themedreview/living-with-covid19/. Accessed 16 Mar. 2021.
12. Lenze E J, Mattar C, Zorumski C F, et al. Fluvoxamine vs placebo and clinical deterioration in outpatients with symptomatic COVID-19: a randomized clinical trial. JAMA 2020; 324:2292-300.
13. Curran P J, Hussong A M. The use of latent trajectory models in psychopathology research. J Abnorm Psychol 2003; 112:526-44.
14. Duncan T E, Duncan S C. The ABC's of LGM: an introductory guide to latent variable growth curve modeling. Soc Personal Psychol Compass 2009; 3:979-91.
15. Henry B, Moffitt T E, Caspi A, et al. On the “remembrance of things past”: a longitudinal evaluation of the retrospective method. Psychological Assessment 1994; 6: 92-101.
16. Shiffman S, Stone A A, Hufford M R. Ecological momentary assessment. Annu Rev Clin Psychol 2008; 4:1-32.
17. Muthen L K, Muthen B O. Mplus User's Guide. Los Angeles, CA: Muthen & Muthen; 1998.
18. Zheng T, Yang C, Wang H Y, et al. Clinical characteristics and outcomes of COVID-19 patients with gastrointestinal symptoms admitted to Jianghan Fangcang Shelter Hospital in Wuhan, China. J Med Virol. In press.
19. Powers J H 3rd, Bacci E D, Guerrero M L, et al. Reliability, validity, and responsiveness of InFLUenza Patient-Reported Outcome (FLU-PRO©) scores in influenza-positive patients. Value Health 2018; 21:210-8.
20. Lechien J R, Cabaraux P, Chiesa-Estomba C M, et al. Objective olfactory evaluation of self-reported loss of smell in a case series of 86 COVID-19 patients. Head Neck 2020; 42:1583-90.
21. Menni C, Valdes A M, Freidin M B, et al. Real-time tracking of self-reported symptoms to predict potential COVID-19. Nat Med 2020; 26:1037-40.
22. Yan C H, Faraji F, Prajapati D P, et al. Association of chemosensory dysfunction and COVID-19 in patients presenting with influenza-like symptoms. Int Forum Allergy Rhinol 2020; 10:806-13.

### Example 6: Association Between Antidepressant Use and ED or Hospital Visits in Outpatients with SARS-CoV-2

This example describes a retrospective cohort study assessing the association between home use of antidepressants and a composite outcome of emergency department visits or hospital admission within 30 days. After adjusting for confounding variables, antidepressant exposure was associated with reduced incidence of the composite outcome in a dose-dependent manner. In an exploratory secondary analysis, antidepressants with functional inhibition of acid sphingomyelinase (FIASMA) activity properties were also associated with reduced incidence of the outcome in a dose-dependent manner.

This retrospective observational study using the electronic medical record (EMR). This example provides evidence that when taken on an outpatient basis (generally starting prior to infection), multiple antidepressants might have protective effects against need for ER visits and hospitalization. There is some support for antidepressants in general, FIASMA drugs, S1R agonists, and SSRIs. One unexpected drug that was found to be protective is bupropion. It is significantly associated with lower risk even when considered by itself.

It is believed that there is some evidence that bupropion is a S1R agonist. It is also a serotonin 5HT3 receptor antagonist, which may counteract platelet serotonin storm effects. And it is possible it could be a FIASMA drug, but literature support is lacking.

It is possible that other SSRI, FIASMA, or S1R agonist drugs may also be effective in treating COVID19. There are some observational studies suggesting this. There are also unpublished clinical trials ongoing using the S1R agonist, antihistamine, and serotonin antagonist cyproheptadine alone and also cyproheptadine combined with fluvoxamine.

### Abstract

Objective: Antidepressants have previously been associated with better outcomes in patients hospitalized with COVID-19, but their effect on clinical deterioration among ambulatory patients has not been fully explored. The objective of this study was to assess whether antidepressant exposure was associated with reduced emergency department (ED) or hospital visits among ambulatory patients with SARS-CoV-2 infection.

Methods: This retrospective cohort study included adult patients (N=25,034) with a positive SARS-CoV-2 test performed in a non-hospital setting.

Logistic regression analyses tested associations between home use of antidepressant medications and a composite outcome of ED visitation or hospital admission within 30 days. Secondary exposures included individual antidepressants and antidepressants with functional inhibition of acid sphingomyelinase (FIASMA) activity.

Results: Patients with antidepressant exposure were less likely to experience the primary composite outcome compared to patients without antidepressant exposure (adjusted odds ratio [aOR] 0.88, 95% CI 0.79-0.98, p=0.02). This association was only observed with daily doses of at least 20 mg fluoxetine-equivalent (aOR 0.88, 95% CI 0.77-0.99, p=0.04), but not with daily doses lower than 20 mg fluoxetine-equivalent (aOR 0.89, 95% CI 0.76-1.05, p=0.16). In exploratory secondary analyses, the outcome incidence was also reduced with exposure to selective serotonin reuptake inhibitors (aOR 0.84, 95% CI 0.74-0.96, p=0.01), bupropion (aOR 0.65, 95% CI 0.51-0.82, p<0.001), and FIASMA antidepressant drugs (aOR 0.87, 95% CI 0.77-0.98, p=0.02).

Conclusions: Antidepressant exposure was associated with a reduced incidence of emergency department visitation or hospital admission among SARS-CoV-2 positive patients, in a dose-dependent manner. These data support the FIASMA model of antidepressants' effects against COVID-19.

### Introduction

The novel coronavirus SARS-CoV-2 and its variants have created a worldwide infectious disease crisis in the form of the coronavirus disease 2019 (COVID-19) pandemic (1). Because many individuals around the world have not been fully vaccinated (2) and breakthrough infections can sometimes cause severe COVID-19 in vaccinated individuals (3), effective treatments with favorable tolerability profiles are urgently needed. Ideal treatments would be easy to use, low in cost, produced in oral formulations, and rapidly available globally, including in low and middle income countries. Repurposing of existing drugs may be imperative to identifying such therapeutics (4).

Certain antidepressants, particularly selective serotonin reuptake inhibitors (SSRIs), have shown promise as early treatments for COVID-19 (5, 6). Multiple preclinical studies have demonstrated in vitro efficacy of both SSRIs and non-SSRI antidepressants against SARS-CoV-2 in human and non-human host cells (7-9). Potential mechanisms of action include immunomodulatory activity via sigma-1 receptor (S1R) agonism and non-S1R pathways (e.g., NF-κB, inflammasomes, TLR4, PPARγ) (10), antiviral and anti-inflammatory actions via functional inhibition of acid sphingomyelinase (FIASMA) activity (9, 11), as well as serotonin modulatory and anti-platelet activity of these agents. Among patients with COVID-19 treated in acute care settings, three large retrospective observational cohort studies have reported reduced death or mechanical ventilation among patients with antidepressant exposure (12-14). In these studies, the association with improved outcomes appeared to be strongest among patients taking the SSRIs fluoxetine or fluvoxamine (12-14). In the ambulatory setting, the use of fluvoxamine for 10-15 days was associated with a reduced risk of clinical deterioration in two randomized, placebo-controlled trials (15, 16) as well as in one non-randomized observational study (17). However, the impact of antidepressants other than fluvoxamine have not been investigated in the ambulatory setting. In addition, prior studies did not examine a potential dose-response relationship between antidepressant use and reduced risk of developing severe COVID-19. Therefore, the goal of this study was to test the hypothesis that exposure to antidepressants would be associated with reduced incidence of clinical deterioration, defined as emergency department (ED) visitation or hospital admission, among ambulatory patients with COVID-19.

### Methods

This retrospective cohort study was approved by the Human Research Protection Office at Washington University School of Medicine. A waiver of informed consent was obtained to retrieve data from the electronic health record. This study was conducted at BJC Healthcare, a network of university-affiliated hospitals and community hospitals located in Missouri and Illinois. This manuscript has been written according to STROBE guidelines.

The population included patients age 18 or older with a positive SARS-CoV-2 test (either polymerase chain reaction or antigen test) performed within the BJC Healthcare network between 3/1/2020 and 5/16/2021. Because the outcome was ED visitation or hospital admission, patients who were admitted to a hospital at the time of the test or admitted on the same day as the test were excluded from the analysis.

Outpatient medication exposure was determined by extracting the home medication list as documented in the electronic health record of all ambulatory or inpatient encounters within the study period. For each medication of interest, exposure was defined as inclusion of the medication on the home medication list at any encounter. The full list of medications retrieved is found in TABLE 8 in the online supplement. For antidepressants, the strength of the oral formulation was also recorded.

TABLE 8
Medications Retrieved from Home Medication List.

| Medication Class | Medications Included |
|---|---|
| Selective Serotonin | Citalopram, Escitalopram, Fluoxetine, |
| Reuptake Inhibitors | Fluvoxamine, Paroxetine, Sertraline |
| Tricyclic Antidepressants | Amitriptyline, Clomipramine, Desipramine, |
| | Doxepin, Imipramine, Nortriptyline |
| Phenylpiperazines | Nefazodone, Trazodone |
| Serotonin Norepinephrine | Desvenlafaxine, Duloxetine, |
| Reuptake Inhibitors | Levomilnacipran, Venlafaxine |
| Other Antidepressants | Bupropion, Mirtazapine |
| Benzodiazepines | Alprazolam, Chlordiazepoxide, Clorazepate, |
| | Diazepam, Flurazepam, Lorazepam, |
| | Oxazepam, Temazepam, Triazolam |
| Z Drugs | Eszopiclone, Zaleplon, Zolpidem |
| Antipsychotics | Aripiprazole, Asenapine, Brexpiprazole, |
| | Cariprazine, Chlorpromazine, Clozapine, |
| | Droperidol, Fluphenazine, Haloperidol, |
| | Iloperidone, Loxapine, Lurasidone, |
| | Olanzapine, Paliperidone, Perphenazine, |
| | Pimavanserin, Pimozide, Quetiapine, |
| | Risperidone, Thiothiene, Ziprasidone |

To explore potential mechanisms of action, antidepressants were first stratified by class (i.e., SSRIs, serotonin norepinephrine reuptake inhibitors, tricyclic antidepressants, phenylpiperazines). Then, SSRIs were grouped based on their activity at the S1R (18): high-affinity agonists (fluoxetine, fluvoxamine), intermediate-affinity agonists (escitalopram, citalopram), low-affinity agonist (paroxetine), and antagonist (sertraline). Finally, antidepressants were classified as drugs with FIAMA activity, defined as showing an in vitro functional inhibition effect on acid sphingomyelinase (ASM—i.e., a residual ASM activity <100%) (9, 19), including amitriptyline, citalopram, clomipramine, desipramine, doxepin, escitalopram, fluoxetine, fluvoxamine, imipramine, nortriptyline, paroxetine, sertraline, and venlafaxine, and antidepressants with unknown FIASMA, defined as unknown residual ASM activity after prolonged (e.g., >6 hours) incubation times, including bupropion, desvenlafaxine, duloxetine, levomilnacipran, mirtazapine, nefazodone, trazodone, vilazodone, and vortioxetine.

The primary outcome was a composite of emergency department (ED) visitation or hospital admission within 30 days after the positive SARS-CoV-2 test. Encounters at any ED or hospital in the BJC network were identified.

### Statistical Methods

All analyses were conducted using R version 4.0.3 (R, Vienna, Austria). P values less than 0.05 were considered to be statistically significant. No sample size calculation was performed, as all patients with available data were included. The cohort was divided into two groups: patients with outpatient exposure to an antidepressant and those with no outpatient exposure to an antidepressant. Demographic characteristics, elements of the medical history, and home medication characteristics were compared between these two groups using chi-square tests. The dose range for each individual antidepressant was described using median, interquartile range, and range. To permit comparisons of dose ranges across medications with different potencies, strengths were converted to fluoxetine-equivalents using the conversion factors based on prior work (20) (TABLE 9).

TABLE 9
Conversion Factors Used to Calculate Fluoxetine-Equivalent Dose

| | Conversion |
|---|---|
| Antidepressant | Factor |
| Fluoxetine | 1 |
| Fluvoxamine | 0.3 |
| Citalopram | 1.11 |
| Escitalopram | 2.22 |
| Paroxetine | 1.17 |
| Sertraline | 0.42 |
| Vilazodone | 1.5 |
| Vortioxetine | 3 |
| Amitriptyline | 0.33 |
| Clomipramine | 0.35 |
| Desipramine | 0.21 |
| Doxepin | 0.29 |
| Imipramine | 0.29 |
| Nortriptyline | 0.40 |
| Nefazodone | 0.08 |
| Trazodone | 0.10 |
| Desvenlafaxine | 0.40 |
| Duloxetine | 0.67 |
| Levomilnacipran | 0.33 |
| Venlafaxine | 0.28 |
| Bupropion | 0.11 |
| Mirtazapine | 0.79 |

Crude and adjusted odds ratios for ED or hospital encounters within 30 days were estimated using logistic regression. The primary analysis compared patients exposed to any antidepressant against patients exposed to no antidepressant. Medical comorbidity and concurrent use of other psychotropic medications are more prevalent among patients hospitalized for COVID-19 with psychiatric disorders (12, 21), and these patients are more likely both to take antidepressants and to develop severe COVID-19 (22). Therefore, the logistic regression adjusted for sex, age, race and ethnicity, obesity, number of diagnoses, history of mood or anxiety disorder, history of other psychiatric disorders, number of home medications, concurrent exposure to benzodiazepines or Z drugs (eszopiclone, zaleplon, or zolpidem), and to antipsychotic drugs. In secondary analyses, crude and adjusted odds ratios for each individual antidepressant and for each class of antidepressants were estimated using the above-mentioned methods. As an additional secondary analysis, we examined a potential dose-effect relationship by grouping patients according to their daily antidepressant dose in fluoxetine-equivalents (<20 mg, ≥20 mg, and ≥40 mg). Crude and adjusted odds ratios for the primary outcome were also calculated for each of these groups. In each secondary analysis, the exposed patients were compared to patients with no antidepressant exposure (excluding patients exposed to different antidepressants).

### Results

Between Mar. 1, 2020 and May 16, 2021, 25,034 outpatients were found to have a positive SARS-CoV-2 test result. Of these patients, 5,997 cases (24%) had exposure to at least one antidepressant documented in the home medication list, at a median fluoxetine-equivalent dose of 22.5 mg (interquartile range 20.0-44.4 mg). Patients with antidepressant exposure were older, more likely to be female, and more likely to be of White race and non-Hispanic ethnicity compared to patients without antidepressant exposure (TABLE 10). Additionally, the patients with antidepressant exposure were more likely to carry a greater number of medical diagnoses, to take a greater number of home medications, including any benzodiazepine or Z-drug and any antipsychotic, and to have diagnoses of mood or anxiety disorder or other psychiatric disorders. The most common classes of antidepressants included SSRIs (3,756 patients, 62.6%), serotonin norepinephrine reuptake inhibitors (1,292 patients, 21.5%), and phenylpiperazines (1,007 patients, 16.8%) (TABLE 11).

TABLE 10
Characteristics of Patients with and without Antidepressant Exposure.

| | | | | With | | Without | | |
|---|---|---|---|---|---|---|---|---|
| | Number | | | Antidepressant | | Antidepressant | | |
| | with | Overall | | Exposure | | Exposure | | |
| | Missing | (N = 25,034) | | (N = 5,991) | | (N = 19,043) | | |
| Variable | Data | N | % | N | % | N | % | P |
| Sex | 18 | | | | | | | <0.001 |
| Female | | 15,030 | 60.1 | 4,391 | 73.3 | 10,639 | 55.9 | |
| Male | | 9,986 | 39.9 | 1,600 | 26.7 | 8,386 | 44.1 | |
| Race | 1,250 | | | | | | | <0.001 |
| American Indian/ | | 51 | 0.2 | 11 | 0.2 | 40 | 0.2 | |
| Alaska Native | | | | | | | | |
| Asian | | 275 | 1.2 | 40 | 0.7 | 235 | 1.3 | |
| Black or African | | 6,389 | 26.9 | 929 | 15.6 | 5460 | 30.6 | |
| American | | | | | | | | |
| Other | | 7 | <0.1 | 0 | 0 | 7 | <0.1 | |
| Other Pacific | | 54 | 0.2 | 12 | 0.2 | 42 | 0.2 | |
| Islander | | | | | | | | |
| White | | 17,008 | 71.5 | 4,946 | 83.3 | 12062 | 67.6 | |
| Ethnicity | 2,909 | | | | | | | <0.001 |
| Hispanic | | 440 | 2.0 | 66 | 1.1 | 374 | 2.3 | |
| Non-Hispanic | | 21,685 | 98.0 | 5,713 | 98.9 | 15972 | 97.7 | |
| Age Category | 0 | | | | | | | <0.001 |
| 18-31 | | 6,034 | 24.1 | 921 | 15.4 | 5,113 | 26.8 | |
| 32-45 | | 5,738 | 22.9 | 1,311 | 21.9 | 4,427 | 23.2 | |
| 46-61 | | 7,041 | 28.1 | 1,935 | 32.3 | 5,106 | 26.8 | |
| 62+ | | 6,221 | 24.9 | 1,824 | 30.4 | 4,397 | 23.1 | |
| Obese | 3,234 | 11,197 | 51.4 | 3,327 | 56.0 | 7,870 | 49.6 | <0.001 |
| Number of | 1854 | | | | | | | <0.001 |
| Diagnoses | | | | | | | | |
| 01-04 | | 6,152 | 26.5 | 343 | 5.7 | 5,809 | 33.8 | |
| 05-10 | | 5,410 | 23.3 | 986 | 16.5 | 4,424 | 25.7 | |
| 11-20 | | 5,623 | 24.3 | 1,733 | 28.9 | 3,890 | 22.6 | |
| 21+ | | 5,995 | 25.9 | 2,928 | 48.9 | 3,067 | 17.8 | |
| Mood or Anxiety | 1,854 | 5,884 | 25.4 | 4,088 | 68.2 | 1,796 | 10.4 | <0.001 |
| Disorder | | | | | | | | |
| Other Psychiatric | 1,854 | 2,417 | 10.4 | 1,287 | 21.5 | 1,130 | 6.6 | <0.001 |
| Disorder | | | | | | | | |
| Number of Home | 0 | | | | | | | <0.001 |
| Medications | | | | | | | | |
| 0 | | 6,658 | 26.6 | 0 | 0 | 6,658 | 35.0 | |
| 01-05 | | 6,351 | 25.4 | 966 | 16.1 | 5,385 | 28.3 | |
| 06-13 | | 6,143 | 24.5 | 1,940 | 32.4 | 4,203 | 22.1 | |
| 14+ | | 5,882 | 23.5 | 3,085 | 51.5 | 2,797 | 14.7 | |

TABLE 11
Antidepressants Used and Range of Formulation Strengths

| | | | | | Strength (mg | | |
|---|---|---|---|---|---|---|---|
| | Number | Strength (mg) | | | Fluoxetine-Equivalent) | | |
| | of | | | Min to | | | Min to |
| Antidepressant | Patients | Median | IQR | Max | Median | IQR | Max |
| Any | 5991 | | | | 22.5 | 20-44.4 | 2.9 to 212.7 |
| Antidepressant | | | | | | | |
| Any SSRI | 3749 | | | | 22.2 | 21-42 | 8.4 to 127.6 |
| Fluoxetine | 585 | 20 | 20-40 | 10 to 90 | 20 | 20-40 | 10 to 90 |
| Fluvoxamine | 19 | 50 | 50-100 | 50 to 100 | 15 | 15-30 | 15 to 30 |
| Citalopram | 727 | 20 | 20-40 | 10 to 40 | 22.2 | 22.2-44.4 | 11.1 to 44.4 |
| Escitalopram | 1084 | 10 | 10-20 | 5 to 20 | 22.2 | 22.2-44.4 | 11.1 to 44.4 |
| Paroxetine | 258 | 20 | 10-30 | 10 to 40 | 23.4 | 11.7-35.1 | 11.7 to 46.8 |
| Sertraline | 1180 | 50 | 50-100 | 20 to 100 | 21 | 21-42 | 8.4 to 42 |
| Vilazodone | 51 | 20 | 20-40 | 10 to 40 | 30 | 30-60 | 15 to 60 |
| Vortioxetine | 84 | 10 | 10-20 | 5 to 20 | 30 | 30-60 | 15 to 60 |
| Any Non-SSRI | 3398 | | | | 16.5 | 8.3-33 | 0.87 to 108.8 |
| Any TCA | 788 | | | | 8.25 | 4-16.5 | 0.87 to 63.5 |
| Amitriptyline | 462 | 25 | 10-50 | 10 to 150 | 8.25 | 3.3-16.5 | 3.3 to 49.5 |
| Clomipramine | 11 | 50 | 25-50 | 25 to 75 | 17.5 | 8.75-17.5 | 8.75 to 26.25 |
| Desipramine | 10 | 50 | 25-50 | 10 to 75 | 10.5 | 5.25-10.5 | 2.1 to 15.75 |
| Doxepin | 51 | 25 | 10-25 | 3 to 150 | 7.25 | 2.9-7.25 | 0.87 to 43.5 |
| Imipramine | 17 | 25 | 10-50 | 10 to 100 | 7.25 | 2.9-14.5 | 2.9 to 29 |
| Nortriptyline | 259 | 25 | 10-25 | 10 to 75 | 10 | 4-10 | 4 to 30 |
| Any | 1007 | | | | 5 | 5-10 | 5 to 30 |
| Phenylpiperazine | | | | | | | |
| Nefazodone | 3 | 100 | 100-150 | 100 to 200 | 8 | 8-12 | 8 to 16 |
| Trazodone | 1005 | 50 | 50-100 | 50 to 300 | 5 | 5-10 | 5 to 30 |
| Any SNRI | 1292 | | | | 40 | 20.1-40.2 | 7 to 103.2 |
| Desvenlafaxine | 75 | 50 | 50-100 | 25 to 100 | 20 | 20-40 | 10 to 40 |
| Duloxetine | 726 | 60 | 30-60 | 20 to 60 | 40.2 | 20.1-40.2 | 13.4 to 40.2 |
| Levomilnacipran | 5 | 80 | 40-120 | 40 to 120 | 26.4 | 13.2-39.6 | 13.2 to 39.6 |
| Venlafaxine | 519 | 75 | 75-150 | 25 to 225 | 21 | 21-42 | 7 to 63 |
| Other | 1024 | | | | | | |
| Antidepressant | | | | | | | |
| Bupropion | 805 | 150 | 150-300 | 75 to 450 | 16.5 | 16.5-33 | 8.25 to 49.5 |
| Mirtazapine | 229 | 15 | 15-30 | 7.5 to 45 | 11.9 | 11.9-23.7 | 5.9 to 35.6 |

SSRIs GROUPED BY S1R AFFINITY ^{a}

| | | | | |
|---|---|---|---|---|
| High Affinity | 602 | 20 | 20-40 | 10 to 90 |
| Intermediate | 1784 | 22.2 | 22.2-44.4 | 11.1 to 88.8 |
| Affinity | | | | |
| Low Affinity | 258 | 23.4 | 11.7-35.1 | 11.7 to 46.8 |

ANTIDEPRESSANTS GROUPED BY FIASMA ACTIVITY

| | | | | |
|---|---|---|---|---|
| With FIASMA | 4592 | 22.2 | 20-42 | 0.87 to 135.9 |
| Activity ^{b} | | | | |
| With Unknown | 2519 | 16.5 | 10-36.6 | 5 to 158 |
| FIASMA Activity ^{c} | | | | |

Abbreviations: FIASMA, functional inhibition of acid sphingomyelinase. IQR, interquartile range. SNRI, serotonin-norepinephrine reuptake inhibitor. SSRI, selective serotonin reuptake inhibitor. S1R, sigma-1 receptor. TCA, tricyclic antidepressant.
^{a} SSRIs with high affinity agonist activity at S1R included fluoxetine and fluvoxamine. SSRIs with intermediate affinity agonist activity at S1R included escitalopram and citalopram. SSRIs with low affinity agonist activity at S1R included paroxetine.
^{b} Antidepressants with FIASMA activity included amitriptyline, citalopram, clomipramine, desiparmine, doxepin, escitalopram, fluoxetine, fluvoxamine, imipramine, nortriptyline, paroxetine, sertraline, and venlafaxine.
^{c} Antidepressants with unknown FIASMA activity included bupropion, desvenlafaxine, duloxetine, levomilnacipran, mirtazapine, nefazodone, trazodone, vilazodone, and vortioxetine.

Of the 25,034 patients, 2,867 patients (11%) had an ED or hospital encounter within 30 days of the positive SARS-CoV-2 test result. In a crude analysis, the incidence of an ED or hospital encounter within 30 days was significantly greater among patients with antidepressant exposure than among patients without antidepressant exposure (1,034/5,991=17%, versus 1,833/19,043=10%, respectively, p<0.001). However, after adjusting for demographic characteristics, medical history, and exposure to other medications, antidepressant exposure was associated with decreased odds of an ED or hospital encounter (adjusted odds ratio [AOR] 0.88, 95% CI 0.79-0.98, p=0.02—TABLE 12), in a dose-dependent manner (in those taking <20 mg fluoxetine-equivalent daily: AOR 0.89, 95% CI 0.76-1.05, p=0.16; in those taking ≥20 mg fluoxetine-equivalent daily: AOR 0.88, 95% CI 0.77-0.99, p=0.04; in those taking ≥40 mg fluoxetine-equivalent daily: AOR 0.77, 95% CI 0.66-0.91, p=0.002) (TABLE 13). Other independent predictors of an ED or hospital encounter in the logistic regression analysis included female sex, older age, greater number of diagnoses, greater number of home medications, non-White race or Hispanic ethnicity, benzodiazepine or Z drug exposure, and antipsychotic exposure (TABLE 14 in the online supplement). In the adjusted secondary analyses, SSRIs, antidepressants with FIASMA activity, and bupropion were associated with decreased odds of an ED or hospital encounter (TABLE 12). When patients were stratified according to their daily fluoxetine-equivalent doses, SSRIs and antidepressants with FIASMA activity were associated with decreased odds of the composite outcome only at daily fluoxetine-equivalent doses of at least 20 mg (TABLE 13). Bupropion was associated with decreased odds of the outcome at all doses (TABLE 13).

TABLE 12
Associations between Exposure Variables and ED or Hospital Encounter within 30 Days

| | | | | Unadjusted | | | Adjusted | | |
|---|---|---|---|---|---|---|---|---|---|
| | | Encounter | | Odds | 95% | | Odds | 95% | |
| Exposure | N ^{a} | n | % | Ratio | CI | p | Ratio | CI | p |
| No Antidepressant | 19043 | 1833 | 9.6 | Ref | Ref | Ref | Ref | Ref | Ref |
| Any Antidepressant | 5991 | 1034 | 17.3 | 1.96 | 1.80- | <0.001 | 0.88 | 0.79- | 0.02 |
| | | | | | 2.13 | | | 0.98 | |
| Any SSRI | 3749 | 615 | 16.4 | 1.84 | 1.67- | <0.001 | 0.84 | 0.74- | 0.01 |
| | | | | | 2.03 | | | 0.96 | |
| Fluoxetine | 585 | 95 | 16.2 | 1.82 | 1.45- | <0.001 | 0.80 | 0.62- | 0.09 |
| | | | | | 2.28 | | | 1.04 | |
| Fluvoxamine | 19 | 4 | 21.1 | 2.50 | 0.83- | 0.10 | 0.80 | 0.26- | 0.70 |
| | | | | | 7.55 | | | 2.51 | |
| Citalopram | 727 | 115 | 15.8 | 1.76 | 1.44- | <0.001 | 0.84 | 0.67- | 0.14 |
| | | | | | 2.17 | | | 1.06 | |
| Escitalopram | 1084 | 176 | 16.2 | 1.82 | 1.54- | <0.001 | 0.77 | 0.63- | 0.01 |
| | | | | | 2.15 | | | 0.95 | |
| Paroxetine | 258 | 39 | 15.1 | 1.67 | 1.19- | 0.003 | 0.64 | 0.44- | 0.02 |
| | | | | | 2.36 | | | 0.93 | |
| Sertraline | 1180 | 211 | 17.9 | 2.04 | 1.75- | <0.001 | 0.89 | 0.74- | 0.21 |
| | | | | | 2.39 | | | 1.07 | |
| Vilazodone | 51 | 15 | 29.4 | 3.91 | 2.14- | <0.001 | 1.36 | 0.71- | 0.35 |
| | | | | | 7.16 | | | 2.61 | |
| Vortioxetine | 84 | 12 | 14.3 | 1.56 | 0.85- | 0.15 | 0.58 | 0.30- | 0.10 |
| | | | | | 2.89 | | | 1.11 | |
| Any Non-SSRI | 3398 | 669 | 19.7 | 2.30 | 2.09- | <0.001 | 0.92 | 0.81- | 0.18 |
| | | | | | 2.54 | | | 1.04 | |
| Any TCA | 788 | 184 | 23.4 | 2.86 | 2.41- | <0.001 | 1.04 | 0.86- | 0.70 |
| | | | | | 3.40 | | | 1.26 | |
| Amitriptyline | 462 | 109 | 23.6 | 2.90 | 2.33- | <0.001 | 1.09 | 0.86- | 0.48 |
| | | | | | 3.61 | | | 1.38 | |
| Clomipramine | 11 | 2 | 18.2 | 2.09 | 0.45- | 0.35 | 0.59 | 0.12- | 0.51 |
| | | | | | 9.66 | | | 2.88 | |
| Desipramine | 10 | 1 | 10.0 | 1.04 | 0.13- | 0.97 | 0.24 | 0.03- | 0.18 |
| | | | | | 8.24 | | | 1.92 | |
| Doxepin | 51 | 14 | 27.5 | 3.55 | 1.92- | <0.001 | 1.10 | 0.57- | 0.77 |
| | | | | | 6.58 | | | 2.11 | |
| Imipramine | 17 | 4 | 23.5 | 2.89 | 0.94- | 0.06 | 1.07 | 0.34- | 0.91 |
| | | | | | 8.87 | | | 3.40 | |
| Nortriptyline | 259 | 63 | 24.3 | 3.02 | 2.26- | <0.001 | 1.08 | 0.79- | 0.63 |
| | | | | | 4.02 | | | 1.46 | |
| Any | 1007 | 227 | 22.5 | 2.73 | 2.34- | <0.001 | 0.99 | 0.82- | 0.91 |
| Phenylpiperazine | | | | | 3.19 | | | 1.19 | |
| Nefazodone | 3 | 0 | 0.0 | NA | NA | NA | NA | NA | NA |
| Trazodone | 1005 | 227 | 22.6 | 2.74 | 2.34- | <0.001 | 0.99 | 0.82- | 0.93 |
| | | | | | 3.20 | | | 1.20 | |
| Any SNRI | 1292 | 256 | 19.8 | 2.32 | 2.01- | <0.001 | 0.90 | 0.76- | 0.26 |
| | | | | | 2.68 | | | 1.08 | |
| Desvenlafaxine | 75 | 14 | 18.7 | 2.15 | 1.20- | 0.010 | 0.95 | 0.51- | 0.88 |
| | | | | | 3.86 | | | 1.78 | |
| Duloxetine | 726 | 158 | 21.8 | 2.61 | 2.18- | <0.001 | 0.95 | 0.77- | 0.60 |
| | | | | | 3.14 | | | 1.17 | |
| Levomilnacipran | 5 | 1 | 20.0 | 2.35 | 0.26- | 0.45 | 0.89 | 0.09- | 0.92 |
| | | | | | 21.01 | | | 8.46 | |
| Venlafaxine | 519 | 89 | 17.1 | 1.94 | 1.54- | <0.001 | 0.78 | 0.60- | 0.07 |
| | | | | | 2.45 | | | 1.02 | |
| Other | | | | | | | | | |
| Antidepressant | | | | | | | | | |
| Bupropion | 805 | 115 | 14.3 | 1.56 | 1.28- | <0.001 | 0.65 | 0.51- | <0.001 |
| | | | | | 1.92 | | | 0.82 | |
| Mirtazapine | 229 | 64 | 27.9 | 3.64 | 2.72- | <0.001 | 1.09 | 0.79- | 0.62 |
| | | | | | 4.88 | | | 1.50 | |
| SSRIs GROUPED BY | | | | | | | | | |
| S1R AFFINITY ^{b} | | | | | | | | | |
| High Affinity | 602 | 99 | 16.4 | 1.85 | 1.48- | <0.001 | 0.81 | 0.63- | 0.10 |
| | | | | | 2.30 | | | 1.04 | |
| Intermediate Affinity | 1784 | 285 | 16.0 | 1.79 | 1.56- | <0.001 | 0.80 | 0.68- | 0.01 |
| | | | | | 2.04 | | | 0.95 | |
| Low Affinity | 258 | 39 | 15.1 | 1.67 | 1.19- | 0.003 | 0.64 | 0.44- | 0.02 |
| | | | | | 2.36 | | | 0.93 | |
| ANTIDEPRESSANTS | | | | | | | | | |
| GROUPED BY | | | | | | | | | |
| FIASMA ACTIVITY | | | | | | | | | |
| With FIASMA Activity ^{c} | 4592 | 778 | 16.9 | 1.92 | 1.75- | <0.001 | 0.87 | 0.77- | 0.02 |
| | | | | | 2.10 | | | 0.98 | |
| With Unknown | 2519 | 505 | 20.0 | 2.35 | 2.11- | <0.001 | 0.92 | 0.80- | 0.24 |
| FIASMA Activity ^{d} | | | | | 2.62 | | | 1.06 | |

Abbreviations: CI, confidence interval. FIASMA, functional inhibition of acid sphingomyelinase. SNRI, serotonin-norepinephrine reuptake inhibitor. SSRI, selective serotonin reuptake inhibitor. S1R, sigma-1 receptor. TCA, tricyclic antidepressant.
^{a} N represent the number of patients included during calculation of the unadjusted odds ratio. In the multivariable logistic regression used to obtain the adjusted odds ratios, no imputation of missing values was performed and only complete cases were included. For example, in the primary analysis of exposure to any antidepressant versus exposure to no antidepressant, the total number of patients in the multivariable logistic regression was N = 21,051.
^{b} SSRIs with high affinity agonist activity at S1R included fluoxetine and fluvoxamine. SSRIs with intermediate affinity agonist activity at S1R included escitalopram and citalopram. SSRIs with low affinity agonist activity at S1R included paroxetine.
^{c} Antidepressants with FIASMA activity included amitriptyline, citalopram, clomipramine, desiparmine, doxepin, escitalopram, fluoxetine, fluvoxamine, imipramine, nortriptyline, paroxetine, sertraline, and venlafaxine.
^{d} Antidepressants with unknown FIASMA activity included bupropion, desvenlafaxine, duloxetine, levomilnacipran, mirtazapine, nefazodone, trazodone, vilazodone, and vortioxetine.

TABLE 13
Associations between Selected Antidepressant Exposures and ED or Hospital
Encounter within 30 Days, Stratified by Fluoxetine-Equivalent Dose ^{a}

| | | | | Unadjusted | | | Adjusted | | |
|---|---|---|---|---|---|---|---|---|---|
| | | Encounters | | Odds | | | Odds | 95% | |
| Exposure | N | n | % | Ratio | 95% CI | P | Ratio | CI | p |
| Any Antidepressant | | | | | | | | | |
| No Antidepressant | 19043 | 1833 | 9.6 | Ref | Ref | Ref | Ref | Ref | Ref |
| <20 mg Fluoxetine- | 1473 | 269 | 18.3 | 2.10 | 1.82- | <0.001 | 0.89 | 0.76- | |
| Equivalent | | | | | 2.41 | | | 1.05 | 0.16 |
| ≥20 mg Fluoxetine- | 4518 | 765 | 16.9 | 1.91 | 1.75- | <0.001 | 0.88 | 0.77- | 0.04 |
| Equivalent | | | | | 2.10 | | | 0.99 | |
| ≥40 mg Fluoxetine- | 2306 | 390 | 16.9 | 1.91 | 1.70- | <0.001 | 0.77 | 0.66- | 0.002 |
| Equivalent | | | | | 2.15 | | | 0.91 | |
| Any SSRI | | | | | | | | | |
| No Antidepressant | 19043 | 1833 | 9.6 | Ref | Ref | Ref | Ref | Ref | Ref |
| <20 mg Fluoxetine- | 453 | 71 | 15.7 | 1.75 | 1.35- | <0.001 | 0.76 | 0.57- | 0.05 |
| Equivalent | | | | | 2.26 | | | 1.01 | |
| ≥20 mg Fluoxetine- | 3296 | 544 | 16.5 | 1.86 | 1.67- | <0.001 | 0.86 | 0.74- | 0.03 |
| Equivalent | | | | | 2.06 | | | 0.98 | |
| ≥40 mg Fluoxetine- | 1500 | 237 | 15.8 | 1.76 | 1.52- | <0.001 | 0.74 | 0.61- | 0.001 |
| Equivalent | | | | | 2.04 | | | 0.89 | |
| Bupropion | | | | | | | | | |
| No Antidepressant | 19043 | 1833 | 9.6 | Ref | Ref | Ref | Ref | Ref | Ref |
| <20 mg Fluoxetine- | 504 | 70 | 13.9 | 1.51 | 1.17- | 0.002 | 0.63 | 0.47- | 0.001 |
| Equivalent | | | | | 1.96 | | | 0.83 | |
| ≥20 mg Fluoxetine- | 301 | 45 | 15.0 | 1.65 | 1.20- | 0.002 | 0.67 | 0.47- | 0.02 |
| Equivalent | | | | | 2.27 | | | 0.95 | |
| ≥40 mg Fluoxetine- | 10 | 2 | 20.0 | 2.35 | 0.50- | 0.28 | 0.57 | 0.12- | 0.48 |
| Equivalent | | | | | 11.06 | | | 2.73 | |
| Antidepressants with | | | | | | | | | |
| FIASMA Activity ^{b} | | | | | | | | | |
| No Antidepressant | 19043 | 1833 | 9.6 | Ref | Ref | Ref | Ref | Ref | Ref |
| <20 mg Fluoxetine- | 983 | 184 | 18.7 | 2.16 | 1.83- | <0.001 | 0.89 | 0.74- | 0.22 |
| Equivalent | | | | | 2.56 | | | 1.07 | |
| ≥20 mg Fluoxetine- | 3609 | 594 | 16.5 | 1.85 | 1.67- | <0.001 | 0.86 | 0.75- | 0.03 |
| Equivalent | | | | | 2.04 | | | 0.98 | |
| ≥40 mg Fluoxetine- | 1661 | 263 | 15.8 | 1.77 | 1.54- | <0.001 | 0.73 | 0.61- | <0.001 |
| Equivalent | | | | | 2.03 | | | 0.88 | |

Abbreviations: CI, confidence interval. FIASMA, functional inhibition of acid sphingomyelinase. SSRI, selective serotonin reuptake inhibitor.
^{a} Analyses stratified by fluoxetine-equivalent dose were performed for classes of antidepressants and individual drugs (if not already included in one of the classes) that were significantly associated with reduced ED or hospital encounters (adjusted p value < 0.05 in TABLE 12).
^{b} Antidepressants with FIASMA activity included amitriptyline, citalopram, clomipramine, desiparmine, doxepin, escitalopram, fluoxetine, fluvoxamine, imipramine, nortriptyline, paroxetine, sertraline, and venlafaxine.

TABLE 14
Logistic Regression for ED or Hospital Encounter within 30 Days.

| | | Encounter | | | | | | | | |
|---|---|---|---|---|---|---|---|---|---|---|
| | | within 30 | | Unadjusted | | | Adjusted | | | |
| | | Days | | Odds | | | Odds | | | |
| Variable | N^{a} | n | % | Ratio | 95% CI | p | Ratio | 95% CI | p | VIF |
| Any Anti- | | | | | | | | | | 1. [symbol] 2 |
| depressant | | | | | | | | | | |
| FALSE | 19,043 | 1,833 | 9.6 | ref | ref | | ref | Ref | | |
| TRUE | 5,991 | 1,034 | 17.3 | 1.9 [symbol] | 1.80-2.132 | <0.001 | 0.88 | 0.79-0.9 [symbol] | 0.02 | |
| Sex | | | | | | | | | | 1.08 |
| Femele | 1 [symbol] ,030 | 1,676 | 11.2 | ref | ref | | Ref | Ref | | |
| Male | 9,98 [symbol] | 1,191 | 11.9 | 1.0 [symbol] | 1.00-1.17 | 0.08 | 1.35 | 1.24-1.47 | <0.001 | |
| Age Category | | | | | | | | | | 1.39 |
| 18-31 | [symbol] ,034 | 454 | 7.5 | ref | ref | | Ref | Ref | | |
| 32-45 | 5,738 | 522 | 9.1 | 1.23 | 1.08-1.40 | [symbol] 0.0 [symbol] 2 | 0.87 | 0.75-1.00 | 0.05 | |
| 46-61 | 7,041 | 813 | 11.5 | 1. [symbol] | 1.42-1.81 | <0.001 | 0.08 | 0.7 [symbol] -0.91 | <0.001 | |
| 62+ | 6,221 | 1,078 | 17. [symbol] | 2. [symbol] 8 | 2.29-2.89 | <0.001 | 0.99 | 0. [symbol] 6-1.13 | 0.83 | |
| Number of | | | | | | | | | | 2.2 |
| Diagnoses | | | | | | | | | | |
| 01-04 | 6,152 | 157 | 2. [symbol] | ref | ref | | Ref | Ref | | |
| 05-10 | 5,410 | [symbol] 02 | 9.3 | 3.91 | 3.25-4. [symbol] 9 | <0.001 | 1.99 | 1.63-2.43 | <0.001 | |
| 11-20 | 5,623 | 53 [symbol] | 13.1 | 5.78 | 4.84- [symbol] .39 | <0.001 | 2.15 | 1.75-2.64 | <0.001 | |
| 21+ | 5,995 | 1,4 [symbol] 4 | 24.4 | 12.34 | 10.42-14.81 | <0.001 | 3.22 | 2.59-4.01 | <0.001 | |
| Number of | | | | | | | | | | 2.421 |
| Medications | | | | | | | | | | |
| 0 | 6,65 [symbol] | 72 | 1.1 | ref | ref | | Ref | Ref | | |
| 01-05 | [symbol] ,351 | 475 | 7.5 | 7.3 [symbol] | 5.78-9.50 | <0.001 | 3.1 [symbol] | 2.41-4.22 | <0.001 | |
| 06-13 | [symbol] ,143 | 80 [symbol] | 13.1 | 13.81 | 10.83-17. [symbol] 3 | <0.001 | 4.64 | 3.47- [symbol] .2 [symbol] | <0.001 | |
| 14+ | 5,8 [symbol] 2 | 1,514 | 25.7 | 31.71 | 24.95-40.28 | <0.001 | 8.24 | 6.09-11.15 | <0.001 | |
| Obese | | | | | | | | | | 1.08 |
| FALSE | 10, [symbol] 0 [symbol] | 1,242 | 11.7 | ref | ref | | Ref | Ref | | |
| TRUE | 11,197 | 1,619 | 14.5 | 1.27 | 1.18-1.38 | <0.001 | 1.02 | 0.93-1.11 | 0.89 | |
| White Mon- | | | | | | | | | | 1.11 |
| Hispanic | | | | | | | | | | |
| FALSE | [symbol] ,524 | 1,173 | 13.8 | ref | ref | | Ref | Ref | | |
| TRUE | 1 [symbol] ,507 | 1,686 | 10. [symbol] | [symbol] .7 [symbol] | 0.71-0.83 | <0.001 | 0. [symbol] 7 | 0.52-0.62 | <0.001 | |
| Mood or Anxiety | | | | | | | | | | 1.64 |
| Disorder | | | | | | | | | | |
| FALSE | 17,29 [symbol] | 1,848 | 10.7 | ref | ref | | Ref | Ref | | |
| TRUE | 5,8 [symbol] 4 | 1,014 | 17.2 | 1.74 | 1. [symbol] 0-1.89 | <0.001 | 1 | 0.89-1.11 | 0.9 [symbol] | |
| Other Psychiatric | | | | | | | | | | 1.13 |
| Disorder | | | | | | | | | | |
| FALSE | 20,7 [symbol] 3 | 2,417 | 11. [symbol] | ref | ref | | Ref | Ref | | |
| TRUE | 2,417 | 44 [symbol] | 18.4 | 1.71 | 1 [symbol] 3-1. [symbol] 1 | <0.001 | 0.92 | 0.82-1.04 | 0.21 | |
| Benzodiazepine or | | | | | | | | | | 1.18 |
| Z Drug | | | | | | | | | | |
| FALSE | 22,412 | 2,310 | 10.3 | ref | ref | | Ref | Ref | | |
| TRUE | 2,622 | 557 | 21.2 | 2.35 | 2.12-2 [symbol] 0 | <0.001 | 1.18 | 1.05-1.32 | 0.00 [symbol] | |
| Antipsychotic | | | | | | | | | | |
| Drug | | | | | | | | | | |
| FALSE | 24,277 | 2,655 | 10.9 | ref | ref | | Ref | Ref | | |
| TRUE | 757 | 212 | 28 | 3.17 | 2. [symbol] -3.73 | <0.001 | 1.6 | 1.34-1.92 | <0.001 | |

Abbreviation: VIF = variance inflation factor
^{a}N represent the number of patients included during calculation of the unadjusted odds ratio. In the multivariable logistic regression used to obtain the adjusted odds ratios, no imputation of missing values was performed and only complete cases were included. The total number of patients in the multivariable logistic regression was N = 21,051. [symbol]
indicates data missing or illegible when filed

### Discussion

In this single-center, retrospective cohort of 25,034 ambulatory patients infected with SARS-CoV-2, exposure to an antidepressant was significantly associated with reduced incidence of ED visitation or hospital admission after adjusting for sociodemographic characteristics, medical comorbidity, psychiatric conditions, and concurrent treatments, with a significant dose-dependent relationship. In secondary analyses examining specific classes of antidepressants, the benefit was observed with exposure to SSRIs, antidepressants with FIASMA activity, and bupropion. This association was only observed among patients exposed to a dose of antidepressants of at least 20 mg of fluoxetine-equivalents per day.

The results of this study are broadly consistent with recently published literature examining clinical deterioration in patients with COVID-19 exposed to pre-illness SSRIs. In a multicenter, retrospective observational study of 7,230 adult patients who were hospitalized for COVID-19 in Paris, antidepressant use, particularly fluoxetine, paroxetine, escitalopram, venlafaxine, and mirtazapine, was associated with reduced risk of intubation or death (12). In a subsequent multicenter, retrospective cohort study of 83,584 patients with COVID-19 treated in hospitals, EDs, or urgent care clinics in the United States, pre-illness SSRI use was associated with a reduced relative risk of mortality, with the largest effects observed in patients taking fluoxetine or fluvoxamine (13). This study similarly demonstrates that pre-illness use of antidepressants as a whole, and SSRIs in particular, is associated with reduced risk of ED visitations or hospital admission in ambulatory patients infected with SARS-CoV-2.

The largest effect was seen with pre-illness exposure to the highest dose of SSRIs, hereby stratified as a daily dose ≥40 mg of fluoxetine-equivalents. This finding could be in line with results of a prior meta-analysis showing that most antidepressants, mainly SSRIs, are significantly associated with reduced levels of several pro-inflammatory cytokines (i.e. IL-6, IL-10, TNF-alpha, CCL-2) among depressed individuals (23). Given that elevations in these cytokines are associated with severe COVID-19, any protective effect of SSRIs may plausibly act through immunomodulation.

Examination of additional subgroups in this study permitted inferences on the mechanisms by which SSRIs and other antidepressants may provide a protective effect during SARS-CoV-2 infection. The incidence of ED visitation or hospital admission was significantly reduced among patients exposed to antidepressants with FIASMA activity (including paroxetine). Acid sphingomyelinase (ASM) plays a key role in transduction of pathological signals into the cell. Hydrolysis of sphingomyelin, catalyzed by ASM, results in production of ceramide, a lipophilic sphingolipid that gathers within the outer layer of the cell membrane. “Rafts” of ceramide allow necessary receptor proteins to aggregate, facilitating pathological signal transduction (19, 24). Antidepressant-mediated inhibition of ASM may provide protection against COVID-19 both via reduced viral entry into cells (due to reduced formation of ceramide-enriched membrane domains) and via reduced inflammatory response (5, 11). Use of antidepressants with FIASMA activity against SARS-CoV-2 infection has been studied in preclinical models (9) and in a multicenter retrospective cohort study. Among individuals hospitalized with severe COVID-19, use of a medication with FIASMA activity was associated with reduced hazards of intubation or death (25). Similar results were observed in another cohort of individuals with psychiatric disorders hospitalized for severe COVID-19 (14). In a retrospective cohort study conducted at an adult psychiatric facility, exposure to antidepressants was associated with reduced incidence of COVID-19 infection (26). Additionally, ceramide levels, sphingomyelinase activity, and ceramidase activity have all been correlated with COVID-19 severity and with inflammatory markers (27), lending further support for FIASMA as an explanatory mechanism behind the associations reported in observational studies.

In contrast, no dose-response effect was observed in this cohort when the SSRIs were stratified according to their level of S1R affinity. The relatively high adjusted odds ratio for the one S1R antagonist, sertraline, suggests it may be less protective than other SSRIs; however, the confidence intervals for all SSRI odds ratios overlap, and therefore, the strengths of the effects cannot be distinguished. Although the S1R-mediated anti-inflammatory effects of fluvoxamine have been studied in some detail, the effects of other SSRIs on the S1R are not as well understood. Additionally, the beneficial effects of SSRIs and non-SSRI agents with serotonin antagonism may be time-dependent in COVID-19, owing to their diverse anti-platelet (28) and serotonin modulating properties (29). On one hand, early or pre-illness use of SSRIs may be beneficial by diminishing the degree of serotonin loading onto mature platelets during the viral replication phase (30), before the onset of severe platelet activation and pathogenic platelet mediator release that are hallmarks of the inflammatory phase in COVID-19. Thus, early or pre-illness SSRI use preemptively reduces a key content of platelet granules, serotonin, which is demonstrated to cause pathogenicity in illnesses driven by immune-mediated platelet activation (31) and also drives immunothrombosis in severe COVID-19 (32). On the other hand, the direct serotonin receptor inhibitory effects of some SSRIs and non-SSRI agents (e.g., bupropion) may be beneficial even after the inflammatory phase has begun (26, 31, 33), by inhibiting the pro-thrombotic and pro-inflammatory action of released serotonin from severely activated platelets in this illness. Further research is necessary to explore the optimal timing of treatment with serotonin modulating agents. If antidepressants are demonstrated to have multiple modalities of action as discussed above, their benefit may not be limited to a specific phase of illness, unlike antivirals (best if given in first 5 days of symptoms) and systemic steroids (if given too early, could suppress the appropriate immune response to the virus) (34).

Interestingly, exposure to bupropion, a non-SSRI dopamine and norepinephrine reuptake inhibitor, was significantly associated with improved outcomes in this study. Although we cannot rule out the possibility that this association may result from multiple testing or that the pattern of confounding may be different for bupropion (often used for indications other than depression compared to other antidepressants), its effect on ASM has never been formally tested, to our knowledge. However, bupropion may have S1R agonist activity, although this has been incompletely evaluated (35). It is also a negative allosteric modulator of serotonin type 3A (5HT3A) receptors (36). Another 5HT3 antagonist, ondansetron, was associated with better outcomes, including reduced mortality in a study of COVID-19 inpatients (33). Therefore, it may be worthwhile to further explore the repurposing of 5HT3A antagonists as treatment for COVID-19. Blocking the 5HT3A receptor with the selective antagonist MDL 72222 totally prevented adverse respiratory effects of exogenous serotonin administration in a study of cats (37). Relevant to this, a majority of patients in the inflammatory phase of COVID-19 experience severe platelet activation and excessive platelet aggregates (38), and consequently platelet serotonin is acutely released from platelet granules into plasma. Since plasma serotonin in patients with COVID-19 reaches levels several folds beyond normal, and even significantly beyond levels seen in other etiologies of acute respiratory distress syndrome (39), early blockage of the 5HT3A receptor may minimize adverse effects of excessive platelet serotonin release, perhaps preventing or ameliorating respiratory deterioration and other potential complications of platelet serotonin excess in COVID-19. Lastly, other mechanisms such as bupropion's ability to lower the levels of the inflammatory mediators TNF-alpha and interferon-gamma in preclinical models may additionally contribute to the beneficial association demonstrated in this study (40).

One of the strengths of this work is the rigorous adjustment for confounding variables employed in the analysis. When compared to patients without antidepressant exposure, patients with antidepressant exposure were older, had more diagnoses including other psychiatric diagnoses, and took more medications at home including other psychoactive medications (TABLE 14). Thus, antidepressant exposure would act as a surrogate marker for overall degree of comorbidity if proper risk adjustments were not performed. This explains why antidepressant exposure was associated with increased risk of ED visitation or hospital admission in the initial unadjusted analysis. A comprehensive risk adjustment, however, not only eliminated this ostensible increased risk, but instead revealed an association in the opposite direction toward protection against ED visitation or hospital admission in those with antidepressant exposure.

This study also has limitations that should be noted. First, this was an observational study. However, the presence of biologically plausible mechanisms for the observed associations and the agreement with other recent studies reinforces the validity of our findings and posits that prospective interventional studies of SSRIs other than fluvoxamine may be appropriate for the early treatment of COVID-19. Second, information on vital status was unavailable unless patients died while in the hospital, which prevented the addition of death to the composite outcome. Third, this analysis was conducted at a single healthcare system. Although the healthcare system in this study has a wide footprint, including academic and non-academic hospitals that serve both urban and rural communities, these findings may not be generalizable to other settings. However, the congruence of our observations with the findings of other recent studies mitigate the concerns about generalizability. Fourth, some patients may have sought ED or inpatient care at facilities outside this healthcare system, and these visits would not have been detected. Fifth, information about vaccination was not available. Finally, the magnitude of the associations may be underestimated given the high rate of antidepressant discontinuation in the clinical outpatient setting.

In conclusion, pre-illness exposure to antidepressants was associated with a decreased odds of ED visitation or hospital admission among ambulatory patients infected with SARS-CoV-2. This association appears to be related to exposure to SSRIs and in particular to the agents with FIASMA activity. When designing future prospective studies, researchers should consider SSRIs and other agents with FIASMA activity as candidate interventions for outpatient therapy of COVID-19.

### REFERENCES

1. Hoertel N, Blachier M, Blanco C, et al: A stochastic agent-based model of the SARS-CoV-2 epidemic in France. Nat Med 2020; 26:1417-1421
2. Ritchie H, Matieu E, Rodes-Guirao L, et al: Coronavirus Pandemic (COVID-19) Vaccinations. Published online at OurWorldlnData.org2020.
3. The Possibility of COVID-19 after Vaccination: Breakthrough Infections. Centers for Disease Control and Prevention; 2021.
4. Singh T U, Parida S, Lingaraju M C, et al: Drug repurposing approach to fight COVID-19. Pharmacol Rep 2020; 72:1479-1508
5. Hoertel N: Do the Selective Serotonin Reuptake Inhibitor Antidepressants Fluoxetine and Fluvoxamine Reduce Mortality Among Patients With COVID-19?JAMA Netw Open 2021; 4:e2136510
6. Hoertel N, Sánchez-Rico M, Cougoule C, et al: Repurposing antidepressants inhibiting the sphingomyelinase acid/ceramide system against COVID-19: current evidence and potential mechanisms. Mol Psychiatry 2021:1-2
7. Zimniak M, Kirschner L, Hilpert H, et al: The serotonin reuptake inhibitor Fluoxetine inhibits SARS-CoV-2 in human lung tissue. Sci Rep 2021; 11:5890
8. Schloer S, Brunotte L, Mecate-Zambrano A, et al: Drug synergy of combinatory treatment with remdesivir and the repurposed drugs fluoxetine and itraconazole effectively impairs SARS-CoV-2 infection in vitro. Br J Pharmacol 2021; 178:2339-2350
9. Carpinteiro A, Edwards M J, Hoffmann M, et al: Pharmacological Inhibition of Acid Sphingomyelinase Prevents Uptake of SARS-CoV-2 by Epithelial Cells. Cell Rep Med 2020; 1:100142
10. Sukhatme V P, Reiersen A M, Vayttaden S J, Sukhatme W: Fluvoxamine: A Review of Its Mechanism of Action and Its Role in COVID-19. Front Pharmacol 2021; 12:652688
11. Kornhuber J, Hoertel N, Gulbins E: The acid sphingomyelinase/ceramide system in COVID-19. Mol Psychiatry 2021:1-8
12. Hoertel N, Sánchez-Rico M, Vernet R, et al: Association between antidepressant use and reduced risk of intubation or death in hospitalized patients with COVID-19: results from an observational study. Mol Psychiatry 2021; 26:5199-5212
13. Oskotsky T, Maric I, Tang A, et al: Mortality Risk Among Patients With COVID-19 Prescribed Selective Serotonin Reuptake Inhibitor Antidepressants. JAMA Netw Open 2021; 4:e2133090
14. Hoertel N, Sánchez-Rico M, Gulbins E, et al: Association between FIASMA Psychotropic Medications and reduced risk of Intubation or Death in Individuals with Psychiatric Disorders Hospitalized for Severe COVID-19: an Observational Multicenter Study. Translational Psychiatry 2022;
15. Lenze E J, Mattar C, Zorumski C F, et al: Fluvoxamine vs Placebo and Clinical Deterioration in Outpatients With Symptomatic COVID-19: A Randomized Clinical Trial. Jama 2020; 324:2292-2300
16. Reis G, Dos Santos Moreira-Silva E A, Silva D C M, et al: Effect of early treatment with fluvoxamine on risk of emergency care and hospitalisation among patients with COVID-19: the TOGETHER randomised, platform clinical trial. Lancet Glob Health 2021;
17. Seftel D, Boulware D R: Prospective Cohort of Fluvoxamine for Early Treatment of Coronavirus Disease 19. Open Forum Infect Dis 2021; 8:ofab050
18. Ishima T, Fujita Y, Hashimoto K: Interaction of new antidepressants with sigma-1 receptor chaperones and their potentiation of neurite outgrowth in PC12 cells. Eur J Pharmacol 2014; 727:167-173
19. Kornhuber J, Muehlbacher M, Trapp S, et al: Identification of novel functional inhibitors of acid sphingomyelinase. PLoS One 2011; 6:e23852
20. Hayasaka Y, Purgato M, Magni L R, et al: Dose equivalents of antidepressants: Evidence-based recommendations from randomized controlled trials. J Affect Disord 2015; 180:179-184
21. Hoertel N, Sánchez-Rico M, Muela P, et al: Risk of death in individuals hospitalized for COVID-19 with and without psychiatric disorders: an observational multicenter study in France. Biol Psychiatry Glob Open Sci 2022;
22. Hoertel N, Sánchez-Rico M, Herrera-Morueco J J, et al: Comorbid medical conditions are a key factor to understand the relationship between psychiatric disorders and COVID-19-related mortality: Results from 49,089 COVID-19 inpatients. Mol Psychiatry 2021:1-3
23. Kohler C A, Freitas T H, Stubbs B, et al: Peripheral Alterations in Cytokine and Chemokine Levels After Antidepressant Drug Treatment for Major Depressive Disorder: Systematic Review and Meta-Analysis. Mol Neurobiol 2018; 55:4195-4206
24. Kornhuber J, Tripal P, Reichel M, et al: Identification of new functional inhibitors of acid sphingomyelinase using a structure-property-activity relation model. J Med Chem 2008; 51:219-237
25. Hoertel N, Sánchez-Rico M, Gulbins E, et al: Association Between FIASMAs and Reduced Risk of Intubation or Death in Individuals Hospitalized for Severe COVID-19: An Observational Multicenter Study. Clin Pharmacol Ther 2021; 110:1498-1511
26. Clelland C L, Ramiah K, Steinberg L, Clelland J D: Analysis of the impact of antidepressants and other medications on COVID-19 infection risk in a chronic psychiatric in-patient cohort. BJPsych Open 2021; 8:e6
27. Marín-Corral J, Rodríguez-Morató J, Gomez-Gomez A, et al: Metabolic Signatures Associated with Severity in Hospitalized COVID-19 Patients. Int J Mol Sci 2021; 22:4794
28. Roweth H G, Yan R, Bedwani N H, et al.: Citalopram inhibits platelet function independently of SERT-mediated 5-HT transport. Sci Rep 2018; 8:3494
29. Herr N, Bode C, Duerschmied D: The Effects of Serotonin in Immune Cells. Front Cardiovasc Med 2017; 4:48
30. Hergovich N, Aigner M, Eichler H G, et al: Paroxetine decreases platelet serotonin storage and platelet function in human beings. Clin Pharmacol Ther 2000; 68:435-442
31. Cloutier N, Allaeys I, Marcoux G, et al: Platelets release pathogenic serotonin and return to circulation after immune complex-mediated sequestration. Proc Natl Acad Sci USA 2018; 115:E1550-e1559
32. Althaus K, Marini I, Zlamal J, et al: Antibody-induced procoagulant platelets in severe COVID-19 infection. Blood 2021; 137:1061-1071
33. Bayat V, Ryono R, Phelps S, et al: Reduced Mortality With Ondansetron Use in SARS-CoV-2-Infected Inpatients. Open Forum Infect Dis 2021; 8:ofab336
34. Horby P, Lim W S, Emberson J R, et al: Dexamethasone in Hospitalized Patients with Covid-19. N Engl J Med 2021; 384:693-704
35. Dhir A, Kulkarni S K: Possible involvement of sigma-1 receptors in the anti-immobility action of bupropion, a dopamine reuptake inhibitor. Fundam Clin Pharmacol 2008; 22:387-394
36. Pandhare A, Pappu A S, Wilms H, Blanton M P, Jansen M: The antidepressant bupropion is a negative allosteric modulator of serotonin type 3A receptors. Neuropharmacology 2017; 113:89-99
37. Kopczyńska B, Szereda-Przestaszewska M: 5HT2 and 5HT3 receptors' contribution to modeling of post-serotonin respiratory pattern in cats. Life Sci 2004; 75:2281-2290
38. Nishikawa M, Kanno H, Zhou Y, et al: Massive image-based single-cell profiling reveals high levels of circulating platelet aggregates in patients with COVID-19. Nat Commun 2021; 12:7135
39. Zaid Y, Guessous F, Puhm F, et al: Platelet reactivity to thrombin differs between patients with COVID-19 and those with ARDS unrelated to COVID-19. Blood Adv 2021; 5:635-639
40. Kast R E: Anti- and pro-inflammatory considerations in antidepressant use during medical illness: bupropion lowers and mirtazapine increases circulating tumor necrosis factor-alpha levels. Gen Hosp Psychiatry 2003; 25:495-496

### Repurposing Antidepressants Inhibiting the Sphingomyelinase Acid/Ceramide System Against COVID-19: Current Evidence and Potential Mechanisms

Following our multicenter observational retrospective study that showed a substantial association between antidepressant use and reduced risk of intubation or death in 7230 patients hospitalized for COVID-19 [3], Salles et al. suggest that combining an antidepressant such as fluoxetine with rimonabant, an inverse agonist of CB1 cannabinoid receptor, may be useful against COVID-19, thanks to the antiviral and anti-inflammatory effects of the former and the potentially complementary anti-inflammatory properties of the later. Stip et al. significantly summarized the growing body of evidence of the potential benefit of different psychotropic medications in COVID-19 and their possible underlying mechanisms. They suggested further elucidation of ways that certain antidepressants may be acting in this indication. These two letters challenge us on the potential mechanisms that may underlie the potential positive effect of certain antidepressants on the course of COVID-19. This knowledge is crucial to help identify the more promising molecules for COVID-19 and help design trials evaluating these molecules.

Since the initial release of our results in July, 2020 [4], several important studies have led to a substantially improved understanding of the mechanisms that may underlie the potential positive effect of certain antidepressants.

First, molecules such as fluoxetine, fluvoxamine, paroxetine, escitalopram, or amitriptyline are antidepressants that belong to the group of functional inhibitors of acid sphingomyelinase (ASM), called FIASMA [5,6,7], that also comprises other medications commonly used in clinical practice, such as antihistamine medications (e.g., hydroxyzine, promethazine), calcium channel blockers (e.g., amlodipine, bepridil), and mucolytics (e.g., ambroxol [7]). These pharmacological compounds in vitro and in vivo inhibit ASM, an enzyme that catalyzes the hydrolysis of sphingomyelin into ceramide and phosphorylcholine [5,6,7]. Preclinical evidence indicates that SARS-CoV-2 activates the ASM/ceramide system, resulting in the formation of ceramide-enriched membrane domains that facilitate viral entry and infection by clustering ACE2, the cellular receptor of SARS-CoV-2 [6, 7]. The inhibition of the ASM/ceramide system by FIASMA antidepressants prevented infection of Vero E6 cells with SARS-CoV-2. Importantly, the reconstitution of ceramides in cells treated with these antidepressants restored the infection [6]. In healthy volunteers, oral administration of a low dose of the FIASMA antidepressant amitriptyline prevented infection of freshly isolated nasal epithelial cells with SARS-CoV-2 spike protein pseudotyped particles within 2 h, which was also restored after the reconstitution of ceramides in these cells [6]. These preclinical data were confirmed by another study that demonstrated an inhibition by fluoxetine of SARS-CoV-2 infection in cultured epithelial cells [8]. The potential benefit of FIASMA treatments among patients hospitalized for severe COVID-19 was recently explored in an observational multicenter retrospective study [9]. Therein, it was reported that taking a FIASMA medication upon hospital admission was associated with substantially reduced likelihood of intubation or death. This association was not specific to one FIASMA class (e.g., FIASMA antidepressants) or medication (e.g., fluoxetine) [9]. A similar significant association was found in another observational multicenter retrospective study conducted in patients with psychiatric disorders and hospitalized for severe COVID-19 [10]. A retrospective observational study also established a positive association between chronic administration of FIASMA and reduced mortality in COVID-19 hospitalized patients that was significant for the FIASMA amlodipine [11]. In a double-blind randomized clinical trial, outpatients treated with the FIASMA antidepressant fluvoxamine compared with placebo had a lower risk of clinical deterioration over 15 days of treatment [12]. The results of a prospective real-world evidence study also support this observation [13]. Finally, plasma markers of ceramide metabolism were found to be associated with respiratory severity and to correlate with inflammation in 49 patients hospitalized for COVID-19 [14]. Taken together, these results show the potentially crucial importance of the ASM/ceramide system as a treatment target in COVID-19, a mechanism likely to be shared by all variants [15]. They also support the continuation of FIASMA medications during SARS-CoV-2 infection [9].

Second, anti-inflammatory properties of several antidepressants may have important value in regulating inflammation by inhibiting cytokine production in COVID-19. These anti-inflammatory effects might be explained (i) by the high affinity of certain antidepressants, such as fluvoxamine or fluoxetine, for Sigma-1 receptors [12, 16], which have been shown to restrict the endonuclease activity of an Endoplasmic Reticulum (ER) stress sensor called Inositol-Requiring Enzyme1 and to reduce cytokine expression without inhibiting classical inflammatory pathways, and/or (ii) by the inhibition of ASM in endothelial cells and the immune system [6, 7].

Finally, other potential mechanisms include reduction in platelet aggregation, decreased mast cell degranulation, interference with endolysosomal viral trafficking and increased melatonin levels [16].

These different but potentially interrelated mechanisms shared by several antidepressants such as fluvoxamine or fluoxetine, might collectively lead to anti-SARS-COV-2 effects while diminishing coagulopathy and cytokine storm consequences, which are known hallmarks of severe COVID-19.

Following these preclinical, observational and clinical converging findings, and as stated by Salles et al. [1] and Stip et al. [2], large-scale double-blind placebo-controlled randomized clinical trials of FIASMA antidepressants for COVID-19 at different stages of the disease, either alone or combined with medications that have shown preliminary evidence of potential efficacy and good tolerability, are urgently needed. Fluoxetine and fluvoxamine, which display high in vitro inhibition effect on ASM, showed potential positive effects at usual antidepressant doses, and are easy to use, including high safety margins, good tolerability, widespread availability and low cost, should be considered compelling treatments to prioritize for phase 3 trials against COVID-19 [9].

### REFERENCES

1. Salles J, Briand-Mésange F, Trudel S, Ausseil J, Salles J-P, Chap H. Can antidepressants unlock prescription of rimonabant in the fight against COVID-19? Mol Psychiatry. 2021:1-2. https://doi.org/10.1038/s41380-021-01221-y. Epub ahead of print.
2. Stip E, Arnone D, Abdel Aziz K, Javaid S. Diversity of mechanism of action of psychotropic drugs in their anti-COVID-19 properties. Mol Psychiatry. 2021:1-5. https://doi.org/10.1038/s41380-021-01222-x. Epub ahead of print.
3. Hoertel N, Sánchez-Rico M, Vernet R, Beeker N, Jannot A-S, Neuraz A, et al. Association between antidepressant use and reduced risk of intubation or death in hospitalized patients with COVID-19: results from an observational study. Mol Psychiatry. 2021. https://doi.org/10.1038/s41380-021-01021-4 Epub ahead of print.
4. Hoertel N, Sánchez-Rico M, Vernet R, Beeker N, Jannot A-S, Neuraz A, et al. SSRIs and SNRIs and Risk of Death or Intubation in COVID-19: Results from an Observational Study. medRxiv 2020.07.09.20143339; https://doi.org/10.1101/2020.07.09.20143339.
5. Kornhuber J, Tripal P, Reichel M, Mühle C, Rhein C, Muehlbacher M, et al. Functional Inhibitors of Acid Sphingomyelinase (FIASMAs): a novel pharmacological group of drugs with broad clinical applications. Cell Physiol Biochem. 2010; 26:9-20.
6. Carpinteiro A, Edwards M J, Hoffmann M, Kochs G, Gripp B, Weigang S, et al. Pharmacological inhibition of acid sphingomyelinase prevents uptake of SARS-CoV-2 by epithelial cells. Cell Rep Med. 2020; 1:100142. https://doi.org/10.1016/j.xcrm.2020.100142. Epub 2020 Oct. 29.
7. Carpinteiro A, Gripp B, Hoffmann M, Pohlmann S, Hoertel N, Edwards M J, et al. Inhibition of acid sphingomyelinase by ambroxol prevents SARS-CoV-2 entry into epithelial cells. J Biol Chem. 2021; 296:100701. https://doi.org/10.1016/j.jbc.2021.100701. Epub 2021 Apr. 23.
8. Schloer S, Brunotte L, Goretzko J, Mecate-Zambrano A, Korthals N, Gerke V, et al. Targeting the endolysosomal host-SARS-CoV-2 interface by clinically licensed functional inhibitors of acid sphingomyelinase (FIASMA) including the antidepressant fluoxetine. Emerg Microbes Infect. 2020; 9:2245-55.
9. Hoertel N, Sánchez-Rico M, Gulbins E, Kornhuber J, Carpinteiro A, Lenze E J, et al. Association between FIASMAs and Reduced Risk of Intubation or Death in Individuals Hospitalized for Severe COVID-19: an observational multicenter study. Clin Pharmacol Ther. 2021:10.1002/cpt.2317. https://doi.org/10.1002/cpt.2317. Epub ahead of print.
10. Hoertel N, Sánchez-Rico M, Gulbins E, Kornhuber J, Carpinteiro A, Abellen M, et al. Association between Psychotropic Medications Functionally Inhibiting Acid Sphingomyelinase and reduced risk of Intubation or Death among Individuals with Mental Disorder and Severe COVID-19: an Observational Study. medRxiv 2021.02.18.21251997; https://doi.org/10.1101/2021.02.18.21251997.
11. Darquennes G, Le Corre P, Le Moine O, Loas G. Association between functional inhibitors of acid sphingomyelinase (Fiasmas) and reduced risk of death in covid-19 patients: a retrospective cohort study. Pharmaceuticals. 2021; 14:226.
12. Lenze E J, Mattar C, Zorumski C F, Stevens A, Schweiger J, Nicol G E, et al. Fluvoxamine vs Placebo and Clinical Deterioration in Outpatients With Symptomatic COVID-19: a Randomized Clinical Trial. JAMA. 2020; 324:2292-2300.
13. Seftel D, Boulware D R. Prospective Cohort of Fluvoxamine for Early Treatment of Coronavirus Disease 19. Open Forum Infect Dis. 2021; 8:ofab050.
14. Marín-Corral J, Rodríguez-Morató J, Gomez-Gomez A, Pascual-Guardia S, Muñoz-Bermüdez R, Salazar-Degracia A, et al. Metabolic Signatures Associated with Severity in Hospitalized COVID-19 Patients. Int J Mol Sci. 2021; 22:4794.
15. Fred S M, Kuivanen S, Ugurlu H, Casarotto P C, Levanov L, Saksela K, et al. Antidepressant and antipsychotic drugs reduce viral infection by SARS-CoV-2 and fluoxetine show antiviral activity against the novel variants in vitro. bioRxiv 2021.03.22.436379; https://doi.org/10.1101/2021.03.22.436379.
16. Sukhatme V P, Reiersen A M, Vayttaden S J, Sukhatme V V. Fluvoxamine: a Review of Its Mechanism of Action and Its Role in COVID-19. Front Pharm. 2021; 12:652688.

### Example 7: Effect of Early Treatment with Fluvoxamine on Risk of Emergency Care and Hospitalization Among Patients with COVID-19: The TOGETHER Randomized, Platform Clinical Trial

### Background

Recent evidence indicates a potential therapeutic role of fluvoxamine for COVID-19. In the TOGETHER trial for acutely symptomatic patients with COVID-19, we aimed to assess the efficacy of fluvoxamine versus placebo in preventing hospitalization defined as either retention in a COVID-19 emergency setting or transfer to a tertiary hospital due to COVID-19.

### Methods

This placebo-controlled, randomized, adaptive platform trial done among high-risk symptomatic Brazilian adults confirmed positive for SARS-CoV-2 included eligible patients from 11 clinical sites in Brazil with a known risk factor for progression to severe disease. Patients were randomly assigned (1:1) to either fluvoxamine (100 mg twice daily for 10 days) or placebo (or other treatment groups not reported here). The trial team, site staff, and patients were masked to treatment allocation. Our primary outcome was a composite endpoint of hospitalization defined as either retention in a COVID-19 emergency setting or transfer to tertiary hospital due to COVID-19 up to 28 days post-random assignment on the basis of intention to treat. Modified intention to treat explored patients receiving at least 24 h of treatment before a primary outcome event and per-protocol analysis explored patients with a high level adherence (>80%). We used a Bayesian analytic framework to establish the effects along with probability of success of intervention compared with placebo. The trial is registered at ClinicalTrials.gov (NCT04727424) and is ongoing.

### Findings

The study team screened 9803 potential participants for this trial. The trial was initiated on Jun. 2, 2020, with the current protocol reporting randomization to fluvoxamine from Jan. 20 to Aug. 5, 2021, when the trial arms were stopped for superiority. 741 patients were allocated to fluvoxamine and 756 to placebo. The average age of participants was 50 years (range 18-102 years); 58% were female. The proportion of patients observed in a COVID-19 emergency setting for more than 6 h or transferred to a teritary hospital due to COVID-19 was lower for the fluvoxamine group compared with placebo (79 [11%] of 741 vs 119 [16%] of 756); relative risk [RR] 0.68; 95% Bayesian credible interval [95% BCI]: 0.52-0.88), with a probability of superiority of 99.8% surpassing the prespecified superiority threshold of 97.6% (risk difference 5.0%). Of the composite primary outcome events, 87% were hospitalizations. Findings for the primary outcome were similar for the modified intention-to-treat analysis (RR 0.69, 95% BCI 0.53-0.90) and larger in the per-protocol analysis (RR 0.34, 95% BCI, 0.21-0.54). There were 17 deaths in the fluvoxamine group and 25 deaths in the placebo group in the primary intention-to-treat analysis (odds ratio [OR] 0.68, 95% CI: 0.36-1.27). There was one death in the fluvoxamine group and 12 in the placebo group for the per-protocol population (OR 0.09; 95% CI 0.01-0.47). We found no significant differences in number of treatment emergent adverse events among patients in the fluvoxamine and placebo groups.

### Interpretation

Treatment with fluvoxamine (100 mg twice daily for 10 days) among high-risk outpatients with early diagnosed COVID-19 reduced the need for hospitalization defined as retention in a COVID-19 emergency setting or transfer to a tertiary hospital.

### Introduction

Although safe and effective vaccines for COVID-19 have been developed and distributed, there remain, particularly in low resource settings, major challenges regarding their production, allocation, and affordability.¹

Identifying inexpensive, widely available, and effective therapies against COVID-19 is, therefore, of great importance. In particular, repurposing existing medicines that are widely available and with well understood safety profiles, has particular appeal.²

TOGETHER is the largest randomized trial to assess the effectiveness of fluvoxamine for patients with COVID-19 in the community. Compared with placebo, patients randomly assigned to fluvoxamine had a lower risk of hospitalization defined as either retention in a COVID-19 emergency setting or transfer to a tertiary hospital due to COVID-19.

There are few effective therapies for patients with COVID-19 in the community. Results provide compelling evidence of fluvoxamine's benefit in reducing acute morbidity from COVID-19 illness.

Fluvoxamine is a selective serotonin reuptake inhibitor (SSRI) and a 6-1 receptor (S1R) agonist.³

There are several potential mechanisms for fluvoxamine in treatment of COVID-19 illness, including anti-inflammatory and possible antiviral effects.⁴

A small placebo-controlled, randomized trial has raised the possibility that fluvoxamine might reduce the risk of clinical deterioration in outpatients with COVID-19, suggesting the need for larger randomized, placebo-controlled studies.^{5,6}

To evaluate the efficacy of fluvoxamine to prevent progression of COVID-19 and hospitalization among outpatients with laboratory-documented SARS-CoV-2, we did a randomized, placebo-controlled, adaptive platform trial in Minas Gerais, Brazil. This flexible platform trial design allows for additional agents to be added and tested with standardized operating procedures outlined in a single overarching master protocol.^{7, 8}

Among eight different interventions evaluated in this platform trial, we report here on the clinical evaluation of fluvoxamine by means of a concurrent placebo control group.

### Methods

### Study Design

The TOGETHER trial is a randomized, adaptive platform trial to investigate the efficacy of repurposed treatments for COVID-19 disease among high-risk adult outpatients.⁹

The trial was designed and done in partnership with local public health authorities from 11 participating cities in Brazil to simultaneously test potential treatments for early disease by means of a master protocol. A master protocol defines prospective decision criteria for discontinuing interventions for futility, stopping because of superiority against placebo, or adding new interventions. Interventions evaluated in the TOGETHER trial, thus far, include, hydroxychloroquine (protocol 1), lopinavir-ritonavir (protocol 1),¹⁰ metformin, ivermectin, fluvoxamine, doxasozin, and pegylated interferon lambda versus matching placebos (protocol 2). The TOGETHER trial is centrally coordinated by Platform Life Sciences (Vancouver, Canada) with local implementation provided by Cardresearch (Belo Horizonte, Brazil). Statistical analyses were done by Cytel (Waltham, MA, USA).

The trial complies with the International Conference of Harmonization—Good Clinical Practices as well as local regulatory requirements. It was approved for research ethics by local and national ethics boards in Brazil (CONEP CAAE: 41174620.0.1001.5120, approval letter 5.501.284) and the Hamilton Integrated Research Ethics Board (approval letter 13390) in Canada. The full protocol and statistical analysis plan have previously been published,⁹ and additional details are in Example 7). The adaptive designs Consolidated Standards of Reporting Trials extension statement guided this trial report.^{10,11} An independent data safety monitoring committee (DSMC) provided trial oversight.

### Participants

The cities and investigators of the 11 clinical sites in Brazil who participated in the trial are listed in Example 7). Local investigators, in partnership with local public health authorities, recruited participants at community health facilities (emergency settings, influenza-symptom referral centers, or primary care community centers). We used several community outreach strategies including physical and social media as per local public health authorities, in order to create awareness of the trial.

On presentation to one of the trial outpatient care clinics, local investigators screened potential participants to identify those who met the eligibility criteria. The key inclusion criteria were patients older than 18 years, presenting to an outpatient care setting with an acute clinical condition consistent with COVID-19 and symptoms beginning within 7 days of the screening date, or positive rapid test for SARS-CoV-2 antigen done at the time of screening or patient with positive SARS-CoV-2 diagnostic test within 7 days of symptom onset. Eligible patients also had at least one additional criterion for high risk: diabetes; systemic arterial hypertension requiring at least one oral medication for treatment; known cardiovascular disease (heart failure, congenital heart disease, valve disease, coronary artery disease, cardiomyopathies being treated, clinically manifested heart disease and with clinical repercussion); symptomatic lung disease or treatment for such (emphysema, fibrosing diseases); symptomatic asthma requiring chronic use of agents to control symptoms; smoking; obesity, defined as body-mass index greater than 30 kg/m² (weight and height information provided by the patient); having had a transplant; stage IV chronic kidney disease or on dialysis; immunosuppression or use of corticosteroid therapy (equivalent to at least 10 mg of prednisone per day) or immunosuppressive therapy; history of cancer in the last 0.5 years or undergoing current cancer treatment or aged 50 years or older; and unvaccinated status.

Patients who met any of the following key criteria were excluded from the trial: diagnostic examination for SARS-CoV-2 negative associated with acute flu-like symptoms (patients with negative test taken early and becoming positive a few days later were eligible, if they were less than 7 days after the onset of flu-like symptoms); acute respiratory condition compatible with COVID-19 treated in primary care and previously requiring hospitalization; acute respiratory condition owing to other causes; received vaccination for SARS-CoV-2; dyspnoea secondary to other acute and chronic respiratory causes or infections (e.g., decompensated chronic obstructive pulmonary disease, acute bronchitis, pneumonia, primary pulmonary arterial hypertension); current use of SSRIs (use of other serotonin reuptake inhibitors were not excluded); uncontrolled psychiatric disorders or suicidal ideation; inability or unwillingness to follow research guidelines and procedures. A full list of exclusion criteria is provided in the trial protocol.

If a patient met the aforementioned eligibility criteria, study personnel obtained written informed consent. After obtaining informed consent a rapid antigen test for COVID-19 (Panbio, Abbott Laboratories Jena, Jena, Germany) and a pregnancy test for women of childbearing age were done. If the COVID-19 test was negative or if the pregnancy test was positive, the participant was not included in the trial. After informed consent, study personnel collected the following data before randomization: demographics, medical history, concomitant medications, comorbidities, exposure to index case information, WHO clinical worsening scale, and the patient-reported outcomes measurement information (PROMIS) Global Health Scale.

### Randomization and Masking

Participants were randomly assigned by means of a centralized core randomization process handled by an independent unmasked pharmacist who was not aware of any protocol-related procedures and contracted specifically for this process. Sites requested randomization by text message to the pharmacist at the coordinating center. This maintained concealment of allocation. Patients were randomly assigned (1:1) by means of a block randomization procedure for each participating site, stratified by age (<50 years or ≥50 years). The trial team, site staff, and patients were masked to treatment allocation. The active drugs and the placebo pills were packaged in identically shaped bottles and labelled with alphabet letters corresponding to the active group or placebo group. Only the third-party pharmacist responsible for releasing the randomization was aware of which letter was associated with which drug or placebo. As this is a multiarm trial and all active interventions have a matching inert placebo, the matching placebo represents the proportion of the control group for the number of arms in the trial at any given time.

### Procedures

All participants received usual standard care for COVID-19 provided by health-care professionals at public health facilities. Patients were randomly assigned to fluvoxamine (Luvox, Abbott) at a dose of 100 mg twice a day for 10 days or corresponding placebo starting directly after randomization (day 1). Research personnel provided participants with a welcome video, which gave information on the trial, study drug, adverse events, and follow-up procedures. Clinicians providing usual care in public health facilities typically focus on the management of symptoms and provide antipyretics or recommend antibiotics only if they suspect bacterial pneumonia.

Study personnel collected outcome data on days 1, 2, 3, 4, 5, 7, 10, 14, and 28 in person or via telephone contact or social media applications using video-teleconferencing. We collected outcome data irrespective of whether participants took study medication. In case of adverse events, unscheduled visits (during the treatment period) outside of clinical care could occur at any time.

Considering the transmissible characteristics of SARS-CoV-2 and the isolation recommendations of positive individuals, we collected few vital sign data. Cardiac safety was assessed by means of a six-lead electrocardiogram (Kardiamobile, Mountain View, CA, USA) at the baseline visit. The digital recordings were deidentified and transferred to a central facility (Cardresearch, Belo Horizonte, Brazil) for reading. Oxygen status was assessed by means of a pulse oximeter for non-invasive arterial oxygen saturation and pulse (Jumper Medical Equipment, Shenzhen, China), and temperature by means of a standard digital oral thermometer administered by research personnel. Mid-turbinate nasal swab kits and sterile recipient storage were provided for collection of nasopharyngeal swab or sputum-saliva. Nasal swabs for PCR testing was completed on the first quarter of participants enrolled in the trial on days 3 and 7. Viral clearance was assessed to establish whether active drugs showed any antiviral effects.

All serious and non-serious adverse events were reported to study personnel as per local regulatory requirements. Reportable adverse events included serious adverse events, adverse events resulting in study medication discontinuation, and adverse events assessed as possibly related to study medication.

### Outcomes

Our primary outcome was a composite endpoint of medical admission to a hospital setting due to COVID-19-related illness defined as COVID-19 emergency setting visits with participants remaining under observation for more than 6 h or referral to further hospitalization due to the progression of COVID-19 within 28 days of randomization. Because many patients who would ordinarily have been hospitalized were prevented from admission due to hospital over-capacity during peak waves, the composite endpoint addresses both hospitalization and a proxy for hospitalization, retention in a COVID-19 emergency hospital setting. This region of Brazil implemented hospital-like services in the emergency settings with 50-80 bed units providing services including multiday stays, oxygenation, and mechanical ventilation. The 6 h threshold referred only to periods of time recommended for observation by a clinician and does not include waiting times. Key secondary outcomes include viral clearance, time to clinical improvement, number of days with respiratory symptoms, time to hospitalization for any cause or due to COVID-19 progression, all-cause mortality and time to death from any causes, WHO clinical worsening scale score, days in hospital and on ventilator and adverse events, adverse reactions to the study medications, and the proportion of participants who are non-adherent with the study drugs. All secondary outcomes were assessed up to 28 days following randomization.

### Statistical Analysis

The Adaptive Design Protocol and the Master Statistical Analysis Plan provide details of sample size calculation and statistical analysis.⁹ This trial is adaptive and applies sample size reassessment approaches. To plan for each arm, we assumed a minimum clinical utility of 37.5% (relative risk reduction) to achieve 80% power with 0.05 two-sided type 1 error for a pairwise comparison against the placebo assuming a control event rate of 15%. This resulted in an initial plan to recruit 681 participants per arm. The statistical team did planned interim analyses. Stopping thresholds for futility were established if the posterior probability of superiority was less than 40% at interim analysis. An arm could be stopped for superiority if the posterior probability of superiority met the threshold of 97.6%.

Baseline characteristics are reported as count (%) or median and IQR for continuous variables. We applied a Bayesian framework for our primary outcome analysis and a frequentist approach for all sensitivity analyses and secondary outcomes. Bayesian analysis allows us to report the posterior probability of treatment efficacy at the end of the trial, independently of the decisions made along the way. Posterior efficacy of fluvoxamine for the primary outcome is calculated by means of the beta-binomial model for event rates, as detailed in the statistical analysis plan,¹² assuming informed priors on the basis of observational data for both placebo and fluvoxamine, for both intention-to-treat and per-protocol analyses (defined as taking >80% of possible doses). Modified intention to treat (mITT) was defined as receiving treatment for at least 24 h before a primary outcome. We accounted for any temporal changes in event rates by means of only the concurrent randomized population. We assessed subgroup effects according to the preplanned statistical analysis plan. We calculated the number needed to treat.

Secondary outcomes were assessed by means of a prespecified frequentist approach. For viral clearance we fitted a longitudinal, mixed-effect logistic regression model with a treatment and time interaction term for binary patient outcomes (COVID-19 positive-negative) reported on day 3 and 7 from randomization, with subject random effect. We assessed time-to-event outcomes using Cox proportional hazard models and binary outcomes using logistic regression. Model assumptions were evaluated by testing for proportionality. We did a subgroup analysis and reported p values for the interactions. Per-protocol analyses were considered sensitivity analyses to assess the robustness of the results. We followed the statistical analysis plan and provided a post-hoc analysis where requested by reviewers. All analyses were done by means of R version 4.0.3. Full details of the statistical analysis plan can be found in the Data Section.

A data and safety monitoring committee provided independent oversight for this trial. We planned a fourth and final interim analysis of the fluvoxamine group based on data up to Aug. 2, 2021. Herein, we present follow-up of all patients up to Sep. 9, 2021. The trial is registered at ClinicalTrials.gov (NCT04727424).

### Results

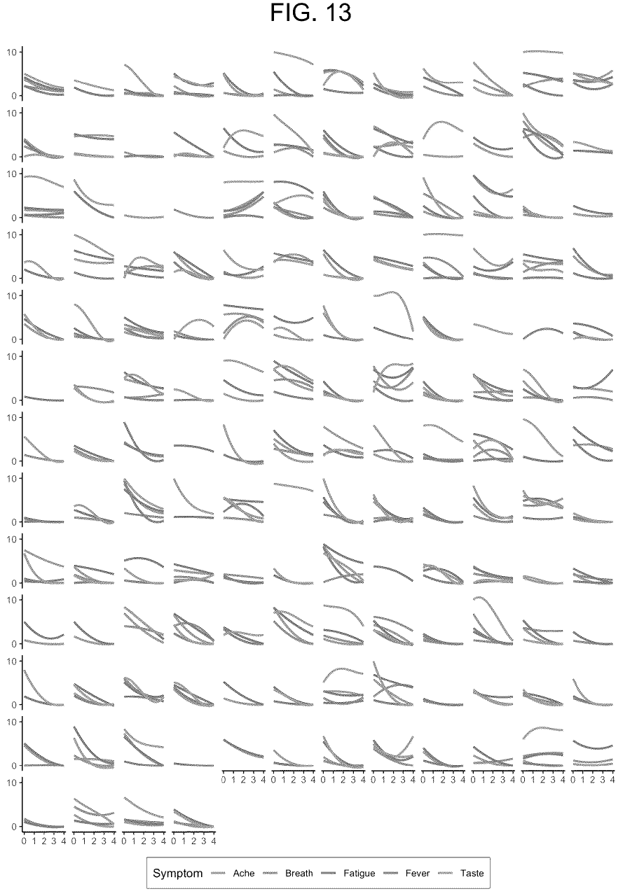

We have screened 9803 potential participants for inclusion in this trial to date. The TOGETHER trial enrolled its first participant on Jun. 2, 2020 and enrolment into the fluvoxamine group began on Jan. 20, 2021. As the trial is ongoing, herein we provide descriptive summaries of only those randomly assigned to fluvoxamine and its concurrent control. By Aug. 5, 2021, 1497 recruited participants were randomly assigned to fluvoxamine (n=741) or placebo (n=756), and 1826 were randomly assigned to other treatment groups (FIG. 14). Herein, we present data on all patients completing 28 days of follow-up as of Sep. 9, 2021. The median age was 50 years (range 18-102) and 862 (58%) were women (TABLE 15). Most participants self-identified as mixed race 1428 (95%), 12 (1%) as white, 10 (1%) as black or African heritage, the rest self-identified as unknown 47 (3%). With respect to covariates of age, body-mass index, and comorbidities, the groups were generally well balanced (TABLE 15). The mean number of days with symptoms before randomization was 3.8 days (SD 1.87).

TABLE 15
Patient characteristics by treatment allocation in the TOGETHER trial

| | |
|---|---|
| Fluvoxamine (n = 741) | Placebo (n = 756) |

Sex

| | | | | |
|---|---|---|---|---|
| Female | 409 | (55%) | 453 | (60%) |
| Male | 332 | (45%) | 303 | (40%) |

Race

| | | | | |
|---|---|---|---|---|
| Mixed race* | 709 | (96%) | 719 | (95%) |
| White | 6 | (1%) | 6 | (1%) |
| Black or African American | 5 | (1%) | 5 | (1%) |
| Unknown | 21 | (3%) | 26 | (3%) |

Age, years

| | | | | |
|---|---|---|---|---|
| <50 | 379 | (51%) | 368 | (49%) |
| ≥50 | 327 | (44%) | 328 | (43%) |
| Unspecified | 46 | (6%) | 49 | (6%) |

Age descriptive statistics

| | | | | |
|---|---|---|---|---|
| Median (IQR) | 50 | (39-56) | 49 | (38-56) |
| Body-mass index | | | | |
| <30 kg/m² | 355 | (48%) | 373 | (49%) |
| ≥30 kg/m² | 376 | (51%) | 375 | (50%) |
| Unspecified | 10 | (1%) | 8 | (1%) |

Time since onset of symptoms, days

| | | | | |
|---|---|---|---|---|
| 0-3 | 328 | (44%) | 310 | (41%) |
| 4-7 | 239 | (32%) | 267 | (35%) |
| Unspecified | 174 | (23%) | 179 | (24%) |

Risk factors

| Chronic cardiac disease | 9 | (1%) | 7 | (1%) |
|---|---|---|---|---|
| Uncontrolled hypertension | 106 | (14%) | 88 | (12%) |
| Chronic pulmonary disease | 6 | (1%) | 3 | (<1%) |
| Asthma | 12 | (2%) | 16 | (2%) |
| Chronic kidney disease | 2 | (<1%) | 2 | (<1%) |
| Rheumatological disorder | 1 | (<1%) | 0 | |
| Chronic neurological disorder | 8 | (1%) | 6 | (1%) |
| Type 1 diabetes | 25 | (3%) | 22 | (3%) |
| Type 2 diabetes | 104 | (14%) | 92 | (12%) |
| Autoimmune disease | 0 | | 2 | (<1%) |
| Any other risk factor(s) or comorbidities | 25 | (3%) | 24 | (3%) |

Data are n (%).
*Self-identified as someone with mixed ancestry.

All patients accessed care via a COVID-19 emergency setting. There were a total of 180 patients in the fluvoxamine group and 251 patients in the placebo group who had any interaction with a COVID-19 emergency setting. The relative risk (RR) for ever visiting a COVID-19 emergency setting was 0.73 (95% CI 0.62-0.88).

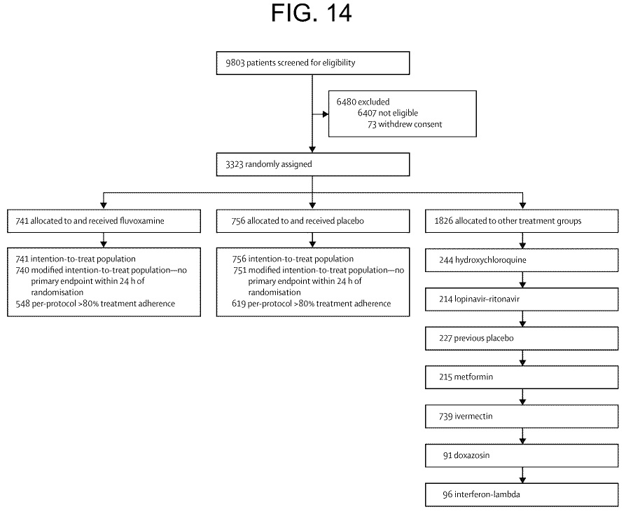

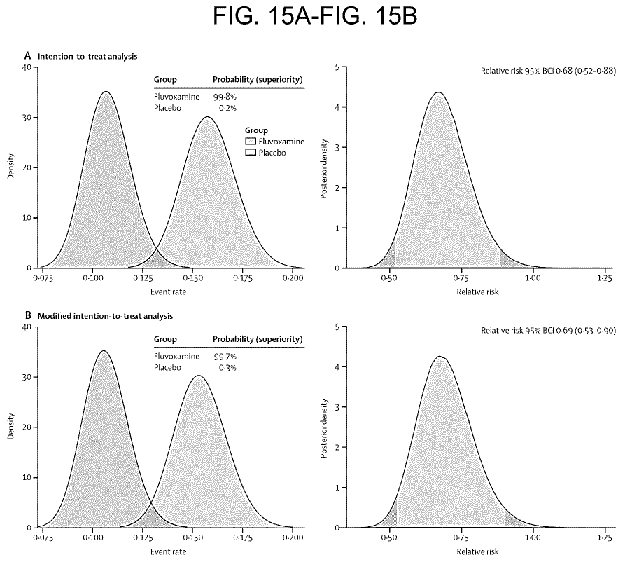

In the fluvoxamine group 79 (11%) participants had a primary outcome event compared with 119 (16%) in the placebo group (TABLE 16). Most events (87%) were hospitalizations. On the basis of the Bayesian beta-binomial model, there was evidence of a benefit of fluvoxamine reducing the composite primary endpoint of hospitalization defined as either retention in a COVID-19 emergency setting or transfer to tertiary hospital due to COVID-19 (RR 0.68; 95% Bayesian credible interval [BCI] 0.52-0.88) in the ITT population (FIG. 15A) and RR 0.69; 95% BCI 0.53-0.90 in a modified ITT population (FIG. 15B). The number needed to treat was 20. Per-protocol analysis showed a larger treatment effect (0.34, 95% BCI 0.21-0.54). The probability that the event rate was lower in the fluvoxamine group compared with placebo was 99.8% for the ITT population and 99.7% for the mITT population (FIG. 15A, FIG. 15B). When the DSMC met on Aug. 5, 2021, it recommended that the TOGETHER trial stop randomly assigning patients to the fluvoxamine group, as this comparison had met the prespecified superiority criterion for the primary endpoint (prespecified superiority threshold 97.6%).

TABLE 16
Proportion of primary outcome events and relative risk of hospitalization defined
as either retention in a COVID-19 emergency setting or transfer to tertiary
hospital due to COVID-19 for patients allocated fluvoxamine versus placebo.

| | Intention-to-treat analysis | | | | | Modified intention-to-treat analysis | | | | |
|---|---|---|---|---|---|---|---|---|---|---|
| | N | n (%) | | Relative risk (95% BCI) | | N | n (%) | | Relative risk (95% BCI) | |
| Fluvoxamine | 741 | 79 | (11%) | 0.68 | (0.52-0.88) | 740 | 78 | (11%) | 0.69 | (0.53-0.90) |
| Placebo | 756 | 119 | (16%) | 1 | (ref) | 752 | 115 | (15%) | 1 | (ref) |

BCI = Bayesian credible interval.

TABLE 17 presents findings from secondary outcome analyses. There were no significant differences between fluvoxamine and placebo for viral clearance at day 7 (p=0.090) and hospitalizations due to COVID (p=0.10), all-cause hospitalizations (p=0.09), time to hospitalization (p=0.11), number of days in hospital (p=0.06), mortality (p=0.24), time to death (p=0.49), number of days on mechanical ventilation (p=0.90), time to recovery (p=0.79) or the PROMIS Global Physical (p=0.55) or Mental Scale (p=0.32; Example 7).

TABLE 17
Secondary outcomes of fluvoxamine versus placebo in the TOGETHER trial.

| | | | | | Estimated treatment | | p |
|---|---|---|---|---|---|---|---|
| | Fluvoxamine | | Placebo | | effect (95% CI) | | value |
| Viral clearance (day 7) | 40/207 | (19%) | 58/221 | (26%) | 0.67 | (0.42-1.06)* | 0.090 |
| Hospitalized for COVID | 75/741 | (10%) | 97/756 | (13%) | 0.77 | (0.55-1.05)* | 0.10 |
| All-cause hospitalization | 76/741 | (10%) | 99/756 | (13%) | 0.76 | (0.58-1.04)* | 0.088 |
| Time to hospitalization, days | 5 | (3-7) | 5 | (3-7.5) | 0.79 | (0.58-1.06)^{†} | 0.11 |
| Period of hospitalization, days | 8 | (5-13) | 6 | (3-10.75) | 1.23 | (0.99-1.53)^{‡} | 0.059 |
| Emergency setting visit for at least | 7/741 | (1%) | 36/756 | (5%) | 0.19 | (0.08-0.41)* | 0.0001 |
| 6 h | | | | | | | |
| Time to the emergency visit for at | 4 | (3-7) | 5 | (3-8.25) | 0.20 | (0.09-0.44)^{†} | 0.002 |
| least 6 h, days | | | | | | | |
| Death, intention to treat | 17/741 | (2%) | 25/756 | (3%) | 0.69 | (0.36-1.27)* | 0.24 |
| Time to death, days | 17 | (9-21) | 14 | (8-20) | 0.80 | (0.43-1.51)^{†} | 0.49 |
| Mechanical ventilation | 26 | | 34 | | 0.77 | (0.45-1.30) | 0.33 |
| Time on mechanical ventilator, | 5.5 | (3-12.75) | 6.5 | (2.25-12) | 1.03 | (0.64-1.67)^{‡} | 0.90 |
| days | | | | | | | |
| Adherence | 548/741 | (74%) | 618/738 | (82%) | 0.62 | (0.48-0.77)* | 0.0003 |
| Death, per protocol | 1/548 | (<1%) | 12/618 | (2%) | 0.09 | (0.01-0.47) | 0.022 |

Treatment emergent adverse event

| | | | | | | | |
|---|---|---|---|---|---|---|---|
| Grade 1 | 20/741 | (3%) | 11/756 | (1%) | 1.88 | (0.91-4.09)* | 0.096 |
| Grade 2 | 72/741 | (10%) | 81/756 | (11%) | 0.91 | (0.64-1.25)* | 0.52 |
| Grade 3 | 38/741 | (5%) | 50/756 | (7%) | 0.76 | (0.49-1.18)* | 0.22 |
| Grade 4 | 21/741 | (3%) | 20/756 | (3%) | 1.07 | (0.58-2.01)* | 0.82 |
| Grade 5 | 18/741 | (2%) | 26/756 | (3%) | 0.70 | (0.37-1.28)* | 0.25 |

Data are n/N (%) or median (IQR) unless otherwise stated.
*Unadjusted odds ratio.
^{†}Unadjusted hazard ratio.
^{‡}Exponentiated unadjusted estimates from a log-transformed linear regression.

84 participants stopped fluvoxamine and 64 participants stopped placebo owing to issues of tolerability. Per-protocol findings among patients who reported optimal adherence (greater than 80% for possible days) indicated a significant treatment effect (RR 0.34; 95% BCI 0.21-0.54 for the primary outcome and for mortality (odds ratio 0.09; 95% CI 0.01-0.47). With respect to adverse events, there were no significant differences in number of treatment emergent adverse events among patients in the fluvoxamine and placebo groups.

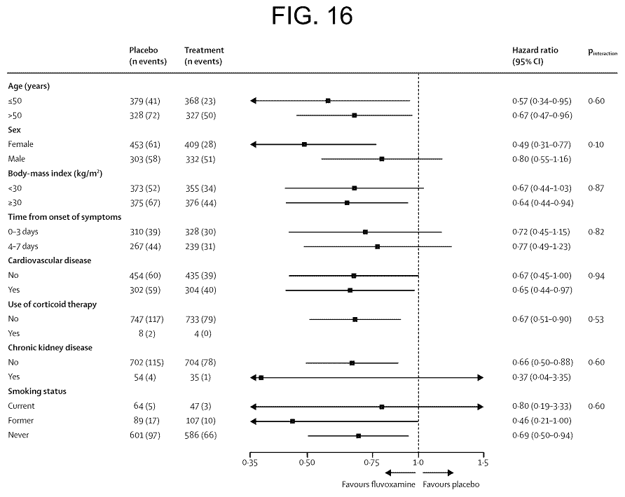

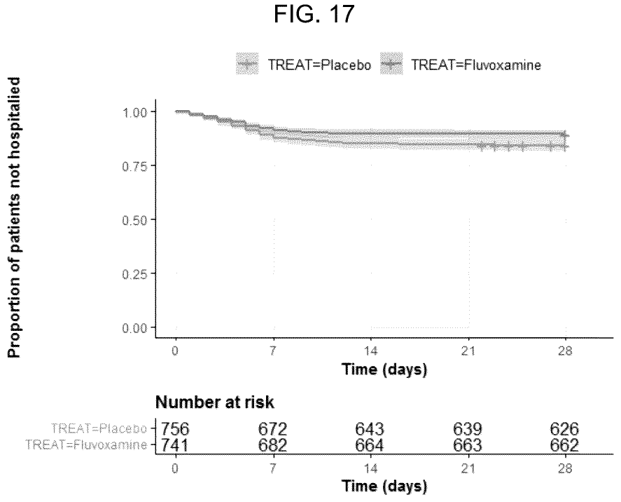

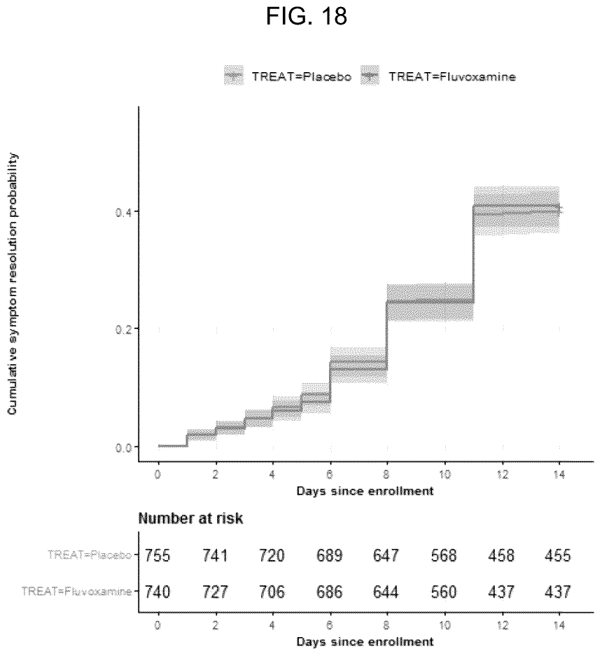

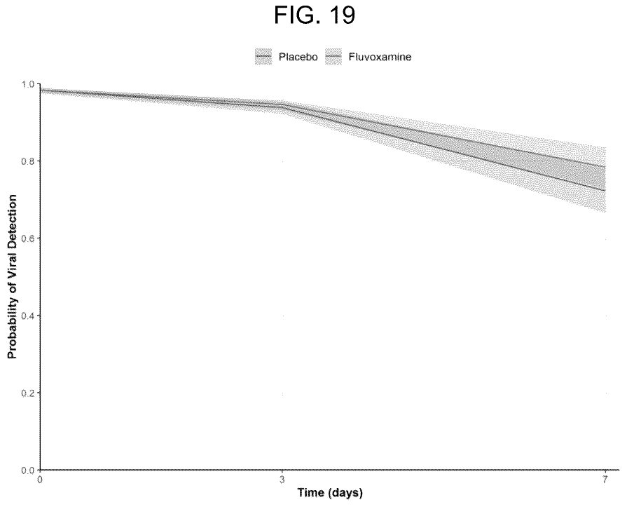

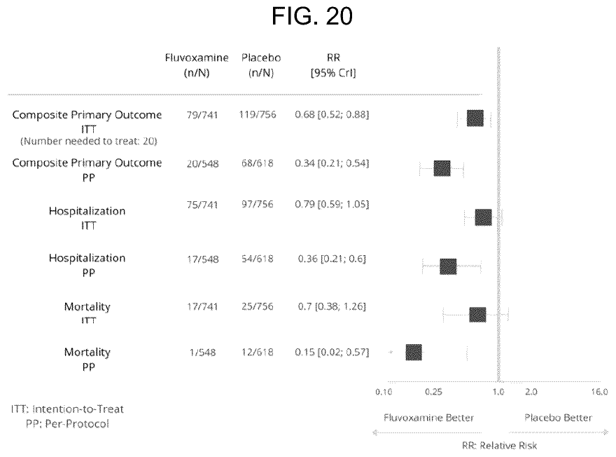

In the prespecified subgroup analysis, we found no evidence of moderation of treatment effect for fluvoxamine compared with placebo, for subgroups of age, sex, days since symptom onset, smoking status, or comorbidities (FIG. 16, Example 7).

### Discussion

This is, to the best of our knowledge, the first large, randomized controlled trial to test the efficacy of fluvoxamine for acute treatment of COVID-19. We found a clinically important absolute risk reduction of 5.0%, and 32% RR reduction, on the primary outcome of hospitalization defined as either retention in a COVID-19 emergency setting or transfer to tertiary hospital due to COVID-19, consequent on the administration of fluvoxamine for 10 days. This study is only the second study to show an important treatment benefit for a repurposed drug in the early treatment population.¹³

Our findings represent the complete analysis of the trial after the DSMC recommended stopping the active fluvoxamine group and all 28-day follow-up of randomly assigned patients. Given fluvoxamine's safety, tolerability, ease of use, low cost, and widespread availability, these findings might influence national and international guidelines on the clinical management of COVID-19.

Our results are consistent with an earlier smaller trial done in the USA (led by EJL and AMR).⁶

That study used a higher dose of fluvoxamine (100 mg three times a day for 15 days) and included a lower risk group for the primary outcome but found no clinical deterioration among 80 patients receiving fluvoxamine versus six cases among 72 patients receiving placebo. A large observational study from France involved a different population, 7230 hospitalized COVID-19 patients, and reported a reduction in use of intubation or death with use of SSRIs.⁵

The underlying mechanism of fluvoxamine for COVID-19 disease remains uncertain. Although hypotheses include several potential mechanisms,⁴ the main reason for the initial study of fluvoxamine as a treatment of COVID-19 was its anti-inflammatory action through activation of the S1R.¹⁴

S1R is an endoplasmic reticulum (ER) chaperone membrane protein involved in many cellular functions,¹⁵ including regulation of ER stress response-unfolded protein response and regulation of cytokine production in response to inflammatory triggers.¹⁶

In the presence of fluvoxamine, S1R might prevent the ER stress sensor inositol-requiring enzyme 1a from splicing and activating the mRNA of X-box protein 1, a key regulator of cytokine production including interleukins IL-6, IL-8, IL-1p, and IL-12. In a 2019 study by Rosen and colleagues, fluvoxamine showed benefit in preclinical models of inflammation and sepsis through this mechanism.¹⁶

A second mechanism might be fluvoxamine's antiplatelet activity.¹⁷ SSRIs can prevent loading of serotonin into platelets and inhibit platelet activation, which might reduce the risk of thrombosis, and these antiplatelet effects can be cardioprotective. Finally, another potential mechanism of action might be related to the effect of fluvoxamine in increasing plasma levels of melatonin.¹⁶

In vitro and animal studies are needed to help clarify the most probable mechanism(s). Biomarker studies included as part of future randomized controlled trials might also help to clarify mechanisms.

Since the start of the COVID-19 pandemic, there have been more than 2800 randomized controlled trials registered on ClinicalTrials.gov. However, fewer than 300 have been reported and most clinical trials have been small and underpowered, with sample sizes less than 100. In many cases, these trials have been unsuccessful at recruiting as the local epidemics occur in waves and sustainable infrastructure to maintain staff or local interest for recruitment is lacking. The trials that provide the clearest medical understanding tend to be the larger platform trials, such as SOLIDARITY,¹⁷ RECOVERY,¹⁶ PRINCIPLE,¹¹ and REMAP-CAP.¹⁹

As a result, we actively collaborate with other investigators running trials with overlapping interventions so that they can be aware of our study decisions and establish whether they should influence their respective trials.

Strengths of our trial include the rapid recruitment and enrolment of high-risk patients for the development of severe COVID-19. Our recruitment strategy involves engagement with the local public health system, thus allowing recruitment that frequently exceeds 20 patients per day. We enrolled only participants with diagnosed COVID-19 and less than 7 days of symptom onset using a commercially available COVID-19 rapid antigen test (Panbio, Abbott Rapid Diagnostics Jena, Jena, Germany). The concordance of COVID-19 positive tests with RT-PCR was evaluated on the group of participants with PCR evaluations and a concordance rate of greater than 99% on both tests collected at baseline was found. In this trial we did not enroll participants without positive COVID-19 tests, nor those who were asymptomatic SARS-CoV-2 positive. Our primary outcome is hospitalization defined as either retention in a COVID-19 emergency setting for more than 6 h or transfer to tertiary hospital due to COVID-19. The event adjudication committee did count patient wait times as contributing to a primary endpoint. Specialized emergency settings were developed to respond to the Brazilian epidemic and we considered prolonged observation and treatment in these settings as equivalent in importance to hospitalization as many patients who typically would be hospitalized were prevented from doing so owing to hospital over-capacity. In our trial, 87% of all primary outcome events eventually resulted in transfer to a tertiary hospital. Patients observed in both the emergency setting and hospital were counted only once. Our sub-group analyses examined pre-determined population groups and tests for interaction did not detect differing effects for any sub-group. Female sex was identified as a significant sub-group favoring fluvoxamine while male sex was not, however we did not detect differing effects between the groups.

Our understanding of the epidemiology of COVID-19 as well as its disease progression and outcomes have evolved since beginning this platform trial in June, 2020. Early studies assessed the effects of interventions on viral load and clearance, whereas later studies also evaluate more clinical outcomes. We made adjustments to the trial according to prespecified rules and in communication with the appropriate ethics review committees that allowed us to respond to the epidemic waves while maintaining high rates of recruitment. Unlike many outpatient clinical trials, our study involves direct patient contact through the use of medical students, nurses, and physicians who do at-home visits as well as follow-up via telecommunications. Given the rapid recruitment of patients in combination with the high event rate of COVID-19 emergency setting visits and hospitalizations, we were able to evaluate the effects of interventions when portions of the planned population had been recruited. The period between first recruitment of a patient on fluvoxamine and the final data cut for our trial was 219 days.

Major limitations of our trial are related to the challenges of doing a trial in a disease that is not well characterized. There is no standard of care that exists for early treatment of COVID-19 and various advocacy groups promote different interventions, including some of those evaluated in this and our previous trials.²⁰ Furthermore, there is little understanding of who is at greatest risk of disease progression from this disease as some patients with numerous risk factors do recover quickly whereas some others with less established risk factors might not. Our population had a higher rate of hospitalization events than observed in most clinical trials,²⁰ thus permitting inferences on treatment effects in this higher-risk population. Although intention-to-treat analysis provides more real-world evidence than per-protocol analysis, we found that patients who reported optimal adherence (greater than 80% for possible days-our per-protocol analysis) had a greater treatment benefit, suggesting that intensifying adherence to treatment might have considerable clinical benefits. However, adherence might be related to tolerability. 84 participants stopped fluvoxamine and 64 participants stopped in the placebo group for this reason. Finally, when the trial began, vaccines were not available in Brazil but became more widely available as the trial progressed. Although we modified inclusion criteria and permitted vaccinated patients during the trial, we believe this had minimal effect on the primary outcome as only 86 (6%) of 1497 reported at least one dose of a COVID-19 vaccine at the end of the trial.

Our trial has found that fluvoxamine, an inexpensive existing drug, reduces the need for advanced disease care in this high-risk population. A 10-day course of fluvoxamine costs approximately 4 USD even in well-resourced settings.²¹ Our study compares favorably with the treatment effects of more expensive treatments including monoclonal antibodies for outpatient treatment.^{20,22,23}

The absolute number of serious adverse events associated with fluvoxamine was lower than for placebo and this might reflect the modulatory effect of fluvoxamine on systemic inflammation in these participants. Lower respiratory tract infections were reported less frequently in patients in the fluvoxamine group than those in the placebo group. This is concordant with the reduction of hospital admissions in patients with confirmed COVID-19 treated with fluvoxamine, and the numerically lower number of patients requiring mechanical ventilation.

Fluvoxamine is widely available but is not on the WHO Essential Medicines List,²⁴ whereas a closely related SSRI, fluoxetine, is on the list. It is now crucial to establish whether a class effect exists and whether these drugs can be used interchangeably for COVID-19. The important findings that inhaled budesonide decreased time to recovery¹¹ among a similar population to our trial and had a trend towards decreased hospitalizations suggests that this as an alternative or additional intervention for outpatient care that should be evaluated. The PRINCIPLE trial evaluated time to recovery by means of self-reported recovery up to 28 days after randomization to budesonide.¹¹

Our trial differed as we evaluated improvement in the WHO categorization of disease disability up to days 14 and then 28 (Example 7). Finally, our study was among primarily unvaccinated patients. Further evidence of treatment benefits are needed to establish the effect of fluvoxamine among vaccinated populations.

Use of interventions, including fluvoxamine, to prevent progression of illness and hospitalization is critically dependent on identifying higher-risk individuals. Unselected populations will have a lower risk. What absolute reduction in risk of clinical deterioration would motivate patients to choose treatment (probably the approximately 5% that we observed, but perhaps not much lower) remains uncertain. These considerations raise the importance of the development of a validated prediction rule for deterioration in patients in the early stages of COVID-19 infection.

TABLE 18
Summary of Sub-Group Analysis for Hospitalization or Extended Emergency Room Visit Due to COVID-19

| Factor | Subgroup | N_Placebo | N_Fluvoxamine | n_Placebo | n_Fluvoxamine | HR (95% CI) | |
|---|---|---|---|---|---|---|---|
| Age | <=50 | 37 [symbol] | 168 | 4 [symbol] | 23 | 0.57 | [0.34; 0.95] |
| Age | >50 | 328 | 327 | 72 | 5 [symbol] | 0.67 | [0.47; 0. [symbol] 6] |
| Sex | Female | 153 | 409 | 61 | 28 | 0.4 [symbol] | [0.11; 0.77] |
| Sex | Male | 303 | 33 [symbol] | 58 | 51 | 0.80 | [0.55; 1. [symbol] 6] |
| BMI (Kg/m²) | <30 | 373 | [symbol] | 52 | [symbol] 4 | 0. [symbol] 7 | [0.44; 1.0 [symbol] ] |
| BMI (Kg/m²) | >30 | 375 | [symbol] 76 | 67 | 44 | 0.64 | [0.44; 0.94] |
| Time from onset of symptoms | 0-3 days | 310 | 328 | 3 [symbol] | 30 | 0.7 [symbol] | [0.45; 1.15] |
| Time from onset of symptoms | 4-7 days | 267 | 239 | 44 | [symbol] 1 | 0.77 | [0. [symbol] 9; 1. [symbol] ] |
| Cardiovascular disease | N | 747 | 7 [symbol] 3 | 117 | 7 [symbol] | 0.67 | [0. [symbol] 1; 0.9 [symbol] ] |
| Cardiovascular disease | Y | 8 | 4 | 2 | 0 | | |
| Chronic kidney disease | N | 702 | 704 | 11 [symbol] | 78 | 0. [symbol] 6 | [0. [symbol] 0; 0.8 [symbol] ] |
| Chronic kidney disease | Y | 54 | [symbol] 5 | 4 | 1 | 0. [symbol] 7 | [0.04; [symbol] .35] |
| Smoking status | Current | 64 | 47 | 5 | [symbol] | 0.80 | [0.1 [symbol] ; 3.3 [symbol] ] |
| Smoking status | Former | 89 | 107 | 17 | 10 | 0.46 | [0. [symbol] 1; 1 [symbol] ] |
| Smoking status | Never | 691 | [symbol] 86 | 97 | 66 | 0. [symbol] 9 | [ [symbol] . [symbol] 0; [symbol] . [symbol] ] |

[symbol]

indicates data missing or illegible when filed

### REFERENCES

1. Torres I Artaza O Profeta B Alonso C Kang J COVID-19 vaccination: returning to WHO's Health For All. Lancet Glob Health. 2020; 8: e1355-e1356.
2. Rayner C R Dron L Park J J H et al. Accelerating clinical evaluation of repurposed combination therapies for COVID-19. Am J Trop Med Hyg. 2020; 103: 1364-1366
3. Omi T Tanimukai H Kanayama D et al. Fluvoxamine alleviates ER stress via induction of sigma-1 receptor. Cell Death Dis. 2014; 5e1332
4. Sukhatme V P Reiersen A M Vayttaden S J Sukhatme V V. Fluvoxamine: a review of its mechanism of action and its role in COVID-19. Front Pharmacol. 2021;-12652688
5. Hoertel N Sánchez-Rico M Vernet R et al. Association between antidepressant use and reduced risk of intubation or death in hospitalized patients with COVID-19: results from an observational study. Mol Psychiatry. 2021; (published online February 4.) https://doi.org/10.1038/s41380-021-01021-4
6. Lenze E J Mattar C Zorumski C F et al. Fluvoxamine vs placebo and clinical deterioration in outpatients with symptomatic COVID-19: a randomized clinical trial. JAMA. 2020; 324: 2292-2300.
7. Park J J H Siden E Zoratti M J et al. Systematic review of basket trials, umbrella trials, and platform trials: a landscape analysis of master protocols. Trials. 2019; 20: 572
8. Woodcock J LaVange L M Master protocols to study multiple therapies, multiple diseases, or both. N Engl J Med. 2017; 377: 62-70
9. Reis G Silva E AdSM Silva D C M et al. A multi-center, adaptive, randomized, platform trial to evaluate the effect of repurposed medicines in outpatients with early coronavirus disease 2019 (COVID-19) and high-risk for complications: the TOGETHER master trial protocol. Gates Open Res. 2021; 5: 117.
10. Anderson G M. Fluvoxamine, melatonin and COVID-19. Psychopharmacology (Berl). 2021; 238: 611
11. Pan H Peto R Henao-Restrepo A M et al. Repurposed antiviral drugs for Covid-19—interim WHO Solidarity Trial results. N Engl J Med. 2021; 384: 497-511
12. Reis G Moreira Silva EADS Medeiros Silva D C et al. Effect of early treatment with hydroxychloroquine or lopinavir and ritonavir on risk of hospitalization among patients with COVID-19: the TOGETHER randomized clinical trial. JAMA Netw Open. 2021; 4e216468
13. Yu L-M Bafadhel M Dorward J et al. Inhaled budesonide for COVID-19 in people at high risk of complications in the community in the UK (PRINCIPLE): a randomized, controlled, open-label, adaptive platform trial. Lancet. 2021; 398: 843-855
14. Pashaei Y Drug repurposing of selective serotonin reuptake inhibitors: could these drugs help fight COVID-19 and save lives?. J Clin Neurosci. 2021; 88: 163-172
15. Ishima T Fujita Y Hashimoto K. Interaction of new antidepressants with sigma-1 receptor chaperones and their potentiation of neurite outgrowth in PC12 cells. Eur J Pharmacol. 2014; 727: 167-173
16. Rosen D A Seki S M Fernéndez-Castañeda A et al. Modulation of the sigma-1 receptor-IRE1 pathway is beneficial in preclinical models of inflammation and sepsis. Sci Transl Med. 2019; Ileaau5266
17. Schlienger R G Meier C R. Effect of selective serotonin reuptake inhibitors on platelet activation: can they prevent acute myocardial infarction?. Am J Cardiovasc Drugs. 2003; 3: 149-162
18. Horby P W Pessoa-Amorim G Peto L et al. Tocilizumab in patients admitted to hospital with COVID-19 (RECOVERY): preliminary results of a randomized, controlled, open-label, platform trial. medRxiv. 2021; (https://doi.org/2021.02.11.21249258 published online February 11 (preprint).)
19. Angus D C Derde L Al-Beidh F et al. Effect of hydrocortisone on mortality and organ support in patients with severe COVID-19: the REMAP-CAP COVID-19 corticosteroid domain randomized clinical trial. JAMA. 2020; 324: 1317-1329
20. Siemieniuk R A Bartoszko J J Ge L et al. Drug treatments for covid-19: living systematic review and network meta-analysis. BMJ. 2020; 370m2980
21. Wang J Levi J Ellis L Hill A. Minimum manufacturing costs, national prices and estimated global availability of new repurposed therapies for COVID-19. medRxiv. 2021; (published online June 3.) (preprint). https://doi.org/10.1101/2021.06.01.21258147
22. Chen P Nirula A Heller B et al. SARS-CoV-2 neutralizing antibody LY-CoV555 in outpatients with Covid-19. N Engl J Med. 2021; 384: 229-237
23. Weinreich D M Sivapalasingam S Norton T et al. REGN-COV2, a neutralizing antibody cocktail, in outpatients with Covid-19. N Engl J Med. 2021; 384: 238-251
24. WHO World Health Organization model list of essential medicines: 21st list 2019. World Health Organization, Geneva 2019

### Example 8: Emergency Use Authorization Request for Use of Fluvoxamine for Treatment of COVID-19

Inventors have submitted an Emergency Use Authorization (EUA) request to add the currently unapproved indication for the existing FDA-approved medicine of fluvoxamine.

This request is affiliated with FDA IND 152439, but this EUA application utilizes existing published data from two double-blind, randomized, placebo-controlled clinical trials which showed clinical benefit in the early outpatient treatment of mild/moderate COVID-19 (including variants) within the first 7 days of symptomatic illness.

An urgent clinical need exists for an oral medicine that can treat early symptomatic COVID-19 disease to stop clinical deterioration and prevent hospitalization. The omicron variant, which is not neutralized by most currently available monoclonal antibodies, is rapidly spreading in the USA thus defeating the only currently authorized outpatient interventions shown to reduce hospitalization and mortality. Fluvoxamine, whose putative mechanism is not contingent upon variant type and is widely available, is believed to meet an urgent medical need right now.

It is believed, that based on the totality of scientific evidence available, including data from adequate and well-controlled clinical trials, if available, it is reasonable to believe that the product may be effective in treating COVID-19, or a serious or life-threatening disease or condition caused by COVID-19; that the known and potential benefits, when used to treat such disease or condition, outweigh the known and potential risks for the product; and that there are no adequate, approved, and available alternatives.

With the emergence of the SARS-CoV-2 Omicron variant, there is significant loss of activity for both Eli Lilly's bamlanivimab/etesevimab and Regeneron's casirivimab/imdevimab.

While there are oral antivirals presently submitted for FDA EUA approval, such as Nirmatrelvir (Paxlovid) and molnupiravir (Lagevrio), these are not available and may remain unavailable to populations worldwide. There remains a large demand for an inexpensive, orally available, effective outpatient early treatment of COVID-19 with known safety record. Due to the lack of effective oral options, people continue to seek out unproven therapies such as ivermectin.

Fluvoxamine has shown clinical benefit in two double-blind randomized placebo-controlled clinical trials for treatment of mild/moderate outpatient early COVID-19 in the first 7 days of illness with decreasing clinical progression to severe COVID-19 (as per FDA categorization of severe COVID-19) and reducing hospitalizations.

TABLE 19
Summary of the two randomized clinical trial data demonstrate a 25%
reduction in hospitalizations and 36% reduction in progression to severe
disease with hypoxia ≤92%, ER visit >6 hour (with hypoxia), or
hospitalizations by intent to treat analysis of 1649 participants.

| | | Blinded | Relative Risk | Absolute Risk |
|---|---|---|---|---|
| Endpoint | Fluvoxamine | Placebo | (95% CI) | Reduction (95% CI) |
| Progression to | 9.6% | 15.1% | 0.64 | 5.4% |
| Severe COVID-19* | (79/821) | (125/828) | (0.50 to 0.84) | (2.4% to 7.5%) |
| Hospitalization | 9.3% | 12.4% | 0.75 | 3.1% |
| | (76/821) | (103/828) | (0.57-0.99) | (0.1% to 5.3%) |
| Mortality | 2.1% | 3.0% | 0.69 | 0.9% |
| | (17/821) | (17/821) | (0.36-1.27) | (+0.8% more to 1.9%) |

*Hospitalization or ER Visit >6 hr or Hypoxia <92%

Fluvoxamine, sold under the brand name Luvox, among others, is a FDA-approved antidepressant of the selective serotonin reuptake inhibitor (SSRI) class. The drug is currently primarily prescribed for the treatment of obsessive-compulsive disorder (OCD).

The current IND 152439 randomized trial testing fluvoxamine 50 mg twice daily among other therapies is is currently being conducted.

Fluvoxamine maleate is a selective serotonin (5-HT) reuptake inhibitor (SSRI) belonging to the distinct chemical series, the 2-aminoethyl oxime ethers of aralkylketones.

Fluvoxamine maleate does not appear to be chemically related to other SSRIs and clomipramine. It is chemically designated as 5-methoxy-4′-(trifluoromethyl)valerophenone-(E)-O-(2-aminoethyl)oxime maleate (1:1) and has the empirical formula C₁₅H₂₁O₂N₂F₃·C₄H₄O₄. Its molecular weight is 434.41.

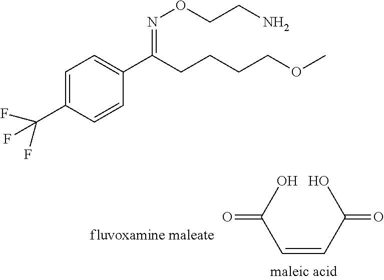 [SMILES: COCCCC/C(=N\OCCN)c1ccc(C(F)(F)F)cc1 ; O=C(O)/C=C\C(=O)O]

Fluvoxamine maleate is a white to off white, odorless, crystalline powder which is sparingly soluble in water, freely soluble in ethanol and chloroform, and practically insoluble in diethyl ether.

Fluvoxamine can be used for the outpatient treatment of persons with positive test results of SARS-CoV-2 viral testing to prevent progression to severe COVID-19 and/or hospitalization.

Fluvoxamine Maleate tablets are currently available in 25 mg, 50 mg and 100 mg strengths for oral administration. In addition to the active ingredient, fluvoxamine maleate, each tablet contains the following inactive ingredients: carnauba wax, hydroxypropyl methylcellulose, mannitol, polyethylene glycol, polysorbate 80, pregelatinized starch (potato), silicon dioxide, sodium stearyl fumarate, starch (corn), and titanium dioxide. The 50 mg and 100 mg tablets also contain synthetic iron oxides.

Exemplary dosage regimen can include Fluvoxamine 50 mg first dose; then 100 mg twice daily for 10-15 days through a recommended duration of 15 days of illness. If intolerance is a problem, dose can be reduced to 50 mg twice daily.

Fluvoxamine Maleate is intended to be taken orally as this is the current route of administration of the approved generic product.

There is a need for available oral medicines which are simple and effective early during symptomatic COVID-19 illness to prevent clinical progression and deterioration to severe disease requiring hospitalization.

First, while oral direct antivirals are presently pending EUA review and rollout, these medicines will have a very limited availability for the foreseeable future and have efficacy only when initiated within 5 days of symptom onset. Persons who do not qualify as high risk per EUA criteria, lack any therapeutic option for COVID-19. As such, these non-EUA persons seek out alternative therapies with little supportive data on benefit (e.g., ivermectin).

Second, there are no oral therapies for those presenting at >5 days of symptoms.

Third, monoclonal antibody therapies are potentially variant dependent with several having loss of activity with the Omicron variant. These monoclonal antibodies have logistical hurdles, such that they do not reach all eligible.

Fourth, FDA serves as an international authority from which other countries take their lead. In low and middle income countries, there is not access to monoclonal antibodies or direct oral antivirals, effective therapies are needed and prioritized.

As a repurposed medicine, there has been a minimal increase in U.S. prescriptions for fluvoxamine, with an increase of ˜6,000 prescriptions per week, or ˜850 daily, over 2020 baseline. Compared to the >100,000 new COVID-19 diagnoses daily, only 850 prescriptions daily reflects a missed opportunity to prevent hospitalization. As a current FDA approved medicine, fluvoxamine can be prescribed by any US healthcare provider, but fluvoxamine is rarely being prescribed. Further, we have heard of many cases where a physician has prescribed fluvoxamine, but the pharmacy has refused to fill it due to the absence of an EUA. We believe that FDA issuing EUA for this new indication will reduce these barriers to this therapy for U.S. physicians AND physicians worldwide.

Fluvoxamine has been approved in the US since 1994, and there is an extensive safety track record.

The dose and route are the same as the approved product. The 10-15 day duration is shorter than typical SSRI use. The intended population is adults >24 years of age, similar to current use. The toxicity risk is either the same or reduced because of the brief duration of treatment.

Fluvoxamine has a well-known safety profile with nausea as the most common adverse event in a global database of 35,368 people who had taken fluvoxamine across 66 studies in 11 countries (15.7% of patients), followed by somnolence (6.4%), asthenia (5.1%), headache (4.8%), and dry mouth (4.8%). Serious adverse events occurred in approximately 2.0% of people who were taking this medication for a psychiatric disorder, with 1.6% requiring hospitalization and <0.4% experiencing another serious adverse event, including a suicide attempt, depression, death, or accidental injury. As the FDA blackbox warning notes, the risk of suicidality (suicidal thoughts or behaviors) is not increased by SSRIs in adults aged ≥24 years old. Further, there is no evidence that SSRIs (including fluvoxamine) have any risk for suicidality in non-psychiatric patients.

The risk of serious adverse events in patients receiving fluvoxamine was not greater than those not receiving fluvoxamine (Relative Risk=0.81; 95% CI: 0.59, 1.12) in the two randomized clinical trials.

TABLE 20
Adverse Events in the StopCovid Trial.

| | FLUVOXAMINE | | PLACEBO | |
|---|---|---|---|---|
| EVENT | (N = 80) | | (N = 72) | |
| Pneumonia | 3 | (3.8%) | 6 | (8.3%) |
| Shortness of breath | 2 | (2.5%) | 4 | (5.6%) |
| Headache or head pain | 2 | (2.5%) | 1 | (1.4%) |
| Gastroenteritis, nausea, or vomiting | 1 | (1.3%) | 5 | (6.9%) |
| muscle aches | 1 | (1.3%) | 0 | (0%) |
| Bacterial infection | 1 | (1.3%) | 0 | (0%) |
| Vasovagal syncope | 1 | (1.3%) | 0 | (0%) |
| Teeth chattering | 1 | (1.3%) | 0 | (0%) |
| Dehydration | 1 | (1.3%) | 0 | (0%) |
| Low oxygen saturation or hypoxia | 0 | (0%) | 6 | (8.3%) |
| Chest pain or tightness | 0 | (0%) | 2 | (2.8%) |
| Fever | 0 | (0%) | 1 | (1.4%) |
| Acute on chronic respiratory failure | 0 | (0%) | 1 | (1.4%) |
| hypercapnia | 0 | (0%) | 1 | (1.4%) |
| Flank pain | 0 | (0%) | 1 | (1.4%) |
| Number with any adverse event | 12 | (15%) | 10 | (13.9%) |
| Number of patients with any serious | 1 | (1.3%) | 5 | (6.9%) |

adverse event

TABLE 21
Adverse Events in the Together Trial by AE Grade.

| AE Grade | Fluvoxamine | | Placebo | | Odds Ratio (95% CI) | | P-value |
|---|---|---|---|---|---|---|---|
| Grade 1 | 20/741 | (3%) | 11/756 | (1%) | 1.88 | (0.91-4.09) | 0.096 |
| Grade 2 | 72/741 | (10%) | 81/756 | (11%) | 0.91 | (0.64-1.25) | 0.52 |
| Grade 3 | 38/741 | (5%) | 50/756 | (7%) | 0.76 | (0.49-1.18) | 0.22 |
| Grade 4 | 21/741 | (3%) | 20/756 | (3%) | 1.07 | (0.58-2.01) | 0.82 |
| Grade 5 | 18/741 | (2%) | 26/756 | (3%) | 0.70 | (0.37-1.28) | 0.25 |

TABLE 22
Adverse Events in the Together Trial
by Body System Classification.

| Adverse event | Fluvoxamine | | Placebo | |
|---|---|---|---|---|
| (by MedDRA | N = 741 | | N = 756 | |
| body system) | Participants | | Participants | |
| N individual AEs | 174 | (100%) | 193 | (100%) |
| Cardiac disorders | 1 | (0.6%) | 3 | (1.6%) |
| Ear and labyrinth disorders | 1 | (0.6%) | 1 | (0.5%) |
| Gastrointestinal disorders | 16 | (9.2%) | 6 | (3.1%) |
| General disorders and | 37 | (21%) | 40 | (21%) |
| administration site conditions | | | | |
| Hepatobiliary disorders | 0 | (0%) | 1 | (0.5%) |
| Infections and infestations | 85 | (49%) | 108 | (56%) |
| Metabolism and nutrition | 4 | (2.3%) | 3 | (1.6%) |
| disorders | | | | |
| Musculoskeletal and connective | 4 | (2.3%) | 5 | (2.6%) |
| tissue disorders | | | | |
| Nervous system disorders | 3 | (1.7%) | 5 | (2.6%) |
| Psychiatric disorders | 5 | (2.9%) | 0 | (0%) |
| Renal and urinary disorders | 1 | (0.6%) | 1 | (0.5%) |
| Reproductive system and breast | 1 | (0.6%) | 0 | (0%) |
| disorders | | | | |
| Respiratory, thoracic and | 11 | (6.3%) | 17 | (8.8%) |
| mediastinal disorders | | | | |
| Skin and subcutaneous tissue | 0 | (0%) | 1 | (0.5%) |
| disorders | | | | |
| Vascular disorders | 2 | (1.1%) | 2 | (1.0%) |

Legend: This table represents all recorded AE. Individual participants may have ≥1 AE episode.

Evidence is steadily accumulating that points to the role of excessive immune response in SARS-CoV-2 infection as a key factor in clinical deterioration or long-term symptoms. A leading theory is that SARS-CoV-2 replicates in an intermediate compartment between the endoplasmic reticulum and Golgi complex, leading to endoplasmic reticulum stress and potentially to cytokine storm due to excessive inflammatory response. The sigma-1 receptor is already well-established as influencing the pathophysiology of multiple psychiatric, neurodegenerative, and central nervous system disorders. The findings of Gordon and colleagues, along with prior research indicating a potential role for Sigma-1 receptor agonists or antagonists in preventing sepsis—also caused by inflammatory cytokine production—has led to an interest in exploring the potential for repurposing of existing drugs that target the sigma-1 receptor as early treatment for SARS-CoV-2 infection.

Fluvoxamine is a selective serotonin reuptake inhibitor (SSRI) that has been widely used globally since the 1990s. Previous studies have shown high affinity for the sigma-1 receptor, and in multiple comparative studies fluvoxamine has been consistently ranked as one of the strongest sigma-1 receptor agonists of existing medicines on the market, therefore, fluvoxamine may effectively reduce cytokine production and prevent clinical deterioration.

Fluvoxamine is inexpensive, easy to use, widely available globally, and highly lipophilic, with rapid intracellular uptake into lung epithelial cells. Sigma-1 receptor agonism as a pathway to preventing the cytokine storm: A 2019 study showed that SSRI fluvoxamine reduces damaging aspects of the inflammatory response during sepsis, and protects mice from lethal septic shock. Fluvoxamine binds to the sigma-1 receptor, which regulates inflammation by inhibiting cytokine production. Fluvoxamine may also induce the sigma-1 receptor.

The sigma-1 receptor restricts the endonuclease activity of an endoplasmic reticulum stress sensor called Inositol—Requiring Enzyme1 and reduces cytokine expression, without inhibiting classical inflammatory pathways.

In a 2019 study by Rosen and colleagues, fluvoxamine showed benefit in preclinical models of inflammation and sepsis. In one model, mice were exposed to the Toll-like receptor 4 (TLR4) ligand lipopolysaccharide (LPS), which can trigger an inflammatory response. In another model, a fecal slurry was injected, which triggers a usually sub-lethal infection and an inflammatory response.

Mice lacking the sigma-1 receptor showed excessive increases in cytokine levels and greatly reduced survival under either of these conditions, suggesting the sigma-1 receptor inhibits excessive inflammatory responses. Wildtype mice exposed to the same inflammatory triggers showed reduced cytokine levels and increased survival when treated with fluvoxamine (a sigma-1 receptor agonist). When investigating the underlying mechanism of this effect, the authors demonstrated that sigma-1 receptor inhibits activity of IRE1, which in turn prevents the excessive cytokine production.

In an experiment using human peripheral blood, they also showed that fluvoxamine can reduce LPS-induced cytokine production by human cells. In the case of COVID-19, fluvoxamine's sigma-1 receptor agonist action may have a similar ability to reduce the excessive inflammatory response induced by the viral infection, thus reducing inflammation-mediated organ damage.

In two randomized clinical trials, different doses of fluvoxamine were utilized (see e.g., Example 2).

Participants received an initial dose of 50 mg fluvoxamine (or matching placebo) in the evening, and then two days at 100 mg twice daily as tolerated, then increasing to 100 mg three times daily as tolerated for 15 days in total therapy. Overall, only half of participants reached 100 mg three times daily dosing.

TABLE 23
STOP COVID trial dosing (Reis et al. Lancet
Global Health Together Trial; Example 2)

| | |
|---|---|
| Maximum Dose Tolerated over 15 days | Number (%) |

| | | | |
|---|---|---|---|
| 200 | mg/day (100 twice daily) | 36 | (45%) |
| 300 | mg/day (100 three times daily) | 40 | (50%) |

| | | |
|---|---|---|
| Not able to reach 200 mg/day | 4 | (5%) |

The TOGETHER trial is the largest placebo-controlled trial of COVID-19 interventions in the world. It currently evaluates drugs for pre-hospitalization. Overall, 741 participants were randomized to receive fluvoxamine 100 mg twice daily for 10 days. In this trial, 74% (548/741) of participants took >80% of study medicine, and 82% (618/756) took >80% of blinded placebo, a difference of 8% for adherence.

### Dosing Summary

First, as tolerability of SSRIs can thereby be an issue, when starting at moderate doses, we propose 50 mg first dose followed by 100 mg twice daily, more similar to the initially dosing present in the Lenze et al trial. This should improve tolerability.

Second, for duration of therapy, the trials studied between 10-15 days of therapy. Both trials investigated fluvoxamine in persons with 57 days of symptoms. As persons may be prescribed fluvoxamine with a varied duration of symptoms, we recommend a prescribing duration of 10-15 days through at least 15 days of illness. This covers the peak time for clinical decompensation balancing benefit with risk of medication side effects.

### Well Organized Study Reports.

Two published peer-reviewed manuscripts of double-blind, placebo controlled randomized clinical trials were performed (see Examples 2, 3, and 7).

(1) Reis G, Dos Santos Moreira-Silva E A, Silva D C M, et al. Effect of early treatment with fluvoxamine on risk of emergency care and hospitalization among patients with COVID-19: the TOGETHER randomized, platform clinical trial. Lancet Glob Health. 2022; 10 (1): e42-51. Published online Oct. 27, 2021. Available at: https://doi.org/10.1016/S2214-109X(21)00448-4. Published Protocol and Statistical Analysis Plan are available at: Reis G, Silva E AdSM, Silva D C M et al. A multi-center, adaptive, randomized, platform trial to evaluate the effect of repurposed medicines in outpatients with early coronavirus disease 2019 (COVID-19) and high-risk for complications: the TOGETHER master trial protocol. Gates Open Res 2021, 5:117. Available at: https://doi.org/10.12688/gatesopenres.13304.2

(2) Lenze E J, Mattar C, Zorumski C F, et al. Fluvoxamine vs placebo and clinical deterioration in outpatients with symptomatic COVID-19: a randomized clinical trial. JAMA. 2020; 324(22): 2292-2300. Available at: http://dx.doi.org/10.1001/jama.2020.22760

(3) Supportive real world evidence via a prospective cohort study, is incorporated herein by reference (Seftel D, Boulware D R. Prospective Cohort of Fluvoxamine for Early Treatment of Coronavirus Disease 19 Open Forum Infect Dis. 2021 Feb. 1; 8(2): ofab050).

TABLE 24
Summary of the two double-blind, placebo-controlled randomized clinical trials.

| | | |
|---|---|---|
| [symbol] | [symbol] | [symbol] |

[symbol]

Adaptive RCT of [symbol] in Non-hospitalized Patients [symbol] COVID-19 in Brazil

[symbol]

| | | |
|---|---|---|
| Key Inclusion Criteria: | Participant Characteristics: | Key Limitations: |
| [symbol] | [symbol] | [symbol] |
| [symbol] | [symbol] | [symbol] |
| [symbol] days of symptoms | [symbol] | [symbol] |
| Key Exclusion Criteria: | Primary Outcome: | [symbol] |
| [symbol] | [symbol] | Interpretation: |
| [symbol] | Secondary Outcomes: | [symbol] |
| [symbol] | [symbol] | [symbol] |
| Interventions: | [symbol] | |
| [symbol] | [symbol] | |
| [symbol] | [symbol] | |
| Primary Endpoint: | Comparative Outcomes used in other trials: | |
| [symbol] | [symbol] | |
| [symbol] | | |
| Key Secondary Endpoints: | | |
| [symbol] | | |
| [symbol] | | |
| [symbol] | | |

[symbol]

RCT of [symbol] in Non-hospitalized Patients [symbol] COVID-19 in the United States

[symbol]

| | | |
|---|---|---|
| Key Inclusion Criteria: | Participant Characteristics: | Key Limitations: |
| [symbol] | [symbol] | [symbol] |
| [symbol] | [symbol] | [symbol] |
| [symbol] | [symbol] | [symbol] |
| Key Exclusion Criteria: | Primary Outcome: | Interpretation: |
| [symbol] | [symbol] | [symbol] |
| [symbol] | Secondary Outcome: | |
| Interventions: | [symbol] | |
| [symbol] | | |
| [symbol] | | |
| Primary Endpoint: | | |
| [symbol] | | |
| [symbol] | | |
| [symbol] | | |
| Key Secondary Endpoint: | | |
| [symbol] | | |

[symbol]

indicates data missing or illegible when filed

### Discussion of Risks and Benefits

Benefits: With fluvoxamine 100 mg twice daily when started within the first 7 days there is a statistically significant 25% reduction in hospitalizations and a 36% reduction in progression to FDA-categorized severe disease with hypoxia and dyspnea and/or hospitalization by intent to treat analysis.

In pooling outcomes from the two randomized clinical trials. Fluvoxamine reduced COVID-related hospitalizations by 25% compared to double-blind placebo (RR=0.75; 95% CI: 0.57, 0.99).

Fluvoxamine showed a reduction of the composite outcome of hospitalizations, emergency room observation lasting >6 hours, and/or oxygen saturation <92% (RR=0.64; 0.50, 0.84), i.e. progression to FDA-classification of severe disease. Persons necessitating ER room observation for >6 hours had hypoxia <93%. The number needed to treat is approximately 20.

TABLE 25
Together Trial Primary Outcomes: Proportion of primary outcome events
and relative risk of hospitalization defined as either retention in
a COVID-19 emergency setting for >6 hours or transfer to tertiary
hospital due to COVID-19 for patients allocated fluvoxamine versus placebo.

| | | | | Relative Risk |
|---|---|---|---|---|
| Analysis Type | Definition | Fluvoxamine | Placebo | (95% CI) |
| Intent to Treat | All randomized | 10.7% | 15.7% | 0.68 (0.52-0.88) |
| Analysis | | (79/741) | (119/756) | |
| Modified Intent to | Received ≥24 hours | 10.5% | 15.3% | 0.69 (0.53-0.90) |
| Treat Analysis | of study medicine | (78/740) | (115/752) | |
| Per Protocol | Received 80% of | 3.6% | 11.0% | 0.34 (0.21-0.54) |
| | study medicine prior | (20/548) | (68/618) | |
| | to any deterioration | | | |

Persons observed for >6 hours in ER generally presented with hypoxia and dyspnea, necessitating interventional care consistent with severe COVID-19 (e.g. dexamethasone, supplemental oxygen, etc.). For a standardized definition per the FDA categorization of progression to severe COVID (e.g. hypoxia <93% with dyspnea and/or requiring hospitalization) as used in vaccine trials, fluvoxamine had a relative risk=0.74 (95% CI, 0.54 to 0.95; P=0.02) by intent to treat analysis with 10.1% (75/741) in fluvoxamine vs. 14.3% (108/756) with placebo.

Per Protocol assesses for effect in those who took ≥80% of study medicine prior to any deterioration. While Per protocol analyses routinely overestimate the effectiveness of any intervention as tolerability is an important component, this does suggest the medicine has a benefit, if it is tolerable. As such we propose to utilize a 50 mg as the first dose before going to 100 mg twice daily in order to possibly improve tolerability. With SSRIs, the majority of side effects are annoying, not life threatening, and generally dissipate with further dosing.

Optimal dose and duration have yet been unoptimized. Based on the timing of potential decompensation, continuation through ˜15 days of symptom duration or symptom resolution (if earlier) is reasonable.

The University of Minnesota Covid-Out trial (IND 152439) and NIH ACTIV-6 trials (IND 155481) are testing low dose of 50 mg twice daily. The 50 mg dose will have better tolerability. Whether the 50 mg twice daily dose will have similar effect (or better effect due to better tolerability) is to be determined.

The ACTIV-6 trial does not have an estimate time for completion of the fluvoxamine arm at this time with n=202 randomized to the fluvoxamine arm or concurrent controls as of 21 Dec. 2021 with a target of n=800 to receive fluvoxamine (plus the concurrent controls).

The utility of combination therapy with oral direct antiviral medicines (e.g. molnupiravir, nirmatrelvir) is unknown (although it is expected to be additive because of different mechanisms of action). The utility of fluvoxamine for treatment of COVID-19 in patients receiving glucocorticoids is unknown, although the TOGETHER trial is now evaluating the combination of fluvoxamine plus inhaled budesonide is now in progress.

### Contraindications and Potential Drug Interactions

Co-administration of tizanidine, thioridazine, alosetron, or pimozide with fluvoxamine is contraindicated. Caution with diazepam, clozapine, methadone, antipsychotics, ramelteon, carbamazepine, sumatriptan, tacrine, and serotonergic drugs. Monitor mexiletine and tricyclic antidepressant levels if either co-administered Monitor PT/INR if warfarin co-administered. Reduce dose of theophylline, propranolol or metoprolol if co-administered. Avoid or minimize caffeine and melatonin as metabolism is inhibited.

### REFERENCES

1. Wagner W, Zaborny B A, Gray T E. Fluvoxamine. A review of its safety profile in world-wide studies. Int Clin Psychopharmacol. 1994; 9:223-7.
2. Altamura A C, Caldiroli A, Buoli M. Pharmacokinetic evaluation of fluvoxamine for the treatment of anxiety disorders. Expert Opin Drug Metab Toxicol. 2015; 11:649-60.
3. Lenze E J, Mattar C, Zorumski C F, Stevens A, Schweiger J, Nicol G E, Miller J P, Yang L, Yingling M, Avidan M S, Reiersen A M. Fluvoxamine vs placebo and clinical deterioration in outpatients with symptomatic COVID-19: A randomized clinical trial. JAMA. 2020; 324:2292-300. PMCID: 7662481.
4. Reis G, Dos Santos Moreira-Silva E A, Silva D C M, Thabane L, Milagres A C, Ferreira T S, Dos Santos C V Q, de Souza Campos V H, Nogueira A M R, de Almeida A, Callegari E D, de Figueiredo Neto A D, Savassi L C M, Simplicio M I C, Ribeiro L B, Oliveira R, Harari O, Forrest J I, Ruton H, Sprague S, McKay P, Glushchenko A V, Rayner C R, Lenze E J, Reiersen A M, Guyatt G H, Mills E J. Effect of early treatment with fluvoxamine on risk of emergency care and hospitalization among patients with COVID-19: The together randomized, platform clinical trial. Lancet Glob Health. 2022; 10:e42-e51. PMCID: 8550952.
5. Hashimoto K. Repurposing of cns drugs to treat COVID-19 infection: Targeting the sigma-1 receptor. EurArch Psychiatry Clin Neurosci. 2021; 271:249-58. PMCID: 7785036.
6. Albayrak Y, Hashimoto K. Sigma-1 receptor agonists and their clinical implications in neuropsychiatric disorders. Adv Exp Med Biol. 2017; 964:153-61.
7. Hashimoto K. Activation of sigma-1 receptor chaperone in the treatment of neuropsychiatric diseases and its clinical implication. J Pharmacol Sci. 2015; 127:6-9.
8. Hashimoto K. Targeting the sigma-1 receptor chaperone in the treatment of perinatal brain injury. Exp Neurol. 2015; 265:118-21.
9. Rosen D A, Seki S M, Fernandez-Castaneda A, Beiter R M, Eccles J D, Woodfolk J A, Gaultier A. Modulation of the sigma-1 receptor-irel pathway is beneficial in preclinical models of inflammation and sepsis. Sci Transl Med. 2019; 11:eaau5266. PMCID: 6936250.
10. Cobos E J, Entrena J M, Nieto F R, Cendan C M, Del Pozo E. Pharmacology and therapeutic potential of sigma(1) receptor ligands. Curr Neuropharmacol. 2008; 6:344-66. PMCID: 2701284.
11. Hallifax D, Houston J B. Saturable uptake of lipophilic amine drugs into isolated hepatocytes: Mechanisms and consequences for quantitative clearance prediction. Drug Metab Dispos. 2007; 35:1325-32.
12. Omi T, Tanimukai H, Kanayama D, Sakagami Y, Tagami S, Okochi M, Morihara T, Sato M, Yanagida K, Kitasyoji A, Hara H, Imaizumi K, Maurice T, Chevallier N, Marchal S, et al. Fluvoxamine eleviates er stress via induction of sigma-1 receptor. Dis. 2014; 5:e1332. PMCID: 4123092.

### Example 9: Fluvoxamine: A Review of its Mechanism of Action and its Role in COVID-19

Fluvoxamine is a well-tolerated, widely available, inexpensive selective serotonin reuptake inhibitor that has been shown in a small, double-blind, placebo-controlled, randomized study to prevent clinical deterioration of patients with mild coronavirus disease 2019 (COVID-19). Fluvoxamine is also an agonist for the sigma-1 receptor, through which it controls inflammation. We review here a body of literature that shows important mechanisms of action of fluvoxamine and other SSRIs that could play a role in COVID-19 treatment. These effects include: reduction in platelet aggregation, decreased mast cell degranulation, interference with endolysosomal viral trafficking, regulation of inositol-requiring enzyme 1α-driven inflammation, and increased melatonin levels, which collectively have a direct antiviral effect, regulate coagulopathy or mitigate cytokine storm, which are known hallmarks of severe COVID-19.

### Introduction

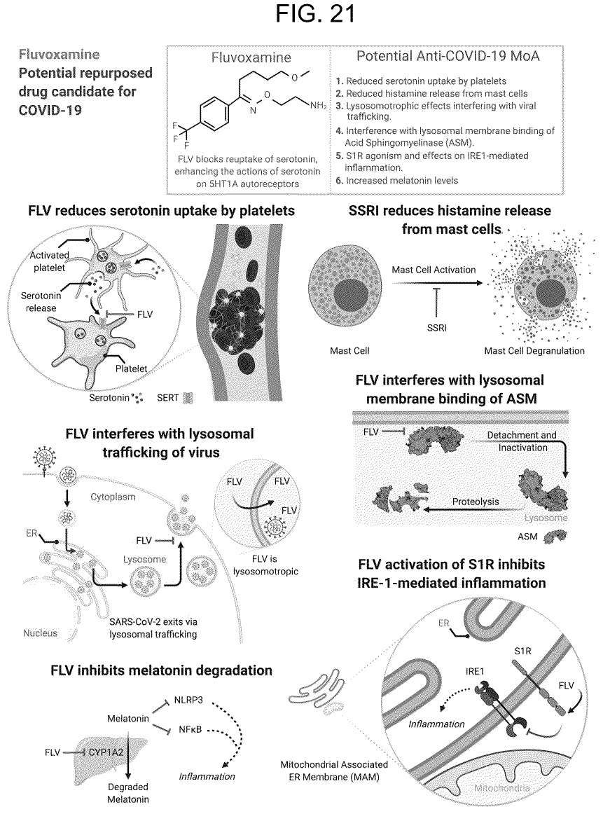

Initially used to treat obsessive-compulsive disorder (OCD), fluvoxamine (FLV) has been shown to have the strongest activity of all SSRIs at the sigma-1 receptor (S1R) with low-nanomolar affinity (Narita et al., 1996). FLV agonism on S1R potentiates nerve-growth factor (NGF)-induced neurite outgrowth in PC 12 cells (Nishimura et al., 2008; Ishima et al., 2014). S1R is a chaperone protein at the endoplasmic reticulum with anti-inflammatory properties (Ghareghani et al., 2017). FLV's anti-inflammatory effects likely stem from its regulation of S1R, which modulates innate and adaptive immune responses (Szabo et al., 2014). S1R is also an important regulator of inositol-requiring enzyme 1α (IRE1)-driven inflammation (Rosen et al., 2019) (FIG. 21).

FLV and other SSRIs regulate inflammatory cytokine activity and gene expression in both cell and animal models of inflammation (Taler et al., 2007; Tynan et al., 2012; Rafiee et al., 2016; Ghareghani et al., 2017; Naji Esfahani et al., 2019; Rosen et al., 2019). The potential of FLV to dampen cytokine storm has implications in COVID-19. COVID-19 severity is associated with an increased level of inflammatory mediators including cytokines and chemokines (Chen G. et al., 2020; Chen N. et al., 2020; Huang et al., 2020; Tay et al., 2020). Other S1R agonists like fluoxetine have been reported to have antiviral activity (Zuo et al., 2012; Bauer et al., 2019). These studies have raised interest in the potential therapeutic role of FLV and S1R agonists in COVID-19 (Vela, 2020; Hashimoto, 2021).

This review illustrates mechanisms of action underlying anti-inflammatory and antiviral properties of FLV. It covers preclinical studies on effects of FLV and S1R agonists on inflammation, and summarizes currently available clinical data for FLV treatment in COVID-19.

### Indications for Fluvoxamine

Fluvoxamine maleate is available as immediate release tablets and controlled-release capsules. FLV is indicated to treat obsessions and compulsions in patients with OCD. The half-life of FLV is 9-28 h depending on its formulation, and the recommended dosage is 100-300 mg/day (FDA, 2012).

### Original Mechanism of Action

### Serotonin Transporter Inhibition

FLV blocks reuptake of serotonin at the sodium-dependent serotonin transporter (SERT) of the neuronal membrane, enhancing actions of serotonin on 5HT1A autoreceptors (Dell'Osso et al., 2005; FDA, 2012). FLV has negligible affinity for α1-, α2-, β-adrenergic, muscarinic, dopamine D2, histamine H1, GABA-benzodiazepine, opiate, 5-HT1, or 5-HT2 receptors (Irons, 2005).

### Likely Mechanisms of Action in COVID-19

### Platelet Aggregation

Platelets lack the enzyme to synthesize serotonin (Ni and Watts, 2006). A SERT enables rapid uptake of serotonin from plasma (Vanhoutte, 1991). During thrombosis platelets release serotonin, facilitating hemostasis through platelet aggregation (Berger et al., 2009) (FIG. 21), and promotes recruitment of neutrophils (Duerschmied et al., 2013). SSRIs can therefore increase bleeding time (Leung and Shore, 1996) or reduce serum serotonin by >80% and reduce neutrophil recruitment (Duerschmied et al., 2013). Platelets from individuals treated with SSRIs, and platelets from SERT knockout mice, show decreased aggregation (Celada et al., 1992; Carneiro et al., 2008; McCloskey et al., 2008). Measures of coagulation and hemostasis were lower in patients with serotonergic antidepressant than in patients without serotonergic antidepressant (Geiser et al., 2011). A hyperserotonergic state distinguishes COVID-19 and non-COVID-19 acute respiratory distress syndrome, biochemically (Zaid et al., 2021) and clinically (Helms et al., 2020a; Helms et al., 2020b). This is likely pathologic across a multitude of organs (akin to serotonin syndrome, F. Jalali—personal observation and communication) and may originate from an immune-mediated (Althaus et al., 2020; Nazy et al., 2021) state of platelet hyperreactivity (Zaid et al., 2021), resulting in florid platelet degranulation of serotonin into plasma.

A concomitant impairment of serotonin reuptake may exacerbate this hyperserotonergic state. Serotonin clearance relies on a healthy pulmonary endothelium (Thomas and Vane, 1967; Joseph et al., 2013), that is injured in COVID-19 (Ackermann et al., 2020). Platelet serotonin liberation can be reduced with chronic or early de novo SSRI use (Cloutier et al., 2018), since SSRIs deplete serotonin content of platelets (Narayan et al., 1998; Javors et al., 2000). Initiation of de novo SSRIs at laterstages of moderate to severe COVID-19, however, may be unpredictably harmful given the existing hyperserotonergic state (Zaid et al., 2021) unless counterbalanced by other beneficial effects of SSRIs. Indeed, direct serotonin antagonism specifically targeting the serotonin 2 A, B and C receptors with drugs such as cyproheptadine or mirtazapine in this stage may be beneficial and is being explored (F. Jalali—personal communication).

Three trials assessing benefit of anticoagulants to treat COVID-19 have paused enrollment of critically ill COVID-19 patients who require intensive care unit (ICU) support (NHLBI, 2020). Therapeutic blood thinners did not reduce need for ICU admission in this patient-group. Since full doses of therapeutic anticoagulants increase risk of internal bleeding, FLV could perhaps inhibit blood clotting more safely.

### Mast Cell Degranulation

Human mast cells (MCs) are a viral reservoir for RNA viruses like HIV (Sundstrom et al., 2004). Retinoic acid-inducible gene-I-like receptors of mast cells can detect RNA viruses (Fukuda et al., 2013). Viruses can cause degranulation of MCs in a Sphingosine-1-Phosphate (S1P)-dependent pathway (Wang et al., 2012). MCs express angiotensin converting enzyme 2 (ACE2), the principal receptor for SARS-CoV-2 entry into cells, thus defining a route by which MCs could become hosts for this virus (Theoharides, 2020). Post-mortem lung biopsies of COVID-19 patients have linked pulmonary edema and thromboses to activated MCs (Motta Junior et al., 2020). Antidepressants also decrease histamine release from MCs (Ferjan and Erjavec, 1996). SSRIs like fluoxetine decreased mRNA levels of protease-1 in MCs (Chen et al., 2008). Therefore, SSRIs like FLV could reduce cytokine storms in COVID-19 patients (FIG. 21) because of atypical response of MCs to SARS-CoV-2.

### Lysosomotropism

S1R agonists like FLV and fluoxetine are lysosomotropic (Hallifax and Houston, 2007; Kazmi et al., 2013). Fluvoxamine has a predicted pKa of 8.86 (DrugBank, 2005; Wishart et al., 2018) and is susceptible to protonation in the physiological pH range. Less polar, unionized form of basic drugs can easily cross membranes. Basic drugs like FLV can get protonated in the lysosome, which hinders the now-charged moieties from crossing membranes. β-coronaviruses, like SARS-CoV-2 and mouse hepatitis virus (MHV), use lysosomal trafficking to escape from infected cells (Ghosh et al., 2020) (FIG. 21). GRP78/BIP, a chaperone that facilitates coronavirus infectivity (Chu et al., 2018; Ha et al., 2020), is co-released with β-coronaviruses through this pathway (Ghosh et al., 2020). The SARS-CoV open reading frame protein 3A (ORF3a) (Gordon et al., 2020) is a viroporin that localizes to lysosomes (Ghosh et al., 2020), disrupts their acidification (Yue et al., 2018), and contributes to viral egress (Lu et al., 2006; Castano-Rodriguez et al., 2018; Yue et al., 2018). Given the lysosomal egress of β-coronaviruses from infected cells, lysosomotropic drugs like FLV could have antiviral effects in the virus laden lysosomes (Homolak and Kodvanj, 2020) (FIG. 21).

### Acid Sphingomyelinase

Lysosomotropic drugs displace acid sphingomyelinase (ASM) from lysosomal membranes leading to its degradation (Breiden and Sandhoff, 2019) (FIG. 21). Treatment of mice with S1R agonists like fluoxetine (Hashimoto, 2015) reduces both acid sphingomyelinase activity and protein levels in neurons (Gulbins et al., 2013). This is consistent with partial proteolysis of acid sphingomyelinase by fluoxetine (Kornhuber et al., 2008). Fluoxetine can efficiently inhibit entry and propagation of SARS-CoV-2 in Vero-E6 cell lines (Schloer et al., 2020). It also exerts antiviral activity against influenza A virus subtypes (Schloer et al., 2020). S1R agonists like escitalopram and fluoxetine (Hashimoto, 2015) can prevent infection of Vero cells with vesicular stomatitis virus pseudoviral particles presenting SARS-CoV-2 spike protein (pp-VSV-SARS-CoV-2 spike) (Carpinteiro et al., 2020). Antidepressants like amitriptyline also prevented infection of human Caco-2 cells with SARS-CoV-2 and treating volunteers with a low dose of amitriptyline prevented infection of freshly isolated nasal epithelial cells with pp-VSV-SARS-CoV-2 spike (Carpinteiro et al., 2020). Inhibition of acid sphingomyelase by these drugs can prevent the conversion of sphingomyelin to phosphorylcholine and ceramide. Because high ceramide in the cell membrane facilitates viral entry, this reduction in ceramide may prevent infection (Carpenteiro et al., 2020). Therefore, functional inhibition of acid sphingomyelinase by lysosomotropic drugs is another avenue of viral control by antidepressants.

### Sigma-1 Receptor Activity

S1R was discovered in 1976 (Martin et al., 1976) and cloned in 1996 (Hanner et al., 1996). It regulates ER-mitochondrial Ca²¹ signaling and cell survival (Hayashi and Su 2007). Targeting S1R with FLV regulates cytokine production in human monocyte-derived dendritic cells (Szabo et al., 2014). S1R knockout (KO) bone marrow-derived macrophages (BMDMs) were proinflammatory in endotoxic shock models. They had higher levels of IL-6 and IL-1β mRNA and increased IL-6 protein secretion compared to wild-type (WT) BMDMs (Rosen et al., 2019). In contrast, anti-inflammatory cytokine IL-10 expression was unaffected in S1R KO BMDMs (Rosen et al., 2019). S1R overexpression in HEKs expressing mTLR4/MD2/CD14 was anti-inflammatory in an endotoxic shock model. Compared to HEKs with normal levels of S1R, cells with higher levels of S1R had lower IL-8 levels on LPS stimulation (p<0.05). In other systems, FLV upregulates IL-10 (Kalkman and Feuerbach, 2016; Nazimek et al., 2017). FLV via the S1R may therefore modulate SARS-CoV-2-induced hyperinflammatory state (FIG. 21).

On the flip side, genetic perturbation screens have shown depletion of S1R, decreases SARS-CoV-2 viral replication in adenocarcinoma human alveolar basal epithelial cell lines expressing Angiotensin I Converting Enzyme 2 (A549-ACE2) (Gordon et al., 2020). Consistent with this genetic data, S1R agonists such as dextromethorphan can increase viral replication (Gordon et al., 2020). However, in contrast, researchers reviewing medical billing data for nearly 740,000 COVID-19 patients in the US showed patients on antipsychotic drugs targeting S1R were half as likely as those on other types of antipsychotic drugs to require mechanical ventilation (Gordon et al., 2020).

Neurotropism is one common feature for human coronaviruses (Bale, 2015; Dube et al., 2018). Various receptors could be involved in neurotropism and neuronal cell entry of SARS-CoV-2 (Armocida et al., 2020). Sigma receptors are widely expressed in the CNS (Yesilkaya et al., 2020). Downregulation of S1R protein expression impairs initiation of hepatitis C virus (HCV) RNA replication in human hepatoma cells (Friesland et al., 2013). BD1047 a selective S1R antagonist blocked cocaine-mediated stimulation of human immune deficiency virus (HIV-1) expression in neuronal mononuclear phagocytes like microglia (Gekker et al., 2006). S1R could therefore be involved in neuronal transmission of other RNA viruses like SARS-CoV-2.

### Inositol-Requiring Enzyme 1α and Autophagy

Endotoxin-stimulated TLR4 activates IRE1 (Martinon et al., 2010) and regulates proinflammatory cytokine production (Qiu et al., 2013). SARS-CoV E protein down-regulates IRE1 pathway and the SARS-CoV lacking the envelope (E) gene (rSARS-CoV-AE) is attenuated in vivo (DeDiego et al., 2011). IRE1 inhibitors like STF-083010 rescued S1R KO mice in a model of endotoxemia (Rosen et al., 2019). IRE1 is essential for autophagy during infection with a gamma coronavirus-Infectious Bronchitis Virus (IBV) (Fung and Liu, 2019). SARS-CoV replicase proteins nsp2, 3 and 8 occur in cytoplasmic complexes and colocalize with LC3, a protein marker for autophagic vacuoles (Prentice et al., 2004). The viral replicase protein nsp6 of IBV activates autophagy in a screen (Cottam et al., 2011). Other studies reviewed here (Yang and Shen, 2020) suggest autophagy is not directly involved in SARS-CoV. These discrepancies are probably because of different viruses and cells tested in various studies.

### Melatonin

SARS-CoV-2 virus can activate NLRP3 inflammasome (van den Berg and Te Velde, 2020), which along with NF-κB activation can induce cytokine storm (Ratajczak and Kucia, 2020). Melatonin can mitigate inflammation through these pathways and melatonin exposure post-intubation is associated with a positive outcome in COVID-19 (and non-COVID-19) patients (Garcia et al., 2015; Ramlall et al., 2020). FLV can elevate melatonin levels via inhibition of CYP1A2, a member of the cytochrome P450 superfamily of enzymes (Hartter et al., 2001) (FIG. 21).

Could Selective Serotonin Reuptake Inhibitors and Sigma-1 Receptor Agonists have Direct Antiviral Effects on Other Viruses?

### Precedent for Using Selective Serotonin Reuptake Inhibitors to Treat Other Viral Infections

Enteroviruses are non-enveloped RNA viruses. Their nonstructural protein 2C is one of their most conserved proteins and contains ATPase activity and putative RNA helicase activity (Cheng et al., 2013). Fluoxetine has in vitro antiviral activity against Enterovirus B and D species (Zuo et al., 2012; Ulferts et al., 2013). Fluoxetine binds nonstructural protein 2C directly (Manganaro et al., 2020). Some fluoxetine resistant variants of enteroviruses like coxsackievirus B3 and B4 have mutations in protein 2C (Ulferts et al., 2013; Alidjinou et al., 2019). This reinforces the idea that interaction between fluoxetine and protein 2C is essential for its antiviral effects.

### Endoplasmic Reticulum Stress Response

Viral infection may trigger the unfolded protein response (UPR). This is an ER stress response because of ER overloading with virus-encoded proteins (Kim et al., 2008), and can also induce autophagy (Bernales et al., 2006; Ogata et al., 2006). ER signaling proteins like IRE1, PRKR-like ER kinase (PERK), and activating transcription factor 6 (ATF6) regulate UPR. The UPR is involved in viral replication and modulates host innate responses (Xue et al., 2018). Virus-induced ER stress is required for autophagy activation, viral replication, and pathogenesis in dengue (Lee et al., 2018). Murine cytomegalovirus activates the IRE1 pathway to relieve repression by X-box binding protein 1 unspliced mRNA (Hinte et al., 2020). Coronavirus infection induces ER stress and triggers UPR (Fung et al., 2016). The S protein in β-coronaviruses modulates UPR to facilitate viral replication (Chan et al., 2006; Versteeg et al., 2007). The α-coronavirus, transmissible gastroenteritis virus (TGEV) triggers UPR-induced ER stress primarily through activation of PERK-eukaryotic initiation factor 2a axis (Xue et al., 2018). Thus ER stress response is critical in host-virus interactions in a variety of infections. We have discussed above how S1R is a regulator of IRE1 and autophagy. S1R agonists like FLV could therefore have a role in regulating viral infections beyond SARS-CoV-2 through its putative regulation of ER stress and UPR.

### Preclinical Effects of Fluvoxamine on Inflammation

S1R KO mice display increased mortality compared to WT in sublethal models of sepsis (Rosen et al., 2019). Peak serum TNF and IL-6 were increased in LPS-challenged S1R KO mice. S1R ligand FLV enhanced survival in mouse models of IRE1-mediated inflammation and fecal-induced peritonitis. FLV treatment protected WT mice from endotoxic shock-induced death, while no significant effect was observed in S1R KO animals suggesting the anti-inflammatory effects of FLV are likely mediated through S1R.

Multiple sclerosis (MS) is a chronic, inflammatory, demyelinating neurodegenerative disease. SSRIs like sertraline have been shown to have immunomodulatory effects in experimental autoimmune encephalomyelitis (EAE), a mouse model of MS (Taler et al., 2011), and in a rat model of rheumatoid arthritis (Baharav et al., 2012). FLV reduces the severity in EAE in rats, even when treatment began 12 days post-induction of EAE (Ghareghani et al., 2017). FLV-treated EAE rats showed a decrease in IFN-γ serum levels and an increase in IL-4, pro- and anti-inflammatory cytokines respectively, compared to untreated EAE rats. The dose of FLV used in these experiments extrapolates (by surface area) to FLV doses approved for human use.

Thus, FLV seems to ameliorate inflammation in different in vivo inflammation models. Data in non-human primates or a hamster model of SARS-CoV-2 infection would shed further light on whether FLV might be a useful drug for COVID-19 patients and on the mechanism(s) at play.

### Clinical Effects of Fluvoxamine in COVID-19

In a double-blind, randomized, preliminary study of adult outpatients with symptomatic COVID-19, 80 patients treated with FLV, compared to 72 treated with placebo, had a lower likelihood of clinical deterioration over 15 days (Lenze et al., 2020). Eligible patients were enrolled within 7 days of symptom development. These data are provocative with none of the FLV-treated patients deteriorating vs. 8.3% patients in the control arm who showed clinical deterioration. Participants received 50 mg FLV QD on day 1, then for 2 days 100 mg FLV BID, and then 100 mg FLV TID as tolerated through day 15 and then stopped. In a prospective study on use of FLV for early treatment of COVID-19 the incidence of hospitalization was 0% (0/65) with FLV and 12.5% (6/48) with observation alone. At 14 days, 0% (0/65) of FLV treated people had persistent residual symptoms compared to 60% (29/48) among people who opted for no therapy (Seftel and Boulware, 2021). Agonists of S1R like escitalopram and fluoxetine were associated with lower risk of intubation or death (p<0.05) because of COVID-19 in a multicenter observational retrospective cohort study (Hoertel et al., 2021).

Given the multiple roles of S1R reviewed here in inflammation, platelet aggregation, antiviral activity, etc., and the recent striking human data, it is likely that S1R agonists like FLV could have a major impact on disease progression of COVID-19 patients in the early stage of the disease.

### Discussion

An 880 patient randomized study is underway and should provide some definitive answers (Lenze, 2020). Patients nationally can join this study from home and at no cost. However, given the current crisis, which is expected to worsen before a vaccine takes effect, one wonders if the FLV evidence in COVID-19 is strong enough to consider a change in practice guidelines, to even more quickly accumulate data on outcomes in COVID-19 patients (Sukhatme and Sukhatme, 2021). A small group of healthcare systems could consider this approach and simultaneously set up tools, e.g., a local or regional repository to track outcomes in real-time. If the efficacy is similar to the small randomized trial (Lenze et al., 2020), it should be evident in such data. Out of caution, the practice guidelines could urge caregivers to consider administering FLV only to those COVID-19+patients at highest risk for disease progression, and who do not have access to one of the monoclonal antibodies that have been given emergency use authorizations by the FDA (FDA, 2020a; FDA, 2020b). Also, these guidelines could be revised at any time.

Small biomarker intensive trials should be planned to assess antiviral, immunomodulatory, anti-thrombotic effects or other effects in patients treated with FLV. One could incorporate tools such as single cell RNA and protein analysis in such studies. While human data is being gathered, additional preclinical data in cell culture systems like co-cultures of human epithelial and immune cells would be useful (Grunwell et al., 2019). Data from non-human primate and hamsters would provide valuable information on optimal timing of drug, amount needed for efficacy, and which among the myriad mechanisms of action might be most relevant.

There may be a role for serotonin modulation in the inpatient setting. Indeed, if this drug is not working primarily as an antiviral but rather through other mechanisms (e.g., immunomodulatory, anti-platelet), it may be efficacious in this setting where hyperinflammatory responses and thrombotic events drive disease pathology. However, there will need to be vigilance for emergence of a hyperserotonergic state with similarities to serotonin syndrome, as noted earlier. Thus it may make sense to initiate fluvoxamine in the less severe hospitalized patients but administer a serotonin 2 A, B and C receptor antagonist such as cyproheptadine or mirtazapine in the more severe patients (along with fluvoxamine). It is also tempting to speculate on a role for FLV in COVID-19 long-haulers. There are likely to be subsets in this heterogeneous group that may have an aberrant immune response that has lingered on, in which FLV may be efficacious. Finally, there may be a role for FLV in the treatment of other viral illnesses in which there is some version of a cytokine storm present (Fajgenbaum and June, 2020).

### Example 10: Fluvoxamine for the Early Treatment of SARS-CoV-2 Infection: A Review of Current Evidence

### Abstract

SARS-CoV-2 infection causes COVID-19, which frequently leads to clinical deterioration and/or long-lasting morbidity. Academic and governmental experts throughout the USA met in 2021 to discuss the potential for use of fluvoxamine as early treatment of SARS-CoV-2 infection. Fluvoxamine is a selective serotonin reuptake inhibitor (SSRI) that is a strong sigma-1 receptor agonist, and this may effectively reduce cytokine production, preventing clinical deterioration. This repurposed psychiatric medication has a well-known safety record, is inexpensive, easy to use, and widely available, all of which are advantages during this global COVID-19 pandemic. At the meeting, experts reviewed the existing published literature on the use of fluvoxamine as experimental COVID-19 treatment, as well as prior research on the potential mechanisms for anti-inflammatory effects of fluvoxamine, including for other conditions including sepsis. Investigators shared current trials underway and existing gaps in knowledge. Two randomized controlled trials and one observational study examining the effect of fluvoxamine in COVID-19 treatment have found high efficacy. Four larger randomized clinical trials are currently underway, including three in the USA and Canada. More data are needed on dosing and mechanisms of effect, however, fluvoxamine appears to have substantial potential as a safe and widely available medication that could be repurposed to ameliorate serious COVID-19-related morbidity and mortality. As of April 2021, fluvoxamine was mentioned in the NIH COVID-19 treatment guidelines, although no recommendation is made for or against use. Available data may warrant clinician discussion of fluvoxamine as a treatment option for COVID-19, using shared decision making.

### Key Points

Fluvoxamine appears to have potential as a safe, inexpensive, and widely available medication that could be effectively repurposed to ameliorate serious COVID-19-related morbidity and mortality.

Fluvoxamine prevented clinical deterioration and long-lasting symptoms related to COVID-19 in initial studies and one large clinical trial, with several large clinical trials underway globally.

Current information may warrant clinician discussion of fluvoxamine as a treatment option for COVID-19, using shared decision making.

### Introduction

SARS-CoV-2 infection causes coronavirus disease 2019 (COVID-19), which frequently leads to clinical deterioration around the second week of illness [1, 2], and/or long-lasting morbidity after initial infection [3, 4]. While some treatment options have come to light for those who have already progressed to severe disease, increasingly there are calls for discovery of effective early treatments that can prevent clinical deterioration and/or long-term morbidity in the first place [5].

Evidence is steadily accumulating that points to the role of excessive immune response in SARS-CoV-2 infection as a key factor in clinical deterioration or long-term symptoms. A leading theory is that SARS-CoV-2 replicates in an intermediate compartment between the endoplasmic reticulum (ER) and Golgi complex, leading to ER stress and increased cytokine production causing an excessive inflammatory response [6]. In late 2020, Gordon and colleagues [7] identified that knockout or knockdown of SIGMAR1 gene, which encodes the sigma-1 receptor (S1R), caused substantial reduction in SARS-CoV-2 replication. The S1R is already well established as influencing the pathophysiology of multiple psychiatric, neurodegenerative, and central nervous system disorders [8,9,10,11,12]. The findings of Gordon and colleagues, along with prior research indicating a potential role for S1R ligands in preventing sepsis—also associated with excessive inflammatory cytokine production [13]—has led to an interest in exploring the potential for the repurposing of existing drugs that target the S1R as early treatment for SARS-CoV-2 infection.

Fluvoxamine is a selective serotonin reuptake inhibitor (SSRI) that has been widely used globally since the 1990s. Previous studies have shown high affinity for the S1R [14], and in multiple comparative studies fluvoxamine has been consistently ranked as one of the most potent S1R agonists clinically available [6, 10, 15]; therefore, it may effectively reduce cytokine production and prevent clinical deterioration. It is inexpensive, easy to use, widely available globally, and highly lipophilic, with rapid intracellular uptake into lung epithelial cells [16].

### Safety Profile

Fluvoxamine has a well-known safety profile [17, 18], with nausea as the most common adverse event. In a global database of 35,368 people who had taken fluvoxamine across 66 studies in 11 countries, nausea was reported in 15.7% of patients, followed by somnolence (6.4%), asthenia (5.1%), headache (4.8%), and dry mouth (4.8%). Serious adverse events occurred in approximately 2.0% of people who were taking this medication for a psychiatric disorder, with 1.6% requiring hospitalization and <0.4% experiencing another serious adverse event, including a suicide attempt, depression, death, or accidental injury [17].

### Current Evidence on Fluvoxamine as COVID-19 Treatment

Several studies have examined the efficacy of fluvoxamine for improvement in the clinical progression of SARS-CoV-2 infection or have explored the potential mechanisms by which fluvoxamine may have a beneficial effect as early COVID-19 treatment (TABLE 26). In a small randomized clinical trial known as STOP COVID, participants were randomly assigned to receive 100 mg of fluvoxamine (n=80) or placebo (n=72) up to three times per day, for 15 days [1]. Lenze and colleagues hypothesized that fluvoxamine would prevent clinical deterioration if prescribed early in COVID-19 (i.e., within 7 days of symptoms onset). All participants were aged ≥18 years, were not already hospitalized or living in an institutional setting (but were self-isolated per public health guidance), and had diagnosed, symptomatic SARS-CoV-2 infection, with symptoms onset of a median of 4 days prior to enrollment (interquartile range, 3-5; maximum 7 days). The primary endpoint of this study was clinical deterioration, as defined by both (1) presence of dyspnea (i.e., shortness of breath) or hospitalization for dyspnea or pneumonia, and (2) presence of hypoxia with pulse oxygen saturation <92% on room air. At study completion, none (0%) of the 80 participants receiving fluvoxamine met the criteria for clinical deterioration, but 6 (8.3%) of the 72 participants receiving placebo deteriorated clinically (absolute difference from survival analysis 8.7%, 95% CI, 1.8% to 16.4%; log-rank p=0.009). Despite the high dose of fluvoxamine administered during this trial, only one serious adverse event occurred, of a person with no respiratory deterioration, hospitalized for dehydration. Overall, 11 other adverse events occurred in the treatment group; the placebo group had 6 serious adverse events (all hospitalizations for COVID-19) and 12 other adverse events, including higher reports of pneumonia and gastrointestinal symptoms.

TABLE 26
Key characteristics of completed studies on fluvoxamine for treatment of SARS-CoV-2 infection.

| Study, first | | | | | | |
|---|---|---|---|---|---|---|
| author | | Sample | | Study population and | Dura- | |
| [references] | Design | size | Intervention | inclusion criteria | tion | Findings |
| Lenze | Randomized | 152 | Fluvoxamine | Aged ≥1≥, not hospitalized or | 15 days | Zero (0%) of the participants receiving |
| [1] | controlled trial | | 100 mg, | living in an institutional | | fluvoxamine [symbol] the criteria for [symbol] ( [symbol] |
| | (participants were | | [symbol] ×/day × 14 | setting, diagnosed symptomatic | | and [symbol] ) within 15 days, but [symbol] ( [symbol] . [symbol] %) |
| | [symbol] 1:1 [symbol] a | | days | SARS-CoV-2 infection <7 | | of the participants receiving placebo |
| | [symbol] or a | | | days from symptoms onset | | deteriorated clinically (absolute |
| | placebo [symbol] ) | | | | | difference of 8.7%, 95% CI 1.8 to 16.4%) |
| Seftel | Observational [symbol] | 113 | Fluvoxamine | Workers with a congregate | 14 days | [symbol] 5 people opted into fluvoxamine |
| [1 [symbol] ] | (participants ware | | 50-100 mg | living environment | | treatment, at 14 days, 0% (0. [symbol] ) of |
| | given a [symbol] choice of | | [symbol] , then | during a mass outbreak | | those receiving fluvoxamine had been |
| | [symbol] treatment | | 50 mg | of COVID-19; patients | | hospitalized for clinical deterioration, |
| | same [symbol] | | 2×/day × 14 | tested positive for SARS- | | and 12.5% ( [symbol] / [symbol] ) of those |
| | diagnosis, or | | days | CoV-2 during mass testing | | declining treatment had been hospitalized |
| | observation only) | | | | | (p = 0.005). By Day 14, 0% (0/ [symbol] ) |
| | | | | | | of those in the fluvoxamine group |
| | | | | | | reposted ongoing symptoms related |
| | | | | | | to COVID-19, whereas 80% (2 [symbol] /48) |
| | | | | | | of those receiving no treatment reported |
| | | | | | | symptoms (p < 0.001), with 21% (10/48) |
| | | | | | | reporting ≥6 ongoing symptoms |
| | | | | | | at 14 days |
| Rels | Phase III: | 3323 | Fluvoxamine | Brazil Aged >1 [symbol] , positive SARS- | 28 days | Primary endpoints were (1) rate of |
| [21] | quadruple-blind | | 100 mg, 2 × | CoV-2 test, acute [symbol] - [symbol] | | emergency visits with observation |
| | [symbol] controlled | | day × 1 [symbol] | symptoms for <7 days, and at | | unit stay > [symbol] h, and (2) rate of |
| | trial (participants | | days | least one of the [symbol] aged >50, | | hospitalization due to lower respiratory |
| | [symbol] 1:1:1:1 to | | | diabetes [symbol] requiring | | tract infection related to COVID-19 |
| | treatment with | | | oral medication or insulin, systemic | | Secondary endpoints included (1) change |
| | [symbol] , | | | arterial hypertension requiring at least | | in [symbol] load on Days 3 and 7 after |
| | [symbol] HCL, | | | one medication for control, known | | randomization, (2) time to clinical |
| | [symbol] , or | | | cardiovascular disease (e.g. heart | | improvement (self-reported |
| | placebo) | | | failure, congenital heart | | improvement >50% compared to baseline |
| | | | | disease, [symbol] symptomatic [symbol] | | symptoms), (3) [symbol] to hospitalization |
| | | | | disease (emphysema or chronic | | due to progression of COVID-19, (4) |
| | | | | bronchitis), symptomatic asthma | | number of days with respiratory |
| | | | | requiring chronic use of agents for | | symptoms since [symbol] , (5) rate |
| | | | | symptoms control, [symbol] , body | | of [symbol] -cause hospitalizations, |
| | | | | mass index >3 [symbol] [symbol] body | | (6) rate of COVID-19-related |
| | | | | weight, organ transplant, stage | | hospitalizations, (7) rate of |
| | | | | IV chronic kidney disease | | all-cause mortality, (8) rate of |
| | | | | or need for dialysis. | | cardiovascular death, (9) rate of |
| | | | | Immunosuppression or receiving | | respiratory death, (10) rating on the [symbol] |
| | | | | corticosteroid therapy | | Global-10 Scale (28), (11) rating on |
| | | | | equivalent to a 10 mg of [symbol] , | | the [symbol] Clinical Progression Scale |
| | | | | history of cancer in the last | | [ [symbol] ] (12) rate of adverse events, |
| | | | | 5 years or currently | | and (13) percent adherence to study drug |
| | | | | undergoing cancer treatment, or | | |
| | | | | SARS-CoV-2 vaccination | | |

[symbol]

indicates data missing or illegible when filed

Inspired by the newly published results from Lenze and colleagues, during a large occupational outbreak of COVID-19 in late 2020, Seftel and Boulware conducted a real-world observational study of early SARS-CoV-2 treatment with fluvoxamine [19]. A total of 113 people in a work-associated congregate living environment were offered fluvoxamine on the same day that they tested positive for SARS-CoV-2. Testing used a rapid antigen test during one of three rounds of mass testing during the outbreak, regardless of symptoms. Participants could choose whether or not to accept the medication, given the limited data in support of its use for this purpose. A total of 65 people (57.5%) opted for treatment with a 50-100 mg loading dose of fluvoxamine, followed by 50 mg twice daily (50 mg was a lower dose than the Lenze trial), and 48 (42.5%) opted for observation alone. Participants received in-person follow-up at 7 and 14 days, with 100% retention. At 14 days, no patients receiving fluvoxamine had been hospitalized for clinical deterioration, however, 12.5% (6/48) of patients who declined treatment had been hospitalized (p=0.005). Notably, by Day 14, 0% (0/65) of those in the fluvoxamine group reported ongoing symptoms related to SARS-CoV-2 infection, whereas 60% (29/48) of those receiving no treatment reported symptoms (p<0.001), with 21% (10/48) reporting five or more ongoing symptoms at 14 days.

Most recently, investigators from the TOGETHER trial in Brazil published results of their Phase III clinical trial. The trial comprised four treatment arms aimed to evaluate the effect of various medications to reduce the need for emergency care requiring observation for >6 h due to the worsening COVID-19, and/or hospitalization due to COVID-19-related lower respiratory tract infection. Adults with a positive SARS-CoV-2 test, acute flu-like symptoms for <7 days, and at least one enhancement factor (e.g., older age, diabetes mellitus, immunosuppression, etc.) were randomized to 10 days of treatment in one of four treatment arms: fluvoxamine (100 mg twice daily), metformin, ivermectin, or placebo. At the second interim analysis in April 2021, metformin was dropped, yet fluvoxamine was retained [20], and on Aug. 6, 2021, the trial arms were stopped for superiority of fluvoxamine, with a total of 3323 patients enrolled. The relative risk (RR) of hospital admittance or emergency room observation for more than 6 hours was determined to be 0.68 (95% Bayesian Credible Interval [BCI] 0.52-0.88) for participants receiving fluvoxamine versus the placebo control in the intention-to-treat sample. In a per-protocol analysis of participants who were adherent to at least 80% of pills, fluvoxamine was effective against both deterioration and mortality, with an RR of 0.34 for hospitalization (95% BCI 0.21-0.54) and an odds ratio of 0.09 (95% CI 0.01-0.47) for mortality [21].

Further study is needed to investigate potential anti-viral and anti-inflammatory, and other mechanisms of fluvoxamine in the context of SARS-CoV-2 infection. One theory of its mechanism of action is a reduction in the ER stress response and reduction in cytokine production as a result of sigma-1 activation, given fluvoxamine's potency as an S1R agonist. The S1R is an ER chaperone protein that regulates the ER stress response as well as production of cytokines in response to infection and other inflammatory triggers, S1R agonists prevent Inositol Requiring Enzyme 1a (IRE1) from activating X-Box Binding Protein-1 (XBP-1) mRNA, therefore regulating the ER stress response, and reducing cytokine production [13]. In a recent study of affinity of various antidepressant drugs for the S1R in rat brains, Ishima and colleagues found that fluvoxamine was the most potent S1R agonist among ten different antidepressants tested [14]. In a 2019 study of fluvoxamine's effectiveness in preventing lethal septic shock in mice, Rosen and colleagues found that only 9% of wild-type (WT) mice died after injection with lipopolysaccharide (LPS), known to rapidly induce proinflammatory cytokines in mice and humans, compared to 62% of mice with sigma-1 knockout (KO). Similar results were found after infection with fecal slurry (1 g/kg of bodyweight), which resulted in septic shock and death in substantially more of the S1R KO mice than WT mice (p<0.05). However, in the presence of an IRE1 inhibitor, survival rates of S1R KO mice and WT mice were similar, further demonstrating the potential mechanistic effects of S1R agonism to dampen inflammatory response [13].

Rather than just anti-inflammatory effects, another possible mechanism of fluvoxamine's effect is antiviral properties through lysosomotropic effects [22]. Cationic amphiphilic drugs (CADs) such as fluvoxamine tend to accumulate in the lysosomes, altering the pH, interfering with viral proteins that accumulate, and/or preventing mature virus from using lysosomes to escape the cell, which is one characteristic of coronaviruses [23]. It is also possible that lysosomotropic antidepressants may interfere with viral entry through inhibition of acid sphingomyelinase activity [24]. Functional inhibitors of acid sphingomyelinase (FIASMAs) prevent sphingomyelin from being converted to ceramide, which makes it more difficult for a virus like SARS-CoV-2 to enter the cell [25].

Others have theorized that the inhibition of platelet aggregation and mast cell degranulation may be likely mechanisms of action [26, 27]. SSRIs like fluvoxamine have been shown to decrease platelet aggregation and increase bleeding time. Since hyperactive platelets can release excessive serotonin, and serotonin clearance requires healthy pulmonary endothelium, beginning SSRIs early in SARS-CoV-2 infection—before the pulmonary endothelium is damaged by COVID-19—may prevent the effect of potentially damaging platelet hyperactivation, inflammatory thrombosis, and platelet serotonin storm, therefore reducing the risk of a hyperserotonergic state leading to acute respiratory distress [26, 27]. Furthermore, SSRIs decrease histamine release from human mast cells, and reduce mRNA levels of protease-1 in mast cells; this is important, as postmortem lung biopsies of patients with COVID-19 have linked activated mast cells to pulmonary thromboses and edema [26].

### Additional Studies Underway

On Dec. 17, 2020, Lenze and colleagues began a new nationwide, fully remote (internet-based) Phase III randomized controlled trial named STOP COVID 2 (StopCovidTrial.com; ClinicalTrials.gov: NCT04668950) to confirm the initial results from their preliminary trial. This trial is taking place in the USA and Canada, with nation-wide internet-based enrollment and telemedicine appointments for all study interactions. The preliminary trial (STOP COVID) resulted in few cases of clinical deterioration overall due to limited sample size and relatively young and healthy participants, resulting in low precision of the effect size estimate [1]. STOP COVID 2 aimed to recruit 1100 participants with eligibility criteria similar to the first trial (SARS-CoV-2 positive and symptomatic within 7 days of symptoms onset, residing in the community rather than in a hospital or other institutional setting); however, in this trial the sample was enriched, with participants needing to have one or more of the following risk factors for more severe COVID-19: aged 40 years, obesity, diabetes, hypertension, heart disease, lung disease, an immune condition, and/or being African American, Latinx, or Native American.

On May 19, 2021, STOP COVID 2 stopped enrolling new participants on the advice of the Data and Safety Monitoring Board, based on an overall lower rate of clinical deterioration than anticipated (leading to a much larger sample size necessary to observe the a priori minimum detectable effect), the decreasing number of volunteers enrolling in the trial, and their review of the unblinded interim results to date. There were no adverse safety signals, but due to successful vaccination roll-out in the USA and Canada, the trial was no longer expected to accrue the needed number of participants. Participants already enrolled in the trial finished their assigned doses of therapy and will complete the planned follow-up questionnaires at 15 and ˜90 days to assess short- and long-term secondary outcomes. This trial has the same duration and primary outcome as the initial trial (clinical deterioration within 15 days of enrollment, defined by dyspnea and hypoxia), but has lower dosing: 100 mg twice/day, instead of three times daily for 15 days, of either fluvoxamine or placebo. With enrollment halted, the trial will not have the required sample size and statistical power to detect an effect of fluvoxamine on the primary outcome. However, this trial also includes a secondary outcome of health functioning and symptoms assessed at 15 days and again at 3 months, measured by the Global Health Scale [28], and an exploratory symptom questionnaire to assess any effect of fluvoxamine on long-term COVID-19 morbidity.

In January 2021, Drs Bramante, Boulware, and colleagues at the University of Minnesota; Northwestern University; University of Colorado, Denver; UCLA Olive View; and OptumLabs began stage 1 of a Phase III, factorial randomized clinical trial known as COVID-19-OUT (covidout.com; ClinicalTrials.gov: NCT04510194). The initial stage of the quadruple-blinded trial enrolled 70 patients, with the fully enrolled trial having a planned 1160 participants. This factorial trial has five experimental arms (fluvoxamine only, metformin only, ivermectin only, metformin plus fluvoxamine, or metformin plus ivermectin), with one placebo arm. Adults aged 30 to 85 years are eligible for inclusion within ≤3 days of a positive PCR test for SARS-CoV-2 infection if they are asymptomatic or have had symptoms for <7 days before randomization, enroll within 3 days of testing, have a body mass index ≥25 kg/m² by self-report height/weight or ≥23 kg/m² for patients who self-identify as South Asian or Latinx. A glomerular filtration rate (GFR) will be obtained in persons older than age 75 years or who have a history of heart, kidney, or liver failure if a GFR is not visible within the electronic health record within 2 weeks, to ensure these high-risk individuals have a GFR>45 mL/min. The primary outcome measures are (1) decreased oxygenation at 14 days (defined as pulse oxygen saturation ≤93% on home monitoring), (2) emergency department utilization for COVID-19 symptoms at 14 days (and/or hospitalization/death), and (3) post-acute sequelae of SARS-CoV-2 infection (PASC) assessment at 6 and 12 months. This trial is currently enrolling.

Another trail also enrolling is ACTIV-6 (ClinicalTrials.gov: NCT04885530), which is a Phase III, placebo-controlled, randomized trial, run by Dr. Naggie at Duke Clinical Research Institute. This trial has three experimental arms (ivermectin, fluvoxamine, and fluticasone), with a placebo comparator arm matched to each experimental arm. Both participants and the study teams know which study drug they have been allocated but are blinded to whether they are in the experimental or placebo comparator arms for that study drug. Adults aged 30 years with SARS-CoV-2 infection confirmed within 10 days of study screening with two or more current symptoms of acute SARS-CoV-2 infection (fatigue, dyspnea, fever, cough, nausea, vomiting, diarrhea, body aches, chills, headache, sore throat, nasal symptoms, or new loss of sense of taste or smell) began enrolling in this study on Jun. 8, 2021, with a goal of enrolling 15,000 participants before a primary completion date of December 2022. The primary outcome measures of this trial are the number of hospitalizations, number of deaths, and number of symptoms within 14 days, as measured by patient reports.

Finally, another randomized controlled trial of fluvoxamine and COVID-19 is underway in Hungary: SigmaDrugs Research Ltd. is currently recruiting for a Phase II trial (ClinicalTrials.gov: NCT04718480), studying the time to clinical recovery after treatment with 74 days of fluvoxamine 100 mg taken twice daily, compared to placebo. Up to 100 adults who have moderately severe cases of COVID-19 (having each of the following: dyspnea without respiratory distress, a respiration rate 22-29 times per minute, resting pulse oxygen saturation 93%, and pneumonia with pulmonary infiltrates occupying ≤50% of the lung-fields) will be enrolled. The primary endpoint of clinical recovery includes resolving to normal any three of the following four clinical indicators: fever, respiratory rate, pulse oxygen saturation, and cough burden. This study has an estimated completion date of December 2021.

Multiple studies of the mechanisms of fluvoxamine's effect on SARS-CoV-2 are also currently underway, including in vitro and animal studies at multiple institutions. Publication of findings from these pending studies is eagerly awaited, as they will offer meaningful contributions to our understanding of how and why early treatment with fluvoxamine may have a beneficial effect on COVID-19-related morbidity and mortality.

TABLE 27
Key characteristics of studies in progress on fluvoxamine for treatment of SARS-CoV-2 infection, September 2021

| | | | | Study population | | |
|---|---|---|---|---|---|---|
| | | Sample | | and inclusion | | |
| Study, PI | Design | size | Intervention | criteria | Duration | Endpoints |
| STOP COVID 2, | Phase III: | 1100 | Fluvoxamine | USA and | 15 | Primary endpoint |
| Lenze | triple-blind, | (planned) | 100 mg, | Canada | days/3 | is clinical |
| (NCT04668950) | fully remote | 677 | 2×/day × 14 | Aged ≥18, not | months | deterioration, |
| | (internet- | (actual at | days | hospitalized or | | defined by both |
| | based) | time trial | | living in an | | (1) dyspnea or |
| | randomized | enrollment | | institutional | | hospitalization and |
| | controlled trial | was | | setting, | | (2) hypoxia with |
| | (participants | stopped) | | diagnosed, | | oxygen |
| | randomized | | | symptomatic | | saturation <92% |
| | 1:1 to a | | | SARS-CoV-2 | | Secondary |
| | fluvoxamine | | | infection <7 | | endpoint is |
| | arm or a | | | days from | | function by the |
| | placebo arm) | | | symptoms onset, | | PROMIS Global |
| | | | | one or more of | | Health Scale [28], |
| | | | | the following risk | | assessed at 15 |
| | | | | factors: aged ≥40, | | days and 3 |
| | | | | obesity, | | months |
| | | | | diabetes, | | |
| | | | | hypertension, | | |
| | | | | heart disease, | | |
| | | | | lung disease, an | | |
| | | | | immune | | |
| | | | | condition, and/or | | |
| | | | | being African | | |
| | | | | American, Latinx, | | |
| | | | | or Native | | |
| | | | | American | | |
| Fluvoxamine | Phase II: | 100 | Fluvoxamine | Hungary. Aged | 74 | Primary endpoint |
| Administration in | quadruple- | | 100 mg, | 18-70, | days | is time to clinical |
| Moderate | blind | | | hospitalized with | | recovery after |
| SARS-CoV-2 | randomized | | 2×/day × 74 | confirmed SARS- | | treatment, defined |
| Infected | controlled trial | | days | COV-2, with | | as having any |
| Patients, Fekete | (participants | | | moderate | | three of the |
| (NCT04718480) | randomized | | | symptoms | | following four |
| | 1:1 to a | | | (dyspnea without | | items: (1) fever |
| | fluvoxamine | | | respiratory | | resolution for at |
| | arm or a | | | distress, | | least 48 hours |
| | placebo arm) | | | respiration rate | | with antipyretics, |
| | | | | 22-29 times per | | (2) respiration rate |
| | | | | minute, resting | | ≤ 20/min, (3) pulse |
| | | | | pulse oxygen | | oxygen saturation ≥95% |
| | | | | saturation ≥93%, | | on room |
| | | | | and | | air, and (4) any |
| | | | | pneumonia on | | reduction on the |
| | | | | medical imaging | | cough-burden |
| | | | | with pulmonary | | visual analogue |
| | | | | infiltrates | | scale, compared |
| | | | | occupying ≤50% | | to baseline |
| | | | | of the lung-fields) | | |
| COVID-19-OUT, | Phase III: | 1160 | Fluvoxamine | USA nationwide; | 12 | Primary endpoints |
| Bramante | quadruple- | | 50 mg, | aged 30-85, not | months | are (1) decreased |
| (NCT04510194) | blind, factorial | | 2×/day × 14 | hospitalized or | | oxygenation at 14 |
| | randomized | | days, with or | symptomatic >7 | | days (defined as |
| | controlled trial | | within 1500 | days from | | pulse oxygen |
| | (participants | | mg daily of | randomization, | | saturation ≤93% |
| | randomized | | metformin | body mass index ≥25 | | on home |
| | 1:1 to one of | | depending | kg/m² by | | monitoring), (2) |
| | five treatment | | on arm | self-report | | emergency |
| | arms or a | | Other arms | height/weight | | department |
| | placebo arm) | | include | or ≥23 kg/m² for | | utilization for |
| | | | treatment | patients who | | COVID-19 |
| | | | with | self-identify as | | symptoms at 14 |
| | | | Ivermectin | South Asian or | | days, and (3) |
| | | | and/or | Latinx, and a | | post-acute |
| | | | metformin) | glomerular | | sequelae of |
| | | | | filtration rate >45 | | SARS-CoV-2 |
| | | | | mL/min within 2 | | infection (PASC) |
| | | | | weeks for | | assessment at 6 |
| | | | | patients older | | and 12 months, to |
| | | | | than aged 75 | | assess long |
| | | | | years or who | | COVID |
| | | | | have a history of | | Secondary |
| | | | | heart, kidney, or | | endpoints include |
| | | | | liver failure | | (1) maximum |
| | | | | | | symptom severity |
| | | | | | | at 14 and 28 days, |
| | | | | | | (2) maximum |
| | | | | | | clinical support |
| | | | | | | needed on the |
| | | | | | | Clinical |
| | | | | | | Progression Scale |
| | | | | | | at 14 and 28 days, |
| | | | | | | (3) time to |
| | | | | | | meaningful |
| | | | | | | recovery, and a |
| | | | | | | series of |
| | | | | | | laboratory |
| | | | | | | outcomes on Days |
| | | | | | | 1, 5, and 10 |
| ACTIV-6: | Phase III: | 15,000 | Fluvoxamine | U.S. nationwide; | 29 | Primary endpoints |
| COVID- | double-blind | | 50 mg, 2 × | aged ≥30 years, | days | are: (1) number of |
| 19 Outpatient | randomized | | day × 10 | not hospitalized | | hospitalizations, |
| Randomized | controlled trial | | days | currently or | | (2) number of |
| Trial to Evaluate | (participants | | | within 10 days of | | deaths, and (3) |
| Efficacy of | randomized | | | screening, or | | number of |
| Repurposed | 1:1:1:1:1:1 to | | | previously | | symptoms, all |
| Medications | treatment with | | | diagnosed with | | within 14 days and |
| (NCT04885530) | ivermectin, | | | COVID-19 | | as measured by |
| | fluvoxamine, | | | infection (>10 | | patient reports |
| | fluticasone, or | | | days from | | Secondary |
| | placebo | | | screening). Must | | endpoints include: |
| | comparators | | | have SARS- | | (1) change in |
| | of each of | | | CoV-2 infection | | COVID Clinical |
| | these | | | confirmed within | | Progression Scale |
| | experimental | | | 10 days of | | [29], (2) Number |
| | arms | | | screening, and | | of hospitalizations |
| | | | | two or more | | within 28 days, (3) |
| | | | | current | | Number of deaths |
| | | | | symptoms of | | within 28 days, (4) |
| | | | | acute infection | | Number of |
| | | | | for at least 7 | | symptom |
| | | | | days (including | | resolutions (first of |
| | | | | fatigue, dyspnea, | | at least 3 |
| | | | | fever, cough, | | consecutive days |
| | | | | nausea, | | without |
| | | | | vomiting, | | symptoms) within |
| | | | | diarrhea, body | | 28 days, (5) |
| | | | | aches, chills, | | Change in quality |
| | | | | headache, sore | | of life as |
| | | | | throat, nasal | | measured by the |
| | | | | symptoms, or | | PROMIS-29 from |
| | | | | new loss of | | baseline, day 7, |
| | | | | sense of taste or | | 14, 28, and 29) |
| | | | | smell | | [30], and (6) |
| | | | | | | Composite score |
| | | | | | | of hospitalizations, |
| | | | | | | urgent care visits, |
| | | | | | | and emergency |
| | | | | | | room visits, within |
| | | | | | | 28 days |

### Efficacy

With publication of the TOGETHER trial results, along with the first STOP COVID trial, the efficacy of fluvoxamine for preventing clinical deterioration and/or long-term morbidity due to SARS-CoV-2 infection has been demonstrated in two randomized placebo-controlled trials. The extent of fluvoxamine's efficacy remains unclear; in both the STOP COVID and Seftel and Boulware studies, no participant who received fluvoxamine experienced the primary adverse outcome—respiratory deterioration (in the case of STOP COVID) or hospitalization (Seftel and Boulware)—for their COVID-19 infection, compared with 8.3% or 12.5% of participants in the control arm, respectively. Similarly, in the TOGETHER trial per-protocol analysis, the fluvoxamine arm was associated with a 66% reduction in clinical deterioration requiring hospitalization or extended emergency contact, and a 91% reduction in mortality [21]. In addition to these primary endpoints, the Seftel study also found that zero participants in the fluvoxamine group had ongoing symptoms at 14 days, compared to 60% of people in the control group; however, STOP COVID found no differences in short-term symptomatic recovery and saw some lingering symptoms reported after 4 months in both the fluvoxamine and placebo arms, although numbers were too small to allow for statistical comparisons. While mortality due to COVID-19 has received the most attention to date among the media and general public, both short-term and long-term morbidity from SARS-CoV-2 infection is a substantial burden worldwide.

### Mechanisms of Action

The anti-inflammatory effects of fluvoxamine are well understood, but the mechanism by which it could have such a strong effect on morbidity and mortality—especially without more clearly demonstrated anti-viral effects—is still unclear. The in vitro and animal studies currently underway will greatly improve our understanding of the biological effect of this drug, and further explore any potential for the prolongation of the viral phase of infection as a result of fluvoxamine's inhibition of the inflammatory response, which would warrant caution with its use. Since fluvoxamine seems to have anti-inflammatory and immune modulatory actions without substantial suppression of the immune response, it may be much safer to use in the earliest stages of COVID-19, as compared to systemic steroids.

### Research Priorities and Future Directions

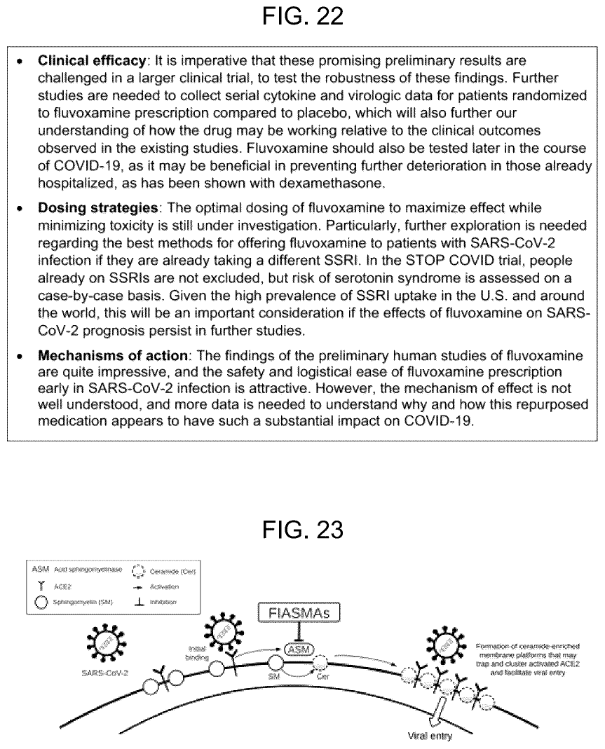

As of April 2021, fluvoxamine is now mentioned in the NIH COVID-19 treatment guidelines, although no recommendation is yet made for or against use, due to insufficient available evidence. Based on our review of the current evidence and studies in progress, there are several key research priorities (FIG. 22). In summary, fluvoxamine is seen as a highly promising drug for COVID-19, but more studies are needed to elucidate its mechanism of action and possible deleterious effects.

### REFERENCES

1. Lenze E J, Mattar C, Zorumski C F, et al. Fluvoxamine vs placebo and clinical deterioration in outpatients with symptomatic COVID-19: a randomized clinical trial. JAMA. 2020; 324(22):2292-300. https://doi.org/10.1001/jama.2020.22760.
2. Cummings M J, Baldwin M R, Abrams D, et al. Epidemiology, clinical course, and outcomes of critically ill adults with COVID-19 in New York City: a prospective cohort study. Lancet. 2020; 395(10239):1763-70. https://doi.org/10.1016/S0140-6736(20)31189-2.
3. Marshall M. The lasting misery of coronavirus long-haulers. Nature. 2020; 585(7825):339-41. https://doi.org/10.1038/d41586-020-02598-6.
4. Del Rio C, Collins L F, Malani P. Long-term Health Consequences of COVID-19. JAMA. 2020. https://doi.org/10.1001/jama.2020.19719.
5. Kim P S, Read S W, Fauci A S. Therapy for early COVID-19: a critical need. JAMA. 2020; 324(21):2149-50. https://doi.org/10.1001/jama.2020.22813.
6. Hashimoto K. Repurposing of CNS drugs to treat COVID-19 infection: targeting the sigma-1 receptor. Eur Arch Psychiatry Clin Neurosci. 2021. https://doi.org/10.1007/s00406-020-01231-x.
7. Gordon D E, Hiatt J, Bouhaddou M, et al. Comparative host-coronavirus protein interaction networks reveal pan-viral disease mechanisms. Science. 2020. https://doi.org/10.1126/science.abe9403.
8. Hayashi T, Su T P. An update on the development of drugs for neuropsychiatric disorders: focusing on the sigma 1 receptor ligand. Expert Opin Ther Targets. 2008; 12(1):45-58. https://doi.org/10.1517/14728222.12.1.45.
9. Albayrak Y, Hashimoto K. Sigma-1 receptor agonists and their clinical implications in neuropsychiatric disorders. Adv Exp Med Biol. 2017; 964:153-61. https://doi.org/10.1007/978-3-319-50174-1_11.
10. Hashimoto K. Activation of sigma-1 receptor chaperone in the treatment of neuropsychiatric diseases and its clinical implication. J Pharmacol Sci. 2015; 127(1):6-9. https://doi.org/10.1016/j.jphs.2014.11.010.
11. Hashimoto K. Targeting the sigma-1 receptor chaperone in the treatment of perinatal brain injury. Exp Neurol. 2015; 265:118-21. https://doi.org/10.1016/j.expneurol.2014.12.019.
12. Couly S, Goguadze N, Yasui Y, et al. Knocking out sigma-1 receptors reveals diverse health problems. Cell Mol Neurobiol. 2020. https://doi.org/10.1007/s10571-020-00983-3.
13. Rosen D A, Seki S M, Fernéndez-Castañeda A, et al. Modulation of the sigma-1 receptor-IRE1 pathway is beneficial in preclinical models of inflammation and sepsis. Science Transl Med. 2019. https://doi.org/10.1126/scitranslmed.aau5266.
14. Ishima T, Fujita Y, Hashimoto K. Interaction of new antidepressants with sigma-1 receptor chaperones and their potentiation of neurite outgrowth in PC12 cells. Eur J Pharmacol. 2014; 727:167-73. https://doi.org/10.1016/j.ejphar.2014.01.064.
15. Cobos E J, Entrena J M, Nieto F R, Cenden C M, Del Pozo E. Pharmacology and therapeutic potential of sigma(1) receptor ligands. Curr Neuropharmacol. 2008; 6(4):344-66. https://doi.org/10.2174/157015908787386113.
16. Hallifax D, Houston J B. Saturable uptake of lipophilic amine drugs into isolated hepatocytes: mechanisms and consequences for quantitative clearance prediction. Drug Metab Dispos. 2007; 35(8):1325-32. https://doi.org/10.1124/dmd.107.015131.
17. Wagner W, Zaborny B A, Gray T E. Fluvoxamine. A review of its safety profile in world-wide studies. Int Clin Psychopharmacol. 1994; 9(4):223-7. https://doi.org/10.1097/00004850-199400940-00001.
18. Altamura A C, Caldiroli A, Buoli M. Pharmacokinetic evaluation of fluvoxamine for the treatment of anxiety disorders. Expert Opin Drug Metab Toxicol. 2015; 11(4):649-60. https://doi.org/10.1517/17425255.2015.1021331.
19. Seftel D, Boulware D R. Prospective cohort of fluvoxamine for early treatment of COVID-19. Open Forum Infect Dis. 2021. https://doi.org/i0.1093/ofid/ofab050.
20. Together Clinical Trials [Website]. http://togethertrial.com. Accessed 3 Jun. 2021.
21. Reis G, dos Santos Moreira Silva E A, Medeiros Silva D C, Thabane L, Milagres A C, Santiago Ferreira T, et al. Effect of early treatment with Fluvoxamine on risk of emergency care and hospitalization among patients with COVID-19: The TOGETHER randomized platform clinical trial. Lancet Glob Health 2021; S2214-109X(21)00448-4. https://doi.org/10.1016/S2214-109X(21)00448-4.
22. Homolak J, Kodvanj I. Widely available lysosome targeting agents should be considered as potential therapy for COVID-19. Int J Antimicrob Agents. 2020; 56(2): 106044. https://doi.org/10.1016/j.ijantimicag.2020.106044.
23. Ghosh S, Dellibovi-Ragheb T A, Kerviel A, et al. β-Coronaviruses use lysosomes for egress instead of the biosynthetic secretory pathway. Cell. 2020; 183(6):1520-35.e14. https://doi.org/10.1016/j.cell.2020.10.039.
24. Carpinteiro A, Edwards M J, Hoffmann M, et al. Pharmacological inhibition of acid sphingomyelinase prevents uptake of SARS-CoV-2 by epithelial cells. Cell Rep Med. 2020; 1(8): 100142. https://doi.org/10.1016/j.xcrm.2020.100142.
25. Kornhuber J, Muehlbacher M, Trapp S, et al. Identification of novel functional inhibitors of acid sphingomyelinase. PLoS ONE. 2011; 6(8): e23852. https://doi.org/10.1371/journal.pone.0023852.
26. Sukhatme V P, Reiersen A M, Vayttaden S J, Sukhatme V V. Fluvoxamine: a review of its mechanism of action and its role in COVID-19. Front Pharmacol. 2021; 12: 652688. https://doi.org/10.3389/fphar.2021.652688.
27. Hashimoto Y, Suzuki T, Hashimoto K. Old drug fluvoxamine, new hope for COVID-19. Eur Arch Psychiatry Clin Neurosci. 2021. https://doi.org/10.1007/s00406-021-01326-z.
28. Hays R D, Bjorner J B, Revicki D A, Spritzer K L, Cella D. Development of physical and mental health summary scores from the patient-reported outcomes measurement information system (PROMIS) global items. Qual Life Res. 2009; 18(7):873-80. https://doi.org/10.1007/s11136-009-9496-9.
29. WHO Working Group on the Clinical Characterisation and Management of COVID-19 Infection. A minimum common outcome measure set for COVID-19 clinical research. Lancet Infect Dis. 2020. https://doi.org/10.1016/S1473-3099(20)30483-7.
30. Cella D, Choi S W, Condon D M, Schalet B, Hays R D, Rothrock N E, et al. PROMIS® adult health profiles: efficient short-form measures of seven health domains. Value Health. 2019; 22(5):537-44. https://doi.org/10.1016/j.jval.2019.02.004.

### Example 11: Association Between FIASMAs and Reduced Risk of Intubation or Death in Individuals Hospitalized for Severe Covid-19: An Observational Multicenter Study

### Abstract

Several medications commonly used for a number of medical conditions share a property of functional inhibition of acid sphingomyelinase (ASM), or FIASMA. Preclinical and clinical evidence suggest that the ASM/ceramide system may be central to severe acute respiratory syndrome-coronavirus 2 (SARS-CoV-2) infection. We examined the potential usefulness of FIASMA use among patients hospitalized for severe coronavirus disease 2019 (COVID-19) in an observational multicenter study conducted at Greater Paris University hospitals. Of 2,846 adult patients hospitalized for severe COVID-19, 277 (9.7%) were taking an FIASMA medication at the time of their hospital admission. The primary end point was a composite of intubation and/or death. We compared this end point between patients taking vs. not taking an FIASMA medication in time-to-event analyses adjusted for sociodemographic characteristics and medical comorbidities. The primary analysis was a Cox regression model with inverse probability weighting (IPW). Over a mean follow-up of 9.2 days (SD=12.5), the primary end point occurred in 104 patients (37.5%) receiving an FIASMA medication, and 1,060 patients (41.4%) who did not. Despite being significantly and substantially associated with older age and greater medical severity, FIASMA medication use was significantly associated with reduced likelihood of intubation or death in both crude (hazard ratio (HR)=0.71, 95% confidence interval (CI)=0.58-0.87, P<0.001) and primary IPW (HR=0.58, 95% CI=0.46-0.72, P<0.001) analyses. This association remained significant in multiple sensitivity analyses and was not specific to one particular FIASMA class or medication. These results show the potential importance of the ASM/ceramide system in COVID-19 and support the continuation of FIASMA medications in these patients. Double-blind controlled randomized clinical trials of these medications for COVID-19 are needed.

### Study Highlights

Current knowledge: Several medications commonly used for a number of medical conditions share a property of functional inhibition of acid sphingomyelinase (ASM), or FIASMA. Preclinical and clinical evidence suggest that the ASM/ceramide system may be central to severe acute respiratory syndrome-coronavirus 2 (SARS-CoV-2) infection.

This study addresses whether there an association between FIASMA medication use and the composite outcome of intubation or death in patients hospitalized for severe coronavirus disease 2019 (COVID-19).

This study shows that taking an FIASMA medication was associated with reduced likelihood of intubation or death in analyses adjusted for sociodemographic characteristics and medical comorbidities.

These results show the potential importance of the ASM/ceramide system as a treatment target in COVID-19 and support the continuation of FIASMA medications in patients with COVID-19. Double-blind controlled randomized clinical trials of these medications for COVID-19 are needed.

Global spread of the novel coronavirus severe acute respiratory syndrome-coronavirus 2 (SARS-CoV-2) and its variants, the causative agents of coronavirus disease 2019 (COVID-19), has created an unprecedented infectious disease crisis worldwide.¹⁻⁴ Although the availability of vaccines has raised hope for a decline of the pandemic, the search for an effective treatment for patients with COVID-19 among all available medications is still urgently needed.

Several medications commonly used for a number of medical conditions, such as depression or high blood pressure, are functional inhibitors of acid sphingomyelinase (ASM)⁵⁻¹⁰ or FIASMA. FIASMA medications, as detailed in TABLE 28, include, for example, certain antidepressants (e.g., fluoxetine, fluvoxamine, and escitalopram), antihistamine medications (e.g., hydroxyzine and promethazine), antipsychotics (e.g., aripiprazole and chlorpromazine), calcium channel blockers (e.g., amlodipine and bepridil), and mucolytics (e.g., ambroxol).⁵⁻¹⁰

TABLE 28
List of medications that have shown to in
vitro inhibit acid sphingomyelinase⁵⁻¹⁰

| Anti-arrhythmics | Amiodarone | Aprindine |
|---|---|---|
| Anticholinergic antiparkinson | Benztropine | Profenamine |
| medications | Biperidene | |
| Antidepressants | Amitriptyline | Lofepramine |
| | Citalopram | Maprotiline |
| | Clomipramine | Mirtazapine |
| | Desipramine | Nortriptyline |
| | Doxepin | Paroxetine |
| | Duloxetine | Protriptyline |
| | Escitalopram | Sertraline |
| | Fluoxetine | Trimipramine |
| | Fluvoxamine | Venlafaxine |
| | Imipramine | |
| Antidiarrheal medication | Loperamide | |
| Antihistamine medications | Astemizole | Loratadine |
| | Clemastine | Mebhydrolin |
| | Cyproheptadine | Pimethixene |
| | Desloratadine | Promethazine |
| | Hydroxyzine | Terfenadine |
| Antimycobacterial | Clofazimine | |
| Antiprotozoal medications | Emetine | Quinacrine |
| Antipsychotics | Aripiprazole | Pimozide |
| | Chlorpromazine | Promazine |
| | Chlorprothixene | Sertindole |
| | Fluphenazine | Thioridazin |
| | Flupenthixol | Trifluoperazine |
| | Penfluridol | Triflupromazine |
| | Perphenazine | |
| Antivertigo medications | Cinnarizine | Flunarizine |
| Beta blocking agents | Carvedilol | |
| Calcium channel blockers | Amlodipine | Mibefradil |
| | Bepridil | Perhexiline |
| | Fendiline | |
| Cough suppressant | Cloperastine | |
| Endocrine therapy medication | Tamoxifen | |
| Medications for functional | Alverine | Dicycloverine |
| gastrointestinal disorders | Camylofin | Mebeverine |
| Medications of the nervous system | Cinnarizine | Flunarizine |
| Mucolytic | Ambroxol | |
| Muscle relaxant | Cyclobenzaprine | |
| Natural products | Conessine | Tomatidine |
| | Solasodine | |
| Vasodilators | Dilazep | Suloctidil |

Preclinical evidence indicates that SARS-CoV-2 activates the ASM/ceramide system, resulting in the formation of ceramide-enriched membrane domains that facilitate viral entry and infection by clustering ACE2, the cellular receptor of SARS-CoV-2.⁵ An in vitro study⁵ showed that several FIASMA⁶ medications, including fluoxetine and amitriptyline, inhibited ASM and the formation of ceramide-enriched membrane domains, and prevented Vero cells from being infected with SARS-CoV-2. Reconstitution of ceramide in cells treated with antidepressant medications having FIASMA properties restored infection with SARS-CoV-2. In healthy volunteers, oral administration of amitriptyline blocked infection of freshly isolated nasal epithelial cells with SARS-CoV-2.⁵ These preclinical data were confirmed by another study that demonstrated an inhibition of the infection of cultured epithelial cells with SARS-CoV-2 by fluoxetine.¹¹ A scheme of the biological mechanisms proposed by Carpinteiro et al.^{5, 10} underlying the potential inhibition by FIASMAs of cell infection with SARS-CoV-2 is summarized in FIG. 23.

Sphingomyelinase (FIASMAs) of cell infection with severe acute respiratory syndrome-coronavirus 2 (SARS-CoV-2). Initial binding of SARS-CoV-2 spike protein to its ACE2 receptor may result in activation of the acid sphingomyelinase (ASM), formation of surface ceramide molecules that spontaneously form ceramide-enriched membrane platforms. These platforms alter membrane properties and thereby may serve to trap and cluster activated ACE2 receptors, and facilitate viral entry; inhibition of the ASM by FIASMAs may result in reduced concentration of ceramides and decreased viral entry.

Findings from clinical and observational studies are consistent with these preclinical data. First, a randomized double-blind controlled study¹² showed significant protective effects of the FIASMA antidepressant fluvoxamine (N=80) vs. placebo (N=72) on COVID-19 progression in outpatients (0 of 80 patients in the fluvoxamine group vs. 6 of 72 patients in the placebo group; absolute difference, 8.7% (95% CI, 1.8%-16.4%) from survival analysis; log-rank P=0.009). These results were confirmed in an open-label prospective cohort,¹³ in which the incidence of hospitalization was 0% (0 of 65) in patients with COVID-19 who opted to receive fluvoxamine and 12.5% (6 of 48) in those who declined. Second, an observational multicenter retrospective study using data from Greater Paris University Hospitals showed that use of antidepressants, mostly FIASMA antidepressants, and of the FIASMA hydroxyzine, were significantly associated with reduced mortality in patients hospitalized for COVID-19.^{14, 15} Third, retrospective clinical investigations among hospitalized patients with COVID-19 either elderly (N=77)¹⁶ or with hypertension as the only comorbidity¹⁷ (N=96) showed that the use of amlodipine (a calcium channel blocker¹⁸ and an FIASMA) may be associated with decreased mortality. Taken together, these results suggest that the ASM/ceramide system may provide a useful framework for better understanding SARS-CoV-2 infection and favoring the possible repurposing of FIASMA medications against COVID-19.

To our knowledge, no clinical study to date has examined the potential usefulness of FIASMA medications as a class in patients hospitalized for severe COVID-19. Observational studies of patients with COVID-19 taking medications for other disorders can help to decide which treatment should be prioritized for randomized clinical trials and to minimize the risk for patients of being exposed to potentially ineffective or harmful treatments.

We used data from Greater Paris University Hospitals to examine the association between FIASMA medication use and the composite outcome of intubation or death among patients hospitalized for laboratory-confirmed severe COVID-19. Our primary hypothesis was that FIASMA medication use would be associated with reduced risk of intubation or death among patients hospitalized for severe COVID-19 in time-to-event analyses adjusting for sociodemographic characteristics and medical comorbidities. Additional exploratory analyses examined whether this association was specific to certain FIASMA classes (e.g., FIASMA antidepressants) or certain individual medications (e.g., fluoxetine or amlodipine).

### Methods

### Setting and Cohort Assembly

A multicenter cohort study was conducted at 36 Assistance Publique-Hôpitaux de Paris (AP-HP) hospitals from the beginning of the epidemic in France (i.e., January 24 until May 1, 2020).^{14,15, 19-21} We included all adults aged 18 years or over who have been hospitalized in these medical centers for severe COVID-19. COVID-19 was ascertained by a positive reverse-transcriptase-polymerase-chain-reaction (RT-PCR) test on nasopharyngeal or oropharyngeal swab specimens. Severe COVID-19 was defined as having at least one of the following criteria at hospital admission: respiratory rate >24 breaths/min or <12 breaths/min, resting peripheral capillary oxygen saturation in ambient air <90%, temperature >40° C., systolic blood pressure <100 mm Hg, or high lactate levels >2 mmol/L.²²

This observational study using routinely collected data received approval from the Institutional Review Board of the AP-HP clinical data warehouse (decision CSE-20-20_COVID19, IRB00011591, Apr. 8, 2020). AP-HP clinical Data Warehouse initiatives ensure patient information and informed consent regarding the different approved studies through a transparency portal in accordance with European Regulation on data protection and authorization no. 1980120 from National Commission for Information Technology and Civil Liberties (CNIL).

### Data Sources

AP-HP Health Data Warehouse (‘Entrepôt de Données de Santé (EDS)’) contains all available clinical data on all inpatient visits for COVID-19 to 36 Greater Paris university hospitals. The data included patient demographic characteristics, vital signs, laboratory tests, and RT-PCR test results, medication administration data during the hospitalization for COVID-19, current diagnoses, discharge disposition, ventilator use data, and death certificates.

### Variables Assessed

We obtained the following data for each patient at the time of the hospitalization: sex; age, which was categorized into four classes based on the OpenSAFELY study results²³ (i.e., 18-50, 51-70, 71-80, and 81+); hospital, which was categorized into four classes following the administrative clustering of AP-HP hospitals in Paris and its suburbs based on their geographical location (i.e., AP-HP Centre—Paris University, Henri Mondor University Hospitals and at home hospitalization; AP-HP Nord and Hôpitaux Universitaires Paris Seine-Saint-Denis; AP-HP Paris Saclay University; and AP-HP Sorbonne University); obesity, which was defined as having a body mass index higher than 30 kg/m² or an International Statistical Classification of Diseases and Related Health Problems (ICD-10) diagnosis code for obesity (E66.0, E66.1, E66.2, E66.8, and E66.9); self-reported current smoking status; and any medication prescribed according to compassionate use or as part of a clinical trial (e.g., hydroxychloroquine, azithromycin, remdesivir, tocilizumab, sarilumab, or dexamethasone). To take into account possible confounding by indication bias for FIASMA medications, we recorded whether patients had any current diagnosis, based on ICD-10 diagnosis codes recorded during the visit, of neoplasms and diseases of the blood (C00-D89); mental disorders (F01-F99); diseases of the nervous system (G00-G99); cardiovascular disorders (I00-I99); respiratory disorders (J00-J99); digestive disorders (K00-K95); dermatological disorders (L00-L99); diseases of the musculoskeletal system (M00-M99); diseases of the genitourinary system (N00-N99); endocrine disorders (E00-E89); and eye-ear-nose-throat disorders (H00-H95).

All medical notes and prescriptions are computerized in Greater Paris University hospitals. Medications including their dose, frequency, date, and mode of administration were identified from medication administration data or scanned hand-written medical prescriptions, through two deep learning models based on BERT contextual embeddings,²⁴ one for the medications and another for their mode of administration. The model was trained on the APmed corpus, a previously annotated dataset for this task. Extracted medications names were then normalized to the Anatomical Therapeutic Chemical (ATC) terminology using approximate string matching.

Medications with Functional Inhibition Effect on Acid Sphingomyelinase

FIASMA medications were defined as having a substantial in vitro functional inhibition effect on ASM (i.e., a residual ASM activity lower than 50%), as detailed elsewhere,⁵⁻⁹ and were divided into the following classes according to their ATC code²⁵: FIASMA alimentary tract and metabolism medications (e.g., loperamide); cardiovascular system medications, subdivided into calcium channel blockers (e.g., amlodipine), and other cardiovascular medications (e.g., carvedilol); nervous system medications, subdivided according to ATC codes into psycho-analeptic (e.g., amitriptyline) and psycholeptic medications (e.g., chlorpromazine); and respiratory system medications (e.g., desloratadine).

FIASMA medication use was defined as receiving at least one FIASMA medication within the first 24 hours of hospital admission. To minimize potential confounding effects of late prescription of FIASMA medications, patients who initiated an FIASMA medication more than 24 hours after hospital admission were excluded from the analyses. Patients who received at study baseline an antipsychotic or a benzodiazepine while being hospitalized in an intensive care unit, possibly as an aid to oral intubation, were also excluded.

### Primary End Point

Study baseline was defined as the date of hospital admission for COVID-19. The primary end point was the occurrence of intubation and/or death. For patients who died after intubation, the timing of the primary end point was defined as the time of intubation. Patients without an end point event had their data censored on May 1, 2020.

### Statistical Analysis

We calculated frequencies of all baseline characteristics described above in patients receiving or not receiving an FIASMA medication and compared them using standardized mean differences (SMD). SMD are useful to evaluate between-group differences in baseline characteristic variables independently of their unit of measurement (i.e., for both continuous and categorical variables²⁶⁻²⁸). We considered SMD greater than 0.1 as reflecting significant differences, a threshold recommended for declaring imbalance.²⁷

To examine the association between FIASMA medication use at baseline and the end point of intubation or death, we performed Cox proportional-hazards regression models.²⁹ To help account for the nonrandomized prescription of medications and reduce the effects of confounders, the primary analysis used propensity score analysis with inverse probability weighting (IPW).³⁰ The individual propensities for receiving an FIASMA medication at baseline were estimated using a multivariable logistic regression model that included sex, age, hospital, obesity, current smoking status, and medical conditions. In the inverse-probability-weighted analyses, the predicted probabilities from the propensity-score models were used to calculate the stabilized inverse-probability-weighting weights. The association between FIASMA medication use and the end point was then estimated using an IPW Cox regression model. In case of non-balanced covariates, an IPW multivariable Cox regression model adjusting for the non-balanced covariates was also performed. Kaplan-Meier curves were performed using the inverse-probability-weighting weights and their pointwise 95% confidence intervals were estimated using the nonparametric bootstrap method.³¹

We conducted two sensitivity analyses. First, we performed a multivariable Cox regression model including as covariates the same variables used in the IPW analysis. Second, we used a univariate Cox regression model in a matched analytic sample using a 1:1 ratio, based on the same variables used for the IPW analysis and the multivariable Cox regression analysis. To reduce the effects of confounding, optimal matching was used in order to obtain the smallest average absolute distance across all clinical characteristics between exposed patients and nonexposed matched controls.

We performed four additional exploratory analyses. First, we examined the relationships between each FIASMA class and each individual FIASMA medication with the composite end point of intubation or death. Second, we reproduced these analyses while comparing patients receiving any FIASMA medication at baseline to those receiving paracetamol at baseline, to mimic an active comparator, as previously done in several ongoing clinical trials.³² This analysis sought to address a potential immortal bias, by including in the control group patients who had the same likelihood as those in the FIASMA group to survive at baseline until being prescribed a treatment. Third, because of discrepancies in the potential FIASMA in vitro effect of venlafaxine, mirtazapine, and citalopram,^{5,6} we reproduced the main analyses while considering these molecules as FIASMAs, contrary to the main analyses. Finally, we reproduced the main analyses among all patients hospitalized for COVID-19, with and without clinical severity criteria at baseline.

For all associations, we performed residual analyses to assess the fit of the data, check assumptions, including proportional hazards assumption using proportional hazards tests and diagnostics based on weighted residuals,²⁹ and examined the potential influence of outliers. To improve the quality of result reporting, we followed the recommendations of The Strengthening the Reporting of Observational Studies in Epidemiology (STROBE) Initiative. Because our main hypothesis focused on the association between FIASMA medication use at baseline and the composite end point of intubation or death, statistical significance was fixed a priori at two-sided Pvalue<0.05. Only if a significant association was found, we planned to perform additional exploratory analyses as described above. All analyses were conducted in R software version 3.6.3 (R Project for Statistical Computing).

### Results

### Characteristics of the Cohort

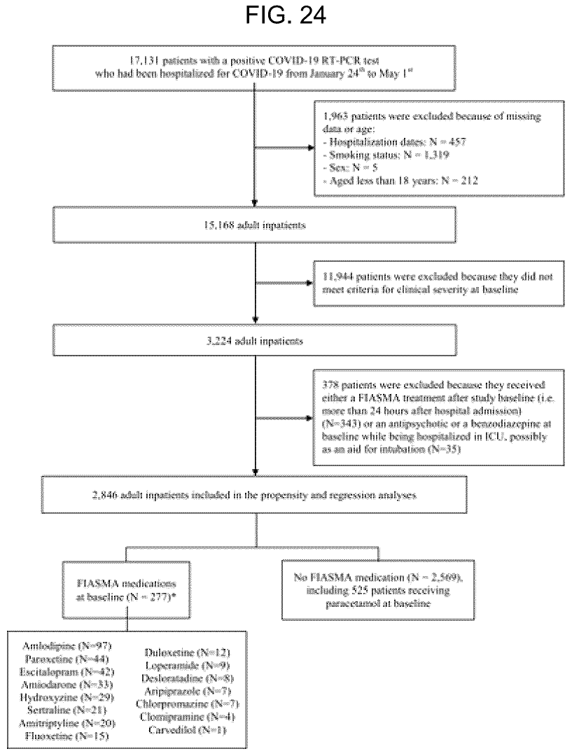

Of the 17,131 patients hospitalized for COVID-19, ascertained with a positive COVID-19 RT-PCR test, 1,963 patients (11.5%) were excluded because of missing data or young age (i.e., <18 years old of age). Of these 15,168 patients, 3,224 (21.3%) met criteria for severe COVID-19 at hospital admission. Of these 3,224 patients, 378 (11.7%) were excluded because they started an FIASMA medication more than 24 hours after hospital admission (N=343) or because they initiated an antipsychotic or a benzodiazepine in an intensive care unit, possibly as an aid for intubation (N=35). Of the remaining 2,846 adult patients, 277 (9.7%) received an FIASMA medication within the first 24 hours of hospitalization, with a mean delay between hospital admission and first FIASMA medication prescription of 0.11 days (SD=0.38; interquartile range (IQR)=0.00-0.19; FIG. 24).

RT-PCR test results were obtained after a median delay of 0.9 days (SD=9.4; IQR=0.5-1.7) from hospital admission date. This median delay was similar (i.e., 0.9 days) in the exposed (SD=11.4; IQR=0.5-1.4) and nonexposed (SD=9.3; IQR=0.5-1.7) groups. Over a mean follow-up of 9.2 days (SD=12.5; median=6 days; IQR=2-11), 1,168 patients (41.0%) had an end point event at the time of data cutoff on May 1, 2020. Among patients who received an FIASMA medication at baseline, the mean follow-up was 12.0 days (SD=12.9, median=8 days; IQR=4-14), whereas it was of 8.9 days (SD=12.4, median=5 days; IQR=1-11) in those who did not.

All patient characteristics, except for current smoking status, diseases of the musculoskeletal system, and Eye-Ear-Nose-Throat disorders were significantly associated with the end point of intubation or death. A multivariable Cox regression model showed that sex, the hospital in which the patient was treated, obesity, medications according to compassionate use or as part of a clinical trial, cardiovascular disorders, respiratory disorders, neoplasms and diseases of the blood, other infectious diseases, and diseases of the genitourinary system were significantly and independently associated with the composite risk of intubation or death (TABLE 29).

TABLE 29
Associations of baseline clinical characteristics with the composite endpoint of intubation or death in
the cohort of adult patients who had been admitted to AP-HP hospitals for severe COVID-19 (N = 2,846).

| | | | | | | | | Multivariable analysis | |
|---|---|---|---|---|---|---|---|---|---|
| | | | With the | | Without the | | | | Collinearity |
| | | | endpoint | | endpoint | | Crude analysis | | diagnostic |
| | Full sample | | (N = 1,168) | | (N = 1,678) | | HR (95% CI; | HR (95% CI; | (variance |
| | (N = 2,846) | | N (%) | | N (%) | | p-value) | p-value) | inflation factor) |
| Age | | | | | | | | | 1.04 |
| 18 to 50 years | 522 | (18.3) | 137 | (26.2) | 385 | (73.8) | Ref. | Ref. | |
| 51 to 70 years | 1115 | (39.2) | 438 | (39.3) | 677 | ( [symbol] 0.7) | 1.27 (1.05- | 1.07 (0.86- | |
| | | | | | | | 1.54; 0.013*) | 1.33; 0.567) | |
| 71 to 80 years | 520 | (18.3) | 242 | (46.5) | 278 | (53.5) | 1.41 (1.14- | 1.08 (0.84- | |
| | | | | | | | 1.74; 0.001*) | 1.38; 0.585) | |
| More than 80 years | 689 | (24.2) | 351 | (50.9) | 338 | (49.1) | 1.37 (1.13- | 1.16 (0.90- | |
| | | | | | | | 1.67; 0.002*) | 1.50; 0.250) | |
| Sex | | | | | | | | | 1.05 |
| Women | 1064 | (37.4) | 375 | (35.2) | 689 | (64.8) | Ref. | Ref | |
| Men | 1782 | (62.6) | 793 | (44.5) | 989 | (55.5) | 1.28 (1.13- | 1.50 (1.26- | |
| | | | | | | | 1.45; <0.001*) | 1.79; <0.001*) | |
| Hospital | | | | | | | | | 1.02 |
| AP-HP Centre - Paris University, Henri | 722 | (25.4) | 349 | (48.3) | 373 | (51.7) | Ref. | Ref | |
| Mondor University Hospitals and at | | | | | | | | | |
| home hospitalization | | | | | | | | | |
| AP-HP Nord end H [symbol] pitaux | 889 | (31.2) | 313 | (35.2) | 576 | (64.8) | 0 87 (0.74- | 1.05 (0. [symbol] 7- | |
| Universitaires Paris Seine-Saint-Denis | | | | | | | 1.01; 0.067) | 1.27; 0.590) | |
| AP-HP Paris Sacley University | 624 | (21.9) | 245 | (39.3) | 379 | (60.7) | 0.77 (0.65- | 0 77 (0.63- | |
| | | | | | | | 0.91; 0.002*) | 0.93; 0.009*) | |
| AP-HP Sorbonne University | 611 | (21.5) | 261 | (42.7) | 350 | (57.3) | 0.94 (0.80- | 1.00 (0.83- | |
| | | | | | | | 1.10; 0.452) | 1.22; 0.953) | |
| Obesity [symbol] | | | | | | | | | 1.08 |
| Yes | 582 | (20.4) | 293 | (50.3) | 289 | (49.7) | 1.34 (1.17- | 1.31 (1.08- | |
| | | | | | | | 1.53; <0.001*) | 1.59; 0.005*) | |
| No | 2264 | (79.5) | 875 | (38.6) | 1389 | (61.4) | Ref. | Ref | |
| Smoking [symbol] | | | | | | | | | 1.02 |
| Yes | 35 [symbol] | (12.5) | 161 | (45.2) | 195 | (54.8) | 0.94 (0.80- | 0.90 (0.71- | |
| | | | | | | | 1.11, 0.4 [symbol] 3) | 1.14; 0.3 [symbol] 9) | |
| No | 2490 | ( [symbol] 7.5) | 1007 | (40.4) | 1483 | (59.5) | Ref | Ref. | |
| Medication according to | | | | | | | | | 1.06 |
| compassionate use or as part of a | | | | | | | | | |
| clinical trial [symbol] | | | | | | | | | |
| Yes | 806 | (28.3) | 343 | (42.6) | 4 [symbol] 3 | (57.4) | 0.95 (0.84- | 0.80 (0.69- | |
| | | | | | | | 1.08; 0.4 [symbol] 0) | 0.93; 0.003*) | |
| No | 2040 | (71.7) | 825 | (40.4) | 1215 | (59.6) | Ref. | Ref | |
| Other infectious diseases [symbol] | | | | | | | | | 1.08 |
| Yes | 356 | (12.5) | 221 | (62.1) | 135 | (37.9) | 1.71 (1.48- | 1.30 (1.08- | |
| | | | | | | | 1.98; <0.001*) | 1.56; 0.006*) | |
| No | 2490 | (87.5) | 947 | (38.0) | 1543 | (62.0) | Ref | Ref. | |
| Neoplasms and diseases of the blood [symbol] | | | | | | | | | 1.07 |
| Yes | 339 | (11.9) | 177 | (52.2) | 162 | (47.8) | 1.22 (1.04- | 0.79 (0.64- | |
| | | | | | | | 1.43, 0.017*) | 0.98; 0.035*) | |
| No | 2507 | (88.1) | 991 | (39.5) | 1516 | (60.5) | Ref. | Ref | |
| Mental disorders [symbol] | | | | | | | | | 1.09 |
| Yes | 340 | (11.9) | 198 | (58.2) | 142 | (41.8) | 1.37 (1.17- | 1.10 (0.92- | |
| | | | | | | | 1.59; <0.001*) | 1.32; 0.291) | |
| No | 2506 | (88.1) | 970 | (38.7) | 1536 | (61.3) | Ref. | Ref | |
| Diseases of the nervous system [symbol] | | | | | | | | | 1.08 |
| Yes | 292 | (10.3) | 174 | (59.6) | 118 | (40.4) | 1.54 (1.31- | 1.05 (0. [symbol] 6- | |
| | | | | | | | 1.81; <0.001*) | 1.27; 0.639) | |
| No | 2554 | (89.7) | 994 | (38.9) | 1560 | (61.1) | Ref. | Ref | |
| Cardiovascular disorders [symbol] | | | | | | | | | 1.24 |
| Yes | 1020 | (35.8) | 557 | (54.6) | 463 | (45.4) | 1.78 (1.59- | 1.29 (1.09- | |
| | | | | | | | 2.00; <0.001*) | 1.53; 0.003*) | |
| No | 1826 | (64.2) | 61 [symbol] | (33.5) | 1215 | (66. [symbol] ) | Ref. | Ref | |
| Respiratory disorders [symbol] | | | | | | | | | 1.25 |
| Yes | 1704 | (59.9) | 828 | (48.6) | 876 | (51.4) | 1.90 (1.67- | 1.38 (1.10- | |
| | | | | | | | 2.16; <0.001*) | 1.72; 0.005*) | |
| No | 1142 | (40.1) | 340 | (29.8) | 802 | (70.2) | Ref. | Ref | |
| Digestive disorders [symbol] | | | | | | | | | 1.05 |
| Yes | 234 | (8.2) | 121 | (51.7) | 113 | (48.3) | 1.22 (1.01- | 1.03 (0.84- | |
| | | | | | | | 1.47; 0.038*) | 1.26; 0.769) | |
| No | 2612 | (91.8) | 1047 | (40.1) | 1565 | (59.9) | Ref. | Ref. | |
| Dermatological disorders [symbol] | | | | | | | | | 1.02 |
| Yes | 71 | (2.5) | 44 | (62.0) | 27 | (38.0) | 1.38 (1.02- | 1.07 (0.75- | |
| | | | | | | | 1.86; 0.036*)) | 1.52; 0.721) | |
| No | 2775 | (97.6) | 1124 | (40.5) | 1651 | (59.5) | Ref. | Ref. | |
| Diseases of the musculoskeletal | | | | | | | | | 1.04 |
| system [symbol] | | | | | | | | | |
| Yes | 143 | (5.0) | 67 | (48.9) | 7 [symbol] | ( [symbol] 3.1) | 1.02 (0.80- | 0.94 (0.73- | |
| | | | | | | | 1.30; 0.890) | 1.22; 0.655) | |
| No | 2703 | (95.0) | 1101 | (40.7) | 1602 | (59.3) | Ref. | Ref. | |
| Diseases of the genitourinary system [symbol] | | | | | | | | | 1.10 |
| Yes | 429 | (15.1) | 288 | (67.1) | 141 | (32.9) | 1.93 (1.69- | 1.25 (1.01- | |
| | | | | | | | 2.20; <0.001*) | 1.56; 0.043*) | |
| No | 2417 | (84.9) | 880 | (36.4) | 1537 | (63.6) | Ref. | Ref. | |
| Endocrine disorders [symbol] | | | | | | | | | 1.27 |
| Yes | 1042 | (36.6) | 542 | (52.0) | 500 | (48.0) | 1.56 (1.39- | 1.12 (0.95- | |
| | | | | | | | 1.75; <0.001*) | 1.33; 0.173) | |
| No | 1804 | (63.4) | 626 | (34.7) | 1178 | (65.3) | Ref. | Ref. | |
| Eye-Ear-Nose-Throat disorders ^{o} | | | | | | | | | 1.03 |
| Yes | 59 | (2.1) | 31 | ( [symbol] 2.5) | 28 | (47.5) | 1.18 (0.82- | 0.97 (0.89- | |
| | | | | | | | 1.68; 0.370) | 1.35; 0.840) | |
| No | 2787 | (97.9) | 1137 | (40.8) | 1650 | (59.2) | Ref. | Ref. | |

[symbol]

indicates data missing or illegible when filed

The distributions of patient characteristics according to FIASMA medication use are shown in TABLE 30. In the full sample, FIASMA medication use at baseline substantially differed according to all patient characteristics, except for medications according to compassionate use or as part of a clinical trial, and the direction of the associations indicated an older age and greater medical severity of patients receiving FIASMA medication at baseline.

TABLE 30
Characteristics of patients hospitalized for severe COVID-19 receiving
or not receiving an FIASMA medication at baseline (N = 2,846)

| | | | | | |
|---|---|---|---|---|---|
| | | | | Exposed to | Exposed to |
| | | | Exposed to | any FIASMA | any FIASMA |
| Exposed | Not | Non- | any FIASMA | medication vs. | medication vs. non- |
| to any | exposed to | exposed | medication vs. | not exposed | exposed matched group |
| FIASMA | FIASMA | matched | not exposed | Analysis weighted by | Matched analytic |
| medication | medication | group | Crude | inverse-probability- | sample analysis |
| (N = 277) | (N = 2,569) | (N = 277) | analysis | weighting weights | using a 1:1 ratio |
| N (%) | N (%) | N (%) | SMD | SMD | SMD |

Age

| | | | | | | |
|---|---|---|---|---|---|---|
| 18 to 50 years | 29 | 493 | 29 | 0.369 | 0.097 | 0.095 |
| | (10.5%) | (19.2%) | (10.5%) | | | |
| 51 to 70 years | 88 | 1,027 | 99 | | | |
| | (31.8%) | (40.0%) | (35.7%) | | | |
| 71 to 80 years | 63 | 457 | 55 | | | |
| | (22.7%) | (17.8%) | (19.9%) | | | |
| More than | 97 | 592 | 94 | | | |
| 80 years | (35.0%) | (23.0%) | (33.9%) | | | |

Sex

| | | | | | | |
|---|---|---|---|---|---|---|
| Women | 131 | 933 | 118 | 0.224 | 0.034 | 0.094 |
| | (47.3%) | (36.3%) | (42.6%) | | | |
| Men | 146 | 1,636 | 159 | | | |
| | (52.7%) | (63.7%) | (57.4%) | | | |

Hospital

| | | | | | | |
|---|---|---|---|---|---|---|
| AP-HP Centre - | 62 | 660 | 70 | 0.171 | 0.087 | 0.097 |
| Paris | (22.4%) | (25.7%) | (25.3%) | | | |
| University, | | | | | | |
| Henri Mondor | | | | | | |
| University | | | | | | |
| Hospitals and | | | | | | |
| at home | | | | | | |
| hospitalization | | | | | | |
| AP-HP Nord | 76 | 813 | 68 | | | |
| and Hôpitaux | (27.4%) | (31.6%) | (24.5%) | | | |
| Universitaires | | | | | | |
| Paris Seine- | | | | | | |
| Saint-Denis | | | | | | |
| AP-HP Paris | 63 | 561 | 68 | | | |
| Saclay | (22.7%) | (21.8%) | (24.5%) | | | |
| University | | | | | | |
| AP-HP | 76 | 535 | 71 | | | |
| Sorbonne | (27.4%) | (20.8%) | (25.6%) | | | |
| University | | | | | | |

Obesity^{a}

| | | | | | | |
|---|---|---|---|---|---|---|
| Yes | 67 | 515 | 63 | 0.100 | 0.008 | 0.034 |
| | (24.2%) | (20.0%) | (22.7%) | | | |
| No | 210 | 2,054 | 214 | | | |
| | (75.8%) | (80.0%) | (77.3%) | | | |

Smoking^{b}

| | | | | | | |
|---|---|---|---|---|---|---|
| Yes | 46 | 310 | 40 | 0.130 | 0.025 | 0.060 |
| | (16.6%) | (12.1%) | (14.4%) | | | |
| No | 231 | 2,259 | 237 | | | |
| | (83.4%) | (87.9%) | (85.6%) | | | |

Medication according to compassionate use or as part of a clinical trial^{c}

| | | | | | | |
|---|---|---|---|---|---|---|
| Yes | 83 | 723 | 80 | 0.040 | 0.020 | 0.024 |
| | (30.0%) | (28.1%) | (28.9%) | | | |
| No | 194 | 1,846 | 197 | | | |
| | (70.0%) | (71.9%) | (71.1%) | | | |

Other infectious diseases^{d}

| | | | | | | |
|---|---|---|---|---|---|---|
| Yes | 55 | 301 | 50 | 0.225 | 0.040 | 0.046 |
| | (19.9%) | (11.7%) | (18.1%) | | | |
| No | 222 | 2,268 | 227 | | | |
| | (80.1%) | (88.3%) | (81.9%) | | | |

Neoplasms and diseases of the blood^{e}

| | | | | | | |
|---|---|---|---|---|---|---|
| Yes | 46 | 293 | 41 | 0.150 | 0.055 | 0.050 |
| | (16.6%) | (11.4%) | (14.8%) | | | |
| No | 23 | 2,276 | 236 | | | |
| | (83.4%) | (88.6%) | (85.2%) | | | |

Mental disorders^{f}

| | | | | | | |
|---|---|---|---|---|---|---|
| Yes | 70 | 270 | 60 | 0.392 | 0.061 | 0.085 |
| | (25.3%) | (10.5%) | (21.7%) | | | |
| No | 207 | 2,299 | 217 | | | |
| | (74.7%) | (89.5%) | (78.3%) | | | |

Diseases of the nervous system^{g}

| | | | | | | |
|---|---|---|---|---|---|---|
| Yes | 49 | 243 | 41 | 0.242 | 0.047 | 0.078 |
| | (17.7%) | (9.5%) | (14.8%) | | | |
| No | 228 | 2,326 | 236 | | | |
| | (82.3%) | (90.5%) | (85.2%) | | | |

Cardiovascular disorders^{h}

| | | | | | | |
|---|---|---|---|---|---|---|
| Yes | 147 | 873 | 144 | 0.392 | 0.099 | 0.022 |
| | (53.1%) | (34.0%) | (52.0%) | | | |
| No | 130 | 1,696 | 133 | | | |
| | (46.9%) | (66.0%) | (48.0%) | | | |

Respiratory disordersⁱ

| | | | | | | |
|---|---|---|---|---|---|---|
| Yes | 195 | 1,509 | 203 | 0.246 | 0.030 | 0.064 |
| | (70.4%) | (58.7%) | (73.3%) | | | |
| No | 82 | 1,060 | 74 | | | |
| | (29.6%) | (41.3%) | (26.7%) | | | |

Digestive disorders^{j}

| | | | | | | |
|---|---|---|---|---|---|---|
| Yes | 42 | 192 | 35 | 0.244 | 0.015 | 0.073 |
| | (15.2%) | (7.5%) | (12.6%) | | | |
| No | 235 | 2,377 | 242 | | | |
| | (84.8%) | (92.5%) | (87.4%) | | | |

Dermatological disorders^{k}

| | | | | | | |
|---|---|---|---|---|---|---|
| Yes | 14 | 57 | 14 | 0.152 | 0.010 | <0.001 |
| | (5.1%) | (2.2%) | (5.1%) | | | |
| No | 263 | 2,512 | 263 | | | |
| | (94.9%) | (97.8%) | (94.9%) | | | |

Diseases of the musculoskeletal system^{l}

| | | | | | | |
|---|---|---|---|---|---|---|
| Yes | 22 | 121 | 22 | 0.133 | 0.027 | <0.001 |
| | (7.9%) | (4.7%) | (7.94%) | | | |
| No | 255 | 2,448 | 255 | | | |
| | (92.1%) | (95.3%) | (92.1%) | | | |

Diseases of the genitourinary system^{m}

| | | | | | | |
|---|---|---|---|---|---|---|
| Yes | 76 | 353 | 70 | 0.344 | 0.055 | 0.049 |
| | (27.4%) | (13.7%) | (25.3%) | | | |
| No | 201 | 2,216 | 207 | | | |
| | (72.6%) | (86.3%) | (74.7%) | | | |

Endocrine disordersⁿ

| | | | | | | |
|---|---|---|---|---|---|---|
| Yes | 134 | 908 | 135 | 0.266 | 0.037 | 0.007 |
| | (48.4%) | (35.3%) | (48.7%) | | | |
| No | 143 | 1,661 | 142 | | | |
| | (51.6%) | (64.7%) | (51.3%) | | | |

Eye-Ear-Nose-Throat disorders^{o}

| | | | | | | |
|---|---|---|---|---|---|---|
| Yes | 12 | 47 | 12 | 0.145 | 0.008 | <0.001 |
| | (4.3%) | (1.8%) | (4.33%) | | | |
| No | 265 | 2,522 | 265 | | | |
| | (95.7%) | (98.2%) | (95.7%) | | | |

SMD >0.1 (in bold) indicate significant differences.
AP-HP, Assistance Publique-Hôpitaux de Paris; COVID-19, coronavirus disease 2019; FIASMA, Functional Inhibitors of Acid Sphingomyelinase Activity; SMD, standardized mean difference.
^{a}Defined as having a body-mass index higher than 30 kg/m² or an International Statistical Classification of Diseases and Related Health Problems (ICD-10) diagnosis code for obesity (E66.0, E66.1, E66.2, E66.8, and E66.9).
^{b}Current smoking status was self-reported.
^{c}Any medication prescribed as part of a clinical trial or according to compassionate use (e.g., hydroxychloroquine, azithromycin, remdesivir, tocilizumab, sarilumab, or dexamethasone).
^{d}Assessed using ICD-10 diagnosis codes for certain infectious and parasitic diseases (A00-B99).
^{e}Assessed using ICD-10 diagnosis codes for neoplasms (C00-D49) and diseases of the blood and blood-forming organs and certain disorders involving the immune mechanism (D50-D89).
^{f}Assessed using ICD-10 diagnosis codes for mental, behavioral, and neurodevelopmental disorders (F01-F99).
^{g}Assessed using ICD-10 diagnosis codes for diseases of the nervous system (G00-G99).
^{h}Assessed using ICD-10 diagnosis codes for diseases of the circulatory system (I00-199).
ⁱAssessed using ICD-10 diagnosis codes for diseases of the respiratory system (J00-J99).
^{j}Assessed using ICD-10 diagnosis codes for diseases of the digestive system (K00-K95).
^{k}Assessed using ICD-10 diagnosis codes for diseases of the skin and subcutaneous tissue (L00-L99).
^{l}Assessed using ICD-10 diagnosis codes for diseases of the musculoskeletal system and connective tissue (M00-M99).
^{m}Assessed using ICD-10 diagnosis codes for diseases of the genitourinary system (N00-N99).
ⁿAssessed using ICD-10 diagnosis codes for endocrine, nutritional and metabolic diseases (E00-E89).
^{o}Assessed using ICD-10 diagnosis codes for diseases of the eye and adnexa (H00-H59) and diseases of the ear and mastoid process (H60-H95).

In the matched analytic sample and in the full sample after applying the propensity score weights, there were no substantial differences in any characteristic (TABLE 30; FIG. 25).

### Study End Point

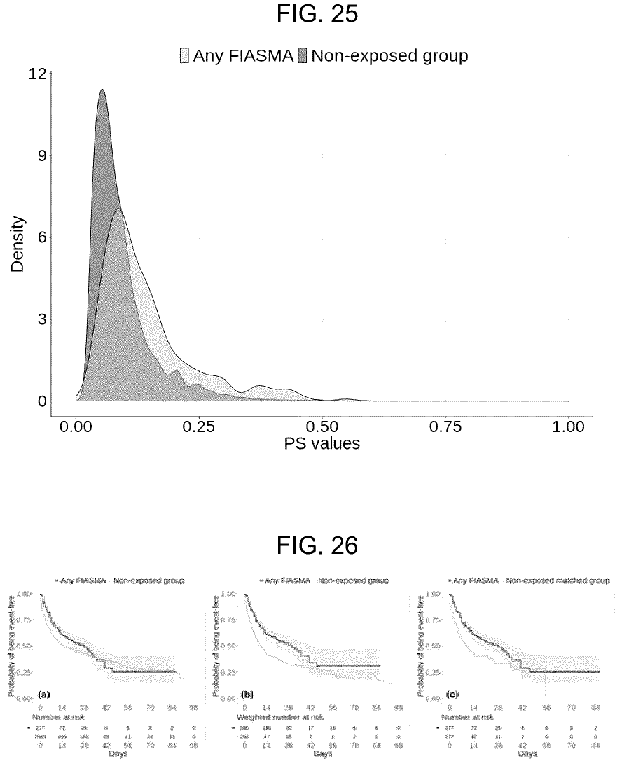

The end point of intubation or death occurred in 104 patients (37.5%) who received an FIASMA medication at baseline and in 1,060 patients (41.4%) who did not. Both the crude unadjusted analysis (hazard ratio (HR)=0.71; 95% confidence interval (CI)=0.58-0.87; P<0.01) and the primary analysis with IPW (HR=0.58; 95% CI=0.46-0.72; P<0.01) showed a significant association between FIASMA medication use at baseline and reduced risk of intubation or death (FIG. 26; TABLE 31). A post hoc analysis indicated that we had 80% power in the crude analysis to detect a HR of at least 0.82/1.21.

TABLE 31
Association between FIASMA medication use at baseline and risk of intubation
or death among patients hospitalized for severe COVID-19 (N = 2,846)

| | | | | Analysis | | |
|---|---|---|---|---|---|---|
| | | | | weighted by | Number of | Univariate |
| | | | Multivariable | inverse- | events/ | Cox regression |
| | Number of | Crude Cox | Cox | probability- | Number of | in a 1:1 |
| | events/ | regression | regression | weighting | patients in | ratio matched |
| | Number of | analysis | analysis | weights | the matched | analytic sample |
| | patients | HR (95% CI; | HR (95% CI; | HR (95% CI; | groups | HR (95% CI; |
| | N/N (%) | P value) | P value) | P value) | N/N (%) | P value) |
| No | 1,064/2,569 | Ref | Ref | Ref | 137/277 | Ref |
| FIASMA | (41.4%) | | | | (49.5%) | |
| medication | | | | | | |
| Any | 104/277 | 0.71 (0.58- | 0.66 (0.53- | 0.58 (0.46- | 104/277 | 0.55 (0.43- |
| FIASMA | (37.5%) | 0.87; 0.001*) | 0.83; <0.001*) | 0.72; <0.001*) | (37.5%) | 0.73; <0.001*) |
| medication | | | | | | |

CI, confidence interval; COVID-19, coronavirus disease 2019; FIASMA, Functional Inhibitor of Acid Sphingomyelinase; HR, hazard ratio.
*Two-sided P value is significant (P < 0.05).

In sensitivity analyses, the multivariable Cox regression model also showed a significant association (HR=0.66; 95% CI=0.53-0.83; P<0.01), as did the univariate Cox regression model in the matched analytic sample using a 1:1 ratio (HR=0.55; 95% CI=0.43-0.73; P<0.01; TABLE 31).

Additional exploratory analyses showed that the use of FIASMA nervous system medications, and specifically FIASMA psycho-analeptic medications, was significantly associated with decreased risk of intubation or death across all analyses (TABLE 32, TABLE 33). Using FIASMA cardiovascular system medication, and specifically FIASMA calcium channel blocker medications, was also significantly associated with reduced risk of intubation or death in the primary IPW analysis, multivariable analysis, and the IPW analysis adjusted for unbalanced covariates. HRs were lower than 1 for most individual FIASMA molecules, but none of them reached statistical significance across all main and sensitivity analyses, except for hydroxyzine and escitalopram (TABLE 33), possibly due to restricted statistical power. Patients receiving any FIASMA medication at baseline, and specifically an FIASMA calcium channel blocker medication, an FIASMA nervous system medication, and specifically an FIASMA psycho-analeptic medication had a significantly reduced risk of intubation or death compared with patients who received paracetamol at baseline (TABLE 34). Reproducing the main analyses while considering venlafaxine, mirtazapine, and citalopram as FIASMA antidepressants did not alter the significance of our results (TABLE 35). Finally, including in the main analyses all patients with and without clinical severity criteria at baseline did not alter the significance of our results (TABLE 36).

TABLE 32
Association of each FIASMA class prescribed at baseline with the composite endpoint
of intubation or death among patients hospitalized for severe COVID-19 (n = 2,846)

| | | | | | Analysis weighted | Number of | Univariate | Cox regression |
|---|---|---|---|---|---|---|---|---|
| | | | | Analysis | by inverse- | events/ | Cox | in 1:2 ratio |
| | | | | weighted | probability- | Number of | regression | matched |
| | | | Multivariable | by inverse- | weighting weights | patients | in 1:2 ratio | analytic samples |
| | Number of | Crude Cox | Cox | probability- | adjusted for | in the | matched | adjusted for |
| | events/ | regression | regression | weighting | unbalanced | matched | analytic | unbalanced |
| | Number of | analysis | analysis | weights | covariates | control | samples | covariates |
| | patients | HR (95% CI; | HR (95% CI; | HR (95% CI; | HR (95% CI; | groups | HR (95% CI; | HR (95% CI; |
| | N/N (%) | P value) | P value) | P value) | P value) | N/N (%) | P value) | P value) |
| No FIASMA | 1,064/2,569 | Ref. | Ref. | Ref. | Ref. | Ref. | Ref. | Ref. |
| medication | (41.4) | | | | | | | |
| FIASMA | 2/9 | 0.39 (0.10- | 0.25 (0.05- | 0.15 (0.02- | NA | 11/18 | 0.24 (0.05- | NA |
| alimentary tract | (22.2) | 1.56; 0.182) | 1.33; 0.104) | 1.21; 0.075) | | (61.1) | 1.12; 0.070) | |
| and metabolism | | | | | | | | |
| medication | | | | | | | | |
| FIASMA | 54/125 | 1.07 (0.81- | 0.82 (0.64- | 0.61 (0.45- | 0.61 (0.46- | 129/250 | 0.80 (0.58- | 0.83 (0.62- |
| cardiovascular | (43.2) | 1.41; 0.650) | 1.06; 0.135) | 0.81; <0.001*) | 0.83; 0.001*)^{a} | (51.6) | 1.10; 0.169) | 1.13; 0.238)^{b} |
| system | | | | | | | | |
| medications | | | | | | | | |
| FIASMA | 38/97 | 0.88 (0.61- | 0.70 (0.49- | 0.56 (0.39- | 0.68 (0.49- | 97/194 | 0.74 (0.48- | 0.75 (0.51- |
| calcium | (39.2) | 1.27; 0.510) | 0.98; 0.037*) | 0.79; <0.001*) | 0.94; 0.020*)^{c} | (50.0) | 1.16; 0.190) | 1.11; 0.149)^{d} |
| channel | | | | | | | | |
| blockers | | | | | | | | |
| Other FIASMA | 19/34 | 1.66 (1.21- | 1.27 (0.96- | NA | NA | 37/68 | 0.91 (0.52- | 0.80 (0.44- |
| cardiovascular | (55.9) | 2.28; 0.002*) | 1.69; 0.100) | | | (54.4) | 1.59; 0.748) | 1.46; 0.469) [symbol] |
| system | | | | | | | | |
| medications | | | | | | | | |
| FIASMA | 61/175 | 0.62 (0.48- | 0.65 (0.49- | 0.51 (0.38- | 0.49 (0.37- | 173/350 | 0.60 (0.44- | 0.61 (0.45- |
| nervous system | (34.9) | 0.80; <0.001*) | 0.88; <0.001*) | 0.69; <0.001*) | 0.64; <0.001*)^{f} | (49.4) | 0.82; 0.002*) | 0.83; 0.002*)^{g} |
| medications | | | | | | | | |
| FIASMA | 59/169 | 0.62 (0.47- | 0.65 (0.48- | 0.51 (0.37- | 0.48 (0.36- | 169/338 | 0.58 (0.42- | 0.60 (0.44- |
| psycho- | (34.9) | 0.80; <0.001*) | 0.87; <0.001*) | 0.70; <0.001*) | 0.63; <0.001*)^{h} | (50.0) | 0.80; <0.001*) | 0.82; 0.001*)ⁱ |
| analeptic | | | | | | | | |
| medications | | | | | | | | |
| FIASMA | 4/13 | 0.68 (0.28- | 0.59 (0.26- | NA | NA | 8/26 | 0.82 (0.25- | 0.80 (0.21- |
| psycholeptic | (30.8) | 1.64; | 1.35; 0.210) | | | (30.8) | 2.73; | 2.98; 0.735) |
| medications | | 0.387) | | | | | 0.746) | |
| FIASMA | 3/7 | 1.02 (0.33- | 0.68 (0.25- | NA | NA | 7/14 | 0.84 (0.22- | NA |
| respiratory | (42.9) | 3.18; 0.970) | 1.81; 0.439) | | | (50.0) | 3.26; 0.798) | |
| system | | | | | | | | |
| medications | | | | | | | | |

CI, confidence interval; FIASMA, Functional Inhibitor of Acid Sphingomyelinase; HR, hazard ratio; NA, not applicable.
^{a}Adjusted for age, cardiovascular disorders, and diseases of the genitourinary system.
^{b}Adjusted for hospital, current smoking status, medication prescribed as part of a clinical trial or according to compassionate use, cardiovascular disorders, respiratory disorders, and diseases of the genitourinary system.
^{c}Adjusted for cardiovascular disorders, diseases of the genitourinary system, and endocrine disorders.
^{d}Adjusted for hospital, current smoking status, diseases of the nervous system, respiratory disorders, and diseases of the genitourinary system.
^{e} Adjusted for hospital, obesity, current smoking status, medication prescribed as part of a clinical trial or according to compassionate use, respiratory disorders, and diseases of the genitourinary system.
^{f}Adjusted for hospital.
^{g}Adjusted for age, sex, current smoking status, and mental disorders.
^{h}Adjusted for hospital.
ⁱAdjusted for age, sex, current smoking status, and mental disorders.
^{j}Adjusted for age, sex, hospital, obesity, current smoking status, medication prescribed as part of a clinical trial or according to compassionate use, mental disorders, and endocrine disorders.
*Two-sided Pvalue is significant (P < 0.05). [symbol]
indicates data missing or illegible when filed

TABLE 33
Association of each FIASMA class and molecule prescribed at baseline with the composite endpoint
of intubation or death among patients hospitalized for severe COVID-19 (N = 2,846).

| | | | | | | Number of | Univariate | |
|---|---|---|---|---|---|---|---|---|
| | | | | Analysis | Analysis | events/ | Cox | Cox regression |
| | | | | weighted by | weighted by | Number of | regression | in 1:2 ratio |
| | | | Multivariable | inverse- | inverse-probability- | patients | in 1:2 ratio | matched analytic |
| | Number of | Crude Cox | Cox | probability- | weighting weights | in the | matched | samples adjusted |
| | events/ | regression | regression | weighting | adjusted for | matched | analytic | for unbalanced |
| | Number of | analysis | analysis | weights | unbalanced covariates | control | samples | covariates |
| | patients | HR (95% CI; | HR (95% CI; | HR (95% CI; | HR (95% CI; | groups | HR (95% CI; | HR (95% CI; |
| | N/N (%) | p-value) | p-value) | p-value) | p-value) | N/N (%) | p-value) | p-value) |
| No FIASMA | 10 [symbol] 4/2 [symbol] | Ref. | Ref. | Ref. | Ref. | Ref. | Ref. | Ref. |
| medication | (41.4) | | | | | | | |
| FIASMA | 2/9 | 0.39 (0.10- | 0.25(0.05- | 0.15 (0.02- | NA | 11/18 | 0.24 (0.05- | NA |
| alimentary tract | (22.2) | 1.58; 0.182) | 1. [symbol] ; 0.104) | 1.21; 0.075) | | ( [symbol] 1.1) | 1.12; 0.070) | |
| and metabolism | | | | | | | | |
| medication | | | | | | | | |
| Loperamide | 2/9 | 0.39 (0.10- | 0.25 (0.05- | 0.15 (0.02- | NA | 11/18 | 0.24 (0.05- | NA |
| | (22.2) | 1.58; 0.182) | 1.33; 0.104) | 1.21; 0.075) | | ( [symbol] 1.1) | 1.12; 0.070) | |
| FIASMA | [symbol] 4/125 | 1.07 (0.81- | 0.82 (0. [symbol] 4- | 0. [symbol] 1 (0.45- | 0. [symbol] 1 (0.46- | 129/256 | 0.80 (0. [symbol] 8- | 0.83 (0. [symbol] 2- |
| cardiovascular | (43.2) | 1.41; [symbol] . [symbol] 0) | 1.0 [symbol] ; 0.135) | 0. [symbol] ; <0.001*) | 0.83; 0.001*) [symbol] | (51.6) | 0.1 [symbol] ; 0.1 [symbol] ) | 1.13; 0.238) [symbol] |
| system | | | | | | | | |
| medications | | | | | | | | |
| FIASMA | 38/97 | 0.88 (0. [symbol] 1- | 0.70 (0.49- | 0.58 (0.39- | 0.68 (0.49- | 97/184 | 0.74 (0.48- | 0 75 (0.51- |
| calcium | (39.2) | 1.27; [symbol] . [symbol] 10) | 0.98; 0.037*) | 0.79; <0.001*) | 0. [symbol] 4; 0.020*) [symbol] | (50.0) | 1.1 [symbol] ; 0.190) | 1.11; 0.14 [symbol] ) [symbol] |
| channel | | | | | | | | |
| blockers | | | | | | | | |
| [symbol] | 38/97 | 0.88 (0. [symbol] 1- | 0.70 (0.49- | 0.58 (0.39- | 0.68 (0.49- | 97/184 | 0.74 (0.48- | 0 75 (0.51- |
| | (39.2) | 1.27; [symbol] . [symbol] 10) | 0.98; 0.037*) | 0.79; <0.001*) | 0. [symbol] 4; 0.020*) [symbol] | (50.0) | 1.1 [symbol] ; 0.190) | 1.11; 0.14 [symbol] ) [symbol] |
| Other FIASMA | 19/34 | 1.68 (1.21- | 1.27 (0. [symbol] - | NA | NA | 37/ [symbol] 8 | 0.91 (0.52- | 0.80 (0.44- |
| cardiovascular | (56. [symbol] ) | 2.28; 0.002*) | 1. [symbol] ; 0.100) | | | (54.4) | 1. [symbol] ; 0.748) | 1.48; 0.4 [symbol] ) [symbol] |
| system | | | | | | | | |
| medications | | | | | | | | |
| [symbol] | 18/33 | 1.61 (1.14- | 1.2 [symbol] (0. [symbol] - | NA | NA | 37/ [symbol] | 0.90 (0.51- | 0.80 (0.44- |
| | ( [symbol] 4. [symbol] ) | 2.27; 0.007*) | 1.70; 0.140) | | | ( [symbol] .1) | 1.58; 0.712) | 1.48; 0.479) [symbol] |
| [symbol] | 1/1 | NA | NA | NA | NA | NA | NA | NA |
| | (100) | | | | | | | |
| FIASMA | [symbol] 1/175 | 0. [symbol] 2 (0.48- | 0. [symbol] 5 (0.4 [symbol] - | 0. [symbol] 1 (0.38- | 0.4 [symbol] (0.37- | 173/35 [symbol] | 0.60 (0.44- | 0 [symbol] .1 (0.45- |
| nervous system | (34.9) | 0.80; <0.001*) | 0.88; <0.001*) | 0. [symbol] ; <0.001*) | 0. [symbol] ; <0.001*) | (49.4) | 0.82; 0.002*) | 0.83; 0.002*) [symbol] |
| medications | | | | | | | | |
| FIASMA | 59/16 [symbol] | 0.62 (0.47- | 0. [symbol] 5 (0.45- | 0.51 (0.37- | 0.4 [symbol] (0.3 [symbol] - | 16 [symbol] /338 | 0.58 (0.42- | 0. [symbol] 0 (0.44- |
| psycho- | (34.9) | 0. [symbol] 0; <0.001*) | 0.87; <0.001*) | 0.70; <0.001*) | 0. [symbol] ; <0.001*) [symbol] | ( [symbol] 0.0) | 0.80; <0.001*) | 0. [symbol] 2; 0.001*) [symbol] |
| analeptic | | | | | | | | |
| medications | | | | | | | | |
| [symbol] | 8/20 | 0.6 [symbol] (0.33- | 0.54 (0.25- | 0.61 (0.29- | NA | 21/40 | 0.54 (0.24- | 0.81 (0.27- |
| | (40.0) | 1.31; 0.22 [symbol] ) | 1.17; 0.120) | 1.38; 0.188) | | (52.5) | 1.21; 0.134) | 1.3 [symbol] ; 0.227) [symbol] |
| [symbol] | 1/4 | 0.58 (0.08- | NA | NA | NA | NA | NA | NA |
| | (25.0) | 4.14; 0. [symbol] 89) | | | | | | |
| [symbol] | 5/12 | 0.69 (0.29- | 0. [symbol] 5 (0.25- | 0.50 (0.20- | NA | 14/24 | 0.44 (0.1 [symbol] - | 0.19 (0.04- |
| | (41.7) | 1. [symbol] ; 0.413) | 1.64; 0.3 [symbol] 8) | 1.2 [symbol] ; 0.151) | | ( [symbol] 8.3) | 1.23; 0.11 [symbol] ) | 0.84; 0.029*) [symbol] |
| [symbol] | 12/42 | 0.51 (0.29- | 0.44 (0.2 [symbol] - | 0.4 [symbol] (0.27- | 0. [symbol] 0 (0.28- | 40/84 | 0.42 (0.22- | 0.27 (0.13- |
| | (28.8) | 0. [symbol] 0; 0.021*) | 0.78; 0.005*) | 0.80; 0.00 [symbol] *) | 1.00; 0.050) [symbol] | (47. [symbol] ) | 0. [symbol] 0; 0.008*) | 0. [symbol] ; <0.001*) |
| [symbol] | 4/14 | 0.4 [symbol] (0.17- | 0.30 (0.0 [symbol] - | 0.34 (0.11- | NA | 1 [symbol] /2 [symbol] | 0.24 (0.08- | 0.18 (0.05- |
| | (2 [symbol] . [symbol] ) | 1.1 [symbol] ; 0.10 [symbol] ) | 1.17; 0.082) | 1.0 [symbol] ; 0.0 [symbol] 2) | | (57.1) | 0.7 [symbol] ; 0.013*) | 0.70; 0.013*) |
| Paroxetine | 1 [symbol] /41 | 0.69 (0.38- | 0. [symbol] (0.38- | 0.57 (0.33- | 0. [symbol] 2 (0.40- | 42/82 | 0.54 (0.31- | 0.48 (0.2 [symbol] - |
| | (3 [symbol] .0) | 1. [symbol] ; 0.2 [symbol] 2) | 1.13; 0.130) | 0. [symbol] ; 0.041*) | 0. [symbol] 7; 0.0 [symbol] *) [symbol] | (51.2) | 0. [symbol] 4; 0.0 [symbol] 0*) | 0. [symbol] 9; 0.028*) |
| Sertraline | 7/21 | 0.58 (0.27- | 0.67 (0.29- | 0. [symbol] 3 (0.23- | 0.82 (0.34- | 24/42 | 0.42 (0.18- | 0.42 (0.1 [symbol] - |
| | (33.3) | 1.21; 0.147) | 1.12; 0.110) | 1. [symbol] 9; 0.357) | 1. [symbol] , 0. [symbol] ) [symbol] | (57.1) | 0. [symbol] ; 0.04 [symbol] *) | 0.91; 0.02 [symbol] *) [symbol] |
| Hydroxyzine | 11/31 | 0. [symbol] 0 (0.33- | 0.43 (0.1 [symbol] - | 0.4 [symbol] (0.2 [symbol] - | NA | 2 [symbol] / [symbol] 2 | 0.4 [symbol] (0.24- | 0.4 [symbol] (0.22- |
| | (3 [symbol] . [symbol] ) | 1.0 [symbol] ; 0.0 [symbol] 0) | 0. [symbol] ; 0.040*) | [symbol] .84; 0.012*) | | (48. [symbol] ) | 0.047*) | 0. [symbol] ; 0.04 [symbol] *) [symbol] |
| FIASMA | 4/13 | 0.68 (0.28- | 0. [symbol] (0.2 [symbol] - | NA | NA | 8/28 | 0.82 (0.25- | 0.80 (0.21- |
| psycholeptic | (30.8) | 1. [symbol] 4; 0.387) | 1.35; 0.210) | | | (30.8) | 2.73; 0.74 [symbol] ) | 2. [symbol] ; 0.735) [symbol] |
| medications | | | | | | | | |
| [symbol] | 1. [symbol] | 0.27 (0.04- | NA | NA | NA | NA | NA | NA |
| | (1 [symbol] .7) | 1. [symbol] 5; 0.1 [symbol] 7) | | | | | | |
| [symbol] | 3/7 | 1.0 [symbol] (0.48- | NA | NA | NA | NA | NA | NA |
| | (42.9) | 2.52; 0.82 [symbol] ) | | | | | | |
| FIASMIS | 3/7 | 1.02 (0.33- | 0.58 (0.2 [symbol] - | NA | NA | 7/14 | 0.84 (0.22- | NA |
| respiratory | (42.9) | 3.18; 0.97 [symbol] ) | 1.81; 0.439) | | | (50.0) | 3.2 [symbol] ; 0.7 [symbol] 8) | |
| system | | | | | | 7/14 | | |
| medications | | | | | | | | |
| [symbol] | 3/7 | 1. [symbol] 2 (0.33- | 0. [symbol] (0.25- | NA | NA | (50.0} | 0.84 (0.22- | NA |
| | (42.9) | [symbol] .1 [symbol] ; 0.97 [symbol] ) | 1.81; 0.439) | | | | 3.2 [symbol] ; 0.7 [symbol] 8) | |

[symbol]

indicates data missing or illegible when filed

TABLE 34
Association between FIASMA medication use at baseline and the endpoint of intubation or death
as compared with paracetamol use at baseline among patients hospitalized for severe COVID-19.

| | | | | | Analysis weighted | | Univariate | Cox |
|---|---|---|---|---|---|---|---|---|
| | | | | Analysis | by inverse- | Number of | Cox | regression in |
| | | | | weighted by | probability- | events/ | regression in | matched analytic |
| | | | Multivariable | inverse- | weighting | Number of | 1:1 or 1:2 | samples |
| | Number of | Crude Cox | Cox | probability- | weights adjusted | patients | ratio matched | adjusted for |
| | events/ | regression | regression | weighting | for unbalanced | in the | analytic | unbalanced |
| | Number of | analysis | analysis | weights | covariates | matched | samples | covariates |
| | patients | HR (95% CI; | HR (95% CI; | HR (95% CI; | HR (95% CI; | groups | HR (95% CI; | HR (95% CI; |
| | N/N (%) | p-value) | p-value) | p-value) | p-value) | N/N (%) | p-value) | p-value) |
| Any FIASMA | 104/277 | 0. [symbol] (0. [symbol] - | 0. [symbol] (0. [symbol] - | 0. [symbol] (0.47- | 0. [symbol] (0.48- | 104/277 | 0.72 (0. [symbol] - | 0.70 (0. [symbol] - |
| medication | (17.5) | 1.12; 0.242) | 0. [symbol] ; 0.0 [symbol] 0*) | 0. [symbol] ; 0.00 [symbol] 9) | 0.8 [symbol] ; 0.00 [symbol] *) | (37. [symbol] ) | 0.94; 0.01 [symbol] *) | 0. [symbol] ; 0.0 [symbol] *) |
| Paracetamol | 192/ [symbol] 2 [symbol] | Ref. | Ref. | Ref. | Ref. | 110/ [symbol] | Ref. | Ref. |
| | ( [symbol] . [symbol] ) | | | | | ( [symbol] .7) | | |
| FIASMA | 2/9 | 0. [symbol] (0. [symbol] - | 0.12 (0.01- | 0.35 (0.0 [symbol] - | 0. [symbol] (0.1 [symbol] - | 2/ [symbol] | 0.37 (0.0 [symbol] - | |
| [symbol] tract | (22.2) | 1. [symbol] ; 0.218) | 1.40; 0. [symbol] 1) | 1.49; 0. [symbol] 7) | 0. [symbol] ; <0.001*) [symbol] | (22.2) | 1.7 [symbol] ; 0.21 [symbol] ) | NA |
| and [symbol] | | | | | | | | |
| medications | | | | | | | | |
| Paracetamol | 192/ [symbol] | Ref. | Ref. | Ref. | Ref. | 8/18 | Ref. | Ref. |
| | (1 [symbol] . [symbol] ) | | | | | (44.4) | | |
| FIASMA | [symbol] 4/125 | 1.22 (0.83- | 0.7 [symbol] (0.52- | 0.74 (0.40- | 0.71 (0. [symbol] - | [symbol] /12 [symbol] | 0.8 [symbol] (0.82- | 0.72 (0. [symbol] 1- |
| cardiovascular | (43.2) | 1.79; 0.219) | 1.10; 0.141) | 1. [symbol] ; 0.331) | 1. [symbol] ; 0. [symbol] ) [symbol] | (43.2) | 1.21; 0.397) | 1.0 [symbol] ; 0.074) |
| system | | | | | | | | |
| medications | | | | | | | | |
| Paracetamol | 1 [symbol] / [symbol] 2 [symbol] | Ref. | Ref. | Ref. | Ref. | 71/ [symbol] | Ref. | Ref. |
| | ( [symbol] . [symbol] ) | | | | | (4 [symbol] . [symbol] ) | | |
| FIASMA [symbol] | 38/97 | 0. [symbol] (0. [symbol] - | 0. [symbol] (0. [symbol] 0- | 0. [symbol] 0 (0. [symbol] - | 0. [symbol] 4 (0.42- | [symbol] / [symbol] | 0. [symbol] 7 (0.4 [symbol] - | 0. [symbol] (0. [symbol] - |
| [symbol] | ( [symbol] . [symbol] ) | 1.22; 0. [symbol] ) | 1. [symbol] ; 0.0 [symbol] ) | 0. [symbol] 3; 0.021*) | 0.97; 0.037*) [symbol] | (39.2) | 0. [symbol] 8; 0.01 [symbol] *) | 1.00; 0.04 [symbol] *) [symbol] |
| Paracetamol | 192/ [symbol] | Ref. | Ref. | Ref. | Ref. | [symbol] 1/ [symbol] 4 | Ref. | Ref. |
| | ( [symbol] . [symbol] ) | | | | | ( [symbol] . [symbol] ) | | |
| [symbol] FIASMA | [symbol] /34 | 1. [symbol] (1. [symbol] - | 1.02 (0. [symbol] - | 0. [symbol] (0. [symbol] 3- | 1.1 [symbol] (0. [symbol] - | [symbol] / [symbol] | 0.74 (0.41- | 0.7 [symbol] (0. [symbol] - |
| cardiovascular | ( [symbol] . [symbol] ) | 2. [symbol] 7; <0.00 [symbol] *) | 1 [symbol] 0; 01. [symbol] 18) | 1. [symbol] 0; 0. [symbol] 4) | 2.13; 0. [symbol] 3) | ( [symbol] . [symbol] ) | 1.34; 0. [symbol] 22) | 1. [symbol] ; [symbol] . [symbol] ) [symbol] |
| system | | | | | | | | |
| medications | | | | | | | | |
| Paracetamol | 192/ [symbol] | Ref. | Ref. | Ref. | Ref. | 33/ [symbol] | Ref. | Ref. |
| | ( [symbol] . [symbol] ) | | | | | (48.5) | | |
| FIASMA [symbol] | [symbol] 1/1 [symbol] | 0.77 (0. [symbol] - | 0. [symbol] (0.4 [symbol] - | 0. [symbol] (0. [symbol] - | 0. [symbol] (0. [symbol] - | [symbol] 1/17 [symbol] | 0.72 (0. [symbol] 2- | 0. [symbol] (0. [symbol] - |
| system | ( [symbol] . [symbol] ) | 1.07; 0.11 [symbol] ) | 0. [symbol] 1; [symbol] .01 [symbol] *) | 0.83; 0.004*) | 0.78; 0.00 [symbol] *) [symbol] | ( [symbol] . [symbol] ) | 1.00; 0.047*) | 0.8 [symbol] ; 0.00 [symbol] *) [symbol] |
| medications | | | | | | | | |
| Paracetamol | 192/ [symbol] | Ref. | Ref. | Ref. | Ref. | 1 [symbol] / [symbol] 0 | Ref. | Ref. |
| | ( [symbol] . [symbol] ) | | | | | (3 [symbol] . [symbol] ) | | |
| FIASMA | [symbol] / [symbol] | 0.77 (0. [symbol] - | 0. [symbol] 4 (0. [symbol] - | 0. [symbol] ( [symbol] . [symbol] - | 0. [symbol] (0. [symbol] - | [symbol] /1 [symbol] | 0.70 (0.51- | 0. [symbol] 3 (0.37- |
| psycholeptic | ( [symbol] 4.9) | 1.07; 0.11 [symbol] ) | 0.91; [symbol] .01 [symbol] *) | 0. [symbol] 0; 0.00 [symbol] *) | 0. [symbol] ; 0.00 [symbol] *) | ( [symbol] . [symbol] ) | 0.97; 0.001*) | 0.74; <0.001*) [symbol] |
| medications | | | | | | | | |
| Paracetamol | 122/ [symbol] | Ref. | Ref. | Ref. | Ref. | 134/ [symbol] | Ref. | Ref. |
| | ( [symbol] . [symbol] ) | | | | | ( [symbol] . [symbol] ) | | |
| FIASMA | 4/ [symbol] | 0. [symbol] (0.22- | 0.41 (0.13- | 0. [symbol] (0.07- | 0. [symbol] (0.12- | 4/13 | 0.54 (0.1 [symbol] - | 0.1 [symbol] (0. [symbol] - |
| [symbol] | ( [symbol] 0.8) | 1. [symbol] ; 01. [symbol] ) | 1.27; 0.122) | 1.72; 0.1 [symbol] 4) | 0.87; 0.0 [symbol] ) [symbol] | (30.8) | 1. [symbol] 7; 0.2 [symbol] ) | 10.4 [symbol] ; 0. [symbol] ) [symbol] |
| medications | | | | | | | | |
| Paracetamol | 1 [symbol] / [symbol] | Ref. | Ref. | Ref. | Ref. | 1 [symbol] /2 [symbol] | Ref. | Ref. |
| | ( [symbol] . [symbol] ) | | | | | ( [symbol] 0.0) | | |
| FIASMA | 3/7 | 1.13 (0. [symbol] - | 0. [symbol] 7 (0. [symbol] - | 0.87 (0.25- | 0.19 (0.04- | 3/7 | 2.23 (0.45- | NA |
| respiratory | (42.9) | [symbol] . [symbol] ; 0. [symbol] ) | [symbol] . [symbol] ; 0. [symbol] 2) | [symbol] .0 [symbol] ; 0.83 [symbol] ) | 0. [symbol] ; 0.04 [symbol] *) [symbol] | (42. [symbol] ) | [symbol] 1.14; 0. [symbol] ) | |
| system | | | | | | | | |
| medication | | | | | | | | |
| Paracetamol | 122/ [symbol] | Ref. | Ref. | Ref. | Ref. | 3/14 | Ref. | Ref. |
| | ( [symbol] . [symbol] ) | | | | | (21.4) | | |

[symbol]

indicates data missing or illegible when filed

TABLE 35
Association between FIASMA medication use at baseline and the composite endpoint of intubation or death among patients
hospitalized for severe COVID-19, while considering venlafaxine, citalopram, and mirtazapine as FIASMAs (N = 2,846).

| | | | | | Analysis weighted | | Univariate | |
|---|---|---|---|---|---|---|---|---|
| | | | | Analysis | by inverse- | Number of | Cox | Cox regression |
| | | | | weighted by | probability- | events/ | regression | in a 1:1 ratio |
| | | | Multivariable | inverse- | weighting weights | Number of | in a 1:1 ratio | matched analytic |
| | Number of | Crude Cox | Cox | probability- | adjusted for | patients | matched | sample adjusted |
| | events/ | regression | regression | weighting | unbalanced | in the | analytic | for unbalanced |
| | Number of | analysis | analysis | weights | covariates [symbol] | matched | sample | covariates [symbol] |
| | patients | HR (95% CI; | HR (95% CI; | HR (95% CI; | HR (95% CI; | group | HR (95% CI; | HR (95% CI; |
| | N/N (%) | p-value) | p-value) | p-value) | p-value) | N/N (%) | p-value) | p-value) |
| No FIASMA | 1,059/ [symbol] , [symbol] | Ref. | Ref. | Ref. | Ref. | 141/287 | Ref. | Ref. |
| medication | (41.4) | | | | | (49.1) | | |
| Any FIASKA | 109/287 | 0.7 [symbol] (0. [symbol] - | 0. [symbol] 7 (0. [symbol] 4- | 0. [symbol] (0.4 [symbol] - | 0. [symbol] (0. [symbol] - | 10 [symbol] /287 | 0. [symbol] 9 (0.45- | 0. [symbol] 0 (0.45- |
| medication | (38.0) | [symbol] . [symbol] ; 0.002*) | 0. [symbol] 4; <0.001*) | 0. [symbol] ; <0.001*) | 0.68; <0.001*) | ( [symbol] . [symbol] ) | 0.78; <0.001*) | 0. [symbol] ; <0.001*) |

^{a} All covariates were balanced.
^{b} Adjusted for hospital.
*Two-sided p-value is significant (p < 0.05).
Abbreviations: HR, hazard ratio; CI, confidence interval. [symbol]
indicates data missing or illegible when filed

TABLE 36
Association between FIASMA medication use at baseline and the composite endpoint of intubation or death
among all patients hospitalized for COVID-19 whatever their baseline clinical severity (N = 14,429).

| | | | | | Analysis weighted | | Univariate | Cox |
|---|---|---|---|---|---|---|---|---|
| | | | | Analysis | by inverse- | Number of | Cox | regression in |
| | | | | weighted by | probability- | events/ | regression | a matched |
| | | | Multivariable | inverse- | weighting weights | Number of | in a 1:1 ratio | analytic sample |
| | Number of | Crude Cox | Cox | probability- | adjusted for | patients | matched | adjusted |
| | events/ | regression | regression | weighting | unbalanced | in the | analytic | for unbalanced |
| | Number of | analysis | analysis | weights | covariates | matched | sample | covariates |
| | patients | HR (95% CI; | HR (95% CI; | HR (95% CI; | HR (95% CI; | groups | HR (95% CI; | HR (95% CI; |
| | N/N (%) | p-value) | p-value) | p-value) | p-value) | N/N (%) | p-value) | p-value) |
| No FIASMA | 1 [symbol] /130 [symbol] | Ref. | Ref. | Ref. | Ref. | 247/ [symbol] 24 | Ref. | Ref. |
| medication | ( [symbol] . [symbol] ) | | | | | (30.0) | | |
| Any FIASKA | 1 [symbol] / [symbol] | 1. [symbol] (1.27- | 0. [symbol] (0.57- | 0. [symbol] 7 (0. [symbol] 7- | 0. [symbol] 2 (0. [symbol] 1- | [symbol] / [symbol] 24 | 0. [symbol] (0. [symbol] 2- | 0. [symbol] (0. [symbol] - |
| medication | ( [symbol] . [symbol] ) | 2.17; 0.00 [symbol] *) | 0. [symbol] 0.041*) | 0. [symbol] 8; <0.001*) | 0. [symbol] ; 0.0 [symbol] ) [symbol] | ( [symbol] . [symbol] ) | 0. [symbol] ; 0.00 [symbol] ) | 0. [symbol] ; 0.0 [symbol] *) |

^{a} Adjusted for age, medication prescribed as part of a clinical trial or according to compassionate use, mental disorders, cardiovascular disorders, respiratory disorders, diseases of the genitourinary system, and endocrine disorders.
*Two-sided p-value is significant (p < 0.05).
Abbreviations: NA, not applicable; HR, hazard ratio; CI, confidence interval. [symbol]
indicates data missing or illegible when filed

TABLE 37
Association between FIASMA medications with low or no affinity for Sigma-1-receptors (i.e., amlodipine, paroxetine, duloxetine,
aripiprazole) and the composite endpoint of intubation or death among adult patients hospitalized for severe COVID-19.

| | | | | | | | | Cox |
|---|---|---|---|---|---|---|---|---|
| | | | | | Analysis weighted | | Univariate | regression in |
| | | | | Analysis | by inverse- | Number of | Cox | a 1:1 ratio |
| | | | | weighted by | probability- | events/ | regression | matched |
| | | | Multivariable | inverse- | weighting weights | Number of | in a 1:1 ratio | analytic sample |
| | Number of | Crude Cox | Cox | probability- | adjusted for | patients | matched | adjusted |
| | events/ | regression | regression | weighting | unbalanced | in the | analytic | for unbalanced |
| | Number of | analysis | analysis | weights | covariates [symbol] | matched | sample | covariates [symbol] |
| | patients | HR (95% CI; | HR (95% CI; | HR (95% CI; | HR (95% CI; | groups | HR (95% CI; | HR (95% CI; |
| | N/N (%) | p-value) | p-value) | p-value) | p-value) | N/N (%) | p-value) | p-value) |
| No FIASMA | 1 [symbol] 84/266 [symbol] | Ref | Ref. | Ref. | Ref. | 77/145 | Ref | Ref. |
| medication | (41.4) | | | | | ( [symbol] .1) | | |
| FIASMA | 55/145 | 0.78 (0. [symbol] - | 0. [symbol] (0. [symbol] - | 0. [symbol] (0.42- | 0. [symbol] (0. [symbol] - | [symbol] /14 [symbol] | 0.47 (0.31- | 0.4 [symbol] (0.32- |
| medications with low | (37.0) | 1.08; 0.1 [symbol] ) | 0. [symbol] ; 0.004*) | 0.74; <0.001*) | 0. [symbol] ; 0.002*) | ( [symbol] . [symbol] ) | 0. [symbol] ; <0.001*) | 0. [symbol] ; <0.0 [symbol] 1*) |
| or no affinity for | | | | | | | | |
| Sigma-1-receptors | | | | | | | | |

*Two-sided p-value is significant (p < 0.05).
^{a} Adjusted for cardiovascular disorders, diseases of the genitourinary system, endocrine disorders, and eye-ear-nose-throat disorders.
^{b} Adjusted for hospital, mental disorders, and respiratory disorders.
Abbreviations: HR, hazard ratio; CI, confidence interval. [symbol]
indicates data missing or illegible when filed

### Discussion

In this multicenter retrospective observational study involving 2,846 adult patients hospitalized for severe COVID-19, we found that FIASMA medication use at hospital admission was significantly and substantially associated with reduced risk of intubation or death, independently of sociodemographic characteristics and medical comorbidities. This association remained significant in multiple sensitivity analyses. Additional exploratory analyses suggest that this association was not explained by one specific FIASMA class or one specific FIASMA medication.

We found that FIASMA medication use among patients hospitalized for severe COVID-19 was significantly and substantially associated with reduced risk of intubation or death, with a 42% risk reduction in the main analysis. This association was not specific to one FIASMA psychotropic class or medication. These findings are in line with prior preclinical^{5,11} and clinical¹⁴⁻¹⁷ evidence that FIASMA antidepressant medications may substantially prevent cells from being infected with SARS-CoV-2 in vitro,^{5, 11} and that several FIASMA medications, such as fluoxetine, hydroxyzine, and amlodipine at their usual respective antidepressant, antihistaminic, and antihypertensive doses, may reduce mortality among patients hospitalized for COVID-19.¹⁴⁻¹⁷

Several other mechanisms could be proposed to explain this association besides the involvement of the ASM/ceramide system.³³ First, antiviral effects (i.e., inhibition of viral replication), of FIASMA medications might underlie this relationship, as suggested by in vitro studies for fluoxetine,⁵ chlorpromazine,³⁴ and amlodipine.¹⁷ However, inhibition of viral replication was not observed with several other FIASMA medications, including paroxetine and escitalopram.³⁵

Second, several FIASMA medications, such as escitalopram or hydroxyzine, have high affinity for Sigma-1 receptors (S1R),^{36, 37} which have been suggested to have potential value in regulating inflammation by inhibiting cytokine production in COVID-19.³⁸ The S1R has been shown to restrict the endonuclease activity of an endoplasmic reticulum stress sensor called Inositol-Requiring Enzyme1 (IRE1) and to reduce cytokine expression, without inhibiting classical inflammatory pathways.^{12, 38} Because several FIASMA medications are S1R agonists in our sample, this mechanism might have overlapped their inhibition effect on ASM. However, when examining the association between the endpoint and several FIASMA medications with low or no affinity for S1R (e.g., amlodipine, paroxetine, duloxetine, and aripiprazole),^{36, 39-42} the main results remained statistically significant (TABLE 37), suggesting that inhibition of ASM could underlie this association independently of S1R.

Finally, this association may be partly mediated by the anti-inflammatory effects of FIASMA medications, which could be explained by inhibition of ASM in endothelial cells and the immune system, and might be independent of Sigma-1 receptors. First, a recent meta-analysis⁴³ of studies conducted in individuals with major depressive disorder following antidepressant treatment, mostly including selective serotonin reuptake inhibitors (SSRIs), supports that, overall, antidepressants may be associated with decreased plasma levels of 4 of 16 tested inflammatory mediators, including IL-10, TNF-α, CCL-2, which are associated with COVID-19 severity,⁴⁴ as well as IL-6, which is highly correlated with disease mortality.^{44, 45} Second, prior in vitro and in vivo studies⁴⁶⁻⁴⁶ suggest that some antipsychotics may have anti-inflammatory effects via glia activation, but that this activity may not be shared by all antipsychotics. However, this anti-inflammatory effect was observed for both FIASMA antipsychotics (e.g., chlorpromazine) and non-FIASMA ones (e.g., haloperidol and risperidone). If the association between FIASMA psychotropic medication use and reduced risk of intubation or death is confirmed, future studies aiming at disentangling these potentially interrelated mechanisms would be needed.

Our study has several limitations. First, there are two possible major inherent biases in observational studies: unmeasured confounding and confounding by indication. However, in the case of FIASMA medications, including several antidepressants and cardiovascular system medications, confounding by indication may typically result in increased adverse medical outcomes associated with these medications,⁴⁹ not better outcomes as suggested by our findings. We tried to minimize the effects of confounding in several different ways. First, we used an analysis with inverse probability weighting to minimize the effects of confounding by indication,³⁰ resulting in nonsubstantial between-group differences in clinical characteristics (all SMD <0.1) in both the IPW primary analysis and the Cox regression analysis in the matched analytic sample. Second, we performed multiple sensitivity analyses, which showed similar results. Finally, although some amount of unmeasured confounding may remain, our analyses adjusted for numerous potential confounders. Other limitations include missing data for some baseline characteristic variables (i.e., 11.5%), which might be explained by the overwhelming of all hospital units during the COVID-19 peak incidence, and different results might have been observed during a lower COVID-19 incidence period. However, imputation of missing data did not alter the significance of our results (data available on request). Second, inflation of type I error might have occurred in exploratory analyses due to multiple testing. Third, data on several FIASMA medications were not available because no patients hospitalized for severe COVID-19 in AP-HP hospitals received them at study baseline during the first epidemic wave. Fourth, this study cannot establish a causal relationship between FIASMA medication use and reduced risk of intubation or death. Fifth, data to approximate the time to onset or duration of the potential effect of FIASMA medications and data on medications taken by patients prior to their hospital admission were not available. Although this may constitute a bias (i.e., considering patients taking an FIASMA medication just before hospital admission as not taking it instead of excluding them from the analyses), the direction of this bias is likely to be toward the null hypothesis, as it may have led to underestimate the association between FIASMAs and reduced risk of intubation or mortality by including individuals taking FIASMAs just before hospital admission in the control group. Sixth, data cutoff is nearly a year ago and represents only the first few months of the COVID-19 pandemic. Furthermore, the relatively limited sample size reduced our ability to examine with adequate statistical power each individual FIASMA medication. Future studies would benefit in replicating our analyses while including a greater number of patients hospitalized for severe COVID-19 at a more recent time of the pandemic when care has substantially progressed as compared with its beginning. Finally, despite the multicenter design, our results may not be generalizable to outpatients or other regions.

In conclusion, in this multicenter observational retrospective study, FIASMA medication use was significantly and substantially associated with reduced risk of intubation or death among adult patients hospitalized for severe COVID-19. These findings show the potential importance of the ASM/ceramide system framework in COVID-19 treatment. They also support the continuation of FIASMA medications in patients with COVID-19. Double-blind controlled randomized clinical trials (RCTs) of these medications in patients with COVID-19 are needed, starting with FIASMA molecules such as fluoxetine, fluvoxamine, escitalopram, or hydroxyzine, which have high in vitro inhibition effect on ASM and are ease of use, including high safety margin, good tolerability, widespread availability, and low cost such that primary care physicians and other providers could prescribe them as soon as onset of symptoms, if their usefulness against COVID-19 was confirmed in RCTs.

### REFERENCES

1. Hoertel, N. et al. A stochastic agent-based model of the SARS-CoV-2 epidemic in France. Nat. Med. 26, 1417-1421 (2020). https://doi.org/10.1038/s41591-020-1001-6.
2. Hoertel, N. et al. Facing the COVID-19 epidemic in NYC: a stochastic agent-based model of various intervention strategies. medRxiv https://doi.org/10.1101/2020.04.23.20076885. [e-pub ahead of print].
3. Chevance, A. et al. Ensuring mental health care during the SARS-CoV-2 epidemic in France: A narrative review. L'Encephale 46, 193-201 (2020).
4. Hoertel, N., Blachier, M., Sánchez-Rico, M., Limosin, F. & Leleu, H. Impact of the timing and adherence to face mask use on the course of the COVID-19 epidemic in France. J. Travel Med. 28(2), taab016 (2021). https://doi.org/10.1093/jtm/taab016.
5. Carpinteiro, A. et al. Pharmacological inhibition of acid sphingomyelinase prevents uptake of SARS-CoV-2 by epithelial cells. Cell Rep. Med. 1, 100142 (2020). https://doi.org/10.1016/j.xcrm.2020.100142.
6. Kornhuber, J. et al. Functional Inhibitors of Acid Sphingomyelinase (FIASMAs): a novel pharmacological group of drugs with broad clinical applications. Cell. Physiol. Biochem. 26, 9-20 (2010). https://doi.org/10.1159/000315101.
7. Gulbins, E. et al. Acid sphingomyelinase-ceramide system mediates effects of antidepressant drugs. Nat. Med. 19, 934-938 (2013). https://doi.org/10.1038/nm.3214.
8. Kornhuber, J. et al. Identification of novel functional inhibitors of acid sphingomyelinase. PLoS One 6, e23852 (2011). https://doi.org/10.1371/journal.pone.0023852.
9. Kornhuber, J. et al. Identification of new functional inhibitors of acid sphingomyelinase using a structure-property-activity relation model. J. Med. Chem. 51, 219-237 (2008). https://doi.org/10.1021/jm070524a.
10. Carpinteiro, A. et al. Inhibition of acid sphingomyelinase by ambroxol prevents SARS-CoV-2 entry into epithelial cells. J. Biol. Chem. 296, 100701 (2021). https://doi.org/10.1016/j.jbc.2021.100701.
11. Schloer, S. et al. Targeting the endolysosomal host-SARS-CoV-2 interface by clinically licensed functional inhibitors of acid sphingomyelinase (FIASMA) including the antidepressant fluoxetine. Emerg. Microbes Infect. 9, 2245-2255 (2020). https://doi.org/10.1080/22221751.2020.1829082.
12. Lenze, E. J. et al. Fluvoxamine vs placebo and clinical deterioration in outpatients with symptomatic COVID-19: A randomized clinical trial. JAMA 324, 2292-2300 (2020). https://doi.org/10.1001/jama.2020.22760.
13. Seftel, D. & Boulware, D. R. Prospective cohort of fluvoxamine for early treatment of coronavirus disease 19. Open Forum Infect. Dis. 8, ofab050 (2021). https://doi.org/10.1093/ofid/ofab050.
14. Hoertel, N. et al. Association between antidepressant use and reduced risk of intubation or death in hospitalized patients with COVID-19: results from an observational study. Mol. Psychiatry https://doi.org/10.1038/s41380-021-01021-4. [e-pub ahead of print].
15. Hoertel, N. et al. Association between hydroxyzine use and reduced mortality in patients hospitalized for coronavirus disease 2019: results from a multicenter observational study. medRxiv https://doi.org/10.1101/2020.10.23.20154302. [e-pub ahead of print].
16. Solaimanzadeh, I. Nifedipine and amlodipine are associated with improved mortality and decreased risk for intubation and mechanical ventilation in elderly patients hospitalized for COVID-19. Cureus. 12(5), e8069 (2020). https://doi.org/10.7759/cureus.8069.
17. Zhang, L.-K. et al. Calcium channel blocker amlodipine besylate therapy is associated with reduced case fatality rate of COVID-19 patients with hypertension. Cell Discov. 6, 1-12 (2020). https://doi.org/10.1038/s41421-020-00235-0.
18. Chouchana, L. et al. Association of antihypertensive agents with the risk of in-hospital death in patients with COVID-19. Cardiovasc. Drugs Ther. https://doi.org/10.1007/si 0557-021-07155-5. [e-pub ahead of print].
19. Hoertel, N. et al. Observational study of haloperidol in hospitalized patients with COVID-19. PLoS One 16, e0247122 (2021). https://doi.org/10.1371/journal.pone.0247122.
20. Hoertel, N. et al. Observational study of chlorpromazine in hospitalized patients with COVID-19. Clin. Drug Investig. 41(3), 221-233 (2021). https://doi.org/10.1007/s40261-021-01001-0.
21. Hoertel, N. et al. Dexamethasone use and mortality in hospitalized patients with coronavirus disease 2019: a multicenter retrospective observational study. Br. J. Clin. Pharmacol. https://doi.org/10.1111/bcp.14784. [e-pub ahead of print]
22. Haut Conseil de la Santé Publique. Statement on the management at home or in a care facility of suspected or confirmed Covid-19 patients. (2020). <https://www.hcsp.fr>.
23. Williamson, E. J. et al. Factors associated with COVID-19-related death using OpenSAFELY. Nature 584, 430-436 (2020). https://doi.org/10.1038/s41586-020-2521-4.
24. Neuraz, A. et al. Natural language processing for rapid response to emergent diseases: Case study of calcium channel blockers and hypertension in the COVID-19 pandemic. J. Med. Internet Res. 22, e20773 (2020). https://doi.org/10.2196/20773.
25. WHO Collaborating Centre for Drug Statistics Methodology ATC/DDD Index. 2021. (2020). <https://www.whocc.no/atc_ddd_index/>.
26. Zhang, Z., Kim, H. J., Lonjon, G., Zhu, Y. & Written on behalf of AME Big-Data Clinical Trial Collaborative Group. Balance diagnostics after propensity score matching. Ann. Transl. Med. 7, 16 (2019). https://doi.org/10.21037/atm.2018.12.10.
27. Austin, P. C. Using the standardized difference to compare the prevalence of a binary variable between two groups in observational research. Commun. Stat.-Simul. Comput. 38, 1228-1234 (2009). https://doi.org/10.1080/03610910902859574.
28. Stuart, E. A., Lee, B. K. & Leacy, F. P. Prognostic score-based balance measures can be a useful diagnostic for propensity score methods in comparative effectiveness research. J. Clin. Epidemiol. 66, S84-S90.e1 (2013). https://doi.org/10.1016/j.jclinepi.2013.01.013.
29. Therneau, T. M. & Patricia, M. Grambsch Modeling Survival Data: Extending the Cox Model. (Springer, New York, 2000).
30. Robins, J. M., Hernán, M. Á. & Brumback, B. Marginal structural models and causal inference in epidemiology. Epidemiology 11, 550-560 (2000).
31. Efron, B. Nonparametric standard errors and confidence intervals. Can. J. Stat. 9, 139-158 (1981). https://doi.org/10.2307/3314608.
32. U.S. National Library of Medicine ClinicalTrials.gov. (2021). <https://clinicaltrials.gov/ct2/results?cond=Covid19&term=paracetamol&cntry=&s tate=&city=&dist=>.
33. Sukhatme, V. P., Reiersen, A. M., Vayttaden, S. J. & Sukhatme, V. V. Fluvoxamine: A review of its mechanism of action and its role in COVID-19. Front. Pharmacol. 12, 652688 (2021).
34. Plaze, M. et al. Inhibition of the replication of SARS-CoV-2 in human cells by the FDA-approved drug chlorpromazine. Int. J. Antimicrob. Agents 57(3), 106274 (2021). https://doi.org/10.1016/j.ijantimicag.2020.106274.
35. Zimniak, M. et al. The serotonin reuptake inhibitor Fluoxetine inhibits SARS-CoV-2 in human lung tissue. Sci. Rep. 11, 5890 (2021).
36. Ishima, T., Fujita, Y. & Hashimoto, K. Interaction of new antidepressants with sigma-1 receptor chaperones and their potentiation of neurite outgrowth in PC12 cells. Eur. J. Pharmacol. 727, 167-173 (2014). https://doi.org/10.1016/j.ejphar.2014.01.064.
37. Ishikawa, M. et al. High occupancy of sigma-1 receptors in the human brain after single oral administration of fluvoxamine: a positron emission tomography study using [11C] SA4503. Biol. Psychiatry 62, 878-883 (2007). https://doi.org/10.1016/j.biopsych.2007.04.001.
38. Zhou, Y. et al. Network-based drug repurposing for novel coronavirus 2019-nCoV/SARS-CoV-2. Cell Discov. 6, 1-18 (2020). https://doi.org/10.1038/s41421-020-0153-3.
39. Hashimoto, K. Activation of sigma-1 receptor chaperone in the treatment of neuropsychiatric diseases and its clinical implication. J. Pharmacol. Sci. 127, 6-9 (2015). https://doi.org/10.1016/j.jphs.2014.11.010.
40. Krutetskaya, Z., Melnitskaya, A., Antonov, V. & Nozdrachev, A. Sigma-1 receptor antagonists haloperidol and chlorpromazine modulate the effect of glutoxim on Na+ transport in frog skin. Doklady Biochem. Biophys. 484(1), 63-65 (2019). https://doi.org/10.1134/S1607672919010186.
41. Vela, J. M. Repurposing sigma-1 receptor ligands for Covid-19 therapy? Front. Pharmacol. 11, 1716 (2020).
42. Gordon, D. E. et al. Comparative host-coronavirus protein interaction networks reveal pan-viral disease mechanisms. Science 370, eabe9403 (2020). https://doi.org/10.1126/science.abe9403.
43. Kohler, C. A. et al. Peripheral alterations in cytokine and chemokine levels after antidepressant drug treatment for major depressive disorder: systematic review and meta-analysis. Mol. Neurobiol. 55, 4195-4206 (2018).
44. Hojyo, S. et al. How COVID-19 induces cytokine storm with high mortality. Inflamm. Regen. 40, 1-7 (2020).
45. Ye, Q., Wang, B. & Mao, J. The pathogenesis and treatment of the Cytokine Storm’ in COVID-19. J. Infect. 80, 607-613 (2020). https://doi.org/10.1016/j.jinf.2020.03.037.
46. Obuchowicz, E., Bielecka-Wajdman, A. M., Paul-Samojedny, M. & Nowacka, M. Different influence of antipsychotics on the balance between pro- and anti-inflammatory cytokines depends on glia activation: an in vitro study. Cytokine 94, 37-44 (2017). https://doi.org/10.1016/j.cyto.2017.04.004.
47. Sugino, H., Futamura, T., Mitsumoto, Y., Maeda, K. & Marunaka, Y. Atypical antipsychotics suppress production of proinflammatory cytokines and up-regulate interleukin-10 in lipopolysaccharide-treated mice. Prog. Neuropsychopharmacol. Biol. Psychiatry 33, 303-307 (2009). https://doi.org/10.1016/j.pnpbp.2008.12.006.
48. Mengozzi, M. et al. Chlorpromazine specifically inhibits peripheral and brain TNF production, and up-regulates IL-10 production, in mice. Immunology 82, 207 (1994).
49. Dragioti, E. et al. Association of antidepressant use with adverse health outcomes: a systematic umbrella review. JAMA Psychiatry 76, 1241-1255 (2019). https://doi.org/10.1001/jamapsychiatry.2019.2859.

### Example 12: Fluvoxamine for the Treatment of COVID-19

The clinical efficacy of fluvoxamine observed in the TOGETHER trial (although no change in virological outcomes was observed) might be related to anti-inflammatory effects via sigma-1 receptor agonism, but several other possible mechanisms warrant further investigation. After the clinical success of fluvoxamine, extrapolation of other therapeutics is inevitable. A logical candidate is fluoxetine, another selective serotonin reuptake inhibitor. To translate in-vitro findings to the clinic, we apply pharmacokinetic modelling to compare free-drug concentration (unbound to plasma proteins) to in-vitro potency.²⁴

Pharmacokinetic parameter estimates for fluvoxamine and fluoxetine were obtained from US prescribing information (Luvox, Solvay pharmaceuticals, US Food and Drug Administration, 1994; and Prozac, Eli Lilly and Company, US Food and Drug Administration, 1987), with additional fluoxetine parameter estimates from a study by Panchaud and colleagues.⁵

One-compartment models with first-order absorption were built and used to simulate fluvoxamine (association constant [Kₐ]=0·356 1/h, oral clearance [CL/F]=30·2 L/h, volume of distribution [V/F]=928 L) and fluoxetine (Kₐ=0·3 1/h, CL/F=8·42 L/h, V/F=690 L) plasma concentrations. The models were used to simulate dosing over 10 days. Free plasma concentrations were calculated for fluvoxamine (20% free) and fluoxetine (5·5% free). Ratio of plasma concentrations to sigma-1 receptor inhibitor constant⁴ and the half-maximal inhibitory concentration²

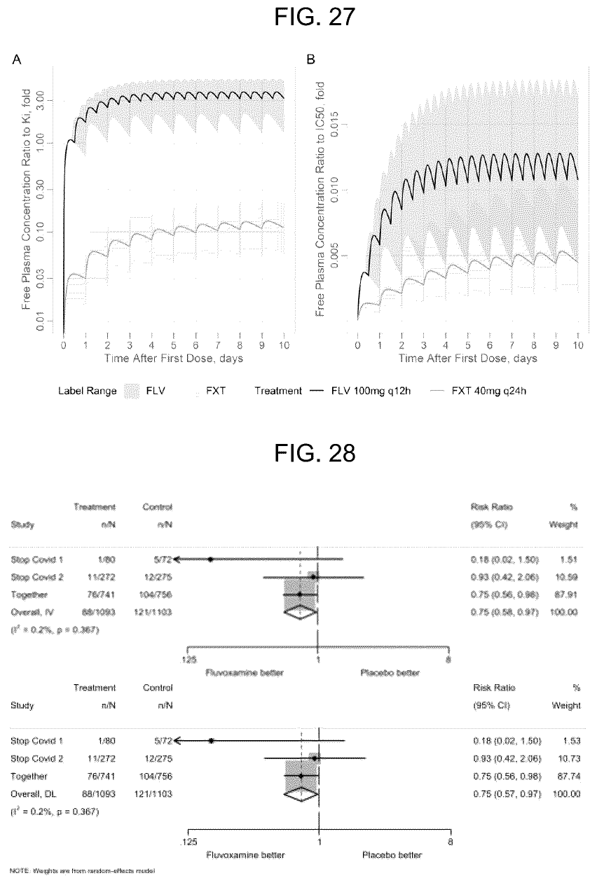

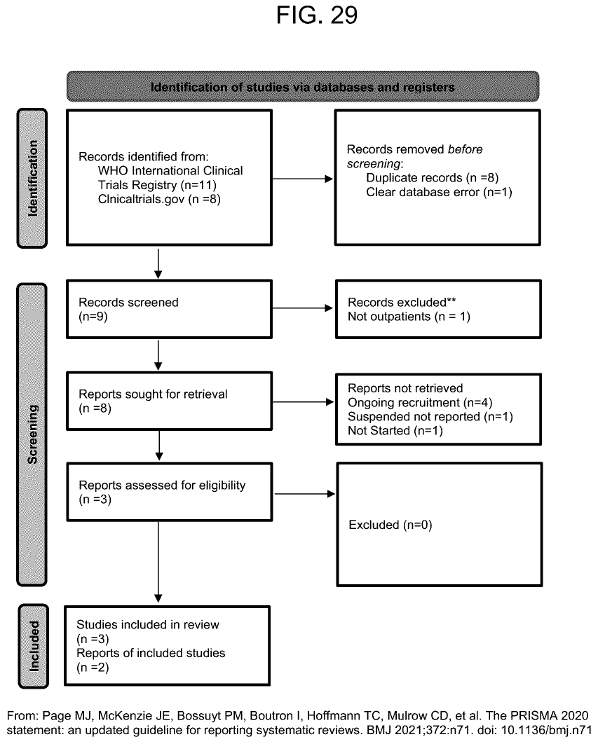

of HEK293T cell lines expressing ACE2 and TMPRSS2 are reported (FIG. 27).

Unbound fluvoxamine concentration is predicted to be three times greater than sigma-1 receptor binding affinity, suggesting anti-inflammatory fluvoxamine concentrations can be reached clinically, in alignment with outcomes from the TOGETHER trial.

¹

By contrast, predicted fluoxetine concentrations were ten times lower than sigma-1 receptor inhibitory constant, suggesting fluoxetine treatment at clinically approved doses might not be successful (FIG. 27). Comparing plasma concentrations to half-maximal inhibitory concentrations of pseudotyped virus, neither fluvoxamine nor fluoxetine appeared promising (concentration or half-maximal inhibitory concentration ratios <1; FIG. 27), which reflects primarily on the relevance of the potential mechanism of action assessed in these experiments.

These observations underscore the challenge of translation of in-vitro experience to clinic. It is important to use non-clinical data that inform the hypothesised primary pharmacology and use pharmacokinetic modelling of anticipated unbound concentrations relative to the non-clinical evaluations in translational efforts.

In conclusion, although the available pharmacological data do not provide a strong translational rationale for fluoxetine for treatment of SARS-CoV-2, available scientific data are very scarce. As such, further exploration of mechanistic and clinical potential of fluoxetine is warranted. Clinical potential should be established within the context of clinical trials where posology can be properly investigated and appropriate safeguards are in place for patients and off-label use should be discouraged.

### REFERENCES

1. Reis G Dos Santos Moreira-Silva E A Silva D C M et al. Effect of early treatment with fluvoxamine on risk of emergency care and hospitalisation among patients with COVID-19: the TOGETHER randomised, platform clinical trial. Lancet Glob Health. 2022; 10: e42-e51
2. Fred S M Kuivanen S Ugurlu H et al. Antidepressant and antipsychotic drugs reduce viral infection by SARS-CoV-2 and fluoxetine show antiviral activity against the novel variants in vitro. bioRxiv. 2021; (published online March 23.) (preprint). https://doi.org/10.1101/2021.03.22.436379
3. Sukhatme V P Reiersen A M Vayttaden S J Sukhatme V V Fluvoxamine: a review of its mechanism of action and its role in COVID-19. Front Pharmacol. 2021; 12652688
4. Brimson J M Prasanth M I Malar D S Brimson S Thitilertdecha P Tencomnao T Drugs that offer the potential to reduce hospitalization and mortality from SARS-CoV-2 infection: the possible role of the sigma-1 receptor and autophagy. Expert Opin Ther Targets. 2021; 25: 435-449
5. Panchaud A Garcia-Bournissen F Csajka C et al. Prediction of infant drug exposure through breastfeeding: population PK modeling and simulation of fluoxetine exposure. Clin Pharmacol Ther. 2011; 89: 830-836

### Example 13: Fluvoxamine for Outpatient COVID-19 to Prevent Hospitalization: A Systematic Review and Meta-Analysis

### Abstract

Importance: Widely available and affordable options for the outpatient management of COVID-19 are needed, particularly therapies that prevent hospitalization.

Objective: Perform a meta-analysis of the available randomized clinical trial evidence for fluvoxamine in the outpatient management of COVID-19.

Data Sources: World Health Organization International Clinical Trials Registry Platform and ClinicalTrials.gov.

Study Selection: Completed outpatient trials with available results which compared fluvoxamine to placebo.

Data Extraction and Synthesis: We followed the PRISMA 2020 guidelines. We extracted study details in terms of inclusion criteria, trial demographics and the pre-specified outcome of all-cause hospitalization. Risk of bias was assessed by the Cochrane Risk of Bias 2 tool. We conducted a frequentist random effects meta-analysis, as well as two sensitivity analyses using a Bayesian random effects meta-analysis with different estimates of prior probability: a weakly neutral prior (50% chance of efficacy with 95% confidence interval for Risk Ratio [RR] between 0.5 and 2) and a moderately optimistic prior (85% chance of efficacy). We contextualized the results by estimating the probability of any effect (RR 51) and moderate effect (RR 50.9) on reducing hospitalization.

Main Outcome(s) and Measure(s): All cause hospitalization.

Results: 2196 participants were included from 3 identified trials. The risk ratios for hospitalization were 0.75 (95% CI, 0.57-0.97) for the frequentist analysis, 0.78 (95% CI 0.58-1.08) for the Bayesian weakly neutral prior, and 0.73 (95% CI, 0.53-1.01) for the Bayesian moderately optimistic prior. Depending on the scenario, the probability of any effect on hospitalization ranged from 94.1% to 98.3% and a moderate effect from 81.6% to 91.1%.

Conclusions and Relevance: Under a variety of assumptions, fluvoxamine shows a high probability of preventing hospitalization in outpatients with COVID-19. While ongoing randomized trials are important to evaluate alternative doses, explore the effectiveness in vaccinated patients, and provide further refinement to these estimates, fluvoxamine could be recommended as a treatment option, particularly in resource-limited settings or persons without access to SARS-CoV-2 monoclonal antibody therapy or direct antivirals.

Question: Does early administration of fluvoxamine prevent hospitalization in symptomatic adult outpatients with confirmed COVID-19?

Findings: In this meta-analysis with Bayesian sensitivity analyses that accounted for varying prior probabilities, there was a high probability (94.1% to 98.3%) that fluvoxamine reduces hospitalization with frequentist risk ratio of 0.75 (95% CI 0.57-0.97).

Meaning: Fluvoxamine is a widely available and inexpensive option that prevents hospitalization in patients with early COVID-19 based on randomized controlled trial evidence to date.

### Introduction

Finding effective outpatient therapies for COVID-19 has been a major research undertaking since the beginning of the pandemic. While therapies such as direct antivirals and engineered monoclonal antibodies represent the current state-of-the art, there are currently challenges with availability, access, administration, and affordability in most areas of the world. Drug repurposing, or the use of existing available and affordable medications for the treatment of COVID-19, is an area of substantial ongoing research interest. The first medication to gain international interest for repurposing was hydroxychloroquine; however, it was ultimately shown to be ineffective in randomized controlled trials¹. A variety of other candidate molecules have been the subject of randomized controlled trials with varying success.²

One such candidate is fluvoxamine, a selective serotonin reuptake inhibitor (SSRI) that is also a potent activator of the sigma-1 receptor which decreases inflammation via reducing endoplasmic reticulum stress.³ In a murine sepsis model, fluvoxamine administration reduced mortality predominantly through this mechanism.⁴ On this preclinical basis, the phase 2 STOP COVID 1 trial (NCT04342663) was conducted.⁵ This 152 patient double-blind, randomized placebo-control trial found fluvoxamine effective at preventing progression to severe COVID-19 defined as: hypoxemia with dyspnea and/or hospital admission. Shortly thereafter, a benefit in preventing hospitalization/death was also reported in an uncontrolled 113 person prospective cohort.⁶

Two larger phase 3 trials have been subsequently completed: STOP COVID2 in the US and Canada (NCT04668950) and the TOGETHER trial in Brazil (NCT04727424).⁷ Both trials were presented to the U.S. National Institutes of Health in August 2021. STOP COVID 2 was stopped for futility in May 2021 after an interim analysis found that the low event rate seen in the trial was associated with a <10% conditional probability of demonstrating efficacy within an attainable sample size based on recruitment rate.⁸ TOGETHER, which had a higher primary outcome event rate, was stopped after demonstrating clinical benefit.⁷ We conducted a systematic review and meta-analysis of outpatient fluvoxamine trials to contextualize the evidence with respect to hospitalization to inform clinical decision making, policy, and guidelines.

### Methods

This systematic review and meta-analysis is reported according to PRISMA 2020.9

Search Strategy, Study selection, and Data Extraction

On Nov. 12, 2021, we searched the World Health Organization International Clinical Trials Registry Platform and ClinicalTrials.gov for all registered clinical trials of fluvoxamine for the treatment of patients with COVID-19. Two independent reviewers screened results for eligibility which included: completed studies of outpatients comparing fluvoxamine to placebo or standard of care. Studies with published or publicly presented data (with the authors' permission) were selected for inclusion.

For included studies we summarized study inclusion criteria and patient demographics. We chose all-cause hospitalization as the primary outcome of interest given the implications for healthcare resource utilization. Two reviewers extracted hospitalization outcome data in each treatment group and these numbers were subsequently verified with the study principal investigators by email. For TOGETHER, the published primary outcome included hospitalization or emergency room observation lasting ≥6 hours. To arrive at a more homogenous outcome, we contacted the TOGETHER trial authors and obtained outcome data on emergency room visits lasting >24 hours and used this as a more representative proxy for hospital admission than ER visit alone. For STOP COVID 2, we obtained demographic and outcomes data directly from the principal investigators (EJL and AMR) based on their presentation to the National Institutes of Health. We only included intention-to-treat analyses even if per protocol analyses suggested larger effect sizes were possible.

### Assessment of Bias

Two independent reviewers assessed each study for bias using the Cochrane risk-of-bias 2 tool for randomized trials.

### Meta-Analysis

We first conducted a frequentist DerSimonian-Laird random effects meta-analysis using STATA version 17 (StataCorp, USA) on the risk ratio scale. With low heterogeneity this would give identical results to a fixed effects model with inverse variance weights. Then, to be more conservative, we conducted two sensitivity analyses with a Bayesian random effects meta-analysis on the log risk ratio scale using bayesmeta in R version 4.1.1.¹⁰ We selected priors based on two estimates of how promising the pre-existing data were. Scenario 1 used a weakly informative neutral prior, given that most treatment effects in medicine fall within this range and because of the STOP COVID 2 results (with a mean mu 0 and a standard deviation 0.355 this corresponds to a 50% chance of efficacy with 95% probability that the risk ratio (RR) would be between 0.5 and 2).¹¹ Scenario 2 used a moderately optimistic prior given the positive results of STOP COVID 1 and the prospective cohort (with a mean mu −0.41 and a standard deviation 0.4 this corresponds to a 85% chance the RR would be 51).¹¹ In both scenarios, the prior for the heterogeneity parameter (tau) used a half-Cauchy distribution with scale 0.10 which is the average heterogeneity for meta-analyses of trials using hospitalization outcomes.¹² Final results were exponentiated to the risk ratio scale for presentation.

The forest plot for the random effects meta-analysis and graphs of the prior and posterior probability distributions based on the pooled RRs were created with STATA version 17 (StataCorp, USA). Probabilities of any effect (RR<1) and a moderate effect (RR 50.9) were calculated by integrating the area under the posterior probability density curves.¹³ Given the low cost of fluvoxamine and decades of established safety, we decided in advance that a moderate effect would correlate to an absolute risk reduction between 0.5% and 1% assuming a 5-10% baseline risk of hospitalization, as observed in the control groups of several outpatient clinical trials. For context, this corresponds to a Number Needed to Treat (NNT) of 100-200.

### Certainty Assessment

Two independent reviewers assessed the certainty of evidence for hospitalization using GRADE methodology (Grading of Recommendations, Assessment, Development and Evaluations)¹⁴.

### Results

The initial search yielded 19 candidate randomized controlled trials and 10 were retained following removal of duplicates (FIG. 28 and TABLE 38). Seven were then excluded for the following reasons: 4 studies because they were still recruiting (NCT04510194, NCT04718480/EUCTR2020-002299-11-HU, NCT04885530, and NCT05087381), 1 study because it recruited only inpatients (IRCT20131115015405N4), 1 study because it was suspended without results (NCT04711863), and 1 was excluded because it had not yet started recruitment (TCTR20210615002).

TABLE 38

| | | | | [symbol] | | | | [symbol] |
|---|---|---|---|---|---|---|---|---|
| | | | | [symbol] | | | [symbol] | [symbol] |
| [symbol] | [symbol] | [symbol] | [symbol] | [symbol] | [symbol] | [symbol] | [symbol] | [symbol] |
| [symbol] | [symbol] | [symbol] | No | [symbol] | [symbol] | [symbol] | [symbol] | No |
| [symbol] COVID-19 [symbol] | | | | [symbol] | [symbol] | | | |
| [symbol] | [symbol] | [symbol] | Yes | [symbol] | [symbol] | [symbol] | Yes | Yes |
| [symbol] | | | | [symbol] | | | | |
| [symbol] | | | | | | | | |
| [symbol] | | | | | | | | |
| [symbol] | | | | | | | | |
| COVID-OUT [symbol] | [symbol] | [symbol] | Yes | [symbol] | [symbol] | [symbol] | No | No |
| [symbol] | | | | [symbol] | | | | |
| [symbol] (COVID- [symbol] ) | | | | | | | | |
| [symbol] | [symbol] | [symbol] | Yes | [symbol] | [symbol] | [symbol] | Yes | Yes |
| [symbol] | | [symbol] | | [symbol] | | | | |
| [symbol] | | | | | | | | |
| [symbol] | [symbol] | [symbol] | Yes | [symbol] | [symbol] | [symbol] | No | No |
| [symbol] COVID-19 | | [symbol] | | [symbol] | | | | |
| [symbol] | [symbol] | [symbol] | Yes | [symbol] | [symbol] | [symbol] | No | No |
| [symbol] | [symbol] | | | [symbol] | | | | |
| (COVID-19) [symbol] | [symbol] | | | | | | | |
| [symbol] | [symbol] | [symbol] | Yes | [symbol] | [symbol] | [symbol] | Yes | Yes |
| [symbol] | | | | [symbol] | | | | |
| [symbol] | | | | | | | | |
| COVID- [symbol] [symbol] | | | | | | | | |
| [symbol] | | | | | | | | |
| [symbol] COVID-19 [symbol] | [symbol] | [symbol] | Yes | [symbol] | [symbol] | [symbol] | No | No |
| [symbol] | | | | [symbol] | | | | |
| [symbol] | [symbol] | [symbol] | Yes | [symbol] | [symbol] | [symbol] | No | No |
| [symbol] COVID-19 | | | | [symbol] | | | | |
| [symbol] | | | | | | | | |
| [symbol] | [symbol] | [symbol] | Yes | [symbol] | [symbol] | [symbol] | [symbol] | No |
| [symbol] | | | | [symbol] | | | | |
| [symbol] | | | | | | | | |
| [symbol] | | | | | | | | |
| [symbol] | | | | | | | | |
| [symbol] COVID- [symbol] | | | | | | | | |
| [symbol] | | | | | | | | |
| [symbol] | | | | | | | | |
| [symbol] | | | | | | | | |
| [symbol] | | | | | | | | |

[symbol]

indicates data missing or illegible when filed

### Included Studies

The remaining 3 trials (STOP COVID 1 (n=152)⁵, STOP COVID 2 (n=547), and TOGETHER (n=1497)⁷) included a total of 2196 analyzed patients (TABLE 39). All 3 were placebo-controlled randomized controlled trials that recruited unvaccinated, symptomatic adults with microbiologically confirmed SARS-CoV-2 infection who were within 6-7 days of infection and not requiring oxygen. Whereas STOP COVID 1 took all patients, STOP COVID 2 and TOGETHER enriched their population for at least one at-risk feature for deterioration. Overall, the median age of subjects was between 46 and 50, 55-72% of participants were female, 44-56% were obese. Most patients in the STOP COVID trials self-reported as Caucasian, in TOGETHER, 96% self-reported as mixed race. The risk of bias was considered low for all trials by both reviewers.

TABLE 39

| | | | | | [symbol] | |
|---|---|---|---|---|---|---|
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| | | [symbol] | [symbol] | [symbol] | | |
| | | [symbol] | [symbol] | [symbol] | | |
| | | | [symbol] | [symbol] | | |
| [symbol] | [symbol] | [symbol] | [symbol] | [symbol] | [symbol] | [symbol] |
| | | [symbol] | [symbol] | [symbol] | | |
| | | [symbol] | [symbol] | [symbol] | | |
| | | | [symbol] | [symbol] | | |
| | | | [symbol] | | | |
| [symbol] | [symbol] | [symbol] | [symbol] | [symbol] | [symbol] | [symbol] |
| | | [symbol] | [symbol] | [symbol] | | |
| | | [symbol] | [symbol] | [symbol] | | |
| | | | [symbol] | [symbol] | | |
| | | | [symbol] | | | |

[symbol]

[symbol]

[symbol]

indicates data missing or illegible when filed

### Meta-Analysis

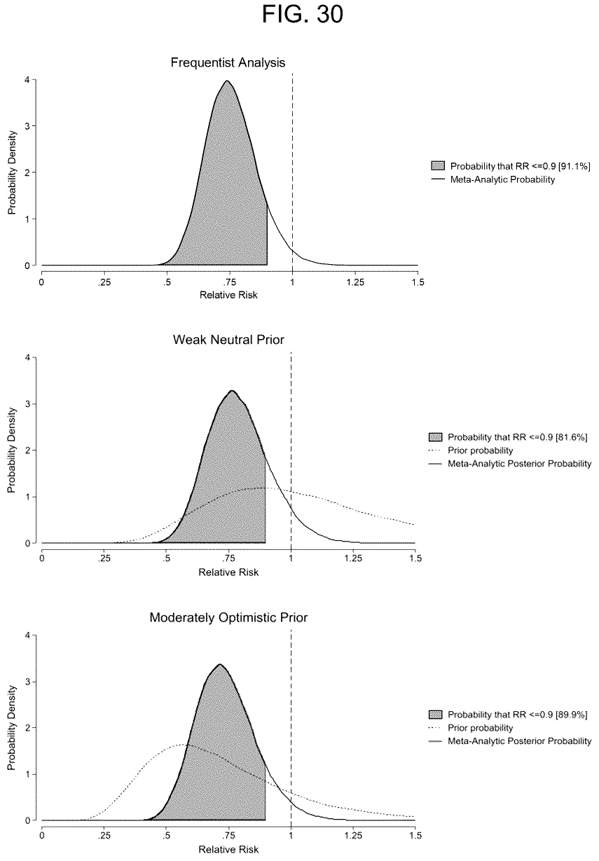

In the frequentist meta-analysis, the pooled RR in favor of fluvoxamine was 0.75 (95% CI 0.57-0.97; I² 0.2%) (FIG. 28). Correspondingly, there was a 98.3% probability that fluvoxamine prevents hospitalization and a 91.1% probability of at least a moderate effect. In the Bayesian sensitivity analysis, the RR in favor of fluvoxamine was 0.78 (95% CI 0.58-1.08) for the weakly neutral prior, and 0.73 (95% CI 0.53-1.01) for the moderately optimistic prior (TABLE 40). The probability of any effect ranged from 94.1% to 98.3%, and of moderate effect from 81.6% to 91.1% (FIG. 30).

TABLE 40

| Scenario | Pooled risk ratio (95% CI) | Probability RR <1 | Probability RR ≤0.9 |
|---|---|---|---|
| Frequentist analysis | 0.75 (0.57-0.97) | 98.3% | 91.1% |
| Weakly neutral | 0.78 (0.58-1.08) | 94.1% | 81.6% |
| Moderately optimistic | 0.73 (0.53-1.01) | 97.2% | 89.9% |

We believe the certainty of the evidence is moderate. Although all three trials were placebo-controlled randomized trials, there is some inconsistency in the findings given STOP COVID 2 was terminated for futility. Although there is some risk of bias in the TOGETHER trial, we attempted to address this by limiting the primary outcome to emergency room visits that were 24 hours or longer, thus removing some of the concern for bias that could have been introduced through the inclusion of shorter visits (6-24 hours in length). A moderate strength recommendation in favor of fluvoxamine could be considered in certain clinical scenarios detailed in the discussion. According to the GRADE methodology, such a recommendation is reasonable when shared decision making is expected. Weaker recommendations usually apply when risks start to approach the benefits, or when significant resources are required for the intervention, neither of which is the case for fluvoxamine.

### Discussion

Based on all currently available clinical trial data, it is very likely that fluvoxamine has at least a moderate effect on preventing COVID-19 hospitalization under a variety of assumptions. By comparison, outpatient trials with hydroxychloroquine¹ and ivermectin¹⁵ have not shown efficacy and yet these agents continue to be prescribed. Further RCT data from the ongoing identified studies like CovidOut (NCT04510194) and ACTIV-6 (NCT04885530) will help refine these estimates, however, both trials are using 50 mg twice daily which, if unsuccessful, could indicate that 100 mg twice daily is the minimum effective dose. Recently, based on STOP COVID 1 and the TOGETHER trial, the Infectious Diseases Society of America recommended against the use of fluvoxamine outside of clinical trial settings.¹⁶ Based on our analysis and coupled with worldwide accessibility, decades of safety data, and a current price of approximately $1/day,^{17,18} fluvoxamine seems reasonable for high-risk outpatients who do not have access to SARS-CoV-2 monoclonal antibodies, direct antivirals, or clinical trials. Even at a number to treat of 200 (absolute risk reduction 0.5%) the corresponding cost to prevent admission would only be $2800. Clinicians who prescribe fluvoxamine for COVID-19 should familiarize themselves with relative contraindications and drug-drug interactions, including the need to limit caffeine (TABLE 41).

TABLE 41
Considerations for Relative Contraindications to Fluvoxamine

| Patient Factors | Reasoning (if not obvious) |
|---|---|
| Allergy to fluvoxamine | |
| Moderate to severe depression within 6 | If the patient would need to be switched to |
| weeks of enrollment | fluvoxamine from another agent due to drug- |
| | interactions, this would ideally be done with |
| | explicit supervision |
| Previous or current diagnosis of manic | If the patient would need to be switched to |
| depression/bipolar disorder | fluvoxamine from another agent or if there would |
| | be concern that adding fluvoxamine might trigger |
| | a manic episode |
| Hepatic impairment defined as known | Fluvoxamine metabolism is altered in patients with |
| Cirrhosis of any severity | cirrhosis |
| Hospitalization for gastrointestinal or other | Fluvoxamine can impact platelet aggregation and |
| non-traumatic bleeding within the last year | these patients were excluded from the trial. This |
| | decision could be individualized. |
| Concurrent Medications | |
| Caffeine | Fluvoxamine leads to substantial increases in |
| | caffeine levels. In the trial, we encouraged no |
| | caffeine for participants. At the very least they |
| | were told avoid more than 1 small cup of coffee's |
| | worth of caffeine (and to stop caffeine if they felt it |
| | was “too energizing”). |
| Patients taking warfarin | Increased bleeding risk due to increased AUC of |
| | warfarin |
| Patients taking clopidogrel | Increased risk of ischemic event due to |
| | metabolism |
| Patients taking 2 or more of the following: | Assuming NSAIDs cannot be held. Fluvoxamine |
| aspirin, NSAIDS, ticlopidine, prasugrel, | can impact platelet aggregation and these patients |
| ticagrelor, direct oral anticoagulants | were excluded from the trial. This decision could |
| | be individualized. |
| Donepezil | This is a Sigma-1-receptor (S1R) agonist and we |
| | excluded patients from the trial given that |
| | fluvoxamine was being used for its S1R activity |
| Other antidepressant medications | For any patient already on a tricyclic |
| | antidepressant, SSRI, or SNRI, we evaluated |
| | whether it could be held or reduced under medical |
| | supervision during the time they were prescribed |
| | fluvoxamine. If the patient was taking a low dose |
| | of another medication (e.g., citalopram 10 mg) and |
| | there was low risk of serotonin syndrome, |
| | concurrent use was allowed. |
| Use within 14 days of an MAO inhibitor [e.g., | Important drug interactions risking serotonin |
| Isocarboxazid (Marplan), Phenelzine (Nardil), | syndrome |
| Selegiline (Emsam), Tranylcypromine | |
| (Parnate)] | |
| Patients taking astemizole, cisapride, | Contraindicated due to hepatic CYP3A4 |
| mesoridazine, ramelteon, or terfenadine | interaction |
| Patients taking phenytoin or valproic acid | Potential interaction leading to seizure |
| Patients who are taking mirtazapine, | If these drugs could not be held, there was a risk |
| melatonin, tramadol, or triptan medications | of drug interaction increasing levels of these |
| | medicines |
| Participants taking alosetron, clozapine, | Drugs are primarily metabolized by CYP1A2, |
| flutamide, mexiletine, olanzapine, rasagiline, | which is inhibited by fluvoxamine. |
| ropinirole, tacrine, theophylline, tizanidine, | |
| triamterene | |
| Diazepam or alprazolam users | Due to interactions, we recommend reducing the |
| | dose by 25% unless the patient has a known |
| | seizure disorder (in which case they were |
| | excluded). |

Strengths of our analysis are the use of all available data and consistency of results by both a classic random effects meta-analysis and a Bayesian approach with multiple prior probability estimates. For all analyses, we quantified the overall probability of any effect and a moderate effect of fluvoxamine on hospitalization to help with decision making. Further, we restricted the TOGETHER trial emergency room visits to those >24 hours to address concerns¹⁶ about whether a six-hour visit is an accurate proxy for healthcare utilization or marker of clinical deterioration.

We utilized the intention-to-treat analysis, reflecting physician prescribing. In the TOGETHER trial, results were better in the per-protocol analysis.⁷

Limitations include variability in healthcare practices, resource availability, and circulating variants between trials, leading to differences in the baseline event rates and the associated absolute risk reduction. While we have attempted to correct for subjectivity by limiting this analysis to hospitalization, indeed hospitalization decisions may vary between geographic areas and even time points based on systemic burden. Nonetheless, all-cause hospitalization is the most common important outcome of outpatient COVID-19 trials because ICU admission or death would require studies that were prohibitively large. Additionally, all three trials excluded fully vaccinated individuals, whose rates of hospitalization are greatly reduced, and therefore any estimates of absolute effect size would likely be an overestimate in vaccinated patients. Another limitation is the inclusion of only 3 trials to date. Using a living systematic review approach will address this clinical question. This is planned in order to address this concern and to rapidly incorporate emerging evidence.

Ongoing randomized controlled trials of fluvoxamine should continue, particularly those studying lower 50 mg doses (which will be better tolerated), evaluating efficacy in vaccinated individuals, or studying the related SSRI fluoxetine which is on the World Health Organization's list of essential medications. In the meantime, fluvoxamine is an immediately available, safe, and inexpensive treatment option with a high probability of moderate efficacy. It could be recommended as a treatment option for patients without contraindication, particularly in resource-limited settings or those without access to monoclonal antibodies or direct antivirals.

### REFERENCES

1. Skipper C P, Pastick K A, Engen N W, et al. Hydroxychloroquine in Nonhospitalized Adults With Early COVID-19□: A Randomized Trial. Ann Intern Med. 2020; 173(8):623-631. doi:10.7326/M20-4207
2. Ezer N, Belga S, Daneman N, et al. Inhaled and intranasal ciclesonide for the treatment of covid-19 in adult outpatients: CONTAIN phase II randomised controlled trial. BMJ. 2021; 375:e068060. doi:10.1136/bmj-2021-068060
3. Sukhatme V P, Reiersen A M, Vayttaden S J, Sukhatme V V. Fluvoxamine: A Review of Its Mechanism of Action and Its Role in COVID-19. Front Pharmacol. 2021; 12:652688. doi:10.3389/fphar.2021.652688
4. Rosen D A, Seki S M, Fernéndez-Castañeda A, et al. Modulation of the sigma-1 receptor-IRE1 pathway is beneficial in preclinical models of inflammation and sepsis. Sci Transl Med. 2019; 11(478):eaau5266. doi:10.1126/scitranslmed.aau5266
5. Lenze E J, Mattar C, Zorumski C F, et al. Fluvoxamine vs Placebo and Clinical Deterioration in Outpatients With Symptomatic COVID-19: A Randomized
6. Seftel D, Boulware D R. Prospective Cohort of Fluvoxamine for Early Treatment of Coronavirus Disease 19. Open Forum Infect Dis. 2021; 8(2):ofab050. doi:10.1093/ofid/ofab050
7. Reis G, Dos Santos Moreira-Silva E A, Silva D C M, et al. Effect of early treatment with fluvoxamine on risk of emergency care and hospitalisation among patients with COVID-19: the TOGETHER randomised, platform clinical trial. Lancet Glob Health. Published online Oct. 27, 2021:S2214-109X(21)00448-4. doi:10.1016/S2214-109X(21)00448-4
8. Lenze E J. Aug. 20, 2021: Fluvoxamine for Early Treatment of COVID-19: The STOP COVID Clinical Trials (Eric Lenze, MD). Rethinking Clinical Trials. Published Aug. 24, 2021. Accessed Nov. 1, 2021. https://rethinkingclinicaltrials.org/news/august-20-2021-fluvoxam ine-for-early-treatment-of-covid-19-the-stop-covid-clinical-trials-eric-lenze-md/
9. Page M J, McKenzie J E, Bossuyt P M, et al. The PRISMA 2020 statement: an updated guideline for reporting systematic reviews. BMJ. 2021; 372:n71. doi:10.1136/bmj.n71
10. Röver C. Bayesian Random-Effects Meta-Analysis Using the bayesmeta R Package. J Stat Softw Vol 1 Issue June 2020. Published online Apr. 27, 2020. https://www.jstatsoft.org/v093/i06
11. Zampieri F G, Casey J D, Shankar-Hari M, Harrell F E, Harhay M O. Using Bayesian Methods to Augment the Interpretation of Critical Care Trials. An Overview of Theory and Example Reanalysis of the Alveolar Recruitment for Acute Respiratory Distress Syndrome Trial. Am J Respir Crit Care Med. 2021; 203(5):543-552. doi:10.1164/rccm.202006-2381CP
12. Turner R M, Jackson D, Wei Y, Thompson S G, Higgins J P T. Predictive distributions for between-study heterogeneity and simple methods for their application in Bayesian meta-analysis. Stat Med. 2015; 34(6):984-998. doi:10.1002/sim.6381
13. Lee T C, McDonald E G, Butler-Laporte G, Harrison L B, Cheng M P, Brophy J M. Remdesivir and systemic corticosteroids for the treatment of COVID-19: A Bayesian re-analysis. Int J Infect Dis. 2021; 104:671-676. doi:10.1016/j.ijid.2021.01.065
14. Guyatt G H, Oxman A D, Vist G E, et al. GRADE: an emerging consensus on rating quality of evidence and strength of recommendations. BMJ. 2008; 336(7650):924-926. doi:10.1136/bmj.39489.470347.AD
15. López-Medina E, López P, Hurtado I C, et al. Effect of Ivermectin on Time to Resolution of Symptoms Among Adults With Mild COVID-19: A Randomized Clinical Trial. JAMA. 2021; 325(14):1426-1435. doi:10.1001/jama.2021.3071
16. Bhimraj A, Morgan R, Hirsch Shumaker A, et al. Infectious Diseases Society of America Guidelines on the Treatment and Management of Patients with COVID-19 Version 5.5.2. Published Nov. 5, 2021. Accessed Nov. 8, 2021. https://www.idsociety.org/practice-guideline/covid-19-guideline-treatment-and-management/
17. Régie de l'assurance maladie du Québec (RAMQ). List of medications, Sep. 29, 2021. List of medications, Sep. 29, 2021. Published Oct. 1, 2021. Accessed Oct. 27, 2021. https://www.ramq.gouv.qc.ca/en/media/12091
18. Fluvoxamine Prices, Coupons & Savings Tips. GoodRx. Accessed Nov. 8, 2021. https://www.goodrx.com/fluvoxamine

What is claimed is:

1. A method of (i) preventing progression to severe COVID-19 or hospitalization; or (ii) treating or preventing SARS-CoV-2 infection, COVID-19, or COVID-19 symptoms in a subject comprising:
administering a therapeutically effective amount of a pharmaceutical agent comprising an antidepressant medication, an anxiolytic medication, a psychotropic, a sigma-1 receptor (S1R) antagonist or agonist, a cationic amphiphilic drug, a selective serotonin reuptake inhibitor (SSRI), or functional inhibitor of acid sphingomyelinase (FIASMA), or a combination thereof.

2. The method of claim 1, wherein the pharmaceutical agent has S1R binding activity.

3. The method of claim 1, wherein the pharmaceutical agent has a Ki value of less than 3000 nM to S1R or IC₅₀ value less than 100 μM.

4. The method of claim 1, wherein the pharmaceutical agent is one or more antidepressant selected from the group consisting of: Fluoxetine; Fluvoxamine; Citalopram; Chlorpromazine; Escitalopram; Paroxetine; Sertraline; Vilazodone; Vortioxetine; Amitriptyline; Clomipramine; Desipramine; Doxepin; Imipramine; Nortriptyline; Nefazodone; Trazodone; Desvenlafaxine; Duloxetine; Levomilnacipran; Venlafaxine; Bupropion; or Mirtazapine.

5. The method of claim 1, wherein the pharmaceutical agent is a selective serotonin reuptake inhibitor (SSRI).

6. The method of claim 5, wherein the pharmaceutical agent is a SSRI selected from Citalopram, Escitalopram, Fluoxetine, Fluvoxamine, Paroxetine, or Sertraline.

7. The method of claim 5, wherein the SSRI is a selective serotonin (5-HT) reuptake inhibitor (SSRI) of the 2-aminoethyl oxime ethers of aralkylketones chemical series.

8. The method of claim 1, wherein the pharmaceutical agent is a sigma-1 receptor (S1R) agonist which activates the sigma-1 receptor (S1R).

9. The method of claim 1, wherein the pharmaceutical agent is:
an antidepressant medication; and
a sigma-1 receptor (S1R) agonist, a cationic amphiphilic drug, a selective serotonin reuptake inhibitor (SSRI), and a functional inhibitor of acid sphingomyelinase (FIASMA).

10. The method of claim 1, wherein the pharmaceutical agent is fluvoxamine.

11. The method of claim 1, wherein the pharmaceutical agent is a norepinephrine-dopamine reuptake inhibitor (NDRI).

12. The method of claim 11, wherein the NDRI is bupropion.

13. The method of claim 1, wherein the antidepressant is a tricyclic antidepressant.

14. The method of claim 13, wherein the tricyclic antidepressant is Amitriptyline, Clomipramine, Desipramine, Doxepin, Imipramine, or Nortriptyline.

15. The method of claim 1, wherein the antidepressant is a phenylpiperazine.

16. The method of claim 15, wherein the phenylpiperazine is nefazodone or Trazodone.

17. The method of claim 1, wherein the antidepressant is a Serotonin Norepinephrine Reuptake Inhibitors (SNRI).

18. The method of claim 17, wherein the SNRI is Desvenlafaxine, Duloxetine, Levomilnacipran, or Venlafaxine.

19. The method of claim 1, wherein the antidepressant is a tetracyclic piperazine-azepine.

20. The method of claim 19, wherein the tetracyclic piperazine-azepine is Mirtazapine.

21. The method of claim 1, wherein the anxiolytic medication is a Benzodiazepine.

22. The method of claim 21, wherein the Benzodiazepine is Alprazolam, Chlordiazepoxide, Clorazepate, Diazepam, Flurazepam, Lorazepam, Oxazepam, Temazepam, or Triazolam.

23. The method of claim 22, wherein the pharmaceutical agent is a psychotropic.

24. The method of claim 23, wherein the psychotropic is rimonabant, an inverse agonist of CB1 cannabinoid receptor.

25. The method of claim 1, wherein the pharmaceutical agent is an S1R ligand selected from high-affinity agonists: fluoxetine or fluvoxamine; intermediate-affinity agonists: escitalopram or citalopram; or low-affinity agonist: paroxetine; or antagonist: sertraline.

26. The method of claim 1, wherein the pharmaceutical agent is antidepressants with FIASMA activity.

27. The method of claim 1, wherein the pharmaceutical agent is functional inhibitor of acid sphingomyelinase (FIASMA) having FIASMA activity, defined as showing an in vitro functional inhibition effect on acid sphingomyelinase (ASM).

28. The method of claim 1, wherein the FIASMA is selected from one or more selected from the group consisting of: amitriptyline, citalopram, clomipramine, desipramine, doxepin, escitalopram, fluoxetine, fluvoxamine, imipramine, nortriptyline, paroxetine, sertraline, and venlafaxine.

29. The method of claim 1, wherein the pharmaceutical agent is selected from bupropion, desvenlafaxine, duloxetine, levomilnacipran, mirtazapine, nefazodone, trazodone, vilazodone, or vortioxetine.

30. The method of claim 1, wherein the pharmaceutical agent is selected from fluoxetine, paroxetine, escitalopram, venlafaxine, and mirtazapine.

31. The method of claim 1, wherein the subject:
has a positive test result for SARS-CoV-2 viral testing;
has, is suspected of having, or is at risk for contracting COVID-19 or developing a SARS-CoV-2 infection;
has early symptomatic COVID-19 disease;
is in an acute phase of COVID-19 illness;
has Post-Acute Sequelae of SARS-CoV-2 infection (PASC);
is exposed to COVID-19 or at risk of COVID-19 exposure;
is ambulatory having COVID-19;
has had COVID-19 symptoms for less than or equal to 7 days;
has had COVID-19 symptoms for less than or equal to 10 days;
has had COVID-19 symptoms for between 7 to 10 days; or
has had COVID-19 symptoms for greater than 5 days.

32. The method of claim 1, wherein the subject is at high risk for progressing to severe disease and/or hospitalization.

33. The method of claim 1, wherein the subject is high risk, wherein a high risk subject has one or more selected from: diabetes; systemic arterial hypertension requiring at least one oral medication for treatment; known cardiovascular disease (optionally, heart failure, congenital heart disease, valve disease, coronary artery disease, cardiomyopathies being treated, clinically manifested heart disease with clinical repercussion); symptomatic lung disease or treatment for such (optionally, emphysema, fibrosing diseases); symptomatic asthma requiring chronic use of agents to control symptoms; smoking; obesity, defined as body-mass index greater than 30 kg/m²; having had a transplant; stage IV chronic kidney disease or on dialysis; immunosuppression or use of corticosteroid therapy (optionally, equivalent to at least 10 mg of prednisone per day) or immunosuppressive therapy; history of cancer in the last 0.5 years or undergoing current cancer treatment or aged 50 years or older; or unvaccinated status.

34. The method of claim 1, wherein the subject has risk factors for disease progression selected from one or more of: age ≥50 years, diabetes, hypertension, obesity, smoking, conditions associated with immunosuppression, unvaccinated status, or comorbidities, optionally selected from cancer, cardiovascular, pulmonary, or kidney disorders.

35. The method of claim 1, wherein the subject is not being prescribed an SSRI or being administered an SSRI at the time of COVID-19 infection or symptom onset.

36. The method of claim 1, wherein the subject is administered the SSRI prophylactically.

37. The method of claim 1, wherein the subject has not been diagnosed with a mental illness, mood disorder, anxiety disorder (optionally, OCD, depression), other psychiatric disorders or prescribed medicine for a mental illness, mood disorder, anxiety disorder (optionally, OCD, depression), or other psychiatric disorders prior to being treated for COVID-19 or prevention of COVID-19.

38. The method of claim 1, wherein the subject has hypoxia or dyspnea.

39. The method of claim 1, wherein the subject has severe COVID-19 necessitating interventional care (optionally, dexamethasone, supplemental oxygen).

40. The method of claim 1, wherein the subject has fever, cough, shortness of breath, fatigue or weakness, chills, nausea, body aches, diarrhea, loss of appetite, difficulty with sense of smell, or difficulty with sense of taste.

41. The method of claim 1, wherein the subject has tinnitus or vaccine-associated tinnitus or post-vaccine long-COVID-like symptoms.

42. The method of claim 1, wherein the therapeutically effective amount of the pharmaceutical agent results in reducing or preventing:
long COVID or long COVID symptoms;
clinical deterioration;
intubation or death;
excessive immune response associated with a COVID-19 infection;
an inflammatory response in the subject;
short or long term complications of COVID-19;
severe lung damage;
damage from an inflammatory response; or
shortness of breath.

43. The method of claim 1, wherein the therapeutically effective amount of the pharmaceutical agent results in reducing the risk of:
developing severe long-term post-COVID symptoms;
developing COVID acute respiratory distress syndrome (ARDS);
developing post-acute sequelae of SARS-CoV-2 infection (PASC);
death;
declining or deteriorating health;
hospitalization;
progression to severe disease with hypoxia <92%;
being admitted in an emergency setting or retention for greater than 6 h;
developing severe disease or illness; or
developing respiratory deterioration.

44. The method of claim 1, wherein the therapeutically effective amount of the pharmaceutical agent results in reducing:
recovery time;
levels of cytokines in the subject;
levels of inflammatory molecules in the subject; or
severity of Post-Acute Sequelae of SARS-CoV-2 infection (PASC).

45. The method of claim 1, wherein administrating the pharmaceutical agent to a subject prior to SARS-CoV-2 infection results in:
reduced relative risk of mortality, wherein the pharmaceutical agent is an SSRI, optionally, fluoxetine or fluvoxamine; or
reduced risk of an emergency department (ED) visit or hospital admission in ambulatory patients infected with SARS-CoV-2 re-illness use of wherein the pharmaceutical agent is an antidepressant or an SSRI.

46. The method of claim 1, wherein administrating the pharmaceutical agent results in the subject having improved recovery between about three months after infection to about 1 year after infection compared to a subject not receiving the pharmaceutical agent.

47. The method of claim 1, wherein hospitalization is defined as either retention in a COVID-19 emergency setting or transfer to tertiary hospital from COVID-19 up to 28 days.

48. The method of claim 1, wherein clinical deterioration is defined as emergency department (ED) visitation or hospital admission.

49. The method of claim 1, wherein progression to severe COVID-19 is defined by hypoxia <93% with dyspnea or requiring hospitalization.

50. The method of claim 1, wherein the therapeutically effective amount is an amount effective to:
prevent or reduce the risk of clinical deterioration, defined as shortness of breath, hospitalization for shortness of breath or pneumonia, oxygen saturation less than 92% on room air, or need for supplemental oxygen to achieve oxygen saturation of 92% or greater.

51. The method of claim 1, wherein the therapeutically effective amount is an amount effective to reduce or prevent symptoms or negative outcomes associated with COVID-19 compared to a placebo.

52. The method of claim 1, wherein the subject is administered the pharmaceutical agent within the first 7 to 10 days of illness with COVID-19 or between day one and day 7 to day 10 of COVID-19 symptom onset.

53. The method of claim 1, wherein the subject is administered the pharmaceutical agent until COVID-19 symptoms resolve.

54. The method of claim 1, wherein the subject is administered the pharmaceutical agent comprising an SSRI prior to contracting SARS-CoV-2 at a dose of at least 20 mg fluoxetine-equivalents or greater than or equal to 40 mg fluoxetine-equivalents.

55. The method of claim 1, wherein the subject is administered the pharmaceutical agent or a duration of 10-15 days through at least 15 days of illness or symptoms.

56. The method of claim 1, wherein the subject is administered the pharmaceutical agent comprising fluvoxamine at
50 mg twice a day;
200 mg/day (100 mg twice daily);
300 mg/day (100 mg three times daily); or
a daily total of 300 mg.

57. The method of claim 1, wherein the subject is administered the pharmaceutical agent comprising fluvoxamine at
50 mg on day one, then 100 mg twice a day;
50 mg on day 1, then for 2 days at a dose of 100 mg twice daily, and for the duration of treatment, a dose of 100 mg 3 times daily;
100 mg three times daily;
50 mg to 100 mg two times daily;
100 mg twice daily;
a single dose of 150-200 mg daily; or
100 mg two times daily for 3 days, then 50 mg two times daily for 3 days.

58. The method of claim 54, wherein the pharmaceutical agent is administered for an amount of time between 10 and 15 days, for a duration of 15 days, or for the duration of illness or until symptom resolution, as tolerated; and if intolerance is a problem, the dose can be reduced to 50 mg twice daily.

59. The method of claim 1, wherein the pharmaceutical agent is administered to the subject in an amount of about 100 mg at a frequency of 3× per day.

60. The method of claim 1, wherein the pharmaceutical agent is administered to the subject for about 15 days or before or at about 14 days post symptom onset.

61. The method of claim 1, wherein the pharmaceutical agent comprises an antidepressant at a dose of:
at least 20 mg fluoxetine-equivalents,
greater than or equal to 20 mg fluoxetine-equivalents,
greater than or equal to 40 mg equivalent, or
between 20.0 mg and 44.4 mg fluoxetine-equivalents.

62. The method of claim 58, wherein the antidepressant is selected from the following and the fluoxetine-equivalent is calculated using a conversion factor associated with the antidepressant according to:

| | Conversion |
|---|---|
| Antidepressant | Factor |
| Fluoxetine | 1 |
| Fluvoxamine | 0.3 |
| Citalopram | 1.11 |
| Escitalopram | 2.22 |
| Paroxetine | 1.17 |
| Sertraline | 0.42 |
| Vilazodone | 1.5 |
| Vortioxetine | 3 |
| Amitriptyline | 0.33 |
| Clomipramine | 0.35 |
| Desipramine | 0.21 |
| Doxepin | 0.29 |
| Imipramine | 0.29 |
| Nortriptyline | 0.40 |
| Nefazodone | 0.08 |
| Trazodone | 0.10 |
| Desvenlafaxine | 0.40 |
| Duloxetine | 0.67 |
| Levomilnacipran | 0.33 |
| Venlafaxine | 0.28 |
| Bupropion | 0.11 |
| Mirtazapine | 0.79 |

63. The method of claim 1, wherein the pharmaceutical agent is fluvoxamine and is administered in tablet form in 25 mg, 50 mg, or 100 mg strengths.

64. The method of claim 1, wherein the pharmaceutical agent comprises Bupropion at any dose.

65. The method of claim 1, wherein the pharmaceutical agent is administered orally or intravenously.

66. The method of claim 1, further comprising administering a second pharmaceutical agent comprising a: S1R agonist (optionally, Chlorpromazine, Fluoxetine), S1R antagonist, antihistamine, bromhexine, serotonin antagonist (optionally, cyproheptadine, bromohexine), an antibiotic, an anti-inflammatory, a steroid, a glucocorticoid, a serotonin antagonist, a virus entry inhibitor, an anti-viral (optionally, remdesivir, Nirmatrelvir (Paxlovid), molnupiravir (Lagevrio), lopinavir, ritonavir, favipiravir), anti-SARS-CoV-2 mAb product (optionally, Bamlanivimab (LY-CoV555), etesevimab, casirivimab, imdevimab (REGEN-COV), sotrovimab, or combinations thereof), itraconazole, hydroxychloroquine, metformin, niclosamide, ivermectin, fluvoxamine, doxasozin, pegylated interferon lambda, anti-inflammatory drugs, such as colchicine, corticosteroids, or budesonide.
